SEQUENCE LISTING (246 sequences)

SEQ ID NO: 1 - DNA, 1962 bp, homo sapiens
  IDUA polynucleote sequence
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatga

SEQ ID NO: 2 - protein, 653 aa, homo sapiens
  IDUA polypeptide sequence
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 3 - DNA, 1962 bp, homo sapiens; identical to SEQ ID NO: 1
  IDS polynucleotide sequence

SEQ ID NO: 4 - protein, 653 aa, homo sapiens; identical to SEQ ID NO: 2
  IDS polypeptide sequence

SEQ ID NO: 5 - DNA, 1956 bp, homo sapiens
  ARSB polynucleotide sequence
atggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcctgtttacttga

SEQ ID NO: 6 - protein, 533 aa, homo sapiens
  ARSB polypeptide sequence
MGPRGAASLPRGPGPRRLLLPVVLPLLLLLLLAPPGSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 7 - DNA, 1956 bp, homo sapiens; identical to SEQ ID NO: 5
  GUSB polynucleotide sequence

SEQ ID NO: 8 - protein, 651 aa, homo sapiens
  GUSB polypeptide sequence
MARGSAVAWAALGPLLWGCALGLQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSLFT

SEQ ID NO: 9 - DNA, 2238 bp, homo sapiens
  ABCD1 polynucleotide sequence
atgccggtgctctccaggccccggccctggcgggggaacacgctgaagcgcacggccgtgctcctggccctcgcggcctatggagcccacaaagtctaccccttggtgcgccagtgcctggccccggccaggggtcttcaggcgcccgccggggagcccacgcaggaggcctccggggtcgcggcggccaaagctggcatgaaccgggtattcctgcagcggctcctgtggctcctgcggctgctgttcccccgggtcctgtgccgggagacggggctgctggccctgcactcggccgccttggtgagccgcaccttcctgtcggtgtatgtggcccgcctggacggaaggctggcccgctgcatcgtccgcaaggacccgcgggcttttggctggcagctgctgcagtggctcctcatcgccctccctgctaccttcgtcaacagtgccatccgttacctggagggccaactggccctgtcgttccgcagccgtctggtggcccacgcctaccgcctctacttctcccagcagacctactaccgggtcagcaacatggacgggcggcttcgcaaccctgaccagtctctgacggaggacgtggtggcctttgcggcctctgtggcccacctctactccaacctgaccaagccactcctggacgtggctgtgacttcctacaccctgcttcgggcggcccgctcccgtggagccggcacagcctggccctcggccatcgccggcctcgtggtgttcctcacggccaacgtgctgcgggccttctcgcccaagttcggggagctggtggcagaggaggcgcggcggaagggggagctgcgctacatgcactcgcgtgtggtggccaactcggaggagatcgccttctatgggggccatgaggtggagctggccctgctacagcgctcctaccaggacctggcctcgcagatcaacctcatccttctggaacgcctgtggtatgttatgctggagcagttcctcatgaagtatgtgtggagcgcctcgggcctgctcatggtggctgtccccatcatcactgccactggctactcagagtcagatgcagaggccgtgaagaaggcagccttggaaaagaaggaggaggagctggtgagcgagcgcacagaagccttcactattgcccgcaacctcctgacagcggctgcagatgccattgagcggatcatgtcgtcgtacaaggaggtgacggagctggctggctacacagcccgggtgcacgagatgttccaggtatttgaagatgttcagcgctgtcacttcaagaggcccagggagctagaggacgctcaggcggggtctgggaccataggccggtctggtgtccgtgtggagggccccctgaagatccgaggccaggtggtggatgtggaacaggggatcatctgcgagaacatccccatcgtcacgccctcaggagaggtggtggtggccagcctcaacatcagggtggaggaaggcatgcatctgctcatcacaggccccaatggctgcggcaagagctccctgttccggatcctgggtgggctctggcccacgtacggtggtgtgctctacaagcccccaccccagcgcatgttctacatcccgcagaggccctacatgtctgtgggctccctgcgtgaccaggtgatctacccggactcagtggaggacatgcaaaggaagggctactcggagcaggacctggaagccatcctggacgtcgtgcacctgcaccacatcctgcagcgggagggaggttgggaggctatgtgtgactggaaggacgtcctgtcgggtggcgagaagcagagaatcggcatggcccgcatgttctaccacaggcccaagtacgccctcctggatgaatgcaccagcgccgtgagcatcgacgtggaaggcaagatcttccaggcggccaaggacgcgggcattgccctgctctccatcacccaccggccctccctgtggaaataccacacacacttgctacagttcgatggggagggcggctggaagttcgagaagctggactcagctgcccgcctgagcctgacggaggagaagcagcggctggagcagcagctggcgggcattcccaagatgcagcggcgcctccaggagctctgccagatcctgggcgaggccgtggccccagcgcatgtgccggcacctagcccgcaaggccctggtggcctccagggtgcctccacctga

SEQ ID NO: 10 - protein, 745 aa, homo sapiens [sequence omitted]

SEQ ID NO: 11 - DNA, 2058 bp, homo sapiens
  GALC polynucleotide sequence
atggctgagtggctactctcggcttcctggcaacgccgagcgaaagctatgactgcggccgcgggttcggcgggccgcgccgcggtgcccttgctgctgtgtgcgctgctggcgcccggcggcgcgtacgtgctcgacgactccgacgggctgggccgggagttcgacggcatcggcgcggtcagcggcggcggggcaacctcccgacttctagtaaattacccagagccctatcgttctcagatattggattatctctttaagccgaattttggtgcctctttgcatattttaaaagtggaaataggtggtgatgggcagacaacagacggcactgagccctcccacatgcattatgcactagatgagaattatttccgaggatacgagtggtggttgatgaaagaagctaagaagaggaatcccaatattacactcattgggttgccatggtcattccctggatggctgggaaaaggtttcgactggccttatgtcaatcttcagctgactgcctattatgtcgtgacctggattgtgggcgccaagcgttaccatgatttggacattgattatattggaatttggaatgagaggtcatataatgccaattatattaagatattaagaaaaatgctgaattatcaaggtctccagcgagtgaaaatcatagcaagtgataatctctgggagtccatctctgcatccatgctccttgatgccgaactcttcaaggtggttgatgttataggggctcattatcctggaacccattcagcaaaagatgcaaagttgactgggaagaagctttggtcttctgaagactttagcactttaaatagtgacatgggtgcaggctgctggggtcgcattttaaatcagaattatatcaatggctatatgacttccacaatcgcatggaatttagtggctagttactatgaacagttgccttatgggagatgcgggttgatgacggcccaggagccatggagtgggcactacgtggtagaatctcctgtctgggtatcagctcataccactcagtttactcaacctggctggtattacctgaagacagttggccatttagagaaaggaggaagctacgtagctctgactgatggcttagggaacctcaccatcatcattgaaaccatgagtcataaacattctaagtgcatacggccatttcttccttatttcaatgtgtcacaacaatttgccacctttgttcttaagggatcttttagtgaaataccagagctacaggtatggtataccaaacttggaaaaacatccgaaagatttctttttaagcagctggattctctatggctccttgacagcgatggcagtttcacactgagcctgcatgaagatgagctgttcacactcaccactctcaccactggtcgcaaaggcagctacccgcttcctccaaaatcccagcccttcccaagtacctataaggatgatttcaatgttgattacccattttttagtgaagctccaaactttgctgatcaaactggtgtatttgaatattttacaaatattgaagaccctggcgagcatcacttcacgctacgccaagttctcaaccagagacccattacatgggctgccgatgcatccaacacaatcagtattataggagactacaactggaccaatctgactataaagtgtgatgtatacatagagacccctgacacaggaggtgtgttcattgcaggaagagtaaataaaggtggtattttgattagaagtgccagaggaattttcttctggatttttgcaaatggatcttacagggttacaggtgatttagctggatggattatatatgctttaggacgtgttgaagttacagcaaaaaaatggtatacactcacgttaactattaagggtcatttcacctctggcatgctgaatgacaagtctctgtggacagacatccctgtgaattttccaaagaatggctgggctgcaattggaactcactcctttgaatttgcacagtttgacaactttcttgtggaagccacacgctaa

SEQ ID NO: 12 - protein, 685 aa, homo sapiens
  GALC  polypeptide sequence
MAEWLLSASWQRRAKAMTAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFTSGMLNDKSLWTDIPVNFPKNGWAAIGTHSFEFAQFDNFLVEATR

SEQ ID NO: 13 - DNA, 1530 bp, homo sapiens
  ARSA polynucleotide sequence
atgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctga

SEQ ID NO: 14 - protein, 509 aa, homo sapiens
  ARSA  polypeptide sequence
MSMGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 15 - DNA, 1584 bp, homo sapiens
  PSAP polynucleotide sequence
atgtacgccctcttcctcctggccagcctcctgggcgcggctctagccggcccggtccttggactgaaagaatgcaccaggggctcggcagtgtggtgccagaatgtgaagacggcgtccgactgcggggcagtgaagcactgcctgcagaccgtttggaacaagccaacagtgaaatcccttccctgcgacatatgcaaagacgttgtcaccgcagctggtgatatgctgaaggacaatgccactgaggaggagatccttgtttacttggagaagacctgtgactggcttccgaaaccgaacatgtctgcttcatgcaaggagatagtggactcctacctccctgtcatcctggacatcattaaaggagaaatgagccgtcctggggaggtgtgctctgctctcaacctctgcgagtctctccagaagcacctagcagagctgaatcaccagaagcagctggagtccaataagatcccagagctggacatgactgaggtggtggcccccttcatggccaacatccctctcctcctctaccctcaggacggcccccgcagcaagccccagccaaaggataatggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaggatcagcaacccaaggagatctgtgcgctggttgggttctgtgatgaggtgaaagagatgcccatgcagactctggtccccgccaaagtggcctccaagaatgtcatccctgccctggaactggtggagcccattaagaagcacgaggtcccagcaaagtctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacgcggctgcctgcactgaccgttcacgtgactcagccaaaggacggtggcttctgcgaagtgtgcaagaagctggtgggttatttggatcgcaacctggagaaaaacagcaccaagcaggagatcctggctgctcttgagaaaggctgcagcttcctgccagacccttaccagaagcagtgtgatcagtttgtggcagagtacgagcccgtgctgatcgagatcctggtggaggtgatggatccttccttcgtgtgcttgaaaattggagcctgcccctcggcccataagcccttgttgggaactgagaagtgtatatggggcccaagctactggtgccagaacacagagacagcagcccagtgcaatgctgtcgagcattgcaaacgccatgtgtggaactag

SEQ ID NO: 16 - protein, 527 aa, homo sapiens
  PSAP polypeptide sequence
MYALFLLASLLGAALAGPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQDQQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 17 - DNA, 1611 bp, homo sapiens
  GBA polynucleotide sequence
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 18 - protein, 536 aa, homo sapiens
  GBA polypeptide sequence
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 19 - DNA, 1401 bp, homo sapiens
  FUCA1 polynucleotide sequence
atgcgggctccggggatgaggtcgcggccggcgggtcccgcgctgttgctgctgctgctcttcctcggagcggccgagtcggtgcgtcgggcccagcctccgcgccgctacaccccagactggccgagcctggattctcggccgctgccggcctggttcgacgaagccaagttcggggtgttcatccactggggcgtgttctcggtgcccgcctggggcagcgagtggttctggtggcactggcagggcgaggggcggccgcagtaccagcgcttcatgcgcgacaactacccgcccggcttcagctacgccgacttcggaccgcagttcactgcgcgcttcttccacccggaggagtgggccgacctcttccaggccgcgggcgccaagtatgtagttttgacgacaaagcatcacgaaggcttcacaaactggccgagtcctgtgtcttggaactggaactccaaagacgtggggcctcatcgggatttggttggtgaattgggaacagctctccggaagaggaacatccgctatggactataccactcactcttagagtggttccatccactctatctacttgataagaaaaatggcttcaaaacacagcattttgtcagtgcaaaaacaatgccagagctgtacgaccttgttaacagctataaacctgatctgatctggtctgatggggagtgggaatgtcctgatacttactggaactccacaaattttctttcatggctctacaatgacagccctgtcaaggatgaggtggtagtaaatgaccgatggggtcagaactgttcctgtcaccatggaggatactataactgtgaagataaattcaagccacagagcttgccagatcacaagtgggagatgtgcaccagcattgacaagttttcctggggctatcgtcgtgacatggcattgtctgatgttacagaagaatctgaaatcatttcggaactggttcagacagtaagtttgggaggcaactatcttctgaacattggaccaactaaagatggactgattgttcccatcttccaagaaaggcttcttgctgttgggaaatggctgagcatcaatggggaggctatctatgcctccaaaccatggcgggtgcaatgggaaaagaacacaacatctgtatggtatacctcaaagggatcggctgtttatgccatttttctgcactggccagaaaatggagtcttaaaccttgaatcccccataactacctcaactacaaagataacaatgctgggaattcaaggagatctgaagtggtccacagatccagataaaggtctcttcatctctctaccccagttgccaccctctgctgtccccgcagagtttgcttggactataaagctgacaggagtgaagtaa

SEQ ID NO: 20 - protein, 466 aa, homo sapiens
  FUCA1 polypeptide sequence
MRAPGMRSRPAGPALLLLLLFLGAAESVRRAQPPRRYTPDWPSLDSRPLPAWFDEAKFGVFIHWGVFSVPAWGSEWFWWHWQGEGRPQYQRFMRDNYPPGFSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQHFVSAKTMPELYDLVNSYKPDLIWSDGEWECPDTYWNSTNFLSWLYNDSPVKDEVVVNDRWGQNCSCHHGGYYNCEDKFKPQSLPDHKWEMCTSIDKFSWGYRRDMALSDVTEESEIISELVQTVSLGGNYLLNIGPTKDGLIVPIFQERLLAVGKWLSINGEAIYASKPWRVQWEKNTTSVWYTSKGSAVYAIFLHWPENGVLNLESPITTSTTKITMLGIQGDLKWSTDPDKGLFISLPQLPPSAVPAEFAWTIKLTGVK

SEQ ID NO: 21 - DNA, 3036 bp, homo sapiens
  MAN2B1 polynucleotide sequence
atgggcgcctacgcgcgggcttcgggggtctgcgctcgcggctgcctggactcagcaggcccctggaccatgtcccgcgccctgcggccaccgctcccgcctctctgctttttccttttgttgctggcggctgccggtgctcgggccgggggatacgagacatgccccacagtgcagccgaacatgctgaacgtgcacctgctgcctcacacacatgatgacgtgggctggctcaaaaccgtggaccagtacttttatggaatcaagaatgacatccagcacgccggtgtgcagtacatcctggactcggtcatctctgccttgctggcagatcccacccgtcgcttcatttacgtggagattgccttcttctcccgttggtggcaccagcagacaaatgccacacaggaagtcgtgcgagaccttgtgcgccaggggcgcctggagttcgccaatggtggctgggtgatgaacgatgaggcagccacccactacggtgccatcgtggaccagatgacacttgggctgcgctttctggaggacacatttggcaatgatgggcgaccccgtgtggcctggcacattgaccccttcggccactctcgggagcaggcctcgctgtttgcgcagatgggcttcgacggcttcttctttgggcgccttgattatcaagataagtgggtacggatgcagaagctggagatggagcaggtgtggcgggccagcaccagcctgaagcccccgaccgcggacctcttcactggtgtgcttcccaatggttacaacccgccaaggaatctgtgctgggatgtgctgtgtgtcgatcagccgctggtggaggaccctcgcagccccgagtacaacgccaaggagctggtcgattacttcctaaatgtggccactgcccagggccggtattaccgcaccaaccacactgtgatgaccatgggctcggacttccaatatgagaatgccaacatgtggttcaagaaccttgacaagctcatccggctggtaaatgcgcagcaggcaaaaggaagcagtgtccatgttctctactccacccccgcttgttacctctgggagctgaacaaggccaacctcacctggtcagtgaaacatgacgacttcttcccttacgcggatggcccccaccagttctggaccggttacttttccagtcggccggccctcaaacgctacgagcgcctcagctacaacttcctgcaggtgtgcaaccagctggaggcgctggtgggcctggcggccaacgtgggaccctatggctccggagacagtgcacccctcaatgaggcgatggctgtgctccagcatcacgacgccgtcagcggcacctcccgccagcacgtggccaacgactacgcgcgccagcttgcggcaggctgggggccttgcgaggttcttctgagcaacgcgctggcgcggctcagaggcttcaaagatcacttcaccttttgccaacagctaaacatcagcatctgcccgctcagccagacggcggcgcgcttccaggtcatcgtttataatcccctggggcggaaggtgaattggatggtacggctgccggtcagcgaaggcgttttcgttgtgaaggaccccaatggcaggacagtgcccagcgatgtggtaatatttcccagctcagacagccaggcgcaccctccggagctgctgttctcagcctcactgcccgccctgggcttcagcacctattcagtagcccaggtgcctcgctggaagccccaggcccgcgcaccacagcccatccccagaagatcctggtcccctgctttaaccatcgaaaatgagcacatccgggcaacgtttgatcctgacacagggctgttgatggagattatgaacatgaatcagcaactcctgctgcctgttcgccagaccttcttctggtacaacgccagtataggtgacaacgaaagtgaccaggcctcaggtgcctacatcttcagacccaaccaacagaaaccgctgcctgtgagccgctgggctcagatccacctggtgaagacacccttggtgcaggaggtgcaccagaacttctcagcttggtgttcccaggtggttcgcctgtacccaggacagcggcacctggagctagagtggtcggtggggccgatacctgtgggcgacacctgggggaaggaggtcatcagccgttttgacacaccgctggagacaaagggacgcttctacacagacagcaatggccgggagatcctggagaggaggcgggattatcgacccacctggaaactgaaccagacggagcccgtggcaggaaactactatccagtcaacacccggatttacatcacggatggaaacatgcagctgactgtgctgactgaccgctcccaggggggcagcagcctgagagatggctcgctggagctcatggtgcaccgaaggctgctgaaggacgatggacgcggagtatcggagccactaatggagaacgggtcgggggcgtgggtgcgagggcgccacctggtgctgctggacacagcccaggctgcagccgccggacaccggctcctggcggagcaggaggtcctggcccctcaggtggtgctggccccgggtggcggcgccgcctacaatctcggggctcctccgcgcacgcagttctcagggctgcgcagggacctgccgccctcggtgcacctgctcacgctggccagctggggccccgaaatggtgctgctgcgcttggagcaccagtttgccgtaggagaggattccggacgtaacctgagcgcccccgttaccttgaacttgagggacctgttctccaccttcaccatcacccgcctgcaggagaccacgctggtggccaaccagctccgcgaggcagcctccaggctcaagtggacaacaaacacaggccccacaccccaccaaactccgtaccagctggacccggccaacatcacgctggaacccatggaaatccgcactttcctggcctcagttcaatggaaggaggtggatggttag

SEQ ID NO: 22 - protein, 1011 aa, homo sapiens
  MAN2B1 polypeptide sequence
MGAYARASGVCARGCLDSAGPWTMSRALRPPLPPLCFFLLLLAAAGARAGGYETCPTVQPNMLNVHLLPHTHDDVGWLKTVDQYFYGIKNDIQHAGVQYILDSVISALLADPTRRFIYVEIAFFSRWWHQQTNATQEVVRDLVRQGRLEFANGGWVMNDEAATHYGAIVDQMTLGLRFLEDTFGNDGRPRVAWHIDPFGHSREQASLFAQMGFDGFFFGRLDYQDKWVRMQKLEMEQVWRASTSLKPPTADLFTGVLPNGYNPPRNLCWDVLCVDQPLVEDPRSPEYNAKELVDYFLNVATAQGRYYRTNHTVMTMGSDFQYENANMWFKNLDKLIRLVNAQQAKGSSVHVLYSTPACYLWELNKANLTWSVKHDDFFPYADGPHQFWTGYFSSRPALKRYERLSYNFLQVCNQLEALVGLAANVGPYGSGDSAPLNEAMAVLQHHDAVSGTSRQHVANDYARQLAAGWGPCEVLLSNALARLRGFKDHFTFCQQLNISICPLSQTAARFQVIVYNPLGRKVNWMVRLPVSEGVFVVKDPNGRTVPSDVVIFPSSDSQAHPPELLFSASLPALGFSTYSVAQVPRWKPQARAPQPIPRRSWSPALTIENEHIRATFDPDTGLLMEIMNMNQQLLLPVRQTFFWYNASIGDNESDQASGAYIFRPNQQKPLPVSRWAQIHLVKTPLVQEVHQNFSAWCSQVVRLYPGQRHLELEWSVGPIPVGDTWGKEVISRFDTPLETKGRFYTDSNGREILERRRDYRPTWKLNQTEPVAGNYYPVNTRIYITDGNMQLTVLTDRSQGGSSLRDGSLELMVHRRLLKDDGRGVSEPLMENGSGAWVRGRHLVLLDTAQAAAAGHRLLAEQEVLAPQVVLAPGGGAAYNLGAPPRTQFSGLRRDLPPSVHLLTLASWGPEMVLLRLEHQFAVGEDSGRNLSAPVTLNLRDLFSTFTITRLQETTLVANQLREAASRLKWTTNTGPTPHQTPYQLDPANITLEPMEIRTFLASVQWKEVDG

SEQ ID NO: 23 - DNA, 1041 bp, homo sapiens
  AGA polynucleotide sequence
atggcgcggaagtcgaacttgcctgtgcttctcgtgccgtttctgctctgccaggccctagtgcgctgctccagccctctgcccctggtcgtcaacacttggccctttaagaatgcaaccgaagcagcgtggagggcattagcatctggaggctctgccctggatgcagtggagagcggctgtgccatgtgtgagagagagcagtgtgacggctctgtaggctttggaggaagtcctgatgaacttggagaaaccacactagatgccatgatcatggatggcactactatggatgtaggagcagtaggagatctcagacgaattaaaaatgctattggtgtggcacggaaagtactggaacatacaacacacacacttttagtaggagagtcagccaccacatttgctcaaagtatggggtttatcaatgaagacttatctaccactgcttctcaagctcttcattcagattggcttgctcggaattgccagccaaattattggaggaatgttataccagatccctcaaaatactgcggaccctacaaaccacctggtatcttaaagcaggatattcctatccataaagaaacagaagatgatcgtggtcatgacactattggcatggttgtaatccataagacaggacatattgctgctggtacatctacaaatggtataaaattcaaaatacatggccgtgtaggagactcaccaatacctggagctggagcctatgctgacgatactgcaggggcagccgcagccactgggaatggtgatatattgatgcgcttcctgccaagctaccaagctgtagaatacatgagaagaggagaagatccaaccatagcttgccaaaaagtgatttcaagaatccagaagcattttccagaattctttggggctgttatatgtgccaatgtgactggaagttacggtgctgcttgcaataaactttcaacatttactcagtttagtttcatggtttataattccgaaaaaaatcagccaactgaggaaaaagtggactgcatctaa

SEQ ID NO: 24 - protein, 346 aa, homo sapiens
  AGA polypeptide sequence
MARKSNLPVLLVPFLLCQALVRCSSPLPLVVNTWPFKNATEAAWRALASGGSALDAVESGCAMCEREQCDGSVGFGGSPDELGETTLDAMIMDGTTMDVGAVGDLRRIKNAIGVARKVLEHTTHTLLVGESATTFAQSMGFINEDLSTTASQALHSDWLARNCQPNYWRNVIPDPSKYCGPYKPPGILKQDIPIHKETEDDRGHDTIGMVVIHKTGHIAAGTSTNGIKFKIHGRVGDSPIPGAGAYADDTAGAAAATGNGDILMRFLPSYQAVEYMRRGEDPTIACQKVISRIQKHFPEFFGAVICANVTGSYGAACNKLSTFTQFSFMVYNSEKNQPTEEKVDCI

SEQ ID NO: 25 - DNA, 1236 bp, homo sapiens
  ASAH1 polynucleotide sequence
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 26 - protein, 411 aa, homo sapiens
  ASAH1 polypeptide sequence
MNCCIGLGEKARGSHRASYPSLSALFTEASILGFGSFAVKAQWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 27 - DNA, 1590 bp, homo sapiens
  HEXA polynucleotide sequence
atgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacatagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgaattgctgaggcgaggtgtccaggcccaacccctcaatgtaggcttctgtgagcaggagtttgaacagacctga

SEQ ID NO: 28 - protein, 529 aa, homo sapiens
  HEXA polypeptide sequence
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 29 - DNA, 2859 bp, homo sapiens
  GAA polynucleotide sequence
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 30 - protein, 952 aa, homo sapiens
  GAA polypeptide sequence
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 31 - DNA, 1896 bp, homo sapiens
  SMPD1 polynucleotide sequence
atgccccgctacggagcgtcactccgccagagctgccccaggtccggccgggagcagggacaagacgggaccgccggagcccccggactcctttggatgggcctggtgctggcgctggcgctggcgctggcgctggcgctggctctgtctgactctcgggttctctgggctccggcagaggctcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctag

SEQ ID NO: 32 - protein, 631 aa, homo sapiens
  SMPD1 polypeptide sequence
MPRYGASLRQSCPRSGREQGQDGTAGAPGLLWMGLVLALALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 33 - DNA, 1200 bp, homo sapiens
  LIPA polynucleotide sequence
atgaaaatgcggttcttggggttggtggtctgtttggttctctggaccctgcattctgaggggtctggagggaaactgacagctgtggatcctgaaacaaacatgaatgtgagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataaaactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaattaggacgattaccagatcatctcattaaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtatatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtga

SEQ ID NO: 34 - protein, 399 aa, homo sapiens
  LIPA polypeptide sequence
MKMRFLGLVVCLVLWTLHSEGSGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 35 - DNA, 3093 bp, homo sapiens
  CDKL5 polynucleotide sequence
atgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccagacggtggatgtgatggcagaagacagagacaccattctggaccccaagatagacgcttcatgttaaggacgacagaacaacaaggagaatacttctgctgtggtgacccaaagaagcctcacactccgtgcgtcccaaaccgagcccttcatcgtccaatctccagtcctgctccctatccagtactccaggtccgaggcacttccatgtgcccgacactccaggtccgaggcactgatgctttcagctgcccaacccagcaatccgggttctctttcttcgtgagacacgttatgagggaagccctgattcacagggcccaggtaaaccaagctgcgctcctgacataccatgagaatgcggcactgacgggcaagtga

SEQ ID NO: 36 - protein, 1030 aa, homo sapiens
  CDKL5 polypeptide sequence
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPDGGCDGRRQRHHSGPQDRRFMLRTTEQQGEYFCCGDPKKPHTPCVPNRALHRPISSPAPYPVLQVRGTSMCPTLQVRGTDAFSCPTQQSGFSFFVRHVMREALIHRAQVNQAALLTYHENAALTGK

SEQ ID NO: 37 - protein, 18 aa, homo sapiens
  Albumin peptide
MKWVTFISLLFLFSSAYS

SEQ ID NO: 38 - protein, 18 aa, homo sapiens
  chymotrypsinogen peptide
MAFLWLLSCWALLGTTFG

SEQ ID NO: 39 - protein, 14 aa, homo sapiens
  interleukin-2 peptide
MQLLSCIALILALV

SEQ ID NO: 40 - protein, 15 aa, homo sapiens
  trypsinogen-2 peptide
MNLLLILTFVAAAVA

SEQ ID NO: 41 - protein, 17 aa, homo sapiens
  BM40 peptide
MRAWIFFLLCLAGRALA

SEQ ID NO: 42 - protein, 21 aa, artificial
  Secrecon
MWWRLWWLLLLLLLLWPMVWA

SEQ ID NO: 43 - protein, 20 aa, artificial
  Mouse IgKVIII
METDTLLLWVLLLWVPGSTG

SEQ ID NO: 44 - protein, 22 aa, artificial
  Human IgKVIII
MDMRVPAQLLGLLLLWLRGARC

SEQ ID NO: 45 - protein, 16 aa, artificial
  CD33
MPLLLLLPLLWAGALA

SEQ ID NO: 46 - protein, 23 aa, artificial
  tPA
MDAMKRGLCCVLLLCGAVFVSPS

SEQ ID NO: 47 - protein, 16 aa, artificial
  Consensus
MLLLLLLLLLLALALA

SEQ ID NO: 48 - protein, 17 aa, artificial
  Native
MLLLLLLLGLRLQLSLG

SEQ ID NO: 49 - protein, 16 aa, artificial
  Penetratin
RQIKIWFQNRRMKWKK

SEQ ID NO: 50 - protein, 11 aa, artificial
  TAT
YGRKKRRQRRR

SEQ ID NO: 51 - protein, 18 aa, artificial
  SynB1
RGGRLSYSRRRFSTSTGR

SEQ ID NO: 52 - protein, 10 aa, artificial
  SynB3
RRLSYSRRRF

SEQ ID NO: 53 - protein, 12 aa, artificial
  PTD-4
PIRRRKKLRRLK

SEQ ID NO: 54 - protein, 12 aa, artificial
  PTD-5
RRQRRTSKLMKR

SEQ ID NO: 55 - protein, 15 aa, artificial
  FHV Coat
RRRRNRTRRNRRRVR

SEQ ID NO: 56 - protein, 19 aa, artificial
  BMV Gag
KMTRAQRRAAARRNRWTAR

SEQ ID NO: 57 - protein, 13 aa, artificial
  HTLV-II Rex
TRRQRTRRARRNR

SEQ ID NO: 58 - protein, 13 aa, artificial
  D-Tat
GRKKRRQRRRPPQ

SEQ ID NO: 59 - protein, 13 aa, artificial
  R9-Tat
GRRRRRRRRRPPQ

SEQ ID NO: 60 - protein, 27 aa, artificial
  Transportan
GWTLNSAGYLLGKINLKALAALAKKIL

SEQ ID NO: 61 - protein, 17 aa, artificial
  MAP
KLALKLALKLALALKLA

SEQ ID NO: 62 - protein, 27 aa, artificial
  SBP
MGLGLHLLVLAAALQGAWSQPKKKRKV

SEQ ID NO: 63 - protein, 27 aa, artificial
  FBP
GALFLGWLGAAGSTMGAWSQPKKKRKV

SEQ ID NO: 64 - protein, 27 aa, artificial
  MPG ac
GALFLGFLGAAGSTMGAWSQPKKKRKV

SEQ ID NO: 65 - protein, 27 aa, artificial
  MPG(&#916;NLS)
GALFLGFLGAAGSTMGAWSQPKSKRKV

SEQ ID NO: 66 - protein, 21 aa, artificial
  Pep-1
KETWWETWWTEWSQPKKKRKV

SEQ ID NO: 67 - protein, 21 aa, artificial
  Pep-2
KETWFETWFTEWSQPKKKRKV

SEQ ID NO: 68 - protein, 18 aa, artificial
  ApoE p1
LRKLRKRLLLRKLRKRLL

SEQ ID NO: 69 - protein, 30 aa, artificial
  ApoE p2
LRKLRKRLLRDADDLLRKLRKRLLRDADDL

SEQ ID NO: 70 - protein, 17 aa, artificial
  ApoE p3
LRVRLASHLRKLRKRLL

SEQ ID NO: 71 - protein, 20 aa, artificial
  ApoE p4
TEELRVRLASHLRKLRKRLL

SEQ ID NO: 72 - protein, 34 aa, artificial
  ApoE p5
LRVRLASHLRKLRKRLLLRVRLASHLRKLRKRLL

SEQ ID NO: 73 - protein, 40 aa, artificial
  ApoE p6
TEELRVRLASHLRKLRKRLLTEELRVRLASHLRKLRKRLL

SEQ ID NO: 74 - protein, 10 aa, artificial
  Myc Peptide
EQKLISEEDL

SEQ ID NO: 75 - protein, 39 aa, artificial
  ApoB Peptide
SSVIDALQYKLEGTTRLTRKRGLKLATALSLSNKFVEGS

SEQ ID NO: 76 - DNA, 18242 bp, homo sapiens
  CX3CR1_loci
ggggcgggccgtgcttaccaggccgtggacttaaaccaggatgagagaacccctggaggcgtttaagttggcagacttggatttcaggaagagctctctggcttctgggtggagaatggccagtggggtaagtggtgagaggaaagacagagaacggagaaggttagatgggcttgggaaattatccaggccctggatggaggtagagatgtgtgctcatgaacacggaggggattactgatgtggggtggatgagactgtcgtcaagagtgtgggacaggaagagagggagagtcttggccagatccaagaaaggagccctcagaagaggaggggagtcagaggcaaggaaggggctgaggcagccagcccagctgagtggaccccaggagaggtatcaaggggtggtgtggggtggggaggggccagtgtcagaaagtggatggggagcggcctgactctgcttttgtcctgtggccttctggccaaaggcagggaaaggtggccaaacactgagaccaagaacaaagaaagaaaactgctggtggacttcttccaccatgagcaggccaccaagcccgcagcactgcactgcagcccccagctctgtcctggggttgggggaggtgaggaggggcaaggtggggagcacacagagcacccgctgtcctcggaacaccacagcgactagaggtaagggagcaccggatgtggctgggatgtgggcagcaaggggccagaggggccttgaaggggtcacagaccatttaatgaaggtgtattgaaggccaccatgggccaggccctagttagggatggatcagaattatatagcatatgccaggggtcaggcaggtaatgaagtgatcggaaggtgatgaggcagtggcagttgagattcacgttgcagtcgccccaagctggccaggccagggagcagaagcatggctggatgccggagcccaccaggctccccactgcagggcaagagtggcagggggagagactgtgaaaggagcataggccaggtcctgggtgaaagctgtgtcctcagccttgactgatgggtatagggagccactaaatgccttggggcagagaggtgaggaaaaaaatatttaccgagcatctacaaggtgcaaggtactcactagatgccttcagtaccaaagcttctcaaacttagtatgcatatcactcttctaagaatttcattaaaatgcagattctaattcagcagatatagggcagggcttgaggtgctgtctttaataagctcccagtgcctgggactgcactttgaggagaagagctgtgtgtgccccagtgtggtccagtgagtactctgggctccctctcgtgggcagggaagctgagggccccatgagctctcccagcttcctgaaggctccccattaatgagagctgactgtgctgtgctttgctgactgcagggcctgctccctgccccccacctccaggttggggtaagtggcacctctctccctccagctccgcagtcttccctgaggtttagatcttccaggtttataaagtcaggccctcctgttggcagctggcctccaccctggagtatctgagcttgcctgtggcagcatctaaagatagtctcccttacaggaaacaagatactattggctaactctgcaaataaaatgctcttagagggaaggaaagggaaatactcgtctctggtaaagtctgagcaggacagggtggctgactggcagatccagaggttcccttggcagtccacgccaggtaggtgcacaggactagttgggtacctgtgggtggggtggagcagtggacagctaataggttaataatgcctgtttgcttacgtgcagacaatggaaaccattttcctggggatgttgtagcctaaatatgtccaaggggatggaagagtgggaggcaaggggtgatcagatcatttataatacactcaacctggtggaatagtattagaagcattagtaattacattttagagacatggagaaaagctcatgattttaaactaactgaaaaaagcatgaaaaattgcatctggatgctgttgaggaattattgctaattttttgagatgagaaattgtattaaagtctttttcaaaaaagagtccttaacttttagagatgcacacaagtgtttatgggtgaaattaaataattcagtagagacattaagtgggaaatagaggaaatattgaccatgggttgctaatagttgaagccaggtgttgggtataaggaggttctcgctgcttttatttgaaaagttgctttttatttgaaaatttaataataaagagtttttaaatttgtatctgtattttaatataaatataaatgcacttaatataaatataaaatatgtataacatttagatagagaaaagctaaaagattactgtggttgattctaggaaactgcattgcagatagtttcatgggtttttttttcctttttcttccactttttgtaccatctatgggtttttgtttgttttttgttttttgggttttctttcttgtttgtgttttgttttttgagacggagttttgctcttgttgcctaagctggagtgcaatggcacagtctcggctcactgcaacctctgcctcctgggttcaagtgattctcctgcctcagcctcccaagtagctgggattataggcatgtaccaccgcccggctaattttgtatttttagtagaggcggggtttctccattaataaattcctggcacaaatttagtgttcaattttgatatatgttgttataaccattgtgaggatactcaggctcaggtttgtgtgggtggaaaacatggtcttcagaaagaaattatgagtgcaagacaggaggaaatccatcagaggccccagctgaggactgaccacggcttgttatttctcttgccttgcctctggcaatcacagcctcacagagcctgcaatccttgctttgtgagtttatagctcagtccagagaatggctaagaaagtttaggattctttcaacacccactccacaaaaaaaaaaaaaaaaaagaaaagaaaaaaaaattaatttttgaaatacttgaggtagaaaacttgaggcagaaaaaaattgagccaaaaaaaaaggaaaattgaaccacgtgaaagcaggcaagaaagcttgcattgctcagggcatcccaggcccagagggcgctttggagggagctgggtttcctgagaggaggcagggtgggtgacggacctgtgctggagagccttgaggaccactgtgggttgggaatgggggcagtggattggggttcaaaacccctgggaatgagaaatgggctcaggaaggctagggtggattctttcatcttcctctttgcttggctttattttcacaaaggaaggcagggcaggaaatagtctcagcccaacttcagtgtggttcttcttagtgctcaggcttacctggcacttgccacacctctgggatgggagcacctactatccatcagccacgtgccagtctccacaaagtctgctcctgaaccctgctcctcagctggccccacttcacagatggggacataggcagcttggctttggaatgaaggaatgaagtcaggaatgaagtcctggctctgcacttggtgactgtgcactgggcttgctaagtctgtttcctgcttttaaaatggagattgtccatcagcctttgaagccatgtaatgggtatgtgtcaactttctgcaaggattaaaggcatggtataggaagtcccaaacacactgcctgacccatctttgatgctcaagaaacgatatatgttgttgtcatgaggaaactgagcctcagaaagtttggatacctgaaaaacactgactactattgaatgaggttgtgaagaatccagagctgtaggggcaggaaagcaaagaacgtattagagctgacccagtcaggacgatcgtctatccccttcctcaccccaccccatcccaggaggaagcctgcccggccctaggcagctatggcacagtggcaatgtcaggtatggttctccctagccagagaccctagcctcaaaaaacctccttcttgggatccaggcatccaactgctcctccccagccccagcctctgacccagtatcctgagtccagagacgtttggaaccagcacctgtaatggaggagctgaacaaggaggggaacttctgctgctccacagcaggtcacggtcataggagggagtggaaccagaatggcagaatccagatcttggctgcctttcccaaggacttgttctgattcctagcagcacagcccaggcattccgagaagttgggctctctggcatcactcactctgcccagaagagccaggggaaagttggggcttctagctgaaccttgatcccacctgccctcttgaggggctcagaatctgctggctgcttcacaggtgggattctcacggcacgctggccacagctgatgcttcgaccccctcatcttgtttggccaaagtgcagctttttagcttgtgagtaaggaagaaaagctgtatcatatgtctttaaacatcttcctagaccacctttgttttccccttaaagtgtgcttaaggagaaaatggaaagtctcttttcagtgtgttgtattttgtttattgcaaaatacaacacactgaaagaacaatgtctgggttagcaaagtattagatttaatttcccacattactaccatccaggctgagaaagagaaccccagaagcctctctcatgtcccttcctgaccacagcctcttcctctcccacaaagaaccacaagtctgacttctatggtgatcacttccttgcttttccctatagtttgactgaatctaaatatcatccctaaacaatatagcttagctttgcctatttttgatcttcatatttggattcatactttttggtttggctcaagactaaacttttaaaatgcatccacgttgtaggagatgtacatgcacaaaaattttcacggcaacattgtccaaactagcaacaaactgaaagccacctatgtgtgcattagcagtagaaatggcaataaatcatggactagtcacacagtggaatcctacgcagcaaggagaattagcagtctacagccaaaccaacagcatgggtgagatgcttccagaaatactgagatgaatttagagataaaggcacaattggtccatatctacagggcacttaatccagtgcatctccactagacaaaatttgtatttccatgagcaagagtcatttgcactgctatctgctataacctacggtgctgtaaaattagctcaggcaataaaagggggaggtagccccaaagagtatgccagaaaggactcccagtaatcttcagtgtgtgtctatttcaaagtctttcatcatttttccttgtagttacttagtgtggaatttcagacccctctctatgtccctcttctcccttttcagccttggctttctctgcatccttccccgaagccctgcactcagcctaaactgactttcctgaacactctgggagcatgtgggctcctcccaactccaccatcttcactgccttctgttcctatttccctctgcctggccttggccccaggaaaccttcctatgctcagaccctctgtgcctttgtcttcaaagcccacccactattcactgccacctccatcttggtcaacctggaagactttccccatccttaaaacatcagctcaaacggcagatttttttttttttttttgtgatatcctcttgatcaccctcccccagtttttaaggagacagatatagaggttcaatccttgggcttcccatgacttttcttatattctgttactgagcaataataaacacttctaggaactgtatcttaaacacttttgcctctacagaatctagcacagtgcctagtattggtgaaatataattaacaaagtcttctttcaacacagagattctctccacaaaaggagtagagaaagaacagttttattatggaataagcagtaaaccaaaatatgcagagcattataggccatctactaagaggttgcaagaacagaaagaaatctcccccttttgtatagccaagtagatacaacctgttacatacatgttatcaaggcaaacaataactgttcctcaagtaagaggtcttgccagcaccgtttgccgtacatggttcactctaaatttacctggtaattagggtaaccacttgtgttagctaactggctctacccagaggaaaatcaaatttatctttaagacaaggggtaattttgcagcactgagcaaggctcttcagttaggctcataccttcccacagaaactaagagatagaagcactatctcccttaggtttgtttacatttcaaagagatgacgcccaggtccttgggaaagactagcttagctcataaagctgacaaaaagcctatctagtttcaaaaggatttacacatgttcaaagagaggagaaagtatgtaaaagttttctaggtgggcacggtggctcacacctgtattcccagcactttgagaggctgaggcgggtggatcacctgaagtcaggaatttgagatcagcctggccaacatggtgaaaccttgtctctactaaaaatacaaaaattagctgggcatggtggtgggcgcctgtaatcccagctacttgggaggctgaggcaggagaatcacttgaacccaggaggtggaggtggcagtgagccaagatggcaccattgcactccagcctgggcgatgagagtgaaactccatgtcaaaaaaaaaaaaagttttctaaagtaaatgctctgagaaaaaaacagaaggtgaggagatctctgtccttatttttaacaagaataattaaatgtttttatttttaatttctacttagactagaatatggtacagatgctcaaaaatattgacttaattaataattaaacaattaacaagtctatctctttcctaattattaaaaaaattatgaagtatttcccttcaaaatttcacagtttactttgacataaataaataaataccataaagtattttgaagaggaaagggaagggtggggcttcaaacacaaaataatggcaggttttgtaaaggggcaattcactaaacccaataggattctcctgttgctattttttttttttttttttttttggtggtggttggagggaagggttgggtgtgcatttggcctgtgtgaaaaactggtgggtagacaatgccatgctgttctagatgggctcattgctataggaatagatagtcactgaatttacttgcataggggatggggtataaaatgtttccttatagctccttaacatggaattcagcacccctctccccatcagccttagctctctctgaatcccaagggatatgtgttataaaaatagctgccatcataaatgagaaaaccatcaggacctggtaaaacttggcttcccaaacagtcaggggtctgggcatggcagccgactgagaacctttctatctagtacaaggtaaacaagatttgccaagacttgatcaactgatccagctccctatctccaaacagaacctcatctgaatgctcacaggctgtgccccagaagtgtagaagtgtcatgttccctgggcaggctgctgggcagcctctctgttgacaatagaccacacttttgctgacctaggactcatgttgctctttaagactgcttccttggccaggcatgatggctcacacctgtaatcctggcactttgagaggcccaggcaggaggatctcttgaggccagatgttcaagaccagcctggtcaacatagtaagaccccatatctaccaaaaatagctgggcatggtggtgcacacctatactcccagctacttaggagactgaggtgggaggattgctacatcccaggagttcaaggctgtagtgagctatgatcatgccactgcactccagcctgggcaacagagcgagatcctgtctcaaacaaacagtttccttctgtttgattcttgctgaaaaattgagcatgccagagctagcaaggctcttagaggtggtctggtccaatgctttaatttcatacatcaagaaactgaagcagagcagagtgacctgcccagggtctctcagccattcatgctcagaaatgtatgggctcctgtgaaacatgtggctcttaaaagcactatcatatatttgaaggcagaaaataggctaaaccttcagccttcagacttttcctctccagagaaaatgaccccagtttcctcactatggttgctgggagctagattcctggggatctggcagtgtggaccacctagtggtggctagaggagcaaataatatcccgcattccattttccactcaccaatccctgaggggcagcctgctgggttatgagcccacagggggagaaccccaacgaattcagagatgcatcatggaccagtttctcttaaggggcctgggtctactattttcagttctacttcgagagaagtggcctgcaatatcctgcagatttcccctccagggagaaaagcattgtgcggtgcaaggagcacaggctttgtggaaagaggcatctggggttgagatcctggctcttttgcttccttgaacaagttaaccaaatctctgggcctctgctggttaatttataccatggggatcatcatttctccagtggggttgtgaagagaatgtggtgggatcttgtgtgtggagcatgacacttagcaggcatccgggaatggcagcctcctccctttctaaactggggctttctgagggtgacttcagattccacaatgtcaacagcacaatggcatcctcataaggaaagtttgggttggggctcctcaagcaattctctactctcatttggtacaaagaaaaaaattaagcctcacaattttcttggcaccagactgaacctcaaacccagtcttcacttttactaaaaagccataaacagagaccaggagggtaaaaactaccagaagatacactggatttagaaaacagtaagctggatgtgaagccaaaagagagggagaaagcaacataaaagaatgtcagattgaagaagaaatggattctggccacctaaagaagaagggaagtatagaaaagggaagctgattggaaacaatgggtaatgatgagtgtccctcccctaaaaagttaaaaaataaatgagcaggcatgagaaaaggaagtaggccagaggaagtagaccaggatcagccacggatgtgagtggggaattttacaaatttctacttgggtgtgaaacatgtgtacactcaaagctctgtctaggttcaccgaaactctcccagctcaaggccatttagtaaaaccttggcttaactgaatagtgggtaaggaggcctgaggatggcccagagaaggtttaaattcttactggctgctgcactaagttgtaaagtgcagccttagttgatgatgcctttattagtgcaacatcccagaatgcatgcgtcttacaacccttgtggatatgaaacccagctgggtagacactgcaaagtcttctcatttgattcttccacttggtttctagtgtccctcagggacaaaggaaagccatggtccagtctaagatgaaaaccaattagacctctggcaggcctttgaccaccctgagggccacccccaaggcacggccacactcgcatctgctgccaggaggccctatcttaggctcagctcccaaaccttgaacatcttggaggatccacaaataggaggtacactcagctcaggctcagtcttccttaaaaagcgccttgctaaagctatcaagttcactggaatacttctgcgaaggacacagcttcagcgatgtcagattttttatgtaaatggtgccttcacatgctctggggcatttggctaccaagggcggtttgaactagctccagcacacagaatacaagtctcttcagaaccaggcaaaaccctatgttgcccaatgactcctgctgtttcttgagatttgcacagaagacagagctgcaattacccacgctgatatctatttcatgaccacatatgttcaaaagccacatgtgagaggtatggttgaaatgagaggttgtgtttgccaagttgtcttctgactgtggagagctggtgagcctcttctcatctcctggggctccatattatagagctgacgaatctcttttcttgctccttgaagtctttcattcacttatttattaattcatttaacacatcaggcacctactaacttctcagagttcaaggcttcctagcctatgatcagaaaggacacctgtgtgctcatgagcacccctgggatcagggtgactgatagggtgctgtcatcactactagatgaaactctttggaacaaaggtctaggatataatatttatccttctgcaaatattcatatggcattcgctgtgtaccaggccttctgttgaacattggcctgcagagctgaagaggacgtgggtcttctcttaggaactcctagtcttgggaaaaaggaatggggaagggctgtaatgtgaagttaaaaaaaagtgctgaggccgaacacatctgggccctcactgcagccctattgcacacacactgcatcacctcgaacaagttattcaccctctctgaattcatctatttgcccataaaaatagagatgatccttttatatgttggctaccacttattatgtgcagcatgcttgttattgtactaagttactcattttttagtttttcatataactcatatttttgttgatttttattttaaaattccaaacgaattaattaaactattttccaaaatacgtagtatgtgtacatggtaaaaaacatttccaaaagtttaccataaaaactgtctcctttcccatatcctcaatccttcagaatccctctccagggataattaccaccaccagtgtattccttaagagatatttaaactttatacaaaatacgcacacatcattcttttccacaaatgacagcatactatgtacatggtacctcacctagcttttttcacttaccagtatatcttagagattgttgcatgacagaatatacagatctgcctgtttttgttgtttttcccaagtttccaagagctagaaactgtttggtttttaagcagctgcttggtattccattaattggacttatcgtgctttgatctgtccctagtgatggacattggggttgtttcccttcatttatatttgaagcttgttgcagtgacagtgtacacacttgcttgagcatgtgtgtgtctacgataaatcccaataagcaatttaaagtaatttaattcttatgctctcaacaaacctaagaggttatttttttagaggaggaagctaaggctgttgttttgatgcctttttaccactggacttgggactcaactgtttgatagtcatcgagtccttgactcttctcccctgccaggatttggcccctaagcccagggcccgagtctcctccatcttcaagggagagtgggaacagcacaggccttggcctcagtcctgctccccctgcttctagctgtgggatgggccaggtgcttcacccagctgtgcctcctgtgaggcacagtgtgtgcaaaatggaaatgtgaacatgaagatcaaaaatgtccctcaatgaccagtgctgttcctcagagtcacgtgggaactcatagaaagcgatattggtactgctctttcctctgtagcatggtccagatggctcatagcagggaccatgatatgctgggtgagcacccactgcatgcacccactgtgccagcactgagagactcctgtgggagccacagcaattctagggtcttcactggggactctgagacagcagggagctaggatgagggctgcagagtgttcgtctgccctcactgagcagaccccctggatggcagggagcagtcccaagccagatggatgcccataaccagccatttggctctcaatacataatatcaccacgtatcaggcaaaaccatcctgcccagagcattatctgaatttgcatcccatctgcagaagatacattcacccacttcttccattctgtcttaatcaaagtctttatgtgaattttccccattgagaagacaagccccttcctggcttagactgtacctgactgatcttttcatgagctccttgccaagccagaccacccccagcttatatggagacttggtgcaaattagagatgcccctgtgcacgtggcagccctgagcccaagcacccagtaaggcaaagggcctgatttgggacccctctgccactccaccaggcaatcagttgcttatttctaactttcccttccttctccacatttgtcccattccttcctctcatcatgaatatccccagaggcattcagcagtgcagtgaattaaatatagaacttttttttttcagaattgcagaacggattagatcaatattaatccaaacagagcaatgagcctgacagtttagtaaaagctcaataaagggtggcttacctcccccaaaataatctgaaaagaaagcatgtcttatttcagggggaaaaaaaaataaagtgacctttaaagaccaaattcccaggatacccagggtggaggtggaacatgggagtccacaggcagcctggatgtttccaaagatccaaagggcttttgcttcctcacataatgcaggaaacaaattgaacatgtattaagtgcttgctgtatgtgacacactgtgccaggtgctccccttaaaacagttctgtgggcaggcatgagaatgaatcccgatcttacagacaaggaatgttaggctcagaggtttcaagctcacccatcactcagccagagaggacagatgcaggattcaatctcgggagtgcccgagtccacagaagttcctgtgctgaaggaccgaccacaggcacataaagagatgcgagacaatttttactggatttggccacctctcgaggtcggctttgccagctcttctcactgggggaaggggagggagaaagtagctagctccagggtccctaacatagaaccaccaaggacttgactatttttactcatacagcagcttgtctgggaagatcatgctctgtgacaagctgcaggcactaagtagcaatttctgtttcccacatattagcttgagtcatataaaactgacatggatgtggctcaaaaatagctgtatgtcagccattttataccatttgacttaaatgttattaattaacgtcacagccagagattattctctgagaaaagggcattgtagcctgaagcagagaaagcatacacgttccctggggttgagaactcatcacagcctgagacagcttaggttgtaaagccccggcccacttatcccaggagagtctgggtgagatgcaggccccaaagcagaggctgggaagcgagaagtgacacaccctggctgggtgggccctcatcttggtgagacaccacctgggtaaaaccatcatggaaagggtgtagtggggcgtggaaactccctcggttaaagcgtgagctttgctgtaagttgtggtaaggagggaggcagtgacaaccaggaggcctgttttgagggtttctgagggacccatctgtggtatcacgaggagacgcccagaggagccgtgtgaaagggctgcctcccagccggctctggagtgaatgagcagcaagtcctggctgcgaaaagaaggggagtgcagcctgcagaagtgtcttcttttttcaattcctgctcagaaggaaacaggagataagaatagtggggaagtccaaaccaaagtgaactatagggctggtaatcgtagggggaattagtcacccggagactagcccagcagactaacggagccccatcctccatcttgaatcagtcagcccctctatgactgcagagtcctgaatgatggcaacaccttctcttcacttagcgttgtaggatgaccaacagtcctgatttgcctgggactgaggggttcccaatagatgggactttcagggctaaaaccaggaaagtcctgggcagcccaaaacaaggtagtcactctagagtgtatgactctgtctgatacctgctaagaaagagaaggacttgttgattataaggagaagaggaggtgaaatggttctcaaaaaacaaagatgagggcttccgggtgctgttctgcccaaggctctgggtctgaggcttctctctccaggcctagcttcatggaaaagtaaggggccagagggtggaaaaggtggaaacaaaggaagaggatggagaattgctttggggaagtttggactggaagtgtgaattacagctgcacccccaattcaccccatctcacccccctccccctcctgctcatggttctccctttctcatccacacattggtcaaactagctagcttttggagagattttgggcagtaaaagtaaaacagatctgtctcaagcttcaaaaagcctagagctggctgggcgctgtggctcacgcctgtaatcctagcattttgggaggctgaggcggaaggataatctgaggtcaggagtttgagaccagcctggctaacatgatgaaaccccatctctactaaaaatacaaaaattagccaggcgtggtagtgcacgcctataatcccagctatttgggaggctgaggcaggagaatcgcttgaaccccaggggacagaggttgcagtgagctgagatcgcaccactgcactccagcctgggtgacacagcgagactccatttaaaaaaaaaaaaatgcctagagccaaatgctcacagagccatttactgcatggctttgggcaagtcaaaggagtccgcctctcctgtcagaagagtctgttgcagtcttcatcacaagactgttgtggggattaaacaagatggcaagtgggaagttgggaaatgtagtgtgcacccaaccaatatttgtttcttcctgcctgcctacatatgaggccacacagaattccaactttgtttctctgataactaacacagttacttgtttttctttctgatccaggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggcaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctggcactgctgtgctgtgcccaagttgtggtgctgacaaagcttggaagagcctgcaggtgctgctgcgtggcatagcccagacacagaagaggctggttcttacgatggcacccagtgagcactcccaagtctacagagtgatagccttccgtaacccaactctcctggactgccttgaatatcccctcccagtcaccttgtggcaagcccctgcccatctgggaaaataccccatcattcatgctactgccaacctggggagccagggctatgggagcagcttttttttcccccctagaaacgtttggaacaatctaaaagtttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttcca

SEQ ID NO: 77 - DNA, 49 bp, artificial
  CX3CR1_T01
tctttcctctgtagcatggtccagatggctcatagcagggaccatgata

SEQ ID NO: 78 - DNA, 49 bp, artificial
  CX3CR1_T02
tatgctgggtgagcacccactgcatgcacccactgtgccagcactgaga

SEQ ID NO: 79 - DNA, 49 bp, artificial
  CX3CR1_T03
tgctgggtgagcacccactgcatgcacccactgtgccagcactgagaga

SEQ ID NO: 80 - DNA, 49 bp, artificial
  CX3CR1_T04
tgagagactcctgtgggagccacagcaattctagggtcttcactgggga

SEQ ID NO: 81 - DNA, 49 bp, artificial
  CX3CR1_T05
tcctgtgggagccacagcaattctagggtcttcactggggactctgaga

SEQ ID NO: 82 - DNA, 49 bp, artificial
  CX3CR1_T06
tgggagccacagcaattctagggtcttcactggggactctgagacagca

SEQ ID NO: 83 - DNA, 49 bp, artificial
  CX3CR1_T07
tcgtctgccctcactgagcagaccccctggatggcagggagcagtccca

SEQ ID NO: 84 - DNA, 49 bp, artificial
  CX3CR1_T08
tgccctcactgagcagaccccctggatggcagggagcagtcccaagcca

SEQ ID NO: 85 - DNA, 49 bp, artificial
  CX3CR1_T09
tggatggcagggagcagtcccaagccagatggatgcccataaccagcca

SEQ ID NO: 86 - DNA, 49 bp, artificial
  CX3CR1_T10
tctcaatacataatatcaccacgtatcaggcaaaaccatcctgcccaga

SEQ ID NO: 87 - DNA, 49 bp, artificial
  CX3CR1_T11
tatcaggcaaaaccatcctgcccagagcattatctgaatttgcatccca

SEQ ID NO: 88 - DNA, 49 bp, artificial
  CX3CR1_T12
tcctgcccagagcattatctgaatttgcatcccatctgcagaagataca

SEQ ID NO: 89 - DNA, 49 bp, artificial
  CX3CR1_T13
tctgaatttgcatcccatctgcagaagatacattcacccacttcttcca

SEQ ID NO: 90 - DNA, 49 bp, artificial
  CX3CR1_T14
ttccattctgtcttaatcaaagtctttatgtgaattttccccattgaga

SEQ ID NO: 91 - DNA, 49 bp, artificial
  CX3CR1_T15
tccattctgtcttaatcaaagtctttatgtgaattttccccattgagaa

SEQ ID NO: 92 - DNA, 49 bp, artificial
  CX3CR1_T16
ttctgtcttaatcaaagtctttatgtgaattttccccattgagaagaca

SEQ ID NO: 93 - DNA, 49 bp, artificial
  CX3CR1_T17
tctgtcttaatcaaagtctttatgtgaattttccccattgagaagacaa

SEQ ID NO: 94 - DNA, 49 bp, artificial
  CX3CR1_T18
tttatgtgaattttccccattgagaagacaagccccttcctggcttaga

SEQ ID NO: 95 - DNA, 49 bp, artificial
  CX3CR1_T19
ttcctggcttagactgtacctgactgatcttttcatgagctccttgcca

SEQ ID NO: 96 - DNA, 49 bp, artificial
  CX3CR1_T20
tcctggcttagactgtacctgactgatcttttcatgagctccttgccaa

SEQ ID NO: 97 - DNA, 23 bp, artificial
  CX3CR1_gRNA1
gtacagtctaagccaggaagggg

SEQ ID NO: 98 - DNA, 23 bp, artificial
  CX3CR1_gRNA2
gggagcagtcccaagccagatgg

SEQ ID NO: 99 - DNA, 23 bp, artificial
  CX3CR1_gRNA3
gactgctccctgccatccagggg

SEQ ID NO: 100 - DNA, 23 bp, artificial
  CX3CR1_gRNA4
gtgaatgtatcttctgcagatgg

SEQ ID NO: 101 - DNA, 23 bp, artificial
  CX3CR1_gRNA5
gtctctcagtgctggcacagtgg

SEQ ID NO: 102 - DNA, 23 bp, artificial
  CX3CR1_gRNA6
gacagcagggagctaggatgagg

SEQ ID NO: 103 - DNA, 23 bp, artificial
  CX3CR1_gRNA7
gaaggggcttgtcttctcaatgg

SEQ ID NO: 104 - DNA, 23 bp, artificial
  CX3CR1_gRNA8
gcaaattcagataatgctctggg

SEQ ID NO: 105 - DNA, 23 bp, artificial
  CX3CR1_gRNA9
gagcagaccccctggatggcagg

SEQ ID NO: 106 - DNA, 23 bp, artificial
  CX3CR1_gRNA10
gaactcatagaaagcgatattgg

SEQ ID NO: 107 - DNA, 1599 bp, homo sapiens
  CD11b_loci
gtgcatgggggtggggtgggggactctgggtggggaggagggtaacttttgggtctgtcataaatagagggcccagaatatgtaggagtcagtctggggagaggcaaaggggatttggggaaggagaaagggttcaagaagaagcagggagaacagctagacccagacaggctggccagggaagcctggatgaatgaccacattcatggactgtgcaaggctgcttgccggtccccttgcttcacacatgaggagacggaggcccagggaggagaagtgacatggctcagggtgcgcagcaggtgtgagacccctttcctgagtgcttcctcctggatcccctctcaccatctccactttgcctccggttctattttccaaggtcccgggtgcaaatgtttgttgaatgactgatgaatgaaaatgatttgagtttgttaccttttatgcttatatgttgtggaaaatgaaattctcctcaaaagggaaggaaatacttgagagctgcataggaaggaaattatctaattaagaatgtatagaaacttcactgttgggcaaatcatcgttgtgacaccgggggaagaagccatttaggtgctcagaagggaggctggaattcagagcaggactggacgtgccccacgacggtggttcttaggtcaggagtcagcaaacagtggcctgggggcccgatatggcccacgacctgtttttgcacaacctgccagctagagattgaagatgaacactgataatcgatttgatgatagggagcaccacccccaaagaattctatttgtctcatttgtaaacccgtattacaaacaaattgtactcaatcattatgtttgaaatttccctaatgacaaatttgtggaaaagtattttctgtcttgttatataagtacttgtacaacatattctatcagcctcttggtctgcaaaacctaaaatttactatctggctgtttacagaataagtgtgctaatccccgccccaggctaacagagctggacctgggaggcagacatctggatgctgggttagttagggtgaccgaatggatgggaaagggaatggagcaggaagacatgctgctatcttttttttttttttttttttttgatacagggtctttctctgttgcccaggctgtagtgcagtggcatgatcatggttcactgcagccttgacctcctgggttcaagcaatcctcccacctcagcctcctgagtaccactacacccggctaattttttattttttgtagagatggggtctcactgtgttgcctaggctggtcttaaactcctgagcccaggtgatcctcccacgtcagcctcttaaattattgggataacaggcctgagccaccacacccagccattgctttcttttaaattattattagtttttgttttttttttgtatttaattttgagataggatctctctctgttgctcaggctggagtgcagtggcacaatcagggctcaagtgatcctcctgcctcagccctccaaagtgctaggactacagcccctaactggcatgctactttcctctgcttgatccttcccccattctccctttag

SEQ ID NO: 108 - DNA, 49 bp, artificial
  CD11b_T01
ttcagagcaggactggacgtgccccacgacggtggttcttaggtcagga

SEQ ID NO: 109 - DNA, 49 bp, artificial
  CD11b_T02
tatggcccacgacctgtttttgcacaacctgccagctagagattgaaga

SEQ ID NO: 110 - DNA, 49 bp, artificial
  CD11b_T03
tgatgatagggagcaccacccccaaagaattctatttgtctcatttgta

SEQ ID NO: 111 - DNA, 49 bp, artificial
  CD11b_T04
ttctatttgtctcatttgtaaacccgtattacaaacaaattgtactcaa

SEQ ID NO: 112 - DNA, 49 bp, artificial
  CD11b_T05
tatttgtctcatttgtaaacccgtattacaaacaaattgtactcaatca

SEQ ID NO: 113 - DNA, 49 bp, artificial
  CD11b_T06
tttgtaaacccgtattacaaacaaattgtactcaatcattatgtttgaa

SEQ ID NO: 114 - DNA, 49 bp, artificial
  CD11b_T07
ttgtaaacccgtattacaaacaaattgtactcaatcattatgtttgaaa

SEQ ID NO: 115 - DNA, 49 bp, artificial
  CD11b_T08
tacaaacaaattgtactcaatcattatgtttgaaatttccctaatgaca

SEQ ID NO: 116 - DNA, 49 bp, artificial
  CD11b_T09
ttgtactcaatcattatgtttgaaatttccctaatgacaaatttgtgga

SEQ ID NO: 117 - DNA, 49 bp, artificial
  CD11b_T10
tgtactcaatcattatgtttgaaatttccctaatgacaaatttgtggaa

SEQ ID NO: 118 - DNA, 49 bp, artificial
  CD11b_T11
tactcaatcattatgtttgaaatttccctaatgacaaatttgtggaaaa

SEQ ID NO: 119 - DNA, 49 bp, artificial
  CD11b_T12
tttccctaatgacaaatttgtggaaaagtattttctgtcttgttatata

SEQ ID NO: 120 - DNA, 49 bp, artificial
  CD11b_T13
ttccctaatgacaaatttgtggaaaagtattttctgtcttgttatataa

SEQ ID NO: 121 - DNA, 49 bp, artificial
  CD11b_T14
ttgtggaaaagtattttctgtcttgttatataagtacttgtacaacata

SEQ ID NO: 122 - DNA, 49 bp, artificial
  CD11b_T15
tgttatataagtacttgtacaacatattctatcagcctcttggtctgca

SEQ ID NO: 123 - DNA, 49 bp, artificial
  CD11b_T16
ttatataagtacttgtacaacatattctatcagcctcttggtctgcaaa

SEQ ID NO: 124 - DNA, 49 bp, artificial
  CD11b_T17
tatataagtacttgtacaacatattctatcagcctcttggtctgcaaaa

SEQ ID NO: 125 - DNA, 49 bp, artificial
  CD11b_T18
tacaacatattctatcagcctcttggtctgcaaaacctaaaatttacta

SEQ ID NO: 126 - DNA, 49 bp, artificial
  CD11b_T19
tcttggtctgcaaaacctaaaatttactatctggctgtttacagaataa

SEQ ID NO: 127 - DNA, 49 bp, artificial
  CD11b_T20
tgcaaaacctaaaatttactatctggctgtttacagaataagtgtgcta

SEQ ID NO: 128 - DNA, 49 bp, artificial
  CD11b_T21
tgaaaatgatttgagtttgttaccttttatgcttatatgttgtggaaaa

SEQ ID NO: 129 - DNA, 49 bp, artificial
  CD11b_T22
tttgttaccttttatgcttatatgttgtggaaaatgaaattctcctcaa

SEQ ID NO: 130 - DNA, 49 bp, artificial
  CD11b_T23
ttgttaccttttatgcttatatgttgtggaaaatgaaattctcctcaaa

SEQ ID NO: 131 - DNA, 49 bp, artificial
  CD11b_T24
tgttaccttttatgcttatatgttgtggaaaatgaaattctcctcaaaa

SEQ ID NO: 132 - DNA, 49 bp, artificial
  CD11b_T25
tttatgcttatatgttgtggaaaatgaaattctcctcaaaagggaagga

SEQ ID NO: 133 - DNA, 49 bp, artificial
  CD11b_T26
ttatgcttatatgttgtggaaaatgaaattctcctcaaaagggaaggaa

SEQ ID NO: 134 - DNA, 49 bp, artificial
  CD11b_T27
tatgcttatatgttgtggaaaatgaaattctcctcaaaagggaaggaaa

SEQ ID NO: 135 - DNA, 49 bp, artificial
  CD11b_T28
tgcttatatgttgtggaaaatgaaattctcctcaaaagggaaggaaata

SEQ ID NO: 136 - DNA, 49 bp, artificial
  CD11b_T29
tggaaaatgaaattctcctcaaaagggaaggaaatacttgagagctgca

SEQ ID NO: 137 - DNA, 49 bp, artificial
  CD11b_T30
tacttgagagctgcataggaaggaaattatctaattaagaatgtataga

SEQ ID NO: 138 - DNA, 23 bp, artificial
  CD11b_gRNA1
ggttgtgcaaaaacaggtcgtgg

SEQ ID NO: 139 - DNA, 23 bp, artificial
  CD11b_gRNA2
gggaggctggaattcagagcagg

SEQ ID NO: 140 - DNA, 23 bp, artificial
  CD11b_gRNA3
ggagtcagcaaacagtggcctgg

SEQ ID NO: 141 - DNA, 23 bp, artificial
  CD11b_gRNA4
gagtcagcaaacagtggcctggg

SEQ ID NO: 142 - DNA, 23 bp, artificial
  CD11b_gRNA5
gacctaagaaccaccgtcgtggg

SEQ ID NO: 143 - DNA, 23 bp, artificial
  CD11b_gRNA6
gcaaatcatcgttgtgacaccgg

SEQ ID NO: 144 - DNA, 23 bp, artificial
  CD11b_gRNA7
gagacaaatagaattctttgggg

SEQ ID NO: 145 - DNA, 23 bp, artificial
  CD11b_gRNA8
gccccacgacggtggttcttagg

SEQ ID NO: 146 - DNA, 23 bp, artificial
  CD11b_gRNA9
gaaatacttgagagctgcatagg

SEQ ID NO: 147 - DNA, 23 bp, artificial
  CD11b_gRNA10
ggtcaggagtcagcaaacagtgg

SEQ ID NO: 148 - DNA, 387 bp, homo sapiens
  S100A9_loci
gtaagtgagctgccagcttccccaggcagaagcctgcctgccgattccttctttccttccctgacccaacttccttccaaatcctcctcctagaagccctccttggttggccctgcctactttaaagcttctttcacattttcttaggtcatgttcccctggggcctcctgccctcaaatgctttgctttttggcactctgtagatattctaaaaaatcattttgtacatgtgtgtgacaggccatctcccagttaagttgcagcctgtgctttctttttattttgcacttcccccactatttctgtgagtgcttagtaggaagtgtcaaagaagcttgacagcattttcttctaagtgtcccaactcttggttttccattacacag

SEQ ID NO: 149 - DNA, 49 bp, artificial
  S100A9_T01
tttccccgttgtattggttgaaataagtttcactaattggtaacctcca

SEQ ID NO: 150 - DNA, 49 bp, artificial
  S100A9_T02
tattggttgaaataagtttcactaattggtaacctccagagggaaggga

SEQ ID NO: 151 - DNA, 49 bp, artificial
  S100A9_T03
tttcactaattggtaacctccagagggaagggaagggagggcaggggaa

SEQ ID NO: 152 - DNA, 49 bp, artificial
  S100A9_T04
tggaactggcctctaagtcagatctgaatttgcatgccctcaatagtca

SEQ ID NO: 153 - DNA, 49 bp, artificial
  S100A9_T05
tctaagtcagatctgaatttgcatgccctcaatagtcaagctgtgaaaa

SEQ ID NO: 154 - DNA, 49 bp, artificial
  S100A9_T06
tgcatgccctcaatagtcaagctgtgaaaactaatgaccctctctagga

SEQ ID NO: 155 - DNA, 49 bp, artificial
  S100A9_T07
tgaaaactaatgaccctctctaggactggtttcaagtcttcctccagga

SEQ ID NO: 156 - DNA, 49 bp, artificial
  S100A9_T08
tcttcctccaggaagataccattcctagctgttaaagttgttataagga

SEQ ID NO: 157 - DNA, 49 bp, artificial
  S100A9_T09
tcctccaggaagataccattcctagctgttaaagttgttataaggacca

SEQ ID NO: 158 - DNA, 49 bp, artificial
  S100A9_T10
ttcctagctgttaaagttgttataaggaccaaatgaggtgacatttcca

SEQ ID NO: 159 - DNA, 49 bp, artificial
  S100A9_T11
ttaaagttgttataaggaccaaatgaggtgacatttccaggcttactca

SEQ ID NO: 160 - DNA, 49 bp, artificial
  S100A9_T12
tgaccagggcaagaccctggaactcagcttcctcttctataaatagaga

SEQ ID NO: 161 - DNA, 49 bp, artificial
  S100A9_T13
ttcctcttctataaatagagaatcagcacccaagtcacagggtcatgga

SEQ ID NO: 162 - DNA, 49 bp, artificial
  S100A9_T14
tcttctataaatagagaatcagcacccaagtcacagggtcatggaggga

SEQ ID NO: 163 - DNA, 49 bp, artificial
  S100A9_T15
tctataaatagagaatcagcacccaagtcacagggtcatggagggaata

SEQ ID NO: 164 - DNA, 49 bp, artificial
  S100A9_T16
tataaatagagaatcagcacccaagtcacagggtcatggagggaataaa

SEQ ID NO: 165 - DNA, 49 bp, artificial
  S100A9_T17
tggagagcgtttggtatgtgctcagtgtctgctccattgtgcgcactca

SEQ ID NO: 166 - DNA, 49 bp, artificial
  S100A9_T18
tggtatgtgctcagtgtctgctccattgtgcgcactcagcctatggtca

SEQ ID NO: 167 - DNA, 49 bp, artificial
  S100A9_T19
ttgtgcgcactcagcctatggtcatttttaatttttaaatccagcccca

SEQ ID NO: 168 - DNA, 49 bp, artificial
  S100A9_T20
ttcccttgtacatttgccagctggtcatttactgtgctcccagtcccca

SEQ ID NO: 169 - DNA, 49 bp, artificial
  S100A9_T21
ttttgttttcttttcaaatttggggaaagtcgggaaacagaggcctgca

SEQ ID NO: 170 - DNA, 49 bp, artificial
  S100A9_T22
tttcttttcaaatttggggaaagtcgggaaacagaggcctgcattaaga

SEQ ID NO: 171 - DNA, 49 bp, artificial
  S100A9_T23
ttcttttcaaatttggggaaagtcgggaaacagaggcctgcattaagaa

SEQ ID NO: 172 - DNA, 49 bp, artificial
  S100A9_T24
ttggggaaagtcgggaaacagaggcctgcattaagaagggtggaacaca

SEQ ID NO: 173 - DNA, 49 bp, artificial
  S100A9_T25
taggtccccagccctcccagtgcccctccctccgccttggtaaggtgga

SEQ ID NO: 174 - DNA, 49 bp, artificial
  S100A9_T26
ttcagagttaggggccctgacagctctccataggtggaggcctcaggca

SEQ ID NO: 175 - DNA, 49 bp, artificial
  S100A9_T27
ttaggggccctgacagctctccataggtggaggcctcaggcaggcagga

SEQ ID NO: 176 - DNA, 49 bp, artificial
  S100A9_T28
tccataggtggaggcctcaggcaggcaggatgctgggtggggtaggcaa

SEQ ID NO: 177 - DNA, 49 bp, artificial
  S100A9_T29
taggtggaggcctcaggcaggcaggatgctgggtggggtaggcaagaaa

SEQ ID NO: 178 - DNA, 49 bp, artificial
  S100A9_T30
tgggtggggtaggcaagaaagggcccagcagagaggccgcatggcaaaa

SEQ ID NO: 179 - DNA, 23 bp, artificial
  S100A9_gRNA1
gcacaggagagtgctcgcattgg

SEQ ID NO: 180 - DNA, 23 bp, artificial
  S100A9_gRNA2
ggtaccccacaggttctgggagg

SEQ ID NO: 181 - DNA, 23 bp, artificial
  S100A9_gRNA3
ggagccagacatcctggggtagg

SEQ ID NO: 182 - DNA, 23 bp, artificial
  S100A9_gRNA4
ggagagtgctcgcattggctggg

SEQ ID NO: 183 - DNA, 23 bp, artificial
  S100A9_gRNA5
ggaagcagagcctcatggatggg

SEQ ID NO: 184 - DNA, 23 bp, artificial
  S100A9_gRNA6
ggcttactcatgccatgaccagg

SEQ ID NO: 185 - DNA, 23 bp, artificial
  S100A9_gRNA7
gggaaacacctagaaaaactagg

SEQ ID NO: 186 - DNA, 23 bp, artificial
  S100A9_gRNA8
gtggggggtgaagcgggcatagg

SEQ ID NO: 187 - DNA, 23 bp, artificial
  S100A9_gRNA9
ggggggtgaagcgggcatagggg

SEQ ID NO: 188 - DNA, 23 bp, artificial
  S100A9_gRNA10
gagggctggggacctaccccagg

SEQ ID NO: 189 - DNA, 3424 bp, homo sapiens
  TMEM119
gtgagtcctggggtcccctcctctgtcctgagagcctggagctatcttggagcttagggactggggactgttggagcactctggggggcctctctaagtgtgtgtgggctttgagtgtgtgtttggtgttgtgtgcatgagtgtggtggaatctgagtcccgtgtgcggtgctggggtgggccagggtgggccggagtgttgtgtgtgtgcggctggggccttggtgtagggggtgtgttgcttccacttttctgcaagtggatgccaggctgttgcttccaggatctgtgtgagggtgatgtggacggtattgtcctgtgtgcagggatcggctgtgtgtcagggggttgtgtatgtatatgtgttgtgtgtccgtgtgtgttctctgtgtattgtgtgcatgggccatgagagtgctgttgggtgtctcagtgctgtgtgagtcggtgtgcctgtgttgtgtgtctttatattgtgtgtccatgctgctctgtgtgtgtgtgtgtgtgtggagggggattgtgcccccaggacccccctccccatccagacctccagggcccaggtccctgtccttggctggccctggggtcgggggcatgtgcagtgctccaaccagaacacccctcccccacaaggccactgattgaagccaggtttccgtgggccgcccctcccccaaaggccaccagtctctgggtgggggtggggcagcccccggtcccctagcttcaagtcttggggcccccaggcctcaggggtgcatctgcactcctccacagactcaggaccatcttagggcatttgccacacttacacacggccagccctgcctgacatgtccccaggcagggagtcctgggcaatcacgtggtacctgctaattggcacccactgtgcaccaggcatggccgcatcttgtcctaacagcagtccagggaggtggctcttatccccattgtacagatatggaaaccgaggctcagagaggtgacatgcccagcccaaggacacatagccgggaagtgacctcctctgggatataccaagacaggcttctctgtccagggctctgctgggctcacccctgactttctgtgtgacctgggcaggctgtttccagtctctggacctgagttacctctaaggtgaagcaagaggcgttgggcctgaccccagctcctaatggcctgttagattctgagtccttgaaaaaattagggtccggggggaggtaggcagaataatgacttcagagctgcccagaacagtgcttcattccacagatgggaaactgaggcccagagagggcaggggccgtccagggccacacaaccatcttttttgaggctctgctgactgcaggtggggctgggtcctccatacgttgcccccacctttccctggaagccaggaggggttgtggggtgcaggggactctgagagtcagggtctcagccttaaagagccagtggcagtttgtgtgttctcctttcttgccctctgcctctgacttcgtgtgtgtgtgtgtgtgtgtgttgtatgtatatgtgtgtgtaggtgtatgtgtgcatgtgtgtgtatgtgtatgtgtgtgcatgtgtgtgtttggttctgcattccttggcggccagagctaaagaggtttcactgaacggggtgaaaaggttggtctttcactgggtggcggccggaccagacagtgagcgcatgttcccatccggcgatggtgctcatttgtcgactcagggtgggaaattgctggctgaacctggagttcggctagggaaggaattcagctaccccagaccctatctttcctcggggctcagctacttcgcaccaggcactctctgaaagttgtctcatttggccctaacagctctgagaggtgctacctgtggtcaccctgtcccaacttacggatggggaaagtgaggcccacaggagccagaccacagagctggatgcgatggagccgggattcttgacccagccagaacttgagtgggtctccagccacccatggtcccagctgccctctgcagtgagaagtggtagttgttgccacaaaagctctctcctaggcttgactgggggtgggaggcagggaggtgactggcaggtcacagaccccaaagctcaaggttgtctgtaggaacctttgctaacaggtgccagagaggtgagggggtcctttcctcatttagcatctgacaggtgatgtccaccagcgggaacaagccattcctggctcagagtgtgtggtgtgtgcatgtgtgtgtggttgtgtaggtgagtgtgtgtggtgtatgtgtatggttgtgtgtatgtggagtgtatgcatatatgtgtggtgtgtgcatgtgtgtatgattgtgtgtagtatgtgaatgtgtgtataggggtgtgtgtggggcatgtgtgtacgtggttgtgtgtggggtgtatatgggtatgtgtggcgtgtgtgcatgtttgtgtgtggggtgtgtgtgcatgtgtgtagatgattgtgtgtgtagtatgtgcatgtgtgtatatgggtgtgtgagtgtgtgtgtggtatgtgtgtgcatggttgtgtgtggtgtgtgtgcatgtgtgtatgattgtgtagtctgtgcatgtgtgtatatgggtgtgtgtgtgtgtggcgtgtgtgcatgattatgtgtggggtgtgtatggggatgtgtgtgtgagtgtgtggcgtgtatgaatggttgtgtgtgtggtgagtgtgcatgtgtatgtatggctgtgtatgagtgtgagtgtggtttgtgtgaatgtgcgtgtaaactcctgggatcagacagaggaaggttcagatcccagctcagctacttatcatctgtgtgtccttggggaagtcactgtctgtctctgaacctctgtttcctcatctgtaaaatggggataatgaagctgcctcagggctcctgtaggattaaaggcatgaaaagggcttagatgagtacccgcgaggaaagcacaggtgaccagctcatttaaaatgagcccctgagaagctttgtctcttttttaaaaaattgaaatattttaatatatttcatagagatggaggtttccttatgttgcccaggctggtcttgaactcctgggctcaagcgatcctcccaagtagctgggactacaggcatacaccaccaggcctggctacttttttatttttttgtagaggtggggtcttactatgttgctcaaactcctgccctcaatcaatcctccggcctcggcctcccaaagtgctgggattacaagtgtgagccaccacacctggccaattttgcctctttttatgtcctggaattatttttttattttttccatctaagccagtttgttcacatttaatttcttttttttcttttctttctttctttttttttttgacag

SEQ ID NO: 190 - DNA, 6273 bp, homo sapiens
  MERTK
gtgagtgcgcccggctgggggccaggcgagggggtgggggctcccaggaggaagcaggggcctctggggagggagcgcgtccacaggggcgcgcctggctgctggacaagtttgcaaacaccctccacccactgcggtgccagaggagggggcgtaggcgaacctaccgtccactgaccgcggcgcctcaagcgtcccgagggcacccagcctggcttgcgggtgcgcatcgtggtgaagccgggtcggggtcggcgttgcaggctcccctcttcaccgcagaggaggaaatcgagctcggccgggcgcgctgcaagtcctgaagttccgacgagtggaacgtaggtagcaggtctggtctgggatgcgacgagaactttccactagagttgcatggtggggccccgctctggccggagtcccgggggccacagatccggtctcccgagggaccggcgcggggcgaacgaacgggctgcgggagtcggagccgcagtccgggagccgcgatagactgaggccgagcgacgggccagggggggacggcagtcctggctgctcccaatgggcccttacgggcattattagtatccctttttaggggcacccagagagtgtgggaaaaggggagcgttcttttcactcctgttatggcagaacagaacttacagtgctcggcagcgtttacgtttatctggattcagcgaagtgttgttacaggcttttttttttttaatttgagaccgagtctcgccctgtcgcccaggctggagtgccgcggcgcgatctcggctcactgcaggctccgcctcccgggttcaagcgattctcctgcctcagcctcccgagtagctgagactatagacgcgcgctaccacgtccggctaatttttgtatttttagtagagacggggtttcaccatgttggccaggatggtctcgatctcttgacctcgtgatctgctctccttggcctcccaaagttctgggattacagtcgtgagccaccgcgcccggcttgttccaggcattttaatacaagatgctggactgatacgtggaggcaaggaaggccggtttcattctgcttgaacagcttatttggggagtgagtgatgaacagatttcaagtttgatcgctgatgcacttcttgaggaggctggaataaacttaccaggctttacccaaaagaaaaagaaaggggaaaaatgctaaataaacacattccaagtattttctctcagctaaaaggtttctatttccattcagcatgtagattttctctcttttgatcgagaatgcacttgactagccttcacctattacagtcaaagatgttttttaggaatacctggaaagctcagtttatagtcaagtaagttacttaaaagtttttcctttgagattttcctttgaaagctcatttactcaaatgaaaaattcttaaacagtgcacccactttttgcacgttaaattacataagcaatttataatttggggcaacatagcaatttgaaacaaactttaagagccaggcaaagaactaaagcagttctgttctccactgcccctccctccaccaaaccttcaaaaattcctcagcatttctgccagccccatttttcataatctgctgcaaatctgttatttgttgctactagtgaggactggccgtgaatagtcttggaggtagtgaatgtggtgagaactatggagaaagtaatgggaacaatagttttgtgttaaatacttaaaaattggagtgcttttgtttttctaaaattttaaaaggctgtaataacaaatttttgtagctgacaatcaactttaatcagacaatcataaaaaggccttttaaaatccacccagctggaagaatgaaaagtttcaccaatcgttcttgcactttttgcttttggttttgaactttacctatgagatgtgtgacagttttacatctcaccttgtgaaaagcttaaagaatgggattctcactggcagaattacagcacaaaggagtgctgtatgaagaaattaaacatggaaattagactcttcatctaattttaagattttcctattacacattgtgggtctgagtgctttgctgaccacacaggggagacagagagagagacaatgagaatatgtgtgtagattctacactgttagcagtatagaagaatgggtaaataaaatttaagcatcactttaacccaggaaatcccctctgccccccaacacacatacacactacacgtctcacaaagacatgcacacacagaactggaactgccaaaccaggagcatttcgaggctttccagtctttcctttctgctcagtctctgatgggggtgccattgagcagcaaaatgaggcaagcggtggctctctgaagggccagggagaggatggagcaagataaatataaatgtggataaaaatgaatgcattatgaggacttagctcatgacgaggctgtgtgtgcagctaaactcaacccttagagtaaattaggcttttaagattctttaatgttaatatttcttttccaaactgatattgtaacatgtattccagtatattgtaaacatcttcccagagggaacttttaagttgttctgttgcttgtgggctttattccaaaggcttcaaatgctttttgaaagtacatcgtgcatattttaaaaatgaatacttttagaaaattattctgacccattaaagtgctgagtggaattcttttcttttctttctttcttttttttttttttttggcaggttctcattctgccacccaggctagagtgcagtggcaccatcacggctcactgcaacctcaaccttccgggctcaagtgatcctcccacctcagcctcccaagtagctatgaccacaggcacataccaccatgcctggctaattttttattttttgtagagacaggatctcactatgttgcccaggctggtcttgaactcctgggctcaagcgatcttcccaccttagcctctcagagtgtggggattataggcatgagccaccatgcacagctgaatacaatttttaaaaaattaacaataattatgtttttttgtttttgagatagagtttcactcttgctgtccaggctagagtgcaatggcacaatctcggctcaccgcaacttcccgcctcccgggttcaagcagttctcctgcctcagcctcctgagtagctgggattacaggcatgcaccaccacgcctggctaattttgtatttttggtagagacagggtttctccgtgttggttaggttggtctcgaactcccaacctcaggtgatctgcctgccttggcctcccaaagtgctgggattacaggcatgagccaccacacccagccaataattatatttttaaatcttaatttttgaaataatttccagataattgtagaagggccagtgagttactagtcagcattggtcaagaagaggttcaatctttttatgcctgtgaattgggaacatgctgctgggtagaaaaactgagctgggaccttgaacacacatgtgaaacagtcgctttccatataatagccagaatgagattttggaaacgaatctgattgtattttccatttgaaaatcttccagtggcttcctgttccctactagaatttgcagagactcacagcgaacctctttgacccatttccattcacttccctcttgttcctaccactccagccacctgtgtcttctttctagggcctgagcagcctttgcagtgactcttccctctggctggaacctttttcccaagatctcggcatgctggcttcttctcatcctttgggtttcagttcagaggttctccttgagaagccttccccaataaccaccccctgccaaagtatcatatgcatacaccccactatcccttaaatttaatttcctgataatgcttctcacaacctgatcagttccttccttccttccttccttcctccctccctccctccctctatttctttattgacagagtcttgcaatggcaaattctctgctcactgcaacctccgcctcccaggttcaagcaattctcctgcctcagccttcccagtagctgggattacaggcacctgccaccacagctggctaatttttgtatttttagtagagacagggtttcaccatgttggccaggctggtctcgaactcctgacctcaggtgatccgcctacctcagcctcccaaagtgcagagattacaggcgtgagctatggcacccagccttacctctttgtttactccttttcctccactgtccccccaccacaaggtaagctcctggtggcagggctgctgtctttttcactgctaacacagcatggagctcatacgggtgctcagtgagtatcagagcacagcatgaaggaaccaagcccctgcatgtcttaccaggtgctcttcacccttatcttttcctactggcttttccagaaacagaattgctggagctcccatgccctcacattcggggcttggttaatagaaaacaatcttttattttgctgtgatgccaatgtgatttcattgctggaggagctagtcaattaaaaccagacagatttttgggctgtattaaccccaaacatattttacattgtcctaactattccaactagaaaaaaaaatgtcagaaggtaatttttcatgttaaacacagttctaacaactccttgctcaggggcaggtagccctgggtgccttgactttggcccttgccgttagtttaatcttaacccaaagcacatggctctgaattggtatgtttctcagcaggagattaattattataaaaactgaaaggggtctcacactgtgtgaggcacactatgcctctggggactggaatgaaagtgttaaccgggactggcttctggggctttgggggtggtgattaactttcaggacctctgttcctttttgggtacagtgggaggctcttttcatagaattgttaggaggacgtaatgagctaatggcaggtgatatgtgcttaaaatatatttgccattattaccaatggttctcctccattcttctgaactcaagtatgggggaaatgtttttcctagacaaaaggtgttgggaggagggttcttttcttattgaagtgtatcctgaatggtccaccctccattgttcagaatgctttggtgaaggggccctccggtgtcgccctagagtgcccaagtcctttgctgcagctctttaggtttttgtttcctagtagtttgagcaaaacccacccaggaccaagcagttctccagagaagcagggcccgccctgactgccattcccctcctactcctgcccccataccttcccttatggagttcaaacttgacatagtacaggaatagcttcttgtcatgtgcctctcaggaaggtataaaaatgaagtccagttcctgttcaacttttcccacataactcttttccaaactgggtgtggtttaaatttttttatttttattttttgtgtttgttttgttttagggtttttgagtgtgaacatccagaaaattcttttttttttttttgagatggaatcttgctctgtcgcccaggctggagtgcatgtggcgcgatctcggctcactgcaacctccgcctcctggcttcaagcaattctctgcctcagcctcccaagtagctgggattacaggtgccctccaccagacctggctaatttttgtattttaagtagagacggggtttcaccgtgttgggcaggctggtcttgaactcctgacctcgcgatccacccacctcggcctctcaaagtgctgggattacaggagtgaaccacctcacccagctcagaaaattcttttcctttttaccacatcgagctaggttttcccaatgaggtcagttggctgcaacatagagaaaatgaggctttggttcttagagtagattccgtgtacaggcgtgagtatgtgtccgcaggtggtcttgtaggaaatgcaaagggcgccaggtgcactcctggcttctggtacttcctgcttaatggcatgggtgagccaaccctcaggtgcccgggagggagccctgattactctccctctggcactctgggcattggctaacccgtgaccggagttaaaccagttccctctcttttcattctatgtctcccactcctgcctccagccgaagtgctaaaaatgcagcctggcaggggcagagaattccattaggatcagcagg

SEQ ID NO: 191 - DNA, 2541 bp, homo sapiens
  CD164
gtgggcgcgggccctgtgggcttcccgccctcccctgcccagccgagccgcactgcctcctcgccgtgcgggcggggcccgggttcgggccacggtcgggcgggcgggccgcgctcggggtgcggcggcgactccggcaacatggcggccagcctggagtccactcgtggccgggccgggtggggagcgcgctggggggaagcggacgcgcggttcccagtggctgcggggctggggcccccctcgaagtgttgcaagtcgaggtgccttttaaaagtgcatcgtaattcctagcctcacttggggtcggaaatggggacgtggcttccttccgcgggggcagcggtggggttgaggatggggccgggcgtcgcgcctctgaggttggcggtggggccgagctgcctctccctgccactggttgttcaccctcccggcgcctctctccggaggtagccttctccggaagtccgggaaagagaaacttaaagacggtaatgctcgttcgtgtctcagacttgagtctgagaaggtcgcttgagatttaattacattggttttgcagataggaatagaatgtagctgacgtttgagcacgtagttagtgaaaacttcacacttcaaagtatgcgaaaaggaagaaaagtacccatgtatgtaccgggtgttttgcgtacgtatcctaaggctaaactggaaggtaggtagtgaaacctaggaggtagatgcagagacggagatgagtaaactctcctgacattgtatgtttattcttttttcaattgtttttaaaattttcttaactatgtcctgtgctcagatagcgtatgtttagtaggtcgtggaacgggggtttgtgcccaggtctgtgggttttctaaaacccgatcaggagcgtgtcttttgttatgtgcagtcgcatagggctggttaaggcatacatacattttaaagcagaaattgccatataccgtgacaacacaaaagagagaaggaagataagtttgaggtaataagcgggagaaacactagaggtttaaaacctgcagtctcgtatgattggttattctagttaacattagttaaggagtaatgtttacgtagttaacattggcttcttgaccgaagaatgccgaagtgtaatgatttttatcacggcattcatattctacaaattctcctctagttttagtattttataaaagtttaataaagtatatagtaaacttaatgttttgtttttagtttaaggaagatgtaatggtaacatttcaaaacgagggaagtaaattgcagtataatactgggaattctaaccgcactattttaagatgatagagcagaatatgtttgtctcagtcttaatttgacagtttcctttcagaattgtagtgtgtgtgtaagagaaggttggaagtatcaaattaatttgtctttgtgtaattgaagtgcgtgcaaaggaggtatgtattttctggaaatcataagtggatgtagtgtggtctgaaaggtagcaagtttagttcattgtaaagaacatttcagacatttcactttgctacacccgtcaagcacaaaggtttttaaacagcatgttttatctatacaaacttcagaaaatttatttttactttatgaacccttccccaccccaccgtacatgtcccagtctttaataagtactaatgatgaagtattgaaagcagtgcctctgcagaactattttttaggcaggagcaaagcaaacgagaatgtttggaggcatctagtttctaggaaaatgaatggagcccaattcttttttttcttttattttgctgctttttctcactggaaaatagagcccaattttatagtccgttcttctccagaaattgaattatggtgatgagtacttgtttctggattgtttggcctaggatgaactattgacaaataccttgttaacctgaattgtatgcacttgaatatgggcttttagatgttgtctccagttgtggaggatcctgtcgagagtggatcatttatgaatcgagaatccctgggaaattatctttgtttgcagcagaatgggcatcatcagtgcttgggttcaacctgttaaactactactgttactactaaaaatctcagggttttatctgatgtttgggggtagtggttttcctatgtgtagtgttggtattatatcaaagcctttttctcgatatttttatgtattaaggatgctttgaaatgcctaaatccttagtggctgtgggtgtcttccaatagttgtcctgtgaaaaaggattggttagacagataccatcaggcactgtactgagagtgaccactagcttgtttttctaatgcgtttacttacagctttgtaatgattggaatttcttagtaaaccagtataaagttttatcatttatctgaattagtaattttgatgctgtatgtaatgcttattgttcaaaaagataagtaatacagattcaaataagatactaataaaatcattgatttcatgcatctctccccccaactccccag

SEQ ID NO: 192 - DNA, 357 bp, homo sapiens
  TLR7
gtattttaaacattggaacacatatagataatttaagtaggtagatgtatgtgctgttataaggaagtggggaggagagaagagggaaccgaaatcatatgcacaaaaattttttttagaatataaataaaaaatgtggtagtctaaaatgtcaattcttcaaagataaagttaggctttcagtaacgttagaaatggttttctggaatatgtctccagtctacctaactttgaggaagtaaatactgtaaatagatgtttcaaacgcattttaaagcaatgatcctagcatgtctttaagctacagtattgtgctgtctttgaaatgtaaactttgatgtcttctctttctcttag

SEQ ID NO: 193 - DNA, 359 bp, homo sapiens
  CD14
gtagattctctgggatataaggtagggggattggggggttggatagtgcagagtatggtactggcctaaggcactgaggatcatccttttcccacacccaccagagaaggcttaggctcccgagtcaacagggcattcaccgcctggggcgcctgagtcatcaggacactgccaggagacacagaaccctagatgccctgcagaatccttcctgttacggtccccctccctgaaacatccttcattgcaatatttccaggaaaggaagggggctggctcggaggaagagaggtggggaggtgatcagggttcacagaggagggaactgaatgacatcccaggattacataaactgtcag

SEQ ID NO: 194 - DNA, 855 bp, homo sapiens
  FCGR3A (CD16)
gtaaggagccctggagccccggctcctaggctgacagaccagcccagatccagtggcccggaggggcctgagctaaatccgcaggacctgggtaacacgaggaaggtaaagagttcctgtcctcgcccctccccacccccaccttttctgtgatcttttcagcctttcgctggtgacttgttcttccagggcccatttctctaccctacctgggtttcttctaacctggaaatctaatgatcaaatcacactaaaaagtcagtagctcctgtggattacatatcccaggagcatatagattttgaattttgaattttgaaagaaattctgcgtggagataatattgaggcagagacactgctagtggtctgaagatttgaaaggaccactttctgtgtgcaggcagggcctcagctggagatagatgggtctgggcgaggcaggagagtgacaagttctgaggtgaaatgaaggaagccctcagagaatgctcctcccaccttgaatctcatccccagggtctcactgtcccattcttggtgctgggtggatccaaatccaggagatggggcaagcatcctgggatggctgagggcacactctggcagattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttag

SEQ ID NO: 195 - DNA, 2268 bp, homo sapiens
  TBXAS1
gtaggagtggaatgaatgaatgaaggagtgggcgcgcgccgtgctccggaggagggcgggtgagtggaggagcttcggaaagccacacgaggtcagaggcaccgaagccctgccgtgcacgccgcggaaatgcagcccccggggtggtcgctgggttcctgccggagtctggcttccacgccaccctgatcttctcctcctcgcgtctacactgtcccgagggccgccactccatcacttgaatgggagctcacaactctctgggtcctcgcttttctcagcgctctctcctatcagggcttccgctaaacactctccagactctccccagccgggagcctctccaggtttgcttctccagctgcctcccggatgcttggataggatgccctgccatcccctccaacttaggaagacttgttgagtctcctcttaggcactgtttttaaaactgtgggtcgtgacacagtaatagatcataaaataaatgcagcggaaggcaaccagctttcaaaaagaacgaaatggaatccaatagaaaatattagagtccattgcccttagtaagggcaactattgtttcataaaccttttgtttcgattataggtactggacaagtgtactggaccatgatgtcaattttatttccgtctgtgaatcgtgggtttaaaaaagttgaaaagctatctgtgtaaaataatgatctctaacactgtataactttcttaaaaacaacctcgtgattttctcactacttcttatcctttccggtctccatcctccgttcccatccttcagtccttcccaaaaaaggcaagctctttcacacttttttagtggtacatcacgacaccttcctctgtgtgatcttgtactctgcggtcttccttgcccccataatcaatgctacacttcctatcccttctcttaacctaatgccattcacgtttgcagaatcctagactactgttgctaaaaggacctcagaggtcatctgggccagccctgcccttttagagttgaagaaatggagtccagagaagtaaaatctgttgcccaatgtcacacagtattgaagcttaggaataggtctcgtggctcccactctagtgttttttgctccacaccggtcatttatctccttcccttctatcctccctgcaatctttaactttagtcttggcccaggtcatcctaaatattgccatcttcataatacgtggttctggccatgccacgctcctggctaacatcctctaaggactcaacctctgcaggtggggctgagtgccttggcttttcatcaaggcaggcgagtcctgacttagctttctaaggacatgagttcagcaagtcactgcttctctagccaaactggtttacttgtagcccagtgccagcttcacccatctctgtcactgtgcctttgcccaagcggtcactttcccacccactttcccagaggggtaccttctccatcttctctctttaagcagattctacctgtgcctggctcacctcctccacaaaacagcccgagatagcgtgggagtaagtcagacctaggttcaaatcctagctcttccacttaactatgtgatcttggtcaaaatgcttaacctctctgagcctcagtgtctgtattttgattatgagaaggagaacaatagtaatgttctccttttgcatctgtaaatctctatcatagccttcaccacacaggtatgtaactccctgttaatacataccctacatgcatgtctctctgcccctttctagtgctggccattagaacttgctgccataatggcagtgtagctacttatgtctgtggagcacatgaaatgtggctagtatgactgaggaactgaatttttaattttaataaccatgggtggttagtggctaccattttggacaaggtgtctctagactgtaagctcagtaaggacagaaaccatgtttttcttgttttggattgtatgtccaataactgcattgcctaacgcatggtgagtactgcacacatatttgttgaatggatgatagctgcaatttgttgcatgctcccttgtgtccagcacaacactacaaactcgataaaaggcatttcatatagtcctcatcacagcctctgaggctgggttagattagagagagagttttatatagagggcagacacatagtaggggttcaataaaagttattattattctaactcctccctctgaccagccccactgacttttgtctcatctttccttag

SEQ ID NO: 196 - DNA, 602 bp, homo sapiens
  DOK3
gtgagggggccctggaggggctggcctgggggagatgcaggccagaacacacaggtccctgagtgttgggctgaggagcctgggctgcaccccaggaaggggagagacaggttggtcttgggttgtggagagtgctgcatggaggcccagtggggcagtggcccctggggggatgccaaaggctggtgatgttcagtggctcgtgaggtggagaggagcaggtgaacacgagagagatttcagggagaaagaaaagcagcaagatggggtgaggcgctcaaaccaggacgccgagtgggaaagccaggagttggctagagaagggtcctgggtggaggcagggccagggagtgtcaggcccagggtgcgcagctgcccggggcgggacgggggaggggcgggaactcatgactcggggagccagactgcgatcagacgcgcgtgcccagctgaaccaggtgcgtgagaaggctgccttcaggtggccgggggctccctccaggtaggtgtgggcaccttgggcagccagaaccccagggaggaaatacatgctgcccccagcccctgagctgaagacaggctcccctccccccggcctgcag

SEQ ID NO: 197 - DNA, 14420 bp, homo sapiens
  ABCA1
agtaagcttgggtttttcagcagcggggggttctctcattttttctttgtggttttgagttggggattggaggagggagggagggaaggaagctgtgttggttttcacacagggattgatggaatctggctcttatggacacaggactgtgtggtccggatatggcatgtggcttatcatagagggcagatttgcagccaggtagaaatagtagctttggtttgtgctactgcccaggcatgagttctgatccctaggacctggctccgaatcgcccctgagcaccccactttttccttttgctgcagccctgggagccacctggctctccaaaagcccctaatgggcccctgtatttctggaagctgtgggtgaagtgagttagtggccccactcttagagatcaatactgggtatcttggtgtcaatctggattctttccttcaggcctggaggaatataataactgagacttgttttatttctgcagagggttctaagccattcacttcccagatgggccaataatgctttgagtaatctggagatcatctttaatgcgcaggtgaatggaactcttccacagagggatgtgagggctgtagagcagagtgaactccctgaaactcagacgtcagctctttgtctctctatctctgaacacccttccttagagatcccatctctaggatgcatttctctgtagttagtttctaagtctcttgttcctgttctgcctttatttttttttcctggattctaagccagtatccccacttggctgtcttaatgtagcttaacatgtctgtaatcaaaatgatcatctttctgagattcaaagggctataagggactttggagagaatttcattcagttttcctcaaactagaataatgcttgcactgtctgtaaaagaacaaaagtgtcaaagcatccttttgttcactaaatttccttttttattatagtgttacttaaatattaggaagtaaaagtaggtataaacttcttataggctgttattatacaactatatgacccatacatatttacaaattaagtgcagccaaaattgcaaaatcaataccattcaaattaataccttaaatgtggtgaggcagctgttgttcaactgaaaccaaattataagttgcatggcagtaaatgctatcatgctgatcattttgagtttggccagtctatattatcatgtgctaatgattgaattctccacccatttttctacttgtatgaccttaatttgatggcacctgttccatcctcatgagtttgctacaattatactggtgccaacacaatcataaacacaaatataaacttgggctttgaaatcttgtgccagaacttggctttaaagtaagcatttaaaaaatccatatgtgtttattagactttgtttagatgactgttgaaatgaaaacaaagtgtttaaaatcctcttagagaacttaaatataatccctcagcaatatgtatacagatcttcctttgagaaaaactgattgtgttcagcctctcatgttacaaatggggaacctgaattctgaggtctctagtgagagaacagggactggaatctgtggatcctatctgttttaataataattgtaaagtataatagataatattatattaataaaataaaagcaaacacttagaatgagcttccatgtgtgaggcactaactgattaggcattattaactagatttattccttttaaggccccgcgatgtactgttatttccacatgttgtagctggggaacgtgctactcagagaggttaagtaacttgtctgaggtccacaccactaacaaggagcacaggtagggttcaaatccagataatctgactttggagctggcactctaactcaatgtgcctaatcgcttttcagtggtgtcattattttgcctattctccatctgagaatattgaagtttctgactccttccttgcctttctccctgcctcccgtggttatccccaggtcttggtgttccagtcctctatgtccgtccttactcttattcctttgctacagtgtgatccagggctcctgccccttcttatcctggtagagggggcccacttgctgggaaattgtctccgccatggtttatccatgttgtgtgtccattagtgagtagtgggaagaatcatatcatgttggcaatgaaaggggggctatggctctggggtagtctagtctgaacctcttattttacggatgagaaagctgaggtacaaagcagggaagggatttcttgaggtcacccagccagcaactgagctgcaaccagaagctgagatccccaggactagggccgagcctcattctgtcccatcacagtgacttttcttccctcctccaaactatttttattttttatttttttgcagctgcttagcagcttgaagttagaagaaagggcagggaaaaggttttccgtgcttagccagggaaggaatcctgcaacaggatgtggggttgggtcattcaaattgggccagactccactggtcttgttgcttcttgcttggtattgcagatgggtttaaaagtgttaggattagagagataggcaggtttagccaaaggcagtttgtagccttgtggcagagttctttttaaagaaggaagtgggatgcaacaccctgacacaaaggggcttaagttgttataccactgcctgctaacctgttttccttaactctcttcctgatttctaaaggaagtatattttgctgaatcagaaagaaaagtgatttatttcaggttgctgatgcttagattgttagagttggaaagatctggcttgcatcttgtacagctgacagaactggggctcaggggggcacaggtgcccagagttggtcagtcaggaaagtagcaccagaaccagtctcctggtggccctacagttgcagacccttttttgctttgctctctgtgtatactaaagcttctatgtctctgaatctcaagttctgactggtagctactttccaatccacctggcttagatttctagattatattgtttagacgtcagaacctcttaagggttttggggccacttgttagctcacatagtgagaaccagccctgcccattaggtaggggaagaagttagcagtccatgatagctgttgcctgcagcgtatggatgttcattgcacagttcctgtctcctgagatcctggagtgtatacgcttggcctcagagcccagcacagagcctggcccttgggacatgcttagtaagtatttactgaatgagtgggaaatgtcttaaggcccattagtttgcaggtcttgaggaggctcccttgcactaggaagaatagaaagcatacataaagcctgtgtgctgccgccaggaagactagaaacgctatgttcagcctggagctgaatggtataccccagagcaaccctgttgaaaggcagtgcttgccttttcattctgtgtcctggtttgctggtaactcctgggtcccctgcctctcctgtacccccattgtgcagactgaggggggaccatcagccagggttagttttccgctgtttctgttaggcaaagaataaattgaattgagttgtgaaagttgggtgcaaagctcagtttgggtccaaagtaacagttaacttgtgtgggtggcaggtattcagtacaaacagggctggggacaggaaggggaagagaacttcagagctttcacgatcctcatctggttttaggctgatccagaggccaaggtccccatggaacaaactggacaaagtgagggtggccacatggcctcttttcttttgcctttattattaattttctcaaatagatctgactagtcatgtggctgggaaaatagttaattgtgatttttttttttttaaactgagtctcactctattgcccaggctggagtgcagtggtatgatctcagctcgccgcaacctctgcctcccgggatcaagcaattgtcatgcctcagcctcccgggtagctgggattatgggcacacagcaccacgcctggctaatttttgtatttttagtagagacatggttttagcatgttggccaggctggtcttgaactcctgacctcaagtgatccacccacctcagccttccaatctgctgggattacaggcatgagccactgcacccagccagagtaccactatttgggcattctttaatgaaaaagaatgaactatccaaaaattaaaactcctcatttatgagcttttagagaattttacagagtagatggaaactctctgcatcctttccccacttctagtttcacctgacacatttcttccctgtccttactcctgggccggcagcagtggtcatgattccaatcccagcttggccaccatctgcctcagtggcctaggaaaactcctttctccagagctttagttttctcttctacggaatgaagaaagttaaaacaaatagacatttattgtttcatttggataaatatctattaagcatctattacttgtggtatggttagctgggtatatagtggtgaagcagctgggcatgagtactgctttcgtagagcttacagttcagtgaggccagcagatgtgaaacatatcatcacacaaataaaaatataactatcaactgtgatgaggattatgaaggaaaaaatccggcaaactatggtactggtgttagatactagcaggtgtgggtagggatttcatttagattgacaggttgtcacattaaagctgagagccctgaagttcaagcaatggttagccaggcaaagatcagaggcttagagatagggaaatccattccaggcagagagactgggggtgcctgtcccctaggtcagggaacagaagaaagccagtggcactggtggagtgaataagactggcgggggatgagttggtagtagacatgaccagatcatttagggccaattctcctggggaaggagaatttaatttaatatttatttatttatttatttatttatttatttatttatttatttatttttcaagacggagtctagttctgtcgcccaggctggagtgcagtggagcaatctcggctcactgcaacttttgcctcctggtttcaagcgattctcctgcctcagcctcctagtagctgggattacagacgcccaccaccatgcccagctaatttttgtatttttatagagatgcggtttcaccatattggccaggctggtctgaaactcctgaccttgtgatcctcctacctcggcttcccaaagtgctgggattacaggcgtgacccacagtgccccctgagaatttaattttattttatgtgcaagaggattccctgaggtagtcaggccacattgtctggtgactcttgggatagagggaacttgaatgacaaaggcccaagaaagcaattgtaatcattacatatacatggaccattttatgctgttttcttctttcatttaacattatttagtgtgcgtgttcacatatttctaaatcatcttctgatttagaataatgatttctgatgtgtaggctgtgttttatagttttgaaagtaatactttgatatccattactttcttgattctcacagcaattctgaggtgtatgcgttgcaatttctgtttcacagatgaagagagtattgttaataagttaatggccgggcatggtggctcacacctataattccagcactttgggagaccaaggtgggcggatcacttgaggccaggaatttgagaccagcctggtcaatgtggtgacacccatctctactaaaaatacaaaaattagccaggcgtggtagcacttgcctgtaatcccagctatttgggagggtgaggcaggagaatttgcttgaacctggcaggtggaagttgcagtgagccaagattgcaccactgtactcctgcctgggtgacagagcgagactctgtctcaaaaaataaaaagttgctaagaggagggctgggatcttttggctccaaatctactgtgggatgatgcctttgacattcctgatagctgtgcagtaatccattaacacagtttttataagttcaaaccctgttgccaacatttagattgttccatgtgtgctgttacaaataaattactataaagattctatacatttaatcttttattatttttgtattatttctgtaggccaaaatctgaggaacaggattactaggttgaagggaaatggcccttgaagtgtctgatcagatgtctttccagaggatccaaccaatttaaatagccaccatcaatgcatgagactttgtagttcagggaaggcaggcctggttttaaaaatcatttcccctctctagcatttttctgatgtgatccttaagatttcactttagttttcccaggtctcattggcatgtatgctgttagggatgggtctaaaattaatttttcttcacattcatatcatgtcatcccagtgattatttaataaataatcacttgattaaatagtgattccttttctagttatttttgggacatttattaaaacctggatatggtggctcatgcctgtattcccagcactttgggaggctgaggtggggggattgcttgagactaggagttcaacaccagcctgggcagcatagcaagactccatctctataaaaataaggaaattagtcaggcatggtggtacttgcctggagtcccagctacttggaaggctgaggtaggagaattgcttgagtccaggtggtcaaggctgcagtgagctatgaccatactactgtactccagcctgggcaacagagtgaaactctgtctgaaaaaaaaaaaaaaaaaaaaaaaaaaaaaaaaaaaaagatgtgtagggagcaattttggagttattcatttggtcatttgatatgtagttttagttttggtgctgatagagcccagaatgtaccctgaatttgatgaacattctgatatatgggggagctcattgtcccccacttacctttttgcctctcagaatatcttttgatatttttatctgttttttccccattgaatgttattaccttatcaagctcaaaaaagtaccctatcgctattttaagttcagttgtgttaaatctataaattagcttgggaaatttggatattaaatgaactcatgaagaagcagagtttagctctccttaattctcatcttcctttatttatcctactacagttctgtggttttcttttatgtaagaagcacatgtttggctaagttaatgcctaggtttttttgtttatgtgtccattctcactgtggatagtattctcttttccccacattatattaatttaactggttttcagagactaatagcaatgctattatttaggagaatttaccttggttctgattaacttacccatacttgcaaatcatttgcagctttttagttaactttgtgagttctcttagatttacgaccatgccagaaacagaaaggatattttcatctcttcctttctgatgtttattcttcttgtttcctttttttatcccccattatattctcaagaatctctcaatactaagaaatagcgacttcatttttcagcgcggagtgcattattttggctaccatgattcagaagcctcttgcctaaggcccaattttattctgctagttttctctgttctttgtacatggcccttgcgctgccctaaccttgaattaacgtggctaaatctcaagaatttaagagcaccgtgactgtgtcctcaggctagggagggaaatgggttcacagagtgactggattgtggtctatgaacttcggcagccagcagcaaaagtcaggcatgaataatcaagtggacagtgaacatctgtagtgtgggagatgttggcataactatgaatgatgattcaagagtggtttgatgcatattgaataacatgatgataagtactagactctgtgctaagccttctatgtgaaatacatttaattctcataataactctagagcagtggttctcgaccggggccggttatccccctaccccaccccaccctcacccttccaccagggacataacatctggagatatttttggttgtcacaatcctgggaatgtatgtgctgatatttagaggttgaggtcagggatgctgctgaacttcgtagaattcataggagagtctctcacaacacctatctggccccaaatgtcagtagggtcactatcaagaaaatctgctctagcagtgcctgctcatattatccccatgttgaaatagcaagatgggaagtgcaaagtggtgcttcggtactcttggagcagctttgactttggtgagaaacgccttttaaaaacaatgtttcttcccatcttcccaccccatggggaggtgtggggttgggtgggtaggcaccaaagcaagatttagaagagttttctgtaggaatttataatggtaaaggatcaacttcatttccaagctatttatgagggtttatgtttaggaaaagtgctaagcttagagaaggaggagaaatctgattttattaatgagtgtagccataatggcatatcctggcagaagtcaactttggtttctagagggaggctattatgaaaagaaatacctggaacattcccctgggtttggaaggtgagttctaggttcaatgatgggaagaattttagaggtccaagataaaagggcaaagattaaattttgtctctcatgagttctctggctcaggtggtgtgaactttgcagacagtctctttaattcactcatacatgctagtctcccagctcagcaagggctttgagagagcaggtgtctgtatgctctggtaagtgaaggcaaagtgcataaggaggttggggtccataatggcgaagagaaggagcccttcagtcagagtggctttgaatcttggctctgccatttgccaatcttggaccattgggcagtgtattaactctttgaatctcagcttcctcttctgtaaaatgtgtataacaagagtactaattggattgtttgatgattaaatgagttaatgtgtataaagcactcacaaccctggtacatagtaagacctttcattattattatcatcatcaattttttttaacctcttttcctgatctgcttacactcaccagcttcagctgctccaaatggcttgtaagattttttgtttgccctttgctgtcagttgccatggggaagatccattcatttttttcagtcaaccaacatattttgagcatctgctgccctacaggatcctagatatgggggctgcagagatatccaggaacataagccttgattaattgggtcagatcagtgctcagcagggctggcaagtgctaggtttcttttaagtggcatatcttaaaaggtatatgtcctaaacatagctttgtgatggcagcatgatgggtacaaaagcacacacttaagtgtcagtagatctgggttcaaacattggtgcagtttcttatggctcgtaacttgttcaaacctcagtttcttcacttctaaaacggtaatgatacaacctacctcacagggttattatgaattaaatactggagatgagatacacaaaacgtcttgagtacacagtagctgcccaatattggctgtaagtattataaatctacaagctgtgaattaattttacctctctggatcctgttgatatttctagaccattccacctagtggggccatttcctacctgagtcacccgtggtgtcaaatagaatgtcatgtggcctcctgagttgggtagaattggctgctcatctcaaccccgctactgactatctctgtgatttacccttcctccagccttagccttgctacatataaaatcaagacaataatgtttcctatctcacagggttgtcctgaggattaaattaagtaattaatataaaatgtgccttgtacatattgggccctaaataaacagtagctactatttatccttaaagtacaaatggtagtttcagagcttcaaggctgatggctatttatcttactcatactctttgtttagcttcatttttttcccctaatttcattagtattttcttttctttttttttttttttttttttttttttttttgaggtgaagtctcactctgttgcccaggctggagtgcaatggagcgatcttggctcaccccaacctctgtctcctgggttcaaacagttctcctgcctcagcctcccgagtagctgggattacaggctcccgccaccatgcccagctatttttttgtattttcagtagagatggggtttcacccttttgaccaggctggtcttgaactcctgacctcatgatcaacccacctcagcctcccaaagtgctgggattacaggtgtgagccaccacgcccggcctcataagtattttctaaatttatttacagtcatgccatttaaaaggaaagttgtattcctgtctttgttaatatttataagtgattttattcagctacaagcttggaatggcatataattttgtattctgcttttttcacttaatattacatggctaatgatttctgtgtttcataaacattattctgatgatggcatgatatattgttgagtacatgtaccataattgaatcatttccctattgctatgcaattaagttgtttccaatattttgcaattataatgtttcaatgaatgaataactttatgcatatagctttttgatatcttaagttcagtttcctaggatgaatttccaggaatagtaattgggcaaatgggataaacatgactcttgaatacgtattgttaacattgctttcccaaagggctcaactgatttatatttccgtgttcattatcttttaaaccagctcatttactcaccaaacatttttaaagccattatcatgtggtaggcttagtaagaagaaagtgaccctaagggagaagcttatatataaatagggtccctggtgtaccaagtgctgatacagacacaaagtacctggggaaattgagatgagggagtcctggctcagctgggagaaaagttcattttcatagagtcatggttttgttctttggcagaaagaaaattgctttcttccccacccccacccccagctttattgaggtataattgacaaataaaaattgtatatctttaagatatgcaatgtgatatatatgtatatctcaacttaaaaaataagctacagaataaaaaggtgtttgctattaaaaaaaaagaaaaggctgaatgtcattcccaagcttggaaatttgagtatgttgcctctttgggattatttacagaaatattagcaagaccagccccatctttggtcttgagtactccactgtcagcatgctttcttccagagagggatccatttgcctttatttttcattctgttgtgccgtctatgcaaactattcttgatagttttatggtaacagtgtttttttgttccatgagataatttatacatgctcattgtggaaaatttagaaaagacaggaaagtattaaaaacatcacttttttttttttttttttttttttttttttaagagacagagtcttgctctgtcgcccaggccggagtgcagtggcgtgatctcagctcacagcaacctccgcttcccaggtttaagtgattctcctgcctcagcctcccaagtagctgggagtacaggcatgcaccaccacgcccggctaattttgtatttttagtagagatggggtttcaccatgttggccaggctggtctcaaactcctgacctcaggtgatccgcctgccttggcctcgcaaagttctgggattataggcaggagccactgcgccagccacacctacgttcttatcatcctagtacatccactgtcattatcttgctgtatttccttctgcccagtctcactctgatcatgcagtggcgtgatcatgcagtgatctcggctcactgcaacctaggccttctgggttcgagtgattctcctgccttagcctcctgggttcaagtgattctcttgccttggcctcccaagtagctgggattacaggcatacacccccatgcccatctaatttttgtatttttagtagacacagcgtttcactaaaattttgtatttttagtagagatggggtttcaccatgttggccaggctggtctccaactcctgacctcaggtgatccgcctgccttggcctcacaaagtgattacaggcatgagccactgcatccatcgccaaaaagattttttaaaagagtttaatgtagaaccatatcaaaggtctttggaaataaaaaacagttttttaaaaatatcagaaataaaacaacaaataaataaataaataaaaacacccaaaacaatctgaagcacgagcacctagcagaaaggttcaattatgatctattcatagagtggaatatcaagtagacattacaggacatgttttaagattatattttatgtcatgggaaatgctctcccagtatgatgttaaatgaaaaaacagaatacaaaagtatatatgctgcatagtctcaatattgtagagaaaaaatattatttatgtatgcatgaaaaaagacaaaagatgttaacagagatccattgttacttcagtttactagggattgtctctgggaggtaggattaaggtgatttatatttacctttttaaacttttctgtatttttttattttcaaattttccataaaaatataaggacttgaagatcaagaaaaaatttctgctttggctcagtgcagtggctcacgcctgtaatcccagcagtttgggagccctaggggagaggatcacttgaacccaagagtttgacgttccagtgagctatgatctccggatcgtaccgcctggacgatggagcaagaccctgtctcaaaaaaaaaaatctttgctttttttttttgtttgtttttgagacggagtctctctctgttgccccagctggagtacagtggcacaatctcagctcaccgcaacctctgcctcctgggttcaagcgattctcttgcctcagcctcccaagtacctgggattccatgcacccaccactatgcccagctacttttttgtattttcagtagagacagggtttcaccatgttggccaggctggtctcgaattcctgacctcagctgatccaccggccttggcctcccaaagtgctgggattacaggcatgagccactgtgcccagcccaatcttttgctttttttaaaaaaagaagacaaaaagggattttataccagtattatcttggctgtgtgactctgaagccacagttgtaagttataattactctgaaacacaaggccctgtgactcttttgggctctttggtgtttatcttgattacaacgttggaatatagaaatgaaaggaatgggagaggtgatagacttcaggcagtgtaactagttgtctgaacactactggctcaattatattgtgtctagtgatttccatcttgtccgtctgctaatttatcgcctggtaactcactgaggcagggttttcctttggagaaacctcattgttttaaccagtgtatcatgcttgtttagaagttcaatgatctttttaactcatcggagaagatgatgaccagacctggacagatggggaaggactttgcactctctctttacagtcctgagtgcacacaggtcaatatggaactatgtgtgaattttcattgtctttgagagccctcttctctgccccatagggagcagctttgtgtgcaattagaggagcaagggttgtgtgtatttagcacagcaggttggcctggtcctctcctctcaacatagtcaccacatacctggcactatgctaaggctgggaatgcagacagatgggtgcctgctttcagagtgctcaatgtgctgaggaagccagcaacagaaacagatgatttcaggagctccaggaaaatgctacaggaggagtgtgcctgggttactggagtagcacaggaggagggcttctagctcaggctgagattttagtaaaggaaattatgccacgatgaatcctgaagaatgaatagaagtgaaccagataaagcacgataggaagcatcttcccttacctaagggaagacacagaggtatatggaatggtatgttaaaaggttgggactccaaacagttctgttaaagcttagagagtggtgggagagactggagaagttgattaattagtaaatgaagttgtctgtggatttcccagatcccagtggcattggatatccatattatttttaaatttacagtgttctatcttatttcccactcagt

SEQ ID NO: 198 - DNA, 1448 bp, homo sapiens
  TMEM195
gtgagactttattggaagaggctactcttattcttgctgctttcttaataaatggtaaggtcaatctccttagggttttgttcactttcattattcaaatgattacattctaataataataattcagtttcttaaaggcgtgaagcaaaagtagaatttccacttgaattttcttacaatccatagccatcttcacctgtaaattccctcttttatatgtcactccagaatatgacaaaaacaaaagtttaaagttttcctggactccatggcattgtaaatgtagctattggaagctcaatgacaaatagttacaccacatcagtaaactttgtaacaatttcatttttaaatctttcacttgtccaccccttccagggaaactatttcctctttcatttggtagagtagttcagcagaatgaaggatatgcagcttgaccaggtccagctctgacttcctaagtttgaatttgacccaagttttctttgaaactctgaagtgctcagaggcacatttcacttctggttcctggctatgattggaagtggaaatatccagaattagaacttgaatactgaagttccccaccctatcaaagttattcctacaacactcactagttcatggcagaaatagaaaaaaaatgacatcatagaatatgttattgagagttcagtttagaaagcttcaaaactcatgattagcttccaaatgagtaaaatccgtatctttttcaagattagatattacactttctttctgagtttttaagttaaaaaaagtggcactgatatcttcataaattattattgtttatactgcaatatcaaaccaaattcaatgctccatcaccaatagtggagctttcaaaacatctcttattatgtgaaacaaaaatcaaaaccaaatgaataaaatctgaatcatgttttaagcaaacatgtaaaattaccatattctctaaaattctaagcagaaaattgtctctactctttgaatttcatcattagaaatagttctggtaagtctaatatgaaaaattagaattatgtgcatctttaaggaaagcctcttgaagtgggtctaaattatacatgtagagacaatagacctctaattatctaccttattctaaatgtattttttaaactaatttggaagatgttcctcttaatcttataatattataattatcgtggttatatctatgattacatttcatattcaaattggatgatagaatttgagcacatgtttagtgtatgtctaaaataattagacttctttaccactgtgtaggaagtaatcctctaaaatgagtgtctaccatctcctgcagggcttcccaaatctgggcctttcaattctgaaatgactaatcaggaaatgtaaaataatttcatatggaacataatagcatctcttttctgcaacttcctttccag

SEQ ID NO: 199 - DNA, 3633 bp, homo sapiens
  TLR4
gtatgtggctggagtcagctcctctgaactttccctcacttctgcccagaacttctcactgtgtgccctggtttgtttatttttgcaaaaaaaaaaaagagttaaattaccttaaagactcaagaagccacagagatcaaataattcattgttacagggcactagaggcagccattgggggtttgttccatttggaaattttgagtgctaacaggggcatgagataacatagatctgcttaaggtccctgctctgctaccttgtggctctgtgaagaaattatcaaacctgtctgagactagttttcgcatctgtaagagaattataataccttcttcactagagagtaagcagactgcttcagtgtcatttcttcccactggtggtctttacactcagcttcaagcagtcaccctgctcctttcaatctcaggaaaaagatggcttttgtgtgtgtgtctctagagaaagaactttctaagtgggtgtcagacttctgtatgcagtaatatagtttagtccagaggatgaaaaaaataagagaatgaaaaaggaaaagagagagagagagaagaaaaaagcaagagggaaatatgtataatgtcagctaatgcaacagtttctttcttagtgaaataccaatcagctggttggtaatcttattcatgatggatctcttttgtttttcccctgcgcagacttcacagttgctttagaaacccatagtagagccgaacagctaagaaaatgatttacagtgaggcagggtcagaaactcaagagagaaaaagccagctgcagtcctgaagttgaggatataggagaaaatcaagtaatatttagcaaagactaattcattatcttgaagccatcccttccctcaattccctgcccatagtcctcctccttgtcctcttctctgtatccctctgctgttaggttaatggagatagattttctaattaggctcactgcgagataaaaccacagccaaacttgacttcttttccccatgtaccttttcctgtcagtccctgaagcctgtccatccctgcccatccccttagttccactgtaaggcaggccctcatttcccctggcattgactcttacacactaactgctttcctgattccagtcttcttcctttaattcattctgcacgttcttgtttgttatgtacttgcatttgttgttattatttttccttaggcttcaatctaacaaattactctccttaaaaacttttaataactctccattgccattagaacagctttctaccacagggcctttgcactggctatttcttctacctagaatgctagatcagtgctatccattggcaatattatgtgagccacatatgtacttttaaagtttttagtagcctcattaaaaaaagaaacaagtgaatttaatttcgataatagttttatttaacttagcgtatttaaaataatgtttaaaattttaatatatatttacctattattgatatttttacattccttgtttggtactaagtctggaatttagtatatgttttacatttaccacacttctcaatttacactattcacatttcttgtgtttgataactgtgtatggctagtgactaccgtattggtcagtgcagcccaagtccttttcatgctttaatcactccattcagatctctgattaaatgtcccctcctcagggcagtcttccttgattgccccatgtagagctctccagcctcacttatttgcctcaaatccccttatactgcttaatatttttttttctagagcacaacattttatatttttgtttgtttattttctctctctccctttgtaatggaatcggtaaggaggcaggatcattgctggttttatttaccactatatttccagtggccagcacacagtagccgctagatgtgtaagtgataaatgattgaaataattgctgcaggacaaagtctgaggccctcctgatctggcttgccctcttacttagatttcaccactcccaccactcaccagctaatctgagtttgttttccactctttacgtgctcacgttgtcctctccttaggacatgtttttcttcccctttccacatatctaaaccttactcatcttccaagacccactttaaaatcttccttttctgggaagcctttcctgaatccagacttgatctctgctttctctgaaccacagggcatattttctaagcctattttatggccccttgagatagtgttagctttgctcctatctaaactcttactctagactgtgagtccattgaagtctggagctgcatcatatttttctttgtaatgcccacagcacttggcaggaaatgcctacaatttggacttaagtaaaccttcatttaatcagttattcaatcagttagtgattcagcaaatatttattgagcaccaaccatttgccagacaccattctgagtgctggagacaaagcagtgggcaaacccatcaaacttgcaatggaatacaggagatgaacaatacgatgagaacaatcagatagacaacataatgttagatggttgtgcttcccgtgaaagggaataaaagagggcaaagaaagagtgcctggcactgtttctattagacaatattgtctttgaggctccatggcttgcaacatttaagcagacatacgaatgaagatctgcatgtttgaactctgactttgcgcatattacttcatttctttgaatttccattttcctcatctttaaatgcttatttgaagattaagtgaaagtatataacaaacaagaactatgcaggcatatggtaagggattaatgatagatgataataattaatgttgacatctattgatcacttatactgtagcgggcttttaaataaactctttaaacaccttatctcatttaatccttcaaacattctattggtttcaaacaacagaaaactacaattagctggcttctgcaaggaattttgttggaggaaatgagagcattcagaaattagatgggagcgttagagaattaggcttacaaagaatgtgggaaagtaggctagaaagcagtgtaaaaacaaagacagcataaagcacttgaccttatttactaggttccaccatgggaatccatgcactctaaagatttccccctatttctacatcactttgctcaagggtcaatgagccaagaaaaagaatgcagttgtcaaaatctgggccatgactaaggaaggtctggacatcttgactgccagacagtctccccaatgatatggagtatttaaaatgatactggatattttatttattttttgtattttcaacttttaagttcagaggcacatgtgcagagcatgcaggtttattacataagtaaatgtgtgccatggtgatttgctgcatagatcatgaaaatatggaacgcatcatggatttgtgtgtcatccttgtgcaggggccatgctcatcttctctgtatccttccaattttagtgtatgtgctactgcagcaagcacgatattggatattttattacctacattttacatatgataaaatgaggctcactgaggtttttcttttgttcgttttattttgttttgtttttaaag

SEQ ID NO: 200 - DNA, 422 bp, homo sapiens
  MR1
gtaagtgttttgagcttattgtgctgggtgctagagtagcagggaggacagacctaagttgaagattacgggacaatctaagtgacacctgctatctcagacagagacttcagcatcatagggagccagagcagggagttccaagccgtgggaactgtgactctttcttactgacccttaagcaaaattctaagctccagggaagcagaggcagcagcttgagctccctgattgaccagatatgattaatctttgtttctctgtgctttgaacatgactatgctccataaatatttaaaaattgggaatagggtgaagatagatacaccaaaaggagcaagcagtgtctgaaaccagcaaagacttaaaagttaccgaagaaggtgttcatgctttcattttatcttttttttttaacctag

SEQ ID NO: 201 - DNA, 393 bp, homo sapiens
  FCGR1A (CD64)
gtaagttggactcagaggggacagttagaagggtacaggctgtggctgttgtgagtcaagagttttgtcttcctgtggtaactctgggtagaactcatgagtatgaagcaacttgtatctgtgcttccatggtttattagagcttattttatgaaaaggatgggaagggcaaccctgaggtagcattaagcctggacgcaccgcagtgaagtttccttgataaccacctgtagcttgttcagttctgttagtactggattttgagaaagagaaatagaaactcaagagatctgagttgatccctcagagtctacattaattctgtctccccaattctctcttcctcattattttccttggaccaactgatatctttattctctgatctcttgc

SEQ ID NO: 202 - DNA, 1273 bp, homo sapiens
  CSF3R
gtgagtaggggatccctagagaggggctgagccttgggcttgatggagagctggaatcctggggccactccccacagctctgccaagtgttcatgtccggggtcctaggcctggggcccgggattcgtcctttggaaccccagccctggacactgagtctcttttcacaggatgcccctacttttgggggtattgcaccagggaagtcactcacagccaagtatctccttggggacactgagctgggctggggacaggcataatcattctcccctccccatgcctctgtctctcaggacgtgtgtgtctgtgagttccagccgcgcaacccctccccgtctgggtctctatttcactctctgtttccatctttcctcctcagcttctctctggcttgctatctctctgagtttctctgtctcgcatatttcctgctatctcttgaatcttaaactctcggtaacagacgcttcccgggctccaggcctccgagtgccccccccccgcccactctctgggtcggcgtacattgggccctttttctctgtctctcgatatctctctggcctcaatcgtcctcttggcgagtctctctgtcgtttcagtctgtgtggatttcagtcaccgcctcactctgtcactcttcctgttgctctctctttttctttatctgcagcatatctggaaatgcctctcccctctgtttattcccagccccctcctccctccccacccttcccacagaaagaatctcgaggtgagagtagaaacagcaagggagcgaggggtgtgaagtggggaagagagtgtgaggggagagggctgagcgtgatgagggagaaacagaaagagggaatgtgaaggcagccagcccaggagacatcgagctgtgctgggccacaggagtgagcacctgcagagcccagcagagatggccgcagtgacaggagggccagggggcccaggagttaggaggagaggatccagaaagggggctttggaactgcgcagtgtggcacagacaaaagcaaggtggcagtcctgggaggtgtgaccacacaggctgtgagggaggcaggggtgaggggaaaactgaactgtgctggattctgtgagctgctgacctgctatttatggctcgtgaaaataacctatggttgctgagtgccaactccatgcaaggcacagggctaaatgcctttaccaaaataatctcatttcatcctcacaacaacgctgaggagtgagtgctaatgttatttctttcccacag

SEQ ID NO: 203 - DNA, 29639 bp, homo sapiens
  FGD4
gtaagtggtttcattttctagtagatatatttttttcttccccagcgtgggtcactttataacactgcatgccgagggatgtaaaagtggcagagtttggttttcttttaaagttaataaagctcacattggttttcaagttcaagattaaaatatgtgctattgatgggttttggctctccaggaggcttcttctgggctaaccagatacaaatgagtttgatagttaagtttctctggcttatttttattatcataagtaaagtttggaaatttggtcattgaggggagaaatatagagaaagaaaggaacagttttctctttttcctcccatttctggtgtgaacaaatgttaagaaatatttaccactaatcatttttctagtgctactgcacggagcattgttctgttaaaacaaacaaaaattcccaccatcccagtcttgttttggtgaagttcaggggagtaagtattatttgtttttctgtaaaagacaacattgtagagataggcatgagtttgttttttaaaaattgttagtggtagtagtattaagccactgtggtctctttgagtgttgtagagagcatattaatgagaaattgagtctttgaaatagaaatgtaattcaactgtgtgatagaaatgtaatcatgtgacgggaaatgtctgctaaaaatatttttaatcactgaaatgggttcattttgtttattttcccattcgtttttgactggatataagcctcactgaaaaatgtccacactgctgaacaagataaactacaacttagaaccggcagccttcacagttaccagtattctttctgatggtggtgtggctctttccttccttaagtgtcaggggcatacgctgagtgtggaagcccaaggttggggttggtgacagcactgcctcaggagagctgaggcgccactcctgcagctgcacagctggctccgttcccaggttctagtagctgcgggagggttaagcagcccagtccatgcctgcccctgctccctccatcactcaggcagaagcctgaattcttttgcaggcgttccaccactcttgcataaagtttggaggtcttgtgatctctccaaaatttaccatcatactctaaatatgaaattaaaaaaaaaaaaagaaatgcagcaaaagggaagtgttttctcaccccaagcagtgaagttttaggttctctttttcctgaaatagcttctgtccattctcatttatcttcaaacagttagagatgattggagagacagtattccctcctggaaggcagctcttaattagagaggaaaatatttctagaacaggaagatgaagcttgagaggtgaatgttgataaacatagagaacttgttggctcttgttttctgctttaatggacagcaccctttagagagtggccactacaacaatatggactttataagttcctttttaggtttttaaactgagaagacagtaatatagcacttcctgcctacactactggataattttgttaaatcatctaagaaaaattgtaaatatgcatatttagtgcacatgttattttataacagttttttttttttaatgacctgaaaaaacttctctttctaaagcttacgctttcaggcaaactcctttcacatgaaagaaatgaaatatgatagagtatgatgaatgactcaataggaaatattatgcagttctttggaagaatgataacttggtgttcctttaataaacatccaaatgtacatgtaacctaaactgcaaactaggattaagatttaaatttaatcagttcttcttagatactagaatagacttgaggaggtcaagagtagataggagactcctggaataatccagggaaaagttgaagtcatttatttcaaaaataaaaaggtattttcttagatgttttgtggaccatccctgcccacttcctgagccaccttctgataacctttactgtaggcacctgttttctttataaaataacaagctaatttttattggctaaagtacatttgtaaatgtcttcaccctggtttgtgtgtgttctctgatttatctcccaccacagcacctgctaagctctggctagaacagatccttccatttttattccctggcgaattgacacaagttagttggttatacgttgaagcaggcagaaggaaaacacttaggagaagaaaaatagtgttaatcgtagaatttggtcattctacatggtggttattataataaaacttggcctggggaaaatgagtctttcaccctcttagagaataagtacacacaaatacatttttaaaacatttaaacaaacaaacaaaaaagactgtgtcactgtggtcttgtcttttccatgccattataaagcagttatttcttttttttctctctctcttttcttttttttttttgagacagagtctcgctctgtcacccaggctggagtgcagtggcgctaagctccacctccggggttcacaccattctcctgcctcagcctcccgagtagctgggactacaggcacccgccaccatgcccggctaattttttgcatttttagtagagacagggtttcaccgtgttagccaggatggtctcaatctcctgatctcattatccgcccacctcggcctcccaaagtgctgggattacaggcgtgagccaccacgcctggccataaagcagttattatttcttatgcttagtcccaaaaacctagtcttccttgagtcatttttctcttatgtacaatctggcaacaaatcttgcttcaagatatatcaaactgactatgtcttattgcctctactgttacttgccctgctttaggttacacacagctttcccctggattgcttctactagcctcctatctacccttgacctcctggagtccattgtctacctaacagcctgtgtgcacctctaaaaacggagggcagataattgccacttctctgctcgcagtccttcagtgaattcccatctctttcaaaggaaatcccctgttctttcaatgatgtgcaaggccctaccttctctagcccctatccctatccctaccccttaactttctgacctcagcatcaaatatgctccctagtctatccatgttgtcttccttatttctctttgaacatatcaactgtgcttctaccttaggatttttgtagttttacactttgcttggaatgctaatcctcaaaatatgttatgccttattcccttcatgtctactcagctagcagagtattagtgagacctccttgtcctccctgtatataggatataataatagcctctcacctaatagcacgcctgttccttcacccagctttactttcgttatagcagtttttttgtttgtttgttttgttttgttttgagacagagtctttctgtcatccaggctggagtgcagtggcgcagtcttggctcactgcaacctctgcctctcaggctgaaacgatcctcccacctcagcctccacagtagccaggacttacaggcacacaccaccatgcctggctactttttatatttttagtagagacatgggttttgtcatgttgcccaggcaggtctcgaactcctgagctcaagcaattcacctgccttggtttcccaaaatgctgggattacaggtgtgagccactgtgcctggcccatcatagcacttttaccatttagcatgttatacaggttatttttttgttgtggtgttcactgctatgttcctagggtctagaaaggtaactaatagatactgggtgctttgttaatatttgttgattgaatacatttctaatattcagtgcctttttctgttgggaaatgtaaaatgagcaggaatataagaagtttaataaattataaatatttgtagtgttctccatttcttcccttcagttctcttttaagaataaatatccttgcttgctaacctctggtgtcccttgttgagcagcatgtcaataatgagttcctgctattggaagcctcacaggccaagttgatataaagaagtatgaagaaagtatggaggggttagtgggttagtgagattggaatctgtggctgagcccgtacatgattttggatgattaattaatgggctgtgtcgtaagacagaaaagagatgagggagaagctttggggtgtgaaaccaagaaatctggagcgatactgtctgtgtggagaaaattgtggccaagattttttgctattaaagatgtacttaggccaggcgcagtggctcacacctgtaatcccagcactttgggaggccaaggagggaagatcatgaggtcagaagatccgagaccatcctggctaacaccgtgaaactccgtctctagtaaagatacaaaaaatcagctgggcgtggtggcacgcacctgtagtcccatctactcagtaggttgaggcaggagaattgcttgaacccaggaggtggaggttgcagtgagctgagatcgtgctattgcacttcagcctaggtgacagagggagactctgtctcaataaataaaaagataaaaataaataagaaaaagaggtccttaatattttatattaatgatatacttgccccactttttgttttttctcttcctacatttaagtcatttcactctttgaagcactcttgtaggagggtgataggaaaagtaaaacgattgaataaaaaataatctagaaatatttccttagtagttcctgtaaggcttgatgtggaatagaaaaccaatacacgggccaggtgcggtggctcacatccgtaatcccagcacattgggaggctgaggtgggtggatcatctgaggttgggagttcaagaccagcctgaccaacatggagaaaccgcatctctactaaaaatatcaaattagctgggcgtagtggcacatgcctataatcccagctacttgggaggctgaggcaggacaatcgcttgaacctgagaggcggaggttgcagtgagctgagatcgcgccattgcactccagcctgggcaacaagagcgaaactctgtctcaggggggaaaaaaaaaaaagccagtacatgaattaaagtgtaagaattatttgtgtttacacttcatgtaatttatttccaaatttatctagataattggaaattgacctgcattttaaaatctgtagccaacagttaatgttaaaaaaaaaagttcccttccccaaatgcagttaaaatctatgaactgctgtttgctatttgataatatacttgatctgaatagtaattatacattattagattacatgacttcttagaaatatggaatcaattttgactatagcaaaaagaaattatacaaagcttgaaaataattttgttattttataaatgtgtaagccccttaatgtctatcagtagttactgatttattagaaatatcatgtttttctctaatattgcccagtaaattttttttaaatttatttccatcagagttactaatttagagtgtaaaatagcaaaataagaaatatcaaggaaaaaaggtatttctggtaactttttatctaaaattgaccattttgatacagaatggtaacaagtcatgcttagggttaggaaatgtaactgtattcactatactcaatcctaaaaattgtgtaatacctctatgttcaaggaggtttaagttcgcctccctcatagacctctaaatgcagtgactatgagaaatgtcataggagaggtacaaaatatgtttggagaatagaaaaggaggaacaactgtttcattgattcaaggaaatttcctcaaaaaaggtaacattggagctagatattgataaatttgtttaaacaaagactttctgttttttaagctaaaagaaccaacgtatgcaaaagcagagagaaacatatgaaaagagaactgtgttttccaggaatggagaagatgagaatgtggctagagtgttaggggtgcatagttctgagtagatggatattaggcccttcaggagattgacgcctttttttttctttctttcttttcttttttttttttttgtgagacagagtttcactcttgtcgctcaagctggagtgtgatggtgtgatctcagctcaccgcaacctccacctcccaggttcaagcaattctcctgcctcagcctcccgagtagctgggattacaggcatgtgccaccacccccagctaattttgtatttttagtagagatgggatttctccatgtcggtcaggctggtctcgaactcctgacttcaggtgattcgcccgcctctgcctcccaaagcgctgggattacaggcaagattgatgtcttaatgtgaagggtcctttatgcaagctagaggttttgaggttccccctccccatcgagccttgcagttccattaaaggattttaagcaagggagtaataaaattcgttatatcattgaatgaataaaatagtcagactttttatttgtaggagaaaaattgtattagatgtagccaaatatggcttaagtaatgcagttttaaaattcagaattggaaaattggtttatctttgattaactggttcattttttttcttcctaacttctttcttctgtcagccaaaaaaaaataggaaataatggataagctagaagcagtatttgctttttaagttttgcatgactgaaacaatcccctacactgttagaagcctaacacacaatgctattgtgctctgggtctagaaaaaatgtgaggcctgctagtctgattttgcagcagggagtaaggaaagccaatacttaactttgtaagctgctaaatattaaatgtttagtaaaatggggtttatagtaatgttttttttgagacagagtctcactctgttgccaggctggagtacagtggctcagtctcggctcactgcaacctctgcctccgggttcaagcggttctccttcctcagcctcctgagtagctgggattagaggcgcctgccaccacacccagctaatttttgtatttttagtagagacggggtttcaccacgttggccaggctggtgtcaatctcctgacctggtgatccgcccgcctcagcctcccaaagtgctgggaatacaggcgttagccaccgtgcccggccagtaatattttttcttaaaaagagaatatcaaaataaagaggagagtcaggagtagagggtatcaccaagccttttttagagtcacctaatttgttacatgtttttcttaaaattaccaaccatcaaaaagaagaattttgatctatatctatgcctaagagatgtcagtagagcagtaaaaatatttctctaatttataaggaagttaagtaactttcccacagtcaaaataatcatgtcaaaaatttgacctttcaaccactagttttagtaatccaccctgcttttttcctcagggaaaaaaaaaaatgtacttcccagataaacatcaccttgcgtttcaccgcaggtctgggtgatccttaccttggaacaaatgatagtgttggtgcatttaaagtagcagcttttgggtgactggctggtcctgaggagtttttcctacttagcagtgttcattttatctattaatgccgtgaagcaaaatacaggttgaatgagcagtgttggtaactgcaaaacaaagtgttgcaacacagtaggctgaggtagttttaagcagataaagtctacagaaattagtttacagaaatagtgttaggctgggatgttttagctgctagatttcagatttagagaaatcaagtttagaaaaataaaccagcaaagtagtacaaaattaatagcaacaatgtcaggaagaatttgcttatgaaaaggatctattttcccttttgtgaagagaaacatttgttcacccttggttacttgtgattgagataattaaaaacaattttatttggttttagcagcctccttgtgaaaacttatgaaatattaggcagtttgattttcttctcctctctggacctgcagataaatttgttgtgaataaatggaaaatagtttggcagtgggcagaagcaagggaaaagccaagaatctggtcgatttataaaccagacattgtgaataaatgagttggctacagctcttagaaaaaggcttttgcctttcgagttcttttcctacatctgcttttatttctttcacactttatgaagtcaataaaaattttgatttcattgttacagaagtttctccttgtataggagcttttcatgccctctgttggccataaataatttacttttgatttctcaaggctcctaactggtgacttttcatataaggggatgtctaggttgtttttggtagtgatattatctctttgtacccagagaacacttctgacaccaaatggggggttttcttcacagcaacaaccagttcttcagctctacctggagttagcgtcagatcccataggtgaaacgctgagtcccacaagactatccccactttagatgccagtcacaagtcccaggcatcccgtagttctgactgactcaccgtaaatcagaggttcccatgaactcctcatcagatttaataatttgctagattgggtcacagaactcaggaaagcactttactgacatttgcttgtttattataaaggatactatgaagaatatagatgaacagccagataaagagagattaatacataaggtgaggtccagaagattcctgaacatgggtgctcctgtcccatggggttagggtaggccaccctccctgcacatggatgtattcaccaacttggaagctctctgatccttgtcacttagaggtttcatcatgtagacgtgatcaattattaactcagtctccaggcactctccctctccagaagttgggaggaataggtctgaaagttccatgcttctgatcattacttggtctttctggcagccacctccatcctgaggctacctagggttacgccaagagtcatttcatcaaaagaggcttctaccacccaggaagtgtcaagagatttaagaactctccgttaggaacaaaagatgctcctattatccccaccacttaggaaattacaagagttttgtttttgagacagggtctcactgtgttgcccaggctggagtgcagtggtgcaatctcagctcactgcaagctccgcctcctgggttcatgccattctcctgcctcagcctcccaagtagctgggactacagatgcccgccaccacgcccagctaatttttttgtatttttagtagagacggggtttcaccatcttagccaggatggtcttgatctcctgacctcatgatccgcccgcctcggcctcccaaagtgctgggattacaggtgtgagccaccgcacccagccggaaattacaagagatttaagtgctgtcctttgtcagaaaccagagatcaagtatatatatttttattatgtcacaaatggattctaaaatcagataatcttactattacttatctgtgaagaggacttcatactttgcacagaagtatttgctgtgacttaaacaagagtcacatggtgctatgagcttaaataattactagtaaaatttggccagagccagcagccatggcttatgtctgtaatcccagcactttgggagtccgagatgggtggatctcctgaggtcaggatttcgagaccagcctggccaacatggtgaaacaaaaaatgtctctacaaaaaatgcaaaaatgagctgggcgtggtggtgggtacctgtaatcccagctacttgggagcctgaggcaggagaatcacttgaacctgggagggagaggttgcagtgagccgagatggtgccactgcactccagcctgggtgacaaagcaagactctctcaaaacaaagaaaattgtttttggctgggtgcagtggctcatgcctgtaatcccagcactctgggaggctgaggcaggtggatcactttgaactcagtcgtttgagaccagcctggtcagtatggtgaaatcccgtctctacaaaaaaatacaaaaatgagccgggcgtggtggctcacacctgtggttccagctactcaggaggctgaggctagaggactgcttgagccaggaagtggaggttgcagtgagttgagatcgcaccattgcactccagcctggtgacagaaggatactctgtctccaaaaaaaaaaaattactattaaaattagtcttcatggtgttttggtgaactttagcttaccagctgaaaattatggaattcatttcatatagcttccactataaatactattttgatccaagcctgttaataacactaagcatggtgactctcaaccatgactgcacagtgggattgcctgggaagctttaacatacactgatacctgggtctcatatcccagagattgacttaagtggtttggggtgcaacccaggtattgcagttttctttcttaaagttccacaggtgattctaatgtagtcatggttgagagctactgaccttgagaagcatgattttttagaggtggctaacccgttctggtctactggtttacatgcagtagagatagttggggaagataatttttgcttaaaataggaaagcaactgacacaatcgcatggtaggcatgcagggtttagaacagtaagaaactgaagattataccatataacactgtgaatttattaaatacattccagagtgggttatcagccaagatgtgctagatcaactgtgtatggtgctgctaatgagttcacagaaacctagaggtttttacttgcccaaggtcacaccactagctggggacacaaccaaaactggaaaattatagtggtaattcatagtttgttgccttgattatttaggtcaagttctgtaaactttttctgtgaagggccagattgtaaacattttaggctttgcaggccatatggtccctgttgcaactactcaactctactaacatagtaaaaagcatcatacacattatgtaaatgaatgaatgtgtattctagtaaaactttattgacagaagctggatttggcctgtgacctgtaggctgcagatccctgattgaggattgttttaatcttcccattggaaagttggctccttaagagtaggctgaagatgttactggtcccacgtggtgccttacttcctggtactgttacagtggcctctgttgcaaggtgttagatgagtgcttgttgaattcatatctatgcagctcttaacgtttttctgttgcttctaacccttccagtaggggaaacagttggcacatattccagctttggagaggaaatgcttgaagtctgtagcagggtataattgctgaattcttcaagcgaattaaagatctgaatttgctctagggcattcataattattttaatttcctggaacagattctaagaattcattttaaatctgaaagtttttcttaaaatctgttttatgatacactgtctttatttttgtgaatattgttagtttctcacaacaatatttatttcagttcatttggatctaagatctttgttgtacttatgtgtggccagtagactctttagcaaaattaaacaaatgctaatgggttcccacagccacccaaatattagaagtacagctccagattatgagcctgtttctctttttcttttcttcttctcattattttaaagagatggagtctcagtatgttgcttaggctggtctcaaactcctgggctgaagccattctcccgcctcagccttccaagtagctgggattacaggcaggagccaccaccccagacatgaacccacttctaatgcaaaacacatattgacctgacaacagggccctcagttgtggtggaaagagcagtgaccttggaattaaaagccctgttgtgactcgtggctttgccacttttaaccaaccctacttttatttacctataatgtgcaggtatctgtctcactctttagggatgccaaaggtgaaatgaggtgtgctgtctatgtgacagtgccttgtgagcacttttctgacttctccctgaggaggtttgggtcaggcctagcagaaactgcagccatagagcatggagggaagtaggacttgactagcataaagtgagtctatgagagtgtgcccactggtttccaaagagaatgtcgaagagtctacaatggtgaggcctcatcagcctcgatttgagaagcaaatgtaggtcagaaatggggagcagaacaagaggaaaagaagaaggtcaggagtgtgcagagctgaagaggtggtgagaagtggttcaaagctggaaacaggcaaggtaatctggaattctgtcttgatgtctattgagttatcctaaatcaagactcatgttcctcttaccttatatttcttaaattgatttgtttgatgccctgggtattttgaagctcctagctgcataatcttgcctaataaaggcacagaattagggcaaatagctgcagtgacttgtgtacctgctcagtgaaagccaaagaatcaatagctagaatctgtgtccacaaattgcttacaaaccacatgtagatatgacattcaacatcaggcaaggctgggcacggtggctcagatctgtaattccagcactttgggaggccaaggcaggaggatcactagagttcaggagtttgagaccagcctgggcaagatgatgagactcagtctctacaaaaatgaaaaaaaaaatagctgagtgtggtggcatacacctgtagtcccacctccttgggaggctgaggtgagaggatcacttgagcccaggagtttgaggttgcagtgagatatggtcatgccactatattccagcctgtctgcatgtaaaaacaaaaaatacacacacatacacacacaccccagcctaaccacttaccacctcttaatcatctgtataatggacataaaaatgcctgctatactttacatcatagtattcttgagtggagtaaatgaaataatgcatataaggtgctttgcacaatgtgtgcttgcctcacaataaacactcaatattattagatatgatttctaccattattactaacagtagtaagaatagcagtagtatttaaaagcaagtttgcattagttttaaagcactatgaaatcccaagtcttttttaagacggagtctcactttgttgcccaggctggagtgcagtggtgcagtctctgctcactacaatctccgcctcccgggttcaagcgattctcctgcttcagcctcctgagtagctctgattataggtacccgccaccacacccagctaatttttattttttagtagagacggggttttaccatgttggccagactggtctcaaactgctgacctcaagtgatccacctgcctcggcctcccaaagtgctaggattacagggatgagccactgcacccggcctcaagttatgattgttaatatttttgtgacattcaggataaatattttttagtatgctactgaaggacatgtctcagcatcggtgtatcccttcctgaagctatatgttaccttgcatttacagagttgacttggtgtccagagaacaaatcagttgagaaaaggccattttgaaaacgattttttccagggagcatacgctagtaccctgttgcggcatccatgtgtgagggggtggttggattatagatcttcaatgtgaagtcaagtttcttatcaaatgaagtgttttgtccgatcttagagtttaaacccacaaggaggtaacaaagcaaagggctaatatatcttttcgctacttagaactacaggccagaaatttgcttttgtattctgagatcttatgatacttgtctcaatgaaataaaactgcatgcattattctgcatttccatattactattctctgtattggtaattattatgatacttgcttcctgtacttaaagcagcatgcagatttattttataatcacttcaaggaaagaaccagttcctttggagatttcctgtggttccaaagttgcatgcctccaagtctgttaggttgtgggattttgcatttgagagaaatgagggaggtggagagaatgaagagaaagtggactggcagaattacagaatgtggtttcatctgatcttttaggctgagggctactccatttgtagactccatgcataaaactcaggcatcctgggtcctgccctactgctgctttgtggtcagaacatcgtgagatcatgcagcctcaaggctgggaacatacgggatcaggtccaacagcaggttggagtttgggttcatgatttcaaagtcagggtcgtgagggcacccaagatgattaccgagaagtcaaaagcacgtgccagtaatggcaatgtgaaggtctgggaaacagccaaaacgtagctgtcaggaatgtgggacagagtccctagggcagcttttcccaaaacatgttccaagatatactagctctatgggatgttgacttacaataatatgtggtgaaaaggttttccagacaaacactgagttcagagttaagcaggtttttttctattgcagagcttgtcagagcctctgatatgcccactagtattgtaagtgtacaagatgaagctttaaaattgggcaaataacagatatttcatacagaatcctggatgtggccaggagtgatgggagatgaaggatcctcttgtcagagtagcactccacagaacacacttggggaaaagttgtcccagggcctgtggcatgtgctctgaattcattttcgcctggctcttaattctggggttcggaaggaactggcaaacacccacagaagttatctaggatcttattgagtgcttccccctcgtccttccatccgaatcacttggctgatatttgttgctgctattaacagaattcaggaaatcagcccttcttagaggaagaaggacaagtgagaccacaaacaacaaaacataatgaatggatcagagcctgtgtataattggccctgaattaggcaaataggctttccccactcccttaaaatgaaaacaaaatggtagtttaaagaaagtaccaaagggactatatctaataataacaatgagattgaaaatgagggcacatggtgtgtttagggacggctgaggccagtgaatgtcaaggcagactgcatctttgacagcctcgcctcactggctcctgaaatcctcacacacaaaacagcattcttcttgtgttcttaaagtgaaaggcactgtttccctgtcacactagcaatgttgcgaagtaaatgtcatcctttcctcctccttggccctgccttctacagctagtgcccctgccagttgtttcctcagcacgtctccagcgttcctcttctcttccccctcggcacggccttacttctggtctcatcatcctatgcctgggctgatgcctttgcttcccagctgcagtcggctggggatgaatgtggggttaaggcaggcattccagatatttatagaattgttcatggttcttttcccattagccatgagttacttgagggtgaagatagtgtcttatttgtctctctgtctctagtaattagccaagtacctggcacttttaattaaggagttatcactcagtacatttaaatagggagaaaaagaaaaaaacaaagaatggaataaagtaaaagcatgataggttttgttactctttttgcagttgatcattggctattcttaattcttatagaccagttagtactttagcagcatacagataagatatattaaaatcatacactgagtttttaaaaagtagatgggcatgagtggtggctcatgcctgtaatcccatcactttgggagggcgaggcagtaggatcgcttgagcccaagagttcgagaccagcctgggcaaaatagtgggaccccgtctctacaaaatattttttaaaaattagccgggtgtgatggtgcacctgccgtcctagctgcttgtgaggctgaagtgagaggatcacttgagcctgtaaggtcagagctgcagtgagccatgacagtgccactgcactccagcctgggcgacagagcaagacactgtctcaaaaaaaaaaaaaaaaaaaaagaaagagacaaaaagttcattagcacttaaacatgcttttcagaaaatgactaattatcaaaaactagcaattgtgatactagcaaatgaagaaacgtcaaatggagaagaaatgaaaaacgtttttagaatccaggacacacatttacaagaatgtaaaatattgtgaaaataatttggaggagaattatgattaaattatttgagacttgaattacctgcagtaaattcagctgcgtatcggttgttaggcattttatcattactaatcctgttaccatctttgttgaataagagattcatacttgatatacaaggtccatacatttgtaaataattcattattatattagtttgcaagagtcatgattttgccagaatttatccaaaatggtctattggtaatcccagagagtagcagaggcattgggcagtgaagctaacaggcggtgctaaagccagcatgagcaagtgctgggtggtaggaaaatagaactcaggcagtagtgagggcaaataggagcagacagggcagacctagaaggagacagcaagtaagaggcttaggaggctggaggagcagagactaaggaacgctcccctggcatcatgcagcattattacataagtgttagtaatttcaaatagttataactattttagatataccatgtgctagctactgttgtagtatgctcactagaatttaatacttgaatcctcacagcaacctaggtggatactattatgaatcctatgaattttcatttatagatgacaaaattgaagtacagggaaagtaagtcattcacccagctgatcatcagtaaatggaaaagccatgatttgacctcaggaaaatgagggttatttgacagggctgacttgaagaatgtttagattgctgtagaactgtatttagtgcttaacgatatataataactgatacaattggtatccaaacagccgaatgtcatgcttttagggatcaaggaaagttgcctaagtgtgcatgtgcctagcatttgagttttgatacggatgatcataccagaaaaatataccatcagttttcaggcctatctgcacagtaatatgcgtggagttcttcatgatgtattcactgatattaaagctcaaaactattatgaatcttaggctctggcgaaaaaatgtgatcgtttaaaaattcggcatttatcttatcctgtggaatgggcaacttctcaggcccatttgtcaccaccaacaaagcagtaacacgaggtgttttaataaatctgttgcccagtctcttccatattgctatgcttagctgtaaaagaagggaccaagaaatgggtgttaagctgaattggaaacaattttcagataaagcacagcagtctgcgaacctcctccagctcaagatggtgtcaactagaattgccttttgtaagcaggcagaaagagacacatgtacttgattgtggtttctctttactcccactttccactctggtcatccctgtagacacagagtaatttgtgtactgtaaatagctagatctttctgtaatctttatttgtttctttgtttgttttttgagatggagtctcacactgtcgcccaggctggagtgcagtggctcaatctcggctcactgcaacctccacctcctaggttcaagcgattctcctgcttcagcctcctgagtagctgggattacaggcgcccgccgccatgcccgactaatttttgcatttttttttttttttttttttttttgtagtagagacaatgtttcaccatgttggctaggcttctctcgaactcctgacctcaggtgatccgcctgccttggcctctcaaagtgctgggattacaggcgtgaaccaccactctcggctttaaccaacattcttagttgaacacagcagaaagggaaacctccattagtacatggaactcgggcagtagtgagggcaaatatgagggagagctgccttcctgttgcagtggtaagttcagtctctacctaagaaaacagcatgccaaggtacagcagagcacactggccagctccaatgcaagagtcatcatggagtgtaccttttttttttttgacagagtttcgctcttgttgcctaggctggagtgcaatggcgcgatcttggctcaccgcaacctccacctcctgggttcaagcgattctcctacctcagcctcttgagtagctgggattacaggcatgcgccaccacgcctggctaatttttttttttttttttttttgtatttttagtagagacagggtttctccatgttggtcaggctggtctcaaactccctacctcaggtgatccacccgcctcggcctcccaaagtgctgggattacaagcatgagccactgcacctggccggagtatgcacttttaacatgtctatgcagggacacatgtcctctaacaacaaatggccatcccgagagtggtggagcagagtgttggacctctggagcccaaatttttattgtagcaatacacagtcttcttataacaggcactgtggccatgggtcttatcagctcctattcaaaattggaattaggacaagtatagcaggttgtgttctaacagaattctaacttttaaggtgccacagacaataatattctttagaaatcttagtgttatcctttttatcctttcagggtagcaaacaaaaggcactcagtaaataattatggagtataatttggggaaaacaaaatatttctagtctgaaaattattgttttaaaaattatttttgtcttgagaggctgtacatttctgtcaactgcgtggccacattgttgatatgcttaagaaagataacagtcaaagttgcctttttacactttgtatttgtgctcatccaaccatgagacctttaaaaatattgaatatctgtagttatctcccttcaagtcttattttgagtttatattacctttgaactcaaaagatctcaagccatttgctttacctgacagtatgacattctttaattttaggtagcattgtatgatgagtctcaaatattggcagcctataatgcatataagttagtgatcctagtaaatgtaaataaaataataaacagaaacatgctgggcatggtggcttatgcctgtaattccgtaactttgggaggctgaggcaggaggattgcttcacaccaggagttagagaccagcttgggcaacagaaaccccaatttttaaaaaatcagctgaacgtggtggtgcatgcctgtagtcacagctactcaggaggctgaggtgggaggatcacttgagctcagattgaggctgcagtgagttgtgagtgcacctcagtctaggtggcagagtgaggtcctgtctcaaaaacataatgataaatgaaaataataacagaaacattcagtcaataactgggatttttttttactgttactattttttatcaagacaaacatctggagagttctacaattccagatgcttaccaatctgcttctgggttgtgaaaaaattatttgggtatgtatagcttttaactttttttttgtttgttttaactgaatattgaggattttggtagaataactttgaatgttttggattttagatttaaatggaaatatcaaattttatataggttgggctctagatatttccatattgtcagaatttagaaaacatcttttatgaaatagaaactaagaattttggcaaacagagaaagttgcccaaggtctccagaattcatttgtttgacaaatacatattgagcatctaccctgtgacaggtggtattcctgggatttgtcaggagcacaattgctgccttcctggagcttacattctaaccagggaaaactgacaataaacaataaaaaaagtaagtaaataatgtagtttgttagaaagccataggtgctacagaaaaaaataaactataaagtaggggtcggactagcaataaaactaaatagtgtggtcagggtagtccatgtggacaagatgcaatttgagccaagatttaaaaaagataaagcaattagcctcaaaataatcggggggaagaactttccagcaccaagtagtttgctcggcctgttggaggaatagcaaggagccagtgtgcctggaatggaatgcccaaggggcaatagtggccataaggtaaaagtggtaatggggctaaggccagatcctatagagcttacaaactactgcaaagaacttgagattttactctcagtagaatgtggatccattggagaattttaagcacaggatctgacttgggttttaaaagggtctctctggtcctggtttgacaatatggactgcactgggaacaagggaaggagcaggggtcttactgctgtaattcagttaaaaattatggtagtttaggctggaatagtagcaccagagatagtgctagatggtaaaatctggaaatattttgaaagtagagccaccaggatttcctggtgaataggatgtagagtatgagagaaaaagaggatttggggtgtttccaaggttttagcctgtgcataaatggagttattactattgattcagagaaaaccttaaggcagctacttttagataagagttgggtgatgaggggttcagatatgaatctgttgatagtgagatggctcttagacatactggggaagatgctggtaggcaactgcatgtgtaaatctggagacgggtgaggtctgcagtagagatttaagtgtaggataatctcaacatgtggaaggcatggaagtgtgggtgcaagtgggagacgagaaccaagcactaatccaagtgcacttcaatatttaattttaagagacaaggtctttttttttttttttttttcgagatggagtctcgctctgtcacccaggctgaagtgcagtggcgccatctcggctcactgcaagctccgcctcccggtttcacgccattctcctgcctcagcctcccgagtagctgggaccacaggcgcccaccaccacgcccggctaatttttgtatttttggtagagacggggtttcaccgtgttagccaggatggtctcgatctcctgacctcatgattcacctgcctcagcctcccaaagtgctaggattacaggcgtgagccactgcgcccagccgagacaaggtctttcactctgttacagctggagcacagtggtgcaatcacaggtcactgcaatctcaaactcctgggctcaagtaatcttcccaagtagctaagactataggtgtgcaccaccatgcccagctaatttttttttttactttttgtagagatagtgtcttgctgtgttgccgaagctagtctcaaactcttatcttaagcagtcctctctccgtggcgtccctaagtgctgggattacaggcatgagccatcatgcctggccaggcactttaattttaaagagcctttaatatggtttcttgccatgtcagagataagaacagcttaatatttcttgacttttccttggttaaacaaggggtatgctttaaaattttttttaattttaattgtggtaaaaggtacttaaaatttgccatcatagccatttttaaatgtacatttcactgtcattaaatacattcaccactatcatccacagaactcttcatcttgcaaaactgaattctgcacccactaaacaataactcccttttcttcttcctcccaggtcctcaaaaccttcattccattttctgtttctatgattttaaagtggaattatacagtatttctctttttgtaactggcttatttcacttagcattatgtatgtcctcaagattcatccatgctgtagcatatgatagaatttcctgctgggcgcagtggctcacacctgtaatcccagcactttgggaggccaaggtgggtggatcacctgaagtcaggagttcgagaccagcctggccgacgtggtgaaaccctgtctctactaaaaaatacaaaaattagctgcacatggtggtgggcgacacagcctcccaaagtgttgggattacaggtgtgagctgctgcgccaggcctgcttctactctttgactattgtgaattaatgctgctgtgaacatggatgtacaaatgtaagggatatgtttttgttttttgtttttaagatggagtctcactctgtcacctaggctggtgtgcagtggcacagtcttggctcactgcaacctccgcctcccggattcaagcaattctcccacctcagtctcctgagtagctgagattacaggtacccaccatcatgaccagctactttttgtatttttagtagaaactgggtttcaccatgtttcccaggctggtcttgaactcctgacctcaggtgatctgcccgcctcaccctcccaaagtgttgggattacaggcctgcgccacgatgcccagcagggatatgcttttttaaaagtctctgtgatatgtatacttggtgactttcatagatttttaaaaatataagtgatatataaatacatttttattataaaagatttaaatgatatagctgttaacagaccaaaatatgaaaattctcattcattctctcacctcacctccatctgcaattcccaaagttacatttggtgtgctatcttccctctctttcagtacatttctatacatttgtctataacttattctttttaaaaactgagatattatacatattctacagtttaataatgtcttggagacctctcaattatttatattttggaaatattcccagtctgctgcatatgttctatagtgttatatttacctttacatgtacagggctattactgtagattagagtcctagaagtgcatgcagttcctaggtccaaggaactttctttaactcttcatcagattttctttttgaagaacctttgtggcttgataattatgtttctagaaaatttattattcaggtttagaagtttggggagctgtttgaaataacactcaaataaatttcctggctatggatttgttccccatagcaagagagttgtaaactatgagttttataaccttaatgtcttttttttaaaaaaggcaattgatctcttaatctaggtgttttagctagcatatgggagatgtaatctgcaatctatgggacaaatgggggagttttgggagttatgatagaagactgttgaaaaatgttttataataatgttttacagtagagaaacctggtagatgccacctcatcagccgaccagggttaacatcacatgataatcatattgctatcatgcagtgatattaatataataaagtgagatgggtgcattgcctctgtgatattcttaccccaaacccataaccacagtctcataatgagaaaataacagaaacccaatttgagggacattccacaaaatacctgaccatgttcttcaaaagtgctatggtgccaaaagtcatgcaagacaaatctaagtaattctcacaggttgtagtacactaaggagacagtgaaaactaaatgcaaataaagtggtttttggttttttaaagtatgtttatccttatggacaatatgctcccataaagatcgtctaccctagaaaactgtgtcttccctcttaaatccaagagatgcttgtcccaaagggtggaccccattgagatctcctgggctagagacccagtgcatggatggatgagacagtataagcaagaccgtataaagctgtgatctgagctcatggacactgtggtaagaggtcaggctcgaagaatcagagaactgagtggattttgcaatggatagccaaagagagggtccaaacttaaaatgcgaaacaataattcaagaatgatttggtaaaatgtaatataacagcctataacagagtctagcaaatagtagccaaaagtcaccctaaccaatcgtagggcatatctaccacacaaaagtgtgacttagcgcatgactgtttaaatgccaaaaaagacagataaaggaaagttaagccatagtggcaacctctgcagttggaacgagggagctatatcagtcaacagtgaggctaattgagaatatttgaggttggcgtatgtctagggattcctataggagatacaacccagcaagcataggtcagcaacccaaggtcagagtacaaaatgggttaatactaaaaagtggttaaaaagatgaccagtcctcaagaggagatagtatttaaatcggatacctctccttccccatgctaactaacatactgtaatttgttatggctgtttctaaggtaactgtttataattataaatcagccctactggttcttcttgatattgtaatttgcaatcaaagaacctctagagttaatgtaaaacttcttgaatgagagaccatctggttacaattttggtttcaattccctgttaggcaactgacacccaaataaggactgtttccaaatatgagtaggctaagtcagtcataaacaaacaaaaagtttcagtttttgttgctattatccaagatgttagcagatgaagtgtttaatgaaagggtgcttcttttaggcaaacttcttttaccatgatttgtttaacctataccagtggtccgtacatttttttcctgtaaagggccaaataattattaatagtatttttagccttgcagccacgtatgatctctatgtatttgtcattgcctttttgtttttttaaacaaatgtttaaaaatgtaaatgttctctgtgtccttcaccatgttttcatgctttgtgatagtggctgaaatttagagacttgttttaataaacaatgttctgaaatgttgatctaattgcctctggcaaaagatgtgtaatcatgactttgatgaacttattttaaaaatagatgcacagttgatctcttattgcacgcattttggcacattcgtgggagatattctctgtaaacatgacacttgcgaaacctaccataccctgaaagtttgctgtctagaaattaagtgcctagttttcattcaaattaatatttaggtaagacatgttttatttctaaatagactgtcaaacatttttcatattttaatgtgtaatggtgatgacctttttaaaaagactcaacgtaatttgttttctcatttaacccattaactgccccctttgtgtagtattgttgctgctttctcttataaattgatgatgtctcagggtctcaatttacaattacattagtttcttcctctagtttgtagttgagctgtggctctgcactgttggggcctgtacagccatccttcactagaccccgggaggtggcctttagtaggctcaaaggtggcctttaagccattcttagtatcactgtgaaaaagcatccagattttccccctgaaaggccaagtggttgcatgatgtgttgtggattttagggatatactttgcctatttcacatacttttttccaagaagatagataagcttacacatgtaaaggtttcaactgtatttcttctgtttataattttatttattttgagatggtgtctcgctctgtcactcattttggagtgcagcgcaatggtgtgatcacggctcactgtggcctcaaactcccaggtttaagccatcctcgcacctcggccttctaagtagctgggactacagatgtatgccaccacacccagttaatgttttttgtatttttttgaagagacaggattttgccatggctgcctacctggtctcaaactcctgggcttaagtgatctgcctgcctcagcctcgcaaagtgttgagattacgtgagccactgcacccagcctttttataattttaataataatctgtcaatctttcattacttttgccttctaaattcttgttatattttactaaacaatgcttaatctcgctgataaaccattttagcagaagtatgtctgatgaggaccacccctgcctttttttttcttttctcagagttgcaaggtctccctctgttgaccaggctggagtgcagtggtgtgattatagctcactgcttcctcaacctcctgggctcaagagatcctctcacctcaacctccccagtagctgggaccacaagcgcgcaccaccatgcccagctaatttttattttttttaagtaaaggcagggtcttatgttgcccaggctggtctcaaattcctagcgtcatttaatttctgttttggtattggctctgaaagaactgttgagacaatacttggaactcttgatattttccaaaatctgttttaccaagataaattttaaggagagcaacagaaaataaagcagaaatgtggtttctttgttgggttttgttttttttttttttttttttttttttgagatggagtttcactcttgttgcccaggctggagtgcaatggtgcgatctcagctcactgcaacctccccccatcctgggttcaggtgattctcctgtctcaccctcctgagtagctaggattacaggcgcccaccaccacatctggctaatttttgtatttttagtagagacggggttttaccatgttggccaggctggtcttgaactcctgacctcaggtgatccacccgccttggcctcccagagtgctgggattacaggcatgagccaccgcacctggcccaaatgtggtttctaatcaatattatttaataatgaattggattaagaattttttgggttttttttactcaatagaaagttgattgggttttttaaaaatagcttgttttcaataattaaggcttaaaaaaatctctgagttctctttgagatatttggattctataaaaaatgaaactctttgtttcttaaccaaatgataattccttaaattgcattctttgattcaattatactgaatttcctggtagaaattttattttctctgaagcagagtcagtggtcttaatatcaccatgtcaaccagaagagaaggaaatctaatatgcataatagagaacactgacacatgacttctttaaaagtctctgaaattcaacatataactttttataccagaaagtgaacaaacatttaatccttcctcttcatctatgtctcatgatgtatttgtgtgattttaaagaacattttggagtttttgagttctatataaatgtgaccatgttatctgtataatcttgccatcctttctttggaaaacatgggtcaccgttagcgtatgtcatggatgttgatgtggagtctgagtaagctagtgggcgagagggtgtgtgtgtggacgtgggcgcatgtgtgtatttgttagtttcaaatcgagccattcttctagagtctttaggtcagctgtcacgttgggctttcctcctttgttcttttgagttgctctgtgactagccacagacccaacactaaaaaggaaggcttttaacggattaattccgtcctttccttcgcctacattgtagcagctgcgttctgccctttctgtggccctgcaaggagaggaggcctgtgtataacagtcttctgtcaacatccattataagattcctaaagcacatggaatgagatagttcccaggaaaactctttgtcaaggccagcccatgtgggaaaagtgtttctatgcttagtcttgtaactatgactgtttttgttttatggggtcaggggaaatttaacttattttatttttcttttttattgatcattcttgggtgtttctcgcagagggggatttggcagggtcataggacaatagtggagggaaggtcagcagataaacaagtgaacaaaggtctctggttttcctaggcagaggaccctgcggccttccgcagtgtttgtgtccctgggtacttgagattagggagtggtgatgattcttaacgagcatgctgccttcaagctctgtttaacaaagcacatcctgcaccgcccttaatccatttaaccctgagtggacacagcacatgtttcagagagcacagggttgggggtaaaaggtcaccgatcaacaggatcccaaggcagaagaatttttcttagtacagaacaaaatgaaaagtctcccatgtctacttctttctacacagacacggcaaccatccgacttctcaatcttttccccacctttcccccctttctattccacaaaaccgccattgtcatcccggcccgttctcaatgagctgttgggtacacctcccagacggggtggtggccgggcagaggggcttctcacatcccagtaggggcggccgggcagaggtgcccctcacctcccggacggggcggctggccgggcggggggctgacacccccacctccctcccggacggggcggctggccgggcggggggctgaccccccccacctccctcccggacgggacggctggcctggcgggggctgacccccacctccctcccggatggggtggctgccgggcggagacgctcctcacttcccagagggggtggctgccgggcagaggggctcctcacttctcagacggggcggttgccaggcggagggtctcctcacttctcagacggggcggccgggcagagacgctcctcacctcccagacggggtcgcggccgggcaggggcgctcctcacatcccagacggggtggcggggcagaggtgctccccacatctcagacgatgggcggccgggcagagacgctcctcagttcctagatgggatggcggccgggaagaggcactcctcacttcctagatgagatggcggccaggcagagacactcctcactttccagactgggcagccaggcagaggggctcctcacgtcccagacgatgggcagccgggcagagacgctcctcacttcccagacggggtggcggccgggcagaggctgcaatctcggcactttgggaggccaaggcaggcggctgggaggtggaggttgtagcgagccgcgatcacgccactgcactccagcctgggcaccattgagcactgagtgaaccagactccatctgcaatcccggcacctcgggaggccgaggctggcggatcactcgcggttaggagctggagaccagcccagccaacccagcgaaaccccgtctccaccaaaaaagtacgaaaaccaatcaggcatggcggcgcgggcatgtaatcgcaggcactcggcaggctgaggcaggagaatcaggcagggaggttgcagtgagccgagatggcagcagtacagtccagcttcggctcggcatcagagggagaccgtggaaagagagggagaccgtggaaaggggagagggagagggagggggagggggagggagagggagtgggagaggggaaatttaacttataaggcagtattgtgatatgcctccatacttacctgccctttctttctgaactcttgcagg

SEQ ID NO: 204 - DNA, 8716 bp, homo sapiens
  TSPAN14
gtgagtagagcgcggagacctggctggggcgtcgggccctgcccctttctttgtctgaggccgacttccccgagcgttctccgggggcgagggctgctccttaggtccctgtttggcccctcagcctccctccccgcctgcccaggagcggccccgcgggcggcgctggctttgtctgccaggattgctcgctagaggttggaggcctccgccgggaccccgctgagccgaactccggcgccgcgcggacgagagccgcggcgggcccggggccggcactcctggttcggtagccctcgccccatcagcgcgccccggcgagtgcgcgcccgtgccgggcggggtgagggcgacgggagcctggacggccgctgctgctcggggctctgcttctcgtccccagcagcgagctcggggatgggccaccctttcggctcagtcacccaccctcggctcccaggagatcgggtccggggtgggggtgggcgtaggccggggcgggccgccccttcctcctgccccgccgtggttgtgctcagtctcccacgccgccgtgggtcccactctgcccggcttgggtttgggacggccggagtttgagtaggcggtgacccagcttctccgcgttagtcgtgtggggttgtgtttgggagcaaagcctgacccctgacctagccggagttctggtagagtgaggtttgtggctgcggactgtgcttgtcccgggccgctgctgcttctcctccacccccaggggtggggtgcgggtgggtaggaagtgcctgtgtgagggtgtgtggagggactttggccctgggaccgcaggagcacttgtcttggggacttaaagttgatctccggcgagaaacagtccgtgctagggggagtcgggggaagtcgtgggcccgggtactggcctggggctgggcgcagagcggtggtgagtgcggcggtgtcacacactcgttggctgtgctggcgctgctatccgggaggtcaggcggtgctgggcggctgtgcagagcattgtattggagcctctgcaggtgtgctcttggcagtggtctgctctccaggggatcttcctcccttccgagagggagttgctcttgggctagtgagagagtggcctgccaaatcctcaaaagtagccttccccttgccccttgaccttgaagtccagaagcagttagtgcctgctgagtgctccggccacccttctctatttgtgaagggaaacctggggacaggccatcggagcacccctatgtccttgccacatttcagctagccgagtggttggtgggggcggccgtctttccagggatcttcctgggccctggcacccccagaatgaagcccagactccaccataatggccctgtatgcccttctatccccttagctgctggtgtgatcatagcctcaaattgctgggctcaagtgatcctcccacctcagcctcccaaagcacagggattacaggtgtgagccaccacgcgcagtctgatcccactccgatccctaacgttatagtgacaggccctggaaacccagggccaccagatgtattttccctttccttctcattgtgttgcaccccaacttcagttggagactctggatatatccatccccacatctctgaactccactgcttgggccacctaatgtcatcctcagtttagttaacaaagcatggttgagatttctgtggggtgcgaggcattgttgcaggcactattccaagtctggaaagtccctcaccagctatgaacttggtgtggtcttagaacccaggctggacatggcataggtgctcagtagtgtttattgatctgagcttgatctcagcgtagcaggatcatttagggagacggggttgtggggggggtctgagcccatgggtgcttctgaaggtttgtggctgccccaggaagaaagggcatggagctgcagaatggttggctgcccagggaattgtccccttcaccagctgggcagctgtgctagtctctgaggactggtgaaggtccttctcatgtaggaagtggcagtatgaagtgggagaggagatggctctgccctgaaggttgtggcggcagtgaagtgaagttcccatggaggccggggagcaggaaagccctgggttctggaactatttcctgaggcctcactctatgccaccatgtgctcaggaatttgacccatttaacttcaactgaggtagaattccttttagcagtgcctgcaaagtggcattgtgccaggcctgggaaacctttgctggcttaaaatttagtgaaggacaagattatttcatagtgatactaatgtgagtttattgagacttacaccctagaagtttgacttgtaataactcatttaatccttgaaaacagctgtgatggctgggcgtgatggctcatgcctgtaatcccagcactttgggaggccgaggtgggtggatcacctgaggtcaggagttcaagaccagcctgaccaacacggtgaaaccctgtctctacgaaaaatacaaaattagctgggcgtggtggcacatgcctataatcccagctacttgggaggctgaggcaggagaattgcttcaatctgggaggtggaggttgcagtgagctgagatcgcgccgttgcactccagcctgggcaacaagaatgaaactccatctcaaaaaacaaaaaacaaaaagcaaaacaaccgtgatgtcaacagctccattttacagatgaggaaactgtggtcccgagaggtcacagagtaacttgcttgaagtctcaatatgagctgggttttctcatttctttttttttgatactgagtctcactctattgcccaggctggagtgcagtggcgtgatctcggctcactgcaacctccgcctcctgggttcaagtgattcttctcctgcctcagcctcccaagtagcttggactacaggtgtgcaccaccacacccagctaatttttgtatttttagtagagatggggtttcaccatgttggccaggctggtcttgaactcctgacctcaagtgatccgcccgcctcggcctcccaaagtgctgggattacaggcgtgagccactgtgccggaccaggttttctcatttctaatttagacaatatgttcctctcagaggtgattaaaatggattcatataccaaacaagcctggcccacagtggaactaatgaatgttttcagtgaaggtgcccttgtatccctgcctctgaacacatctcatctgtccagcacagcctgggcacatagcaggtaccccggaacccccgctgattggtgatggagcttctgtctccctaaggccagggaatgggaagaccagcactaccacatgcagaagattggggagtcactgcttgtgtctgtctggacagatcacggtttgctgtggtaacagcaatccccaggtctcagaggcttaaagcaacagaggtttatttcttgttcacactgcatgttcatcacttatctgctttatgttgtcctcactggggggacccatagtgattagtgggaacacccctggtggcttggacagagggacagagaacatgatgatccctgtgtcctgccactgagtcctggtggcctttaagatgcctatgggaaggggattaggttcctgctgcctgtctctgccctgccgtgtcagtccttggatatcttgcatgttccatccgctacctggatgtggcctgtaggggcagccacctttcctagaagcaggagcaggtagcgatacccaggcaggcctggtcctccccacttttgtgggactcggggattgagcttgcaggcataggggtgtagaaagcagtggtagcgaggtgactgaaggtggactcccactggggatgggattgagtgtggggctagatccctggtctgtagaagggagtaggaaccctgttagttaaattttccccctcaggccacgttagtaggattttgaagagtcagccacactgtggctcttgattttgtgcctggtcacctgcttggtctctggtgatttccctcggggccagcagacactgttggaggtggtgaggcctcaggttcttctgcttcatggttaactcactctgcttaggcatatttccaccttttccagggcctccgtttttctgccagtggattgagaggtggtggtggtctccagcagtggtctcactcttggcaggctatcagagtacccagggagctgcgtagaatgaagttttgagagtctttggtctaggtgaatgccagcgtttcagtttgcttatcttccattatagtagtgtcctctgtaactgtgaatcagggaggttattcataatatctccacatccattttctcccttaattgcgtatgcttttgatttagacagggcaagtagccattttggtggagcgcaggccctggcaggggggcagtgtccagtgccgctctcaattaaacgtgaagatgtgaagatgaataacactgtccagcttgcagagtggacggtggtgaggcaagctttcaagttgcacctactttctagttgagtgacctacacaagtctttgattttttttttttttcttcctggaaaaaaaaagttgtctacttttctcatctgtaaagtgagaagaatcaagtctgccttttctgtctcacggggttgcagtgaagcattagtaccctgagaagcaagatccaaagcccctgagttaggcctgacactggtgtgagacagctgccatctctggcggcactcagcaagtgttcaccagcagctgattctgggaacctcacttcctccgcccttgggcttggtggggttgggtagggttgggtggggctgtggttttcttttaggaggcagcaggccaggcctggagaccagagcttaagtgggcctgggcaggctggggttgaaactcttcaccccttgcggtctgtactgcctcccaactgagcagccagggagaaggcctagagcctgtgcctttcagctagatagctggaggaactggctcctccctccttaggctgtgctggcctgagctgggagcctgagagctggggcagttgtctctaaagtggcttctgggattctggtaagaggtgagctcctggtgctgcctcagagtctttgtgtttcctggcatttgggagagctggagttgggctgtcctgcatgggtaaggtttggggagggactggaacaaggggctagtgaaccttctctgggtttttcctgcctgactatgcgttgacagtcccagctgttgggcctgtgctcctgtacactgcacggccttgagaggagttcggagccctaacatccaggagagaggccccacagcagtggaaggaaatgggcctctcccgaatctcttgtttgtaccccgaggtctgagtggtgatcctggggatgctatgggactctcagcagtaggagtgtgtctgtccccagtctgggtgcccaccagctgtgctgagggtcctctcctgtgtccctgggccaggcagacagggtctttttctggggcttgttaggggaggtcagccacagccccagaccaagtaatttacacaccctgagtgaggggtgggagcagtgggtccaggaagacttagggagctggtgaaagaaaaacttaattctgacacttgttaaactggtaaggtagactgtatcaggactattacgatagatgtaggaattatcgcaatggattttgcagtagaggggagagagtgggccaactccaaatacaacaaagaaaagtgggaacttatagccaaggagcggagtgtgggggctcctgaaaatttctaagagggtagggaaatttttactaaacttagctaactgaattcttgctgcaggcaggccagggtgatcaggttttacctgggagatagtacaggatgagaaatacgttcagatatccagggtgattagatattgaggttgggggattctggttaacaggagttttgctaaaactaggctcttagagagaatggagttaggaaccctaggtcaggaccagatgagtagagggctcagaggaataaagtttggtgagggacagaatctctgtcagtatcccgtggttggcagtgtcctgtccattcttcttgcactggcctgtccctcaggagacaggtgtgagtggtcctgagcttgggccagtgtaagagtaggggacaggtttctctccttgtaggtgtcccttcttgtaagtgtcactctctttttttccagaaaccttggtccttttcagcatctgctggccccttgtcccttggctcccttgctcttggcctgctggatttcccctctgcacccaggaaatcgcatggacctgggtccttgtttccctacagcccaccaattttgctgcttttttctgccccctgaagctgagctcctgctgcaatttgtgttccctgctctccctgtcctgggcaaaccagtctgacaactttgtggttctcgctcccgcctccatcagcctggggattgactgtcccatttgtctgagctgggcagagggaggtgctgtgggggatctcttcctcttgcctggactgcacactcctgctgccttctagccggagctcctgggcattttgcctatgggagcttcaccagcttccttctgtctgaggcctacaagtccctgcctccagcctaccttgttcctcctccatgagtgaggctcctccttttctctctggccctcctgtctacttgatcagactctgcctctcttgagggccggctccctccagcctaccctgcacaggtgaccctgtttggcctgcctccttttcttgaggctgatcttgtctcaacagtcagcttttcaggaaccaggcccttgctgttgtaaggaaaacctgtcggctgtggatggggccttccctccttcctaaaggctctgtagccagcttccacccttgcagtggaacagtggtggtgccagaaccctgctctctgcagccatcctgcctaccacagtcattgtgttttgtaactctagtagcttcttgtgaaatacaagtgatggtataaacgtggataggtttttgaggggggcatgccaaaatcagagttggtggtagtggtgggggattcattcccaagggctctggggtgctaagtgtgtgagcaaagaggaacaagtggcatgtgcccaggatggggtggggcgggcaggtttagttgagggctcctctggtgtagggtagccctgacgctcccctccatggcatgactgatgaggtggcaaaggcaggtgccaggatttggtgtgttgaagattagtgcctgggttgggctctgctcactcctgcagaaagacggttggagaggggctggtcttggtttcacagaggattgtgggattacaggcaagacctgctgagggcttgcactgagccaccgagaggagcagaaagagatacgagggcttcaggtgccagtatggttctcactgttgtgagatctcatttgtgccttttttttttttccttcagacagggtctcactctgttgcccaggctggagtgcagtggccctatcacagctcactgaggcctccaccttcctggctcaagtgatcctcccgcctcagcctcctgagtagcagggaccacaggcatgcagcaccacacccagctaatttaaagaattttttgtagagattggatcttgctatgttgcctaggctggtgtcaaactcctgggcacttgatttgtgcttcttaattcaggggctacttattgaacatatttagcagtcttcagaaagcatcttattctgtaaggggaaccaaatatacccattccccctctctggccttctttccctgtggtctgggcggtctttgagcttgaggtcattaggaggtgtttatttctgtccgccctggtggcctagggcgtaggaatggggtgggatgggatgggatggggtggggtggggtggaggagttattctacatagatagagttctgtttatgcagcactccctgccatacacatctctcactcaaatggtctggccagtcctggttctaatggtcattgtgtcattcccctcagcagcaggaaaggctggaatggtttttgtattgttccttagggggaaaaatcacttggaggaagtatatcagtgcttctgagtgcagagtatttattgacacgtcagttaaaggaagcttttaaacaacatatatgtatctctttagtgaggaataaatctacaagtgcatctcacagctccattttgcagcttccatttcctggaaggtacatggagtttagtggttagggaatggggttttgagctgggattttggagaaactgtttttagcctgtccttggaattgggccaggcctagaaatctgttgaaatagctttctttagttgctgaaactgggatgtgttcatagcactaggacctcgagctccactgatgactgagtggggagggctcagagaagggtccctgtggatgcccgggatgcctgggtttcagtcccgtctcctgcatcgcctacattgcctgaatcttagggtcttacagatttatcatgaagtgggcttggcagcagatggtctggtttcttttgtgcctttgtcagaatctgttcttgctcttcttaggaaaactttctatttcag

SEQ ID NO: 205 - DNA, 3812 bp, homo sapiens
  B2M
gcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcagg

SEQ ID NO: 206 - DNA, 2052 bp, homo sapiens
  HBB
ccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcacctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgatgagctgttcaaaccttgggaaaatacactatatcttaaactccatgaaagaaggtgaggctgcaaccagctaatgcacattggcaacagcccctgatgcctatgccttattcatccctcagaaaaggattcttgtagaggcttgatttgcaggttaaagttttgctatgctgtattttacattacttattgttttagctgtcctcatgaatgtcttttcactacccatttgcttatcctgcatctctctcagccttgact

SEQ ID NO: 207 - protein, 147 aa, homo sapiens
  HBB
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 208 - DNA, 1000 bp, homo sapiens
  CD40L
gaattcactggggagagcattcaggaagatgacaacaggataataggtcaacagagtaatagagaggtcgctaaaaataaactctaagaagtattcagccaaaactattattgagctaataatggtgggatcaatttcaggggaatattgtgggcagaagtcagactgtaggaggctggggatcaagaagttgaggcaaggaggttggacaacaactgttttttcaagttggtcacgtgaacaaatctgtgaccttcagcctcccctccctcgggtcttggctgagctgattgcagggcccctgcagctctggcactctcaagttgtataaaactgacagtgcagaagtccttgagcccattttggctctcatgataattttccttcagtggaactaaggttacttgtctaagaaccaaagcctctgacttgactgatcaaagttcatcacgtgcatcgaagccacctacttggcagatgtagtgaaaagctacatagatctgggcccaggacaggatgctggggcgtgggaggggaagaaagcaggtgctaactatatagatagcatgcctatcagagcagtttttacgtttcctatttgtctctcaaaacaattttataggaatcatcaaagcaattttatcatggtttctagaccaggtttggatgtgaggtagggatttccacagctgcttttagtttgaaggaaatctgataagatgatgcaaaagcccttcagaaatgtgtaatcctacacacttcagtgattcaattcattgtcaaaacttaaggtgtttttaatattgttattgttcatttggtttttaccaacatgtaaggagttggcaattatttgttaaactcatgtcttaggctaaataaattccaaaaaattcaggatgagaattgtttattgcttaacgtgtgtcaaatttcttccatgcacatctttattagatcttcacagcaacctacaggataagcaagacaggtgcaagtg

SEQ ID NO: 209 - protein, 261 aa, homo sapiens
  CD40L
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 210 - DNA, 3435 bp, homo sapiens
  ADCY3
atgccgaggaaccagggcttctccgagcccgaatactcggccgagtactcagccgagtactccgtcagcctgccctccgaccctgaccgcggggtgggccggacccatgaaatctcggtccggaactcgggctcctgcctgtgcctgcctcgcttcatgcggctgactttcgtgccggagtccttggagaacctctaccagacctacttcaaaaggcagcgccacgagaccctgctggtgctggtggtctttgcagccctctttgactgctacgtggtggtcatgtgtgctgtggtcttctccagcgacaagctggcttccctcgccgtggctggaattggactggtgttggacatcatcctcttcgtgctctgcaaaaaggggctgctcccggaccgggtcacccgcagagtgctgccctacgtgctgtggctgctcataaccgcccagatcttctcctacctgggcctgaacttcgcgcgtgcccacgcggctagtgacacggtgggctggcaggtcttctttgtcttctccttcttcatcacgctgcccctcagcctcagccccatcgtgatcatctccgtggtctcctgtgtggtgcacacgttggtcctgggggtcaccgtggcccagcagcagcaggaggagctcaaggggatgcagctgctgcgggagatcctggccaacgtcttcctctacctgtgcgccatcgctgtgggcatcatgtcctactacatggctgaccgcaagcaccgcaaggccttcctggaggcccgccagtcgctggaggtgaagatgaacctggaagagcagagccagcagcaggagaacctcatgctttccatcctgcccaagcacgtggctgacgagatgctgaaagacatgaagaaagacgagagccagaaggaccagcagcagttcaacaccatgtacatgtaccgtcacgagaacgtcagcatcctctttgccgacatcgtgggctttacccagctgtcttctgcctgcagtgcccaggagcttgtgaagctgctcaacgagctctttgcccgctttgacaagctggcagctaaataccaccagctgcggattaagatcctgggcgactgctactactgcatctgcggcttgcccgactaccgggaggaccacgccgtctgctccatcctcatggggctggccatggtggaggccatctcgtatgtgcgggagaagaccaagactggggtggacatgcgtgtgggggtgcacacgggcaccgtgctggggggcgtcctgggccagaagcgctggcagtacgacgtgtggtcgactgatgtcactgtagccaacaagatggaggccggcggcatccctgggcgcgtgcacatctcccagagcaccatggactgcctgaaaggggagtttgatgtggagccaggcgatgggggcagccgctgtgattacctagaagagaagggtattgaaacctacctcatcattgcctccaagccagaggtgaagaaaacagccacccagaatggcctcaatggctcggccctgcccaatggagcaccagcttcctcaaagtccagctcccctgccctcattgagaccaaggagcccaacgggagtgcccacagcagtgggtccacgtcggagaagcccgaggagcaggatgcccaggccgacaacccctcattccccaacccacgccggaggctgcgcctgcaggacctggctgaccgagtggtggatgcctctgaagatgagcacgagctcaaccagctgctcaacgaggccctgcttgagcgagagtccgcccaagtagtaaagaagagaaacaccttcctcttgtccatgcggttcatggaccccgagatggaaacccgctactcggtggagaaggagaagcagagtggggctgccttcagctgctcctgcgtcgtcctgctctgcacggccctggtcgagatactcatcgacccctggctaatgacaaactatgtgaccttcatggtgggggagattctgctcctcatcctgaccatctgctccctggctgccatctttccccgggcctttcctaagaagcttgtggccttctcaacttggattgaccggacccgctgggccaggaacacctgggccatgctcgccatcttcatcctggtgatggcaaatgtcgtggacatgctcagctgtctccagtactacacgggacccagcaatgcaacggcagggatggaaacggagggcagctgcctggagaaccccaagtattacaactatgtggccgtgctgtccctcatcgccaccatcatgctggtgcaggtcagccacatggtgaagctcacgctcatgctgctcgtcgcaggcgccgtggccaccatcaacctctatgcctggcgtcccgtctttgatgaatacgaccacaagcgttttcgggagcacgacttacctatggtggccttagagcagatgcaaggattcaaccctgggctcaatggcactgacaggctgcccctggtgccttccaagtactctatgacggtgatggtgttcctcatgatgctcagcttctactacttctcccgccacgtagaaaaactggcacggacacttttcttgtggaagattgaggtccacgaccagaaggaacgtgtctatgagatgcgacgctggaacgaggccttggtcaccaacatgttgcctgagcacgtggcacgccatttcctggggtccaagaagagagatgaggagctgtatagccagacgtatgatgagattggagtcatgtttgcctccctgcccaactttgctgacttctacacagaggagagcatcaacaatggtggtattgagtgtctgcgtttcctcaatgaaatcatctcagattttgactctctcctggacaatcccaagttccgggtgatcaccaagatcaaaaccattggcagcacgtatatggcggcttcaggagtcacccccgatgtcaacaccaatggctttgccagctccaacaaggaagacaagtccgagagagagcgctggcagcacctggctgacctggccgacttcgcgctggccatgaaggatacgctcaccaacatcaacaaccagtccttcaataacttcatgctgcgcataggcatgaacaaaggcggggttctggctggggtcatcggagcccggaaaccacactacgacatctggggcaatacagtcaatgtagccagcaggatggagtccacgggggtcatgggcaacattcaggtggtagaagaaacccaagtcatcctccgagagtacggcttccgctttgtgaggcgaggccccatctttgtgaaggggaagggggagctgctgaccttcttcttgaaggggcgggataagctagccaccttccccaatggcccctctgtcacactgccccaccaggtggtggacaactcctga

SEQ ID NO: 211 - protein, 1144 aa, homo sapiens
  ADCY3
MPRNQGFSEPEYSAEYSAEYSVSLPSDPDRGVGRTHEISVRNSGSCLCLPRFMRLTFVPESLENLYQTYFKRQRHETLLVLVVFAALFDCYVVVMCAVVFSSDKLASLAVAGIGLVLDIILFVLCKKGLLPDRVTRRVLPYVLWLLITAQIFSYLGLNFARAHAASDTVGWQVFFVFSFFITLPLSLSPIVIISVVSCVVHTLVLGVTVAQQQQEELKGMQLLREILANVFLYLCAIAVGIMSYYMADRKHRKAFLEARQSLEVKMNLEEQSQQQENLMLSILPKHVADEMLKDMKKDESQKDQQQFNTMYMYRHENVSILFADIVGFTQLSSACSAQELVKLLNELFARFDKLAAKYHQLRIKILGDCYYCICGLPDYREDHAVCSILMGLAMVEAISYVREKTKTGVDMRVGVHTGTVLGGVLGQKRWQYDVWSTDVTVANKMEAGGIPGRVHISQSTMDCLKGEFDVEPGDGGSRCDYLEEKGIETYLIIASKPEVKKTATQNGLNGSALPNGAPASSKSSSPALIETKEPNGSAHSSGSTSEKPEEQDAQADNPSFPNPRRRLRLQDLADRVVDASEDEHELNQLLNEALLERESAQVVKKRNTFLLSMRFMDPEMETRYSVEKEKQSGAAFSCSCVVLLCTALVEILIDPWLMTNYVTFMVGEILLLILTICSLAAIFPRAFPKKLVAFSTWIDRTRWARNTWAMLAIFILVMANVVDMLSCLQYYTGPSNATAGMETEGSCLENPKYYNYVAVLSLIATIMLVQVSHMVKLTLMLLVAGAVATINLYAWRPVFDEYDHKRFREHDLPMVALEQMQGFNPGLNGTDRLPLVPSKYSMTVMVFLMMLSFYYFSRHVEKLARTLFLWKIEVHDQKERVYEMRRWNEALVTNMLPEHVARHFLGSKKRDEELYSQTYDEIGVMFASLPNFADFYTEESINNGGIECLRFLNEIISDFDSLLDNPKFRVITKIKTIGSTYMAASGVTPDVNTNGFASSNKEDKSERERWQHLADLADFALAMKDTLTNINNQSFNNFMLRIGMNKGGVLAGVIGARKPHYDIWGNTVNVASRMESTGVMGNIQVVEETQVILREYGFRFVRRGPIFVKGKGELLTFFLKGRDKLATFPNGPSVTLPHQVVDNS

SEQ ID NO: 212 - DNA, 744 bp, homo sapiens
  BDNF
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacatgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 213 - protein, 247 aa, homo sapiens
  BDNF
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 214 - DNA, 1512 bp, homo sapiens
  KSR2
aggacttcaacataggaacttgagggggacaaaattcagcccataacatgcattgatgtataacaaaaaagcaaaaatggaaccaaatgcaagtgagtctgcgattccagcgaggaggcagcggcagcccagagccaagaagaagagcaaacccttgaacctcaagatccacagcagcgtaggcagctgcgagaacatcccctctcagcagcgctccccgctgctgtccgagcgctccctccgctccttctttgtgggacacgcacctttcctgccttccacccctcctgttcacactgaggccaacttctctgcaaacacactgtcagtgccacgctggtccccgcagatccctcgcagagatcttggcaactccatcaagcacaggttttccaccaagtactggatgtctcagacgtgcacagtctgtgggaaagggatgctttttggcctcaagtgtaaaaactgcaagttaaagtgccacaacaaatgcaccaaagaagccccaccctgtcatcttctgatcatccaccgaggagatccagcaaggttagtccggacagagtccgttccgtgtgacatcaacaaccctctacggaagccacctcgctattcagacctgcacatcagtcagacgctccccaaaaccaacaaaatcaacaaggaccacatccctgtcccttaccagccagactccagcagcaacccctcctccacgacgtcctccacgccctcctcgccagcaccccccctccctcctagtgccacgccgccttctcccctacacccttccccacagtgcacacggcagcagaagaacttcaacctgccagcatcccactactacaaatacaagcagcagttcatcttcccagatgtggtgccggtgccggagacgccgacccgggcgccccaggtcatcctgcatccggtgacctcgaatccaatcttggaaggaaatccattacttcaaattgaagtggagccaacgtcggagaatgaagaggtccatgatgaggccgaagagtcagaggatgacttcgaggagatgaacctgtccctcctctcggcccggagcttcccacgcaaggccagccagaccagcatcttccttcaggagtgggacatcccctttgagcagctggagattggcgagctcattggaaagggccgctttgggcaagtgtaccacggccgctggcatggcgaggtggccatccggctgattgacattgagagggacaacgaggaccagctcaaggccttcaagcgggaggtgatggcctacaggcagacacggcatgagaacgtggtgcttttcatgggtgcctgcatgagcccgcctcacctggccatcatcaccaggccagtttgattgtcctaaatataacgtacaggagttggggggcctgagcacatgaagattccgtccccatcatcgagtggaagcggcttccttttatgttcaaaaagctgaatcgctttccctttataaagcacagctgaaaaggt

SEQ ID NO: 215 - protein, 950 aa, homo sapiens [sequence omitted]

SEQ ID NO: 216 - DNA, 501 bp, homo sapiens
  LEP
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctga

SEQ ID NO: 217 - protein, 167 aa, homo sapiens
  LEP
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 218 - DNA, 49 bp, homo sapiens
  TRAC TALEN target s
ttcctcctactcaccatcagcctcctggttatggtacaggtaagagcaa

SEQ ID NO: 219 - DNA, 47 bp, homo sapiens
  B2M TALEN target
tccgtggccttagctgtgctcgcgctactctctctttctggcctgga

SEQ ID NO: 220 - DNA, 2550 bp, artificial
  CD22CAR repair template
ggttttcccagtcacgacgttgtaaaacgacggccagtgaattcgagctcggtacctcgcgaatgcatctagatgcggccgcaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagtccggtgagggcagaggaagtcttctaacatgcggtgacgtggaggagaatccgggccccggatccgctctgcccgtcaccgctctgctgctgccactggcactgctgctgcacgctgctaggccccaggtgcagctgcagcagagcggccctggcctggtgaagccaagccagacactgtccctgacctgcgccatcagcggcgattccgtgagctccaactccgccgcctggaattggatcaggcagtccccttctcggggcctggagtggctgggaaggacatactatcggtctaagtggtacaacgattatgccgtgtctgtgaagagcagaatcacaatcaaccctgacacctccaagaatcagttctctctgcagctgaatagcgtgacaccagaggacaccgccgtgtactattgcgccagggaggtgaccggcgacctggaggatgcctttgacatctggggccagggcacaatggtgaccgtgtctagcggaggcggaggctccggaggcggaggatctggcggaggcggaagcgatatccagatgacacagtccccatcctctctgagcgcctccgtgggcgacagagtgacaatcacctgtagggcctcccagaccatctggtcttacctgaactggtatcagcagaggcccggcaaggcccctaatctgctgatctacgcagcaagctccctgcagagcggagtgccatccagattctctggcaggggctccggcacagacttcaccctgaccatctctagcctccaggccgaggacttcgccacctactattgccagcagtcttatagcatcccccagacatttggccagggcaccaagctggagatcaaggctcccaccacaacccccgctccaaggccccctacccccgcaccaactattgcctcccagccactctcactgcggcctgaggcctgtcggcccgctgctggaggcgcagtgcatacaaggggcctcgatttcgcctgcgatatttacatctgggcacccctcgccggcacctgcggggtgcttctcctctccctggtgattaccctgtattgcagacggggccggaagaagctcctctacatttttaagcagcctttcatgcggccagtgcagacaacccaagaggaggatgggtgttcctgcagattccctgaggaagaggaaggcgggtgcgagctgagagtgaagttctccaggagcgcagatgcccccgcctatcaacagggccagaaccagctctacaacgagcttaacctcgggaggcgcgaagaatacgacgtgttggataagagaagggggcgggaccccgagatgggaggaaagccccggaggaagaaccctcaggagggcctgtacaacgagctgcagaaggataagatggccgaggcctactcagagatcgggatgaagggggagcggcgccgcgggaaggggcacgatgggctctaccaggggctgagcacagccacaaaggacacatacgacgccttgcacatgcaggcccttccaccccggtgaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggaattcagtcaatatgttcaccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaactcgagtatcggatcccgggcccgtcgactgcagaggcctgcatgcaagcttggcgtaatcatggtcatagctgtttcctgtgtgaaattgtt

SEQ ID NO: 221 - DNA, 23 bp, artificial
  M13F
cccagtcacgacgttgtaaaacg

SEQ ID NO: 222 - DNA, 23 bp, artificial
  M13R
cctgtgtgaaattgttatccgct

SEQ ID NO: 223 - DNA, 26 bp, artificial
  B2M cross_L
cattcctgaagctgacagcattcggg

SEQ ID NO: 224 - DNA, 26 bp, artificial
  B2M cross_R
gggtaggagagactcacgctggatag

SEQ ID NO: 225 - DNA, 26 bp, artificial
  B2M up_L
cgtgacttcccttctccaagttctcc

SEQ ID NO: 226 - DNA, 26 bp, artificial
  B2M up_R
acgcttatcgacgccctaaactttgt

SEQ ID NO: 227 - DNA, 26 bp, artificial
  TRAC cross_L
gcatttcaggtttccttgagtggcag

SEQ ID NO: 228 - DNA, 26 bp, artificial
  TRAC cross_R
tggcaagtcacggtctcatgctttat

SEQ ID NO: 229 - DNA, 26 bp, artificial
  TRAC up_L
cttgtccatcactggcatctggactc

SEQ ID NO: 230 - DNA, 26 bp, artificial
  TRAC up_R
atcggtgaataggcagacagacttgt

SEQ ID NO: 231 - DNA, 26 bp, artificial
  CD22CAR_F
aagatgtacagtttgctttgctgggc

SEQ ID NO: 232 - DNA, 26 bp, artificial
  CD22CAR_R
acgtcaccgcatgttagaagacttcc

SEQ ID NO: 233 - DNA, 42 bp, artificial
  B2M_NGS_L
ctacacgacgctcttccgatctgtccctctctctaacctggc

SEQ ID NO: 234 - DNA, 54 bp, artificial
  B2M_NGS_R
gtgactggagttcagacgtgtgctcttccgatctgaagggaagtcacggagcga

SEQ ID NO: 235 - DNA, 54 bp, artificial
  TRAC_NGS_L
gtcgactagggataacagggtaattatccagaaccctgaccctgccgtgtacca

SEQ ID NO: 236 - DNA, 54 bp, artificial
  TRAC_NGS_R
aaactgtattataagtaaatgcattggatttagagtctctcagctggtacacgg

SEQ ID NO: 237 - DNA, 203 bp, artificial
  HSC ssODN1
acttcatccacgttcaccttgccccacagggcagtaacggcagacttctcctccctaggagtcagatgcaccatggtgtcggcttgaggttgacagtgaacacagttgtgtcagaagcaaatgtaagcaatagatggctctgccctgacttttatgcccagccctggctcctgccctccctgctcctgggagtagattggcca

SEQ ID NO: 238 - DNA, 203 bp, artificial
  HSC ssODN2
gataccaacctgcccagggcctcaccaccaacttcatccacgttcaccttgccccacagggcagtaacggcagacttctcctccctaggagtcagatgcaccatggtgtcggcttgaggttgacagtgaacacagttgtgtcagaagcaaatgtaagcaatagatggctctgccctgacttttatgcccagccctggctcctg

SEQ ID NO: 239 - DNA, 50 bp, homo sapiens
  HBB Target
ttgcttacatttgcttctgacacaactgtgttcactagcaacctcaaaca

SEQ ID NO: 240 - DNA, 170 bp, artificial
  TRAC ssODN
cctgggttggggcaaagagggaaatgagatcatgtcctaaccctgatcctcttgtcccacagatatccagaaccctaggtgaaagcttagactagtgaccctgccgtgtaccagctgagagactctaaatccagtgacaagtctgtctgcctattcaccgattttgattc

SEQ ID NO: 241 - DNA, 2781 bp, artificial
  HBB TALEN LEFT
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 242 - protein, 925 aa, artificial
  HBB TALEN LEFT PRT
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 243 - DNA, 2781 bp, artificial
  HBB TALEN RIGHT
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 244 - protein, 925 aa, artificial
  HBB TALEN RIGHT PRT
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 245 - protein, 1423 aa, artificial
  Cas9 protein sequence
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 246 - DNA, 99 bp, artificial
  TRAC guideRNA sequence
cagggttctggatatctgtgttttagagctagaaatagcaagttaaaataaggctagtccgttatcaacttgaaaaagtggcaccgagtcggtgctttt

(19) United States
(12) Patent Application Publication
(10) Pub. No.: US 2023/0212613 A1
(43) Pub. Date: Jul. 6, 2023

(54) METHODS FOR TARGETED INSERTION OF EXOGENOUS SEQUENCES IN CELLULAR GENOMES

(71) Applicant: CELLECTIS S.A., Paris (FR)

(72) Inventors: Ming YANG, Great Neck, NY (US); Alexandre JUILLERAT, New York, NY (US); Philippe DUCHATEAU, Saint Louis et Parahou (FR); Patrick HONG, New York, NY (US)

(21) Appl. No.: 17/996,733

(22) PCT Filed: May 6, 2021
(86) PCT No.: PCT/EP2021/061999

§ 371 Date: Oct. 20, 2022

Related U.S. Application Data
(60) Provisional application No. 63/020,900, filed on May 6, 2020.

(30) Foreign Application Priority Data
Jun. 10, 2020 (DK) PA202070366

(51) Int. Cl. C12N 15/90; C07K 14/725; C12N 9/22; C12N 5/0783

(52) U.S. Cl. CPC C12N 15/907; C07K 14/7051; C12N 9/22; C12N 5/0636; C12N 2310/20

(57) ABSTRACT

The present disclosure provides methods for targeted insertion of an exogenous sequence at a genomic locus in a cell, wherein said insertion is induced by a sequence-specific endonuclease that has cleavage activity at said locus, at least 5 hours before the introduction into said cell of a DNA template comprising said exogenous sequence.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 368,372 Byte ASCII (Text) file named “Sequence_Listing.txt,” created on Apr. 30, 2020.

### FIELD OF THE INVENTION

The present invention generally relates to the field of gene therapy, and more specifically to the treatment and prevention of genetic diseases and cancer.

### BACKGROUND

The ability to modify the expression of single genes and proteins has become one of the most important tools in molecular and cellular biology. Several methodologies have been developed to allow for specific gene manipulation in tissue culture cells, which have become colloquially known as “genome-editing.” These methods rely on nucleases that are engineered to cut specific genomic target sequences, including Meganuclease, Zinc Finger Nucleases (ZFN), Transcription Activator-like Effector Nucleases (TALEN) and Clustered Regularly Interspaced Short Palindromic Repeats (CRISPR) nucleases (Gaj, Gersbach, Barbas, & III, 2013) (FIG. 1).

In TALE-Nucleases, a TAL effector DNA-binding domain is fused to a DNA cleavage domain. Transcription activator-like effectors (TALEs) can be engineered to bind to practically any desired DNA sequence, so when combined with a nuclease, DNA can be cut at specific locations. TALEN uses engineered Fold endonuclease as the DNA cleavage domain. This non-specific cleavage domain from the type IIs restriction endonuclease FokI must dimerize in order to cleave DNA and thus a pair of TALEN are required to target non-palindromic DNA sites.

TALEN creates double strand breaks (DSB) on genomic DNA, which will then be repaired by the cellular DSB repair machinery. In mammalian cells, two major pathways exist to repair DSBs—homologous recombination and nonhomologous end joining (NHEJ) (Liang, Han, Romanienko, & Jasin, 1998). NHEJ, the rejoining of DNA ends with the use of little or no sequence homology, involves the processing of ends such that nucleotides are often deleted or inserted at the break site prior to ligation (Paques & Haber, 1999). Such modifications are likely central to the ability of mammalian cells to rejoin DNA ends with a variety of structures. Homology-directed repair (HDR) of a DSB, in contrast, requires significant lengths of sequence homology so that a DNA end from one molecule can invade a homologous sequence and prime repair synthesis (Paques & Haber, 1999).

The scientific community has been taking advantage of these engineered nuclease to create edits (“knock-out” or “knock-in”) in cell lines (Hsu, Lander, & Zhang, 2014)(Hsu et al., 2014) and even primary human T cells (Schumann et al., 2015) aiming to control T cell function (Roth et al., 2018) and in some cases, produce CAR-T cells (chimeric antigen receptor-T cells) as described in WO2013176915 and Eyquem et al., 2017. A better understanding of how these tools function in cells is warranted for better design of gene editing processes in CAR-T engineering.

In a recent study, the kinetics of the CRISPR-Cas9 system has been studied by generating DSB at genomic DNA in cultured cell lines (Brinkman et al., 2018). However, measurements and modeling of the kinetics of broken DNA ends rejoining after a Cas9-induced lesion has indicated that the rate of DSB repair was variable according to the type of repair mechanisms involved. Furthermore, the results indicated that the repair process tend to be error prone.

The kinetics of the repair of DSB induced by TALEN, another important tool for gene editing utilizing a different DNA cutting mechanism, is not clearly understood.

WO 2015/057980 describes compositions and methods for use in gene therapy and genome engineering, in particular a method of integrating one or more transgenes into a genome of an isolated cell, the method comprising sequentially introducing the transgene and at least one nuclease into the cell such that the nuclease mediates targeted integration of the transgene.

WO 2018/007263 describes methods of sequential gene editing aiming to improve the genetic modification of primary human cells, especially immune cells originating from individual donors or patients.

In a context where, the gene editing field is not satisfied by simple gene KO, but is exploring the opportunity of targeted and controllable gene editing such as gene knock-in, which relies more on HDR than the NHEJ repair pathway, the present invention provides means to better harness gene editing tool to improve gene integration efficiency.

This background information is provided for informational purposes only. No admission is necessarily intended, nor should it be construed, that any of the preceding information constitutes prior art against the present invention.

### SUMMARY

It is to be understood that both the foregoing general description of the embodiments and the following detailed description are exemplary, and thus do not restrict the scope of the embodiments.

The present disclosure provides studies that characterized the kinetics of NHEJ of TALEN-induced DSB, and additionally studied the HDR rate of gene edited cells in response to an exogenous DNA repair template. These findings shed new light on the DSB repair mechanism using an engineered nuclease and on how to design non-viral mediated gene knock-in experiments.

In one aspect, the invention provides a method for targeted insertion of an exogenous sequence at a genomic locus in a cell, wherein the insertion is induced by a sequence-specific endonuclease that has cleavage activity at the locus, at least 5 hours before the introduction into the cell of a DNA template comprising the exogenous sequence.

In some embodiments, the exogenous sequence is inserted at the genomic locus in a cell by homologous recombination, non-homologous end joining (NHEJ), homology directed repair (HDR), microhomology-mediated end joining (MMEJ) or homology-mediated end joining (HMEJ).

In some embodiments, the sequence specific endonuclease has cleavage activity for at least 5 hours (i.e. more than 5 hours) until the DNA template is introduced into the cell. In some embodiments, the sequence-specific endonuclease has cleavage activity for at least 15 hours, preferably for at least 18 hours, more preferably at least 20 hours until the DNA template is introduced into the cell.

In some particular aspects, the invention provides a method for targeted insertion of an exogenous sequence at a genomic locus in a cell, wherein the method comprises at least the steps of:
a) transfecting the cell with a sequence-specific endonuclease polypeptide having cleavage activity at the genomic locus;
b) introducing into the cell, between 5 and 25 hours after the transfecting step of a), a DNA template comprising the exogenous sequence to be inserted at the locus by homologous recombination, NHEJ, HDR, MMEJ or HMEJ; and
c) culturing and selecting the cells, in which the exogenous sequence has been inserted at the locus.

In another aspect, the invention provides a method for targeted insertion of an exogenous sequence at a genomic locus in a cell, wherein the method comprises at least the steps of:
a) transfecting the cell with a sequence-specific endonuclease polynucleotide having cleavage activity at the genomic locus;
b) introducing into the cell, between 10 and 30 hours after said transfecting step of a), a DNA template comprising the exogenous sequence to be inserted at the locus by homologous recombination, NHEJ, HDR, MMEJ or HMEJ, and
c) culturing and selecting the cells, in which the exogenous sequence has been inserted at the locus.

In some embodiments, the cells are cultured, at least in step c), between 25 and 40° C., preferably between 28 and 38° C., and more preferably between 30 and 37° C.

In some embodiments, the nucleic acid encoding the sequence-specific endonuclease polynucleotide is a mRNA.

In some embodiments, the DNA template is introduced between 10 and 20 hours after the transfection of the nucleic acid.

In some embodiments, the endonuclease is a TALE-nuclease. In some embodiments, the endonuclease is a RNA-guided endonuclease, such as Cas9 or Cpf1. In some embodiments, a guide-RNA associated with said RNA-guided endonuclease is introduced concomitantly with the RNA-guided endonuclease.

In some embodiments, the exogenous sequence is inserted at the locus by homologous recombination.

In some embodiments, the DNA template is double stranded (dsDNA). In some embodiments, the dsDNA is a PCR product. In some embodiments, the dsDNA has a length of more than 2 kb, preferably more than 2.5 kb, more preferably more than 3 kb, and even more preferably between 2 and 10 kb.

In some embodiments, the DNA template is a single stranded polynucleotide. In some embodiments, the DNA template is a short single-stranded oligodeoxynucleotide (ssODN). In some embodiments, the ssODN has homology arms comprised between 50 and 200 bp, preferably between 80 and 150 bp, and more preferably between 90 and 120 bp.

In some embodiments, the methods of the invention comprise at least two transfection steps, wherein a first transfection step introduces the nucleic acid encoding the sequence-specific endonuclease into the cell, and a second transfection step introduces the DNA template comprising the exogenous sequence to be inserted. In some embodiments, the first transfection step is by electroporation or nanoparticle transformation. In some embodiments, the second transfection step is by electroporation, nanoparticle or viral transformation.

In some embodiments, the cell is a mammalian cell, preferably a primate cell, and more preferably a human cell. In some embodiments, the cell is a primary cell. In some embodiments, the cell is an immune cell, preferably a T-cell or a NK cell. In some embodiments, the cell is a primary T-cell, more preferably a primary T-cell from a patient, such as a tumor infiltrating lymphocyte (TIL), or a primary T-cell from a donor.

In some embodiments, the exogenous sequence is inserted at a locus encoding proteins selected from TCR, β2m, PD1, CTLA4, TIM3, TGFβ, TGFβR, IL-10, IL10R, IL27RA, STAT1, STAT3, ILT2, ILT4, JAK2, AURKA, DNMT3, MT1A, MT2A, PTGER2, miR21, mir26A, miR101 miRNA31, MT1A, MT2A, PTGER2 GCN2, PRDM1, CD52, GR, HPRT, GGH, GM-CSF or DCK.

In some embodiments, the insertion of the exogenous sequence prevents the expression of the endogenous gene present at the locus.

In some embodiments, the exogenous sequence is inserted at a locus selected from CD25, CD69 or one listed in Table 1 (list of gene loci upregulated in tumor exhausted infiltrating lymphocytes), Table 2 (list of gene loci upregulated in hypoxic tumor conditions) or Table 3.

In some embodiments, the exogenous sequence encodes a polypeptide selected from a Chimeric Antigen Receptor (CAR), a recombinant TCR, dnTGFβRII, sgp130, mutated IL6Ra (mutIL6Ra), HLA-G, IL-2, IL-12, IL-15, IL-18, FOXP3 inhibitor, a secreted inhibitor of Tumor Associated Macrophages (TAM), such as a CCR2/CCL2 neutralization agent, immunogenic peptide(s) or a secreted antibody, such as an anti-IDO1, anti-IL10, anti-PD1, anti-PDL1, anti-IL6, anti-GM-CSF or anti-PGE2 antibody.

In some embodiments, the exogenous sequence comprises a sequence for correcting a mutated endogenous gene present at the genomic locus, such as IL7R, CD45, IL2RG, JAK3, RAG1, RAG2, ARTEMIS, ADA, TRAC, CCR5, RFX5, RFXAP, RFXANK(B), CIITA, ZAP-70, CRAC, ORAI1, STIM1, POLA1, MAP3K14, GATA2, MCM4, IRF8, RTEL1, FCGR3A, Ncr1, TAP1, TAP2, RFX5, RFXAP, RFXANK(B), CIITA, ZAP-70, CRAC, ORAI1 and STIM1 (preferably in NK cells).

In some embodiments, the cell is a hematopoietic stem cell (HSC). In some embodiments, the exogenous sequence is inserted at a locus expressed in HSC derived lineage cells such as CCR5, TMEM119, CD11B, β2m, CX3CR1 or S100A9. In some embodiments, the exogenous sequence comprises a sequence encoding or correcting:
HBB for treating Sickle Cell Anemia (SCA);
CD40L for treating X-linked hyper-immunoglobulin M syndrome;
IDUA for treating Mucopolysaccharidosis Type I (Scheie, Hurler-Scheie or Hurler syndrome),
IDS for treating Mucopolysaccharidosis Type II (Hunter),
ARSB for treating Mucopolysaccharidosis Type VI (Maroteaux-Lamy),
GUSB for treating Mucopolysaccharidosis Type VII (Sly),
ABCD1 for treating X-linked Adrenoleukodystrophy,
GALC for treating Globoid Cell Leukodystrophy (Krabbe),
ARSA for treating Metachromatic Leukodystrophy,
GBA for treating Gaucher Disease,
FUCA1 for treating Fucosidosis,
MAN2B1 for treating Alpha-mannosidosis,
AGA for treating Aspartylglucosaminuria,
ASAH1 for treating Farber Disease,
HEXA for treating Tay-Sachs Disease,
GAA for treating Pompe Disease,
SMPD1 for treating Niemann Pick Disease,
DMD for treating Duchenne muscular dystrophy
LIPA for treating Wolman Syndrome,
CDKL5 for treating CDKL5-deficiency related disease, or
ADCY3, BDNF, KSR2, LEP for treating severe obesity.

In another aspect, the invention provides a method for producing therapeutic cells, comprising the steps of:
providing primary immune cells from a donor or a patient or derived from human iPS or hES cells;
performing a targeted insertion according to the methods herein; and
purifying and freezing the cells for subsequent use as a therapeutic composition.

In some embodiments, the methods of the invention do not comprise a step involving a viral vector.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. A. Scheme of T cell activation and transfection protocol. B. Expression of TALEN protein at different time points. Western blot with an anti-RVD antibody (TALEN, upper panel), or an anti-actin antibody (control, lower panel) C. Scheme of qPCR strategy to measure un-joined DSB created by TALEN. D. Fold change of un-joined DSB at either TRAC TALEN target site (upper panel) or B2M TALEN target site (lower panel). Experiments were performed in three different donors.

FIG. 2. A. Time course experiment showing gradual accumulation of indels at TRAC (upper panel) or B2M (lower panel) locus. Experiments were performed in three different donors. B. The size of deletion over time. The abundance of the different sizes of deletion at TALEN target sites TRAC (upper panel) or B2M (lower panel) in same sample was determined by deep-sequencing. Experiments were performed in three different donors.

FIG. 3. A. Design of the 20 bp insert ssODN. LHA: Left Homology Arm. RHA: Right Homology Arm B. Percentage of targeted integration (KI) depending on the timing of ssODN transfection C. Targeted integration fold increased compared to co-transfection.

FIG. 4. A. Scheme of the CD22CAR repair template. B. Detection of dsDNA repair template by qPCR depending on timing of its transfection allowing to calculate the half-life of dsDNA repair template. Experiments were performed in three different donors. C. Scheme of the Two-Step transfection. The cells were transfected with site-specific nuclease targeting TRAC and let rest for various length of times before a second transfection of dsDNA repair template that encodes the CD22CAR insert. D. The percentage of CD22CAR+ cells depending on timing of dsDNA repair template transfection. The experiments were performed with three donors.

FIG. 5. The transfection procedure did not cause increased toxicity to the edited T cells.

FIG. 6: CD22CAR dsDNA Cas9 mediated targeted integration efficiency after co-transfection (0 hr) or delayed dsDNA delivery (at indicated time points). The targeted integration efficiencies are normalized to the co-transfection, error bar represents standard deviation (n=2).

FIG. 7. A. Design of two ssODN integration at HBB locus strategies. B. ssODN targeted integration frequency in HSCs upon cotransfection (Co-TF) or 20 hrs delay ssODN delivery (20 hr delay). C. ssODN targeted integration fold increased compared to co-transfection.

### DETAILED DESCRIPTION

Unless specifically defined herein, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

Where a numerical limit or range is stated herein, the endpoints are included. Also, all values and subranges within a numerical limit or range are specifically included as if explicitly written out.

In one embodiment, the invention provides a method for targeted insertion of an exogenous sequence at a genomic locus in a cell, wherein the insertion is induced by a sequence-specific endonuclease that has cleavage activity at the locus, at least 5 hours before the introduction into the cell of a DNA template comprising the exogenous sequence.

In another embodiment, the invention provides a method for targeted insertion of an exogenous sequence at a genomic locus in a cell, wherein the method comprises at least the steps of:
a) transfecting the cell with a sequence-specific endonuclease polypeptide having cleavage activity at the genomic locus;
b) introducing into the cell, between 5 and 25 hours after the transfecting step of a), a DNA template comprising the exogenous sequence to be inserted at the locus by homologous recombination, NHEJ, HDR, MMEJ or HMEJ; and
c) culturing and selecting the cells, in which the exogenous sequence has been inserted at the locus.

In another aspect, the invention provides a method for targeted insertion of an exogenous sequence at a genomic locus in a cell, wherein the method comprises at least the steps of:
a) transfecting the cell with a sequence-specific endonuclease polynucleotide having cleavage activity at the genomic locus;
b) introducing into the cell, between 10 and 30 hours after the transfecting step of a), a DNA template comprising the exogenous sequence to be inserted at the locus by homologous recombination, NHEJ, HDR, MMEJ or HMEJ, and
c) culturing and selecting the cells, in which the exogenous sequence has been inserted at the locus.

In some embodiments, the cells are cultured, at least in step c), between about 25 and about 40° C., preferably between about 28 and about 38° C., and more preferably between about 30 and about 37° C.

In some embodiments, the methods comprise at least two transfection steps, wherein a first transfection step introduces the sequence-specific endonuclease into the cell, as a polypeptide or polynucleotide, and a second transfection step introduces the DNA template comprising said exogenous sequence to be inserted. In some embodiments, the first transfection step is by electroporation or nanoparticle transformation. In some embodiments, the second transfection step is by electroporation, nanoparticle or viral transformation. In some embodiments, the methods of the invention do not comprise a step involving a viral vector.

In another embodiment, the invention provides a method for producing therapeutic cells, comprising the steps of: providing primary immune cells from a donor or a patient or derived from human iPS or hES cells; performing a targeted insertion according to the methods herein; and purifying and freezing the cells for subsequent use as a therapeutic composition.

### Genomic Locus

As used herein, the term “locus” is the specific physical location of a DNA sequence (e.g. of a gene) into a genome. The term “locus” can refer to the specific physical location of a rare-cutting endonuclease target sequence on a chromosome or on an infection agent's genome sequence. Such a locus can comprise a target sequence that is recognized and/or cleaved by a sequence-specific endonuclease according to the invention.

In some embodiments, the exogenous sequence is inserted at a locus encoding a protein selected from TCR, β2m, PD1, CTLA4, TIM3, TGFβ, TGFβR, IL-10, IL10R, IL27RA, STAT1, STAT3, ILT2, ILT4, JAK2, AURKA, DNMT3, MT1A, MT2A, PTGER2, miR21, mir26A, miR101 miRNA31, MT1A, MT2A, PTGER2 GCN2, PRDM1, CD52, GR, HPRT, GGH, GM-CSF or DCK.

In some embodiments, the insertion of the exogenous sequence prevents the expression of the endogenous gene present at the locus.

In some embodiments, the insertion of the exogenous sequence corrects a mutation at the locus and thereby gene edits a mutation at the locus.

In some embodiments, the insertion of the exogenous sequence enables expression of a protein that is not endogenous to the locus.

In some embodiments, the cells that are genetically modified comprise HSC or iPS cells, and the cells comprise a transgene integrated at a locus that is transcriptionally active in a lineage of HSC or iPS cells, such as microglial cells, wherein the locus is selected from TMEM119, CD11B, B2m, CX3CR1 or S100A9, wherein the transgene is under the transcriptional control of the endogenous promoter of the locus.

In some embodiments, the exogenous sequence is inserted at a locus selected from CD25, CD69 or one listed in Table 1 (list of gene loci upregulated in tumor exhausted infiltrating lymphocytes), or Table 2 (list of gene loci upregulated in hypoxic tumor conditions).

TABLE 1
List of gene loci upregulated in tumor exhausted infiltrating lymphocytes
(compiled from multiple tumors) useful for gene integration of
exogenous coding sequences as per the present invention.

| | Uniprot ID |
|---|---|
| | (www.uniprot.org) |
| Gene names | (human) |
| GM-CSF | P04141 |
| CXCL13 | O43927 |
| TNFRSF1B | P20333 |
| RGS2 | P41220 |
| TIGIT | Q495A1 |
| CD27 | P26842 |
| TNFRSF9 | Q12933 |
| SLA | Q13239 |
| INPP5F | Q01968 |
| XCL2 | Q9UBD3 |
| HLA-DMA | P28067 |
| FAM3C | Q92520 |
| WARS | P23381 |
| EIF3L | Q9Y262 |
| KCNK5 | O95279 |
| TMBIM6 | P55061 |
| CD200 | P41217 |
| C3H7A | O60880 |
| SH2D1A | O60880 |
| ATP1B3 | P54709 |
| THADA | Q6YHU6 |
| PARK7 | Q99497 |
| EGR2 | P11161 |
| FDFT1 | P37268 |
| CRTAM | O95727 |
| IFI16 | Q16666 |

TABLE 2
List of gene loci upregulated in hypoxic tumor conditions
useful for gene integration of exogenous coding
sequences as per the present invention.

| | Strategy | |
|---|---|---|
| | (KO—knock out; | |
| Gene names | KI—knock in) | |
| CTLA-4 | KO/KI | Target shown to be upregulated in T- |
| LAG-3 | KO/KI | cells upon hypoxia exposure and T cell |
| (CD223) | | exhaustion |
| PD1 | KO/KI | |
| 4-1BB | KI | |
| (CD137) | | |
| GITR | KI | |
| OX40 | KI | |
| IL10 | KO/KI | |
| ABCB1 | KI | Loci which expression is under HIF-1 |
| ABCG2 | KI | (Uniprot Q16665) dependency. |
| ADM | KI | |
| ADRA1B | KI | |
| AK3 | KI | |
| ALDOA | KI | |
| BHLHB2 | KI | |
| BHLHB3 | KI | |
| BNIP3 | KI | |
| BNIP3L | KI | |
| CA9 | KI | |
| CCNG2 | KI | |
| CD99 | KI | |
| CDKN1A | KI | |
| CITED2 | KI | |
| COL5A1 | KI | |
| CP | KI | |
| CTGF | KI | |
| CTSD | KI | |
| CXCL12 | KI | |
| CXCR4 | KI | |
| CYP2S1 | KI | |
| DDIT4 | KI | |
| DEC1 | KI | |
| EDN1 | KI | |
| EGLN1 | KI | |
| EGLN3 | KI | |
| ENG | KI | |
| ENO1 | KI | |
| EPO | KI | |
| ETS1 | KI | |
| FECH | KI | |
| FN1 | KI | |
| FURIN | KI | |
| GAPDH | KI | |
| GPI | KI | |
| GPX3 | KI | |
| HK1 | KI | |
| HK2 | KI | |
| HMOX1 | KI | |
| HSP90B1 | KI | |
| ID2 | KI | |
| IGF2 | KI | |
| IGFBP1 | KI | |
| IGFBP2 | KI | |
| IGFBP3 | KI | |
| ITGB2 | KI | |
| KRT14 | KI | |
| KRT18 | KI | |
| KRT19 | KI | |
| LDHA | KI | |
| LEP | KI | |
| LOX | KI | |
| LRP1 | KI | |
| MCL1 | KI | |
| MET | KI | |
| MMP14 | KI | |
| MMP2 | KI | |
| MXI1 | KI | |
| NOS2A | KI | |
| NOS3 | KI | |
| NPM1 | KI | |
| NR4A1 | KI | |
| NT5E | KI | |
| PDGFA | KI | |
| PDK1 | KI | |
| PFKFB3 | KI | |
| PFKL | KI | |
| PGK1 | KI | |
| PH-4 | KI | |
| PKM2 | KI | |
| PLAUR | KI | |
| PMAIP1 | KI | |
| PPP5C | KI | |
| PROK1 | KI | |
| SERPINE1 | KI | |
| SLC2A1 | KI | |
| TERT | KI | |
| TF | KI | |
| TFF3 | KI | |
| TFRC | KI | |
| TGFA | KI | |
| TGFB3 | KI | |
| TGM2 | KI | |
| TPI1 | KI | |
| VEGFA | KI | |
| VIM | KI | |
| TMEM45A | KI | |
| AKAP12 | KI | |
| SEC24A | KI | |
| ANKRD37 | KI | |
| RSBN1 | KI | |
| GOPC | KI | |
| SAMD12 | KI | |
| CRKL | KI | |
| EDEM3 | KI | |
| TRIM9 | KI | |
| GOSR2 | KI | |
| MIF | KI | |
| ASPH | KI | |
| WDR33 | KI | |
| DHX40 | KI | |
| KLF10 | KI | |
| R3HDM1 | KI | |
| RARA | KI | |
| LOC162073 | KI | |
| PGRMC2 | KI | |
| ZWILCH | KI | |
| TPCN1 | KI | |
| WSB1 | KI | |
| SPAG4 | KI | |
| GYS1 | KI | |
| RRP9 | KI | |
| SLC25A28 | KI | |
| NTRK2 | KI | |
| NARF | KI | |
| ASCC1 | KI | |
| UFM1 | KI | |
| TXNIP | KI | |
| MGAT2 | KI | |
| VDAC1 | KI | |
| SEC61G | KI | |
| SRP19 | KI | |
| JMJD2C | KI | |
| SNRPD1 | KI | |
| RASSF4 | KI | |

In some embodiments, the genomic locus is active hematopoietic stem cells or lineage thereof, such as microglial cells. In some embodiments, the locus is selected from the group consisting of TMEM119, S100A9, CD11B, B2m, Cx3cr1, MERTK, CD164, Tlr4, Tlr7, Cd14, Fcgr1a, Fcgr3a, TBXAS1, DOK3, ABCA1, TMEM195, MR1, CSF3R, FGD4, TSPAN14, TGFBRI, CCR5, GPR34, SERPINE2, SLCO2B1, P2ry12, Olfm13, P2ry13, Hexb, Rhob, Jun, Rab3il1, Ccl2, Fcrls, Scoc, Siglech, Slc2a5, Lrrc3, Plxdc2, Usp2, Ctsf, Cttnbp2nl, Atp8a2, Lgmn, Mafb, Egr1, Bhlhe41, Hpgds, Ctsd, Hspa1a, Lag3, Csflr, Adamts1, F11r, Golm1, Nuak1, Crybb1, Ltc4s, Sgce, Pla2g15, Ccl3l1, Abhd12, Ang, Ophn1, Sparc, Pros1, P2ry6, Lair1, Il1a, Epb41l2, Adora3, Rilpl1, Pmepa1, Ccl13, Pde3b, Scamp5, Ppp1r9a, Tjp1, Ak1, B4galt4, Gtf2h2, Trem2, Ckb, Acp2, Pon3, Agmo, Tnfrsf17, Fscn1, St3gal6, Adap2, Ccl4, Entpd1, Tmem86a, Kctd12, Dst, Ctsl2, Abcc3, Pdgfb, Pald1, Tubgcp5, Rapgef5, Stab1, Lacc1, Tmc7, Nrip1, Kcnd1, Tmem206, Hps4, Dagla, Extl3, Mlph, Arhgap22, Cxxc5, P4ha1, Cysltr1, Fgd2, Kcnk13, Gbgt1, C18orf1, Cadm1, Bco2, Adrb1, C3ar1, Large, Leprel1, Liph, Upk1b, P2rx7, Slc46a1, Ebf3, Ppp1r15a, Il10ra, Rasgrp3, Fos, Tppp, Slc24a3, Havcr2, Nav2, Apbb2, Clstn1, Blnk, Gnaq, Ptprm, Frmd4a, Cd86, Tnfrsfl 1a, Spint1, Ppml1, Tgfbr2, Cmklr1, Tlr6, Gas6, Hist1h2ab, Atf3, Acvr1, Abi3, Lrp12, Ttc28, Plxna4, Adamts16, Rgs1, Icam1, Snx24, Ly96, Dnajb4, and Ppfia4.

In some embodiments, the locus corresponds to an intronic polynucleotide sequence. In some embodiments, the exogenous sequence is a coding sequence and is inserted between the first and second endogenous exons of the genomic locus.

In some embodiments, the method has the advantage of preventing disruption of a transcript encoding the endogenous exonic regions, while allowing their transcription together with the exogenous coding sequence.

In general, the method can comprise introducing into the cell the DNA template comprising the exogenous sequence, wherein the exogenous sequence comprises a coding sequence, and the template comprises in the 5′ to 3′ orientation:
a first homologous polynucleotide sequence, which is homologous to the intronic sequence upstream of the insertion site,
while the first polynucleotide sequence does not preferably comprise a branch point;
a first strong splice site sequence, preferably comprising a branch point and a splice acceptor;
a first sequence encoding 2A self-cleaving peptide;
an exogenous sequence coding for a protein of interest;
a second sequence encoding 2A self-cleaving peptide;
a copy of the coding sequence of the first exon, optionally rewritten;
a second strong splice site sequence preferably comprising a splice donor; and a second homologous polynucleotide sequence, which is homologous to the intronic sequence downstream of the insertion site;
   wherein the DNA template comprising the exogenous sequence is integrated into the intronic sequence, preferably by homologous recombination, to have the exogenous coding sequence being transcribed at the endogenous locus along with the first exon and preferably second (endogenous) exon, or a copy thereof.

### Sequence-Specific Endonuclease

In accordance with the methods herein, a cell is provided a sequence-specific endonuclease having cleavage activity at a genomic locus. In some embodiments, the insertion of the exogenous sequence is induced by a sequence-specific endonuclease that has cleavage activity at said locus, at least 5 hours before the introduction into said cell of a DNA template comprising the exogenous sequence.

By “sequence-specific endonuclease” is meant any active molecule, such as a protein that has endonuclease activity and the ability to specifically recognize a selected polynucleotide sequence from a genomic locus, preferably of at least 9 bp, more preferably of at least 10 bp and even more preferably of at least 12 bp in length, in view of modifying the expression of said genomic locus.

The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites.”

The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA RNA hybrid cleavage can result in the production of either blunt ends or staggered ends.

In some embodiments, the sequence-specific endonuclease is provided to the cell as a polypeptide. In some embodiments, the sequence-specific endonuclease is provided to the cell as a nucleic acid that encodes the endonuclease. In some embodiments, the nucleic acid encoding the sequence-specific endonuclease is a mRNA. In some embodiments, the nucleic acid encoding the sequence-specific endonuclease is a DNA.

As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

Endonucleases can be classified as rare-cutting endonucleases when having typically a polynucleotide recognition site greater than 10 base pairs (bp) in length. In some embodiments the rare-cutting endonuclease has a recognition site of from 14-55 bp. Rare-cutting endonucleases significantly increase homologous recombination by inducing DNA double-strand breaks (DSBs) at a defined locus thereby allowing gene repair or gene insertion therapies (Pingoud, A. and G. H. Silva (2007). Nat. Biotechnol. 25(7): 743-4).

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein.

Zinc finger and TALE binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the sequence-specific endonuclease is engineered and is not found in nature. In some embodiments, the endonuclease is generated using a process such as phage display, interaction trap or hybrid selection. See e.g., U.S. Pat. Nos. 5,789,538; 5,925,523; 6,007,988; 6,013,453; 6,200,759; as well as WO 95/19431; WO 96/06166; WO 98/53057; WO 98/54311; WO 00/27878; WO 01/60970; WO 01/88197; WO 02/099084 and U.S. Patent Appl. Publication No. 2011/0301073.

In some embodiments, the sequence-specific endonuclease is a TALE-nuclease, such as TALE-nucleases (commercially available under Cellectis trademark TALEN®). In some embodiments, the endonuclease is a RNA-guided endonuclease, such as Cas9 or Cpf1. In some embodiments, a guide-RNA associated with said RNA-guided endonuclease is introduced concomitantly with said RNA-guided endonuclease. In some embodiments, the sequence-specific endonuclease cleaves one or several of the target sequences reported in Tables 1-3 of the present specification.

In some embodiments, the sequence specific endonuclease can be a chimeric polypeptide comprising a DNA binding domain and another domain displaying catalytic activity. Such catalytic activity can be nickase or double nickase to preferentially perform gene insertion by creating cohesive ends to facilitate gene integration by homologous recombination.

In some embodiments, the sequence specific endonuclease induces NHEJ or homologous recombination mechanisms, which has the advantage of introducing stable and inheritable mutations into the genomic locus expressed in cells.

The nucleic acid sequence which is recognized by the sequence specific endonuclease is within the genomic locus. The sequence that is recognized is usually selected to be rare or unique in the cell's genome, and more extensively in the human genome, as can be determined using software and data available from human genome databases, such as http://www.ensembl.org/index.html.

Exemplary selection methods applicable to DNA-binding domains, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237.

Selection of target sites; nucleases and methods for design and construction of fusion proteins (and polynucleotides encoding same) are known to those of skill in the art and described in detail in U.S. Patent Application Publication Nos. 20050064474 and 20060188987, incorporated by reference in their entireties herein.

DNA domains can be engineered to bind to any sequence of choice in a targeted locus. In some embodiments, the cells are provided with a sequence specific endonuclease that has been engineered to bind a locus that is transcriptionally active in HSC or HSC lineage cells, such as microglial cells. An engineered DNA-binding domain can have a novel binding specificity, compared to a naturally-occurring DNA-binding domain. Engineering methods include, but are not limited to, rational design and various types of selection. Rational design includes, for example, using databases comprising triplet (or quadruplet) nucleotide sequences and individual (e.g., zinc finger) amino acid sequences, in which each triplet or quadruplet nucleotide sequence is associated with one or more amino acid sequences of DNA binding domain which bind the particular triplet or quadruplet sequence. See, for example, U.S. Pat. Nos. 6,453,242 and 6,534,261, incorporated by reference herein in their entireties. Rational design of TAL-effector domains can also be performed. See, e.g., U.S. Patent Appl. Publication No. 2011/0301073.

In addition, as disclosed in these and other references, DNA-binding domains (e.g., multi-fingered zinc finger proteins) may be linked together using any suitable linker sequences, including for example, linkers of 5 or more amino acids. See, e.g., U.S. Pat. Nos. 6,479,626; 6,903,185; and 7,153,949 for exemplary linker sequences 6 or more amino acids in length. The proteins described herein may include any combination of suitable linkers between the individual DNA-binding domains of the protein. See, also, U.S. Patent Appl. Publication No. 2011/0301073.

In some embodiments, the sequence specific endonuclease is a nucleic acid encoding an “engineered” or “programmable” rare-cutting endonuclease, such as a homing endonuclease as described for instance in WO 2004067736, a zing finger nuclease (ZFN) as described, for instance, by Urnov F., et al. (Nature 435:646-651 (2005)), a TALE-Nuclease as described, for instance, by Mussolino et al. (Nucl. Acids Res. 39(21):9283-9293 (2011)), or a MegaTAL nuclease as described, for instance by Boissel et al. (Nucleic Acids Research 42 (4):2591-2601 (2013)).

In some embodiments, the sequence specific endonuclease is transiently expressed into the cells, meaning that the reagent is not supposed to integrate into the genome or persist over a long period of time, such as the case of RNA, more particularly mRNA, proteins or complexes mixing proteins and nucleic acids (e.g.: Ribonucleoproteins).

In some embodiments, the sequence-specific endonuclease is a nuclease that introduces DNA double strand break at a targeted locus, whose subsequent repair is exploited to achieve different outcomes. In some embodiments, a repair pathway based on homologous recombination can be used to copy information from an introduced DNA homology template. Such homology directed repair (HDR) can promote a specific addition of exogenous polynucleotide sequence (See, e.g., U.S. Pat. No. 8,921,332), e.g., a transgene as described herein, that can be expressed under the control of a promoter present on the exogenous polynucleotide sequence. In some embodiments, the transgene as described herein, can be expressed under the control of an endogenous promoter and at the same time that gene disruption is achieved. In some embodiments where gene disruption is not sought, the transgene can be inserted at the stop codon of the endogenous gene and comprises a self-cleaving 2A peptide or IRES sequence. In some embodiments, the transgene is expressed under the control of an endogenous promoter without gene disruption. In some embodiments, the non-homologous end joining (NHEJ) repair pathway can be utilized (See, e.g., U.S. Pat. No. 9,458,439; He et al., Nucleic Acids Research, 44 e85, https://doi.org/10.1093/nar/gkw064).

The sequence-specific endonucleases can target a gene that is active in a particular cell type, for example certain types of immune cells, HSC or progeny thereof such as microglial cells, for the insertion of the transgene. In some embodiments, the sequence-specific endonuclease is non-naturally occurring, i.e., engineered in the DNA-binding domain and/or cleavage domain. For example, the DNA-binding domain of a naturally-occurring sequence-specific endonuclease may be altered to bind to a selected target site (e.g., a meganuclease that has been engineered to bind to site different than the cognate binding site or a CRISPR/Cas system utilizing an engineered single guide RNA). In other embodiments, the sequence-specific endonuclease comprises heterologous DNA-binding and cleavage domains (e.g., zinc finger nucleases; TAL-effector nucleases; meganuclease DNA-binding domains with heterologous cleavage domains).

In some embodiments, the sequence-specific endonuclease is a meganuclease (homing endonuclease). Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

In some embodiments, the sequence-specific endonuclease comprises an engineered (non-naturally occurring) homing endonuclease (meganuclease). In some embodiments, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain.

In some embodiments, the DNA-binding domain comprises a naturally occurring or engineered (non-naturally occurring) TAL effector DNA binding domain. See, e.g., U.S. Patent Application Publication No. 2011/0301073, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (Kay et al. (2007) Science 318:648-651). These proteins contain a DNA binding domain and a transcriptional activation domain. One of the most well characterized TALEs is AvrBs3 from Xanthomonas campestris pv. Vesicatoria (see Bonas et al. (1989) Mol Gen Genet 218: 127-136 and WO 2010/079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al. (2006) J Plant Physiol 163(3): 256-272). In addition, in the phytopathogenic bacteria Ralstonia solancearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al. (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

In some embodiments, the DNA binding domain that binds to a target site in a target locus is an engineered domain from a TAL effector similar to those derived from the plant pathogens Xanthomonas (see Boch et al. (2009) Science 326: 1509-1512 and Moscou and Bogdanove (2009) Science 326: 1501) and Ralstonia (see Heuer et al. (2007) Applied and Environmental Microbiology 73(13): 4379-4384); U.S. Pat. Nos. 8,420,782 and 8,440,431 and U.S. Patent Appl. Publication No. 2011/0301073.

In some embodiments, the DNA binding domain comprises a zinc finger protein. In some embodiments, the zinc finger protein is non-naturally occurring in that it is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Appl. Publication Nos. 2005/0064474; 2007/0218528; 2005/0267061, all incorporated herein by reference in their entireties.

An engineered zinc finger binding or TALE domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

In some embodiments, DNA domains (e.g., multi-fingered zinc finger proteins or TALE domains) may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The DNA binding proteins described herein may include any combination of suitable linkers between the individual zinc fingers of the protein. In addition, enhancement of binding specificity for zinc finger binding domains has been described, for example, in co-owned WO 02/077227.

DNA-binding domains and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496 and U.S. Patent Appl. Publication No. 2011/0301073.

Any suitable cleavage domain can be operatively linked to a DNA-binding domain to form a nuclease. For example, ZFP DNA-binding domains have been fused to nuclease domains to create ZFNs—a functional entity that is able to recognize its intended nucleic acid target through its engineered (ZFP) DNA binding domain and cause the DNA to be cut near the ZFP binding site via the nuclease activity. See, e.g., Kim et al. (1996) Proc Nat'l Acad Sci USA 93(3):1156-1160. More recently, ZFNs have been used for genome modification in a variety of organisms. See, for example, United States Patent Appl. Pub. Nos.: 2003/0232410; 2005/0208489; 2005/0026157; 2005/0064474; 2006/0188987; 2006/0063231; and International Publication WO 07/014275. Likewise, TALE DNA-binding domains have been fused to nuclease domains to create TALENs. See, e.g., U.S. Patent Appl. Publication No. 2011/0301073.

As noted above, the cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

In some embodiments, the dimerized cleavage half domains comprise one inactive cleavage domain and one active cleavage domain such that the targeted DNA is nicked on one strand rather than being completely cleaved (a “nickase”, see U.S. Patent Appl. Publication No. 2010/0047805). In other embodiments, two pairs of such nickases are used to cleave a target that is nicked on both DNA strands.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994) Proc. Natl. Acad. Sci. USA 91:883-887; Kim et al. (1994) J. Biol. Chem. 269:31, 978-31, 982. In one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a Fok I cleavage half-domain, can be used to reconstitute a catalytically active cleavage domain. Alternatively, a single polypeptide molecule containing a DNA binding domain and two Fok I cleavage half-domains can also be used.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In some embodiments, the sequence specific endonuclease is a RNA-guided endonuclease, such as Cas9 or Cpf1, to be used in conjunction with a RNA guide, as per, inter alia, the teaching by Doudna, J. et al., (Science 346 (6213): 1077) (2014)) and Zetsche, B. et al. (Cell 163(3): 759-771 (2015)) the teaching of which is incorporated herein by reference.

In some embodiments, the cells are provided the CRISPR (Clustered Regularly Interspaced Short Palindromic Repeats)/Cas (CRISPR Associated) nuclease system. The CRISPR/Cas is an engineered nuclease system based on a bacterial system that can be used for genome engineering. It is based on part of the adaptive immune response of many bacteria and archea. When a virus or plasmid invades a bacterium, segments of the invader's DNA are converted into CRISPR RNAs (crRNA) by the ‘ immune’ response. This crRNA then associates, through a region of partial complementarity, with another type of RNA called tracrRNA to guide the Cas9 nuclease to a region homologous to the crRNA in the target DNA called a “protospacer”. Cas9 has been reported to cleave the DNA to generate blunt ends at the DSB at sites specified by a 20-nucleotide guide sequence contained within the crRNA transcript. Originally, Cas9 requires both the crRNA and the tracrRNA for site specific DNA recognition and cleavage. This system has now been engineered such that the crRNA and tracrRNA can be combined into one molecule (the “single guide RNA”), and the crRNA equivalent portion of the single guide RNA can be engineered to guide the Cas9 nuclease to target any desired sequence (see Jinek et al. (2012) Science 337, p. 816-821, Jinek et al., (2013), eLife 2:e00471, and David Segal, (2013) eLife 2:e00563).

The CRISPR (clustered regularly interspaced short palindromic repeats) locus, which encodes RNA components of the system, and the cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein. Is also encompassed in RNA-guided endonucleases in the meaning of the present invention, the endonuclease Cpf1 as taught by Zetsche, B. et al. (Cell 163(3): 759-771 (2015)).

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong et al., (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA).

In some embodiments, the sequence-specific endonuclease targets intron of CX3 CR1 preferably the first intron of CX3CR1 located between the first coding exon and second coding exon (SEQ ID NO:76). The invention also provides specific TALE nucleases that preferentially target endogenous polynucleotide sequences of CX3CR1 similar to SEQ ID NO:77 to 87. In some embodiments, the sequence-specific endonucleases are CRISPR-Cas or CRISPR-Cpf using gRNA targeting endogenous sequences similar to SEQ ID NO:97 to 106.

In some embodiments, the sequence-specific endonuclease targets an intron of CD11B preferably the first intron of CD11B. The invention also provides specific TALE nucleases that preferentially target endogenous polynucleotide sequences of CD11B similar to SEQ ID NO:108 to 137. In some embodiments, the sequence-specific endonucleases are CRISPR-Cas or CRISPR-Cpf using gRNA targeting endogenous sequences similar to SEQ ID NO:138 to 147.

In some embodiments, the sequence-specific endonuclease targets an intron of S100A9, preferably the first intron of S100A9. The invention also provides specific TALE nucleases that preferentially target endogenous polynucleotide sequences of S100A9 similar to SEQ ID NO:149 to 178. In some embodiments, the sequence specific reagents are CRISPR-Cas or CRISPR-Cpf using gRNA targeting endogenous sequences similar to SEQ ID NO:179 to 188.

TABLE 3
Target sequences defined to gene edit the cells of the present invention

| SEQ ID | Name of sequence- | |
|---|---|---|
| NO # | specific reagent | Target sequence |
| 77 | CX3CR1_T01 | TCTTTCCTCTGTAGCATGGTCCAGATGGCTCATAGCA |
| | | GGGACCATGATA |
| 78 | CX3CR1_T02 | TATGCTGGGTGAGCACCCACTGCATGCACCCACTGT |
| | | GCCAGCACTGAGA |
| 79 | CX3CR1_T03 | TGCTGGGTGAGCACCCACTGCATGCACCCACTGTGC |
| | | CAGCACTGAGAGA |
| 80 | CX3CR1_T04 | TGAGAGACTCCTGTGGGAGCCACAGCAATTCTAGGG |
| | | TCTTCACTGGGGA |
| 81 | CX3CR1_T05 | TCCTGTGGGAGCCACAGCAATTCTAGGGTCTTCACTG |
| | | GGGACTCTGAGA |
| 82 | CX3CR1_T06 | TGGGAGCCACAGCAATTCTAGGGTCTTCACTGGGGA |
| | | CTCTGAGACAGCA |
| 83 | CX3CR1_T07 | TCGTCTGCCCTCACTGAGCAGACCCCCTGGATGGCA |
| | | GGGAGCAGTCCCA |
| 84 | CX3CR1_T08 | TGCCCTCACTGAGCAGACCCCCTGGATGGCAGGGAG |
| | | CAGTCCCAAGCCA |
| 85 | CX3CR1_T09 | TGGATGGCAGGGAGCAGTCCCAAGCCAGATGGATGC |
| | | CCATAACCAGCCA |
| 86 | CX3CR1_T10 | TCTCAATACATAATATCACCACGTATCAGGCAAAAC |
| | | CATCCTGCCCAGA |
| 87 | CX3CR1_T11 | TATCAGGCAAAACCATCCTGCCCAGAGCATTATCTG |
| | | AATTTGCATCCCA |
| 88 | CX3CR1_T12 | TCCTGCCCAGAGCATTATCTGAATTTGCATCCCATCT |
| | | GCAGAAGATACA |
| 89 | CX3CR1_T13 | TCTGAATTTGCATCCCATCTGCAGAAGATACATTCAC |
| | | CCACTTCTTCCA |
| 90 | CX3CR1_T14 | TTCCATTCTGTCTTAATCAAAGTCTTTATGTGAATTTT |
| | | CCCCATTGAGA |
| 91 | CX3CR1_T15 | TCCATTCTGTCTTAATCAAAGTCTTTATGTGAATTTTC |
| | | CCCATTGAGAA |
| 92 | CX3CR1_T16 | TTCTGTCTTAATCAAAGTCTTTATGTGAATTTTCCCCA |
| | | TTGAGAAGACA |
| 93 | CX3CR1_T17 | TCTGTCTTAATCAAAGTCTTTATGTGAATTTTCCCCAT |
| | | TGAGAAGACAA |
| 94 | CX3CR1_T18 | TTTATGTGAATTTTCCCCATTGAGAAGACAAGCCCCT |
| | | TCCTGGCTTAGA |
| 95 | CX3CR1_T19 | TTCCTGGCTTAGACTGTACCTGACTGATCTTTTCATG |
| | | AGCTCCTTGCCA |
| 96 | CX3CR1_T20 | TCCTGGCTTAGACTGTACCTGACTGATCTTTTCATGA |
| | | GCTCCTTGCCAA |
| 97 | CX3CR1_gRNA1 | GTACAGTCTAAGCCAGGAAGGGG |
| 98 | CX3CR1_gRNA2 | GGGAGCAGTCCCAAGCCAGATGG |
| 99 | CX3CR1_gRNA3 | GACTGCTCCCTGCCATCCAGGGG |
| 100 | CX3CR1_gRNA4 | GTGAATGTATCTTCTGCAGATGG |
| 101 | CX3CR1_gRNA5 | GTCTCTCAGTGCTGGCACAGTGG |
| 102 | CX3CR1_gRNA6 | GACAGCAGGGAGCTAGGATGAGG |
| 103 | CX3CR1_gRNA7 | GAAGGGGCTTGTCTTCTCAATGG |
| 104 | CX3CR1_gRNA8 | GCAAATTCAGATAATGCTCTGGG |
| 105 | CX3CR1_gRNA9 | GAGCAGACCCCCTGGATGGCAGG |
| 106 | CX3CR1_gRNA10 | GAACTCATAGAAAGCGATATTGG |
| 108 | CD11b_T01 | TTCAGAGCAGGACTGGACGTGCCCCACGACGGTGGT |
| | | TCTTAGGTCAGGA |
| 109 | CD11b_T02 | TATGGCCCACGACCTGTTTTTGCACAACCTGCCAGCT |
| | | AGAGATTGAAGA |
| 110 | CD11b_T03 | TGATGATAGGGAGCACCACCCCCAAAGAATTCTATT |
| | | TGTCTCATTTGTA |
| 111 | CD11b_T04 | TTCTATTTGTCTCATTTGTAAACCCGTATTACAAACA |
| | | AATTGTACTCAA |
| 112 | CD11b_T05 | TATTTGTCTCATTTGTAAACCCGTATTACAAACAAAT |
| | | TGTACTCAATCA |
| 113 | CD11b_T06 | TTTGTAAACCCGTATTACAAACAAATTGTACTCAATC |
| | | ATTATGTTTGAA |
| 114 | CD11b_T07 | TTGTAAACCCGTATTACAAACAAATTGTACTCAATCA |
| | | TTATGTTTGAAA |
| 115 | CD11b_T08 | TACAAACAAATTGTACTCAATCATTATGTTTGAAATT |
| | | TCCCTAATGACA |
| 116 | CD11b_T09 | TTGTACTCAATCATTATGTTTGAAATTTCCCTAATGA |
| | | CAAATTTGTGGA |
| 117 | CD11b_T10 | TGTACTCAATCATTATGTTTGAAATTTCCCTAATGAC |
| | | AAATTTGTGGAA |
| 118 | CD11b_T11 | TACTCAATCATTATGTTTGAAATTTCCCTAATGACAA |
| | | ATTTGTGGAAAA |
| 119 | CD11b_T12 | TTTCCCTAATGACAAATTTGTGGAAAAGTATTTTCTG |
| | | TCTTGTTATATA |
| 120 | CD11b_T13 | TTCCCTAATGACAAATTTGTGGAAAAGTATTTTCTGT |
| | | CTTGTTATATAA |
| 121 | CD11b_T14 | TTGTGGAAAAGTATTTTCTGTCTTGTTATATAAGTAC |
| | | TTGTACAACATA |
| 122 | CD11b_T15 | TGTTATATAAGTACTTGTACAACATATTCTATCAGCC |
| | | TCTTGGTCTGCA |
| 123 | CD11b_T16 | TTATATAAGTACTTGTACAACATATTCTATCAGCCTC |
| | | TTGGTCTGCAAA |
| 124 | CD11b_T17 | TATATAAGTACTTGTACAACATATTCTATCAGCCTCT |
| | | TGGTCTGCAAAA |
| 125 | CD11b_T18 | TACAACATATTCTATCAGCCTCTTGGTCTGCAAAACC |
| | | TAAAATTTACTA |
| 126 | CD11b_T19 | TCTTGGTCTGCAAAACCTAAAATTTACTATCTGGCTG |
| | | TTTACAGAATAA |
| 127 | CD11b_T20 | TGCAAAACCTAAAATTTACTATCTGGCTGTTTACAGA |
| | | ATAAGTGTGCTA |
| 128 | CD11b_T21 | TGAAAATGATTTGAGTTTGTTACCTTTTATGCTTATA |
| | | TGTTGTGGAAAA |
| 129 | CD11b_T22 | TTTGTTACCTTTTATGCTTATATGTTGTGGAAAATGA |
| | | AATTCTCCTCAA |
| 130 | CD11b_T23 | TTGTTACCTTTTATGCTTATATGTTGTGGAAAATGAA |
| | | ATTCTCCTCAAA |
| 131 | CD11b_T24 | TGTTACCTTTTATGCTTATATGTTGTGGAAAATGAAA |
| | | TTCTCCTCAAAA |
| 132 | CD11b_T25 | TTTATGCTTATATGTTGTGGAAAATGAAATTCTCCTC |
| | | AAAAGGGAAGGA |
| 133 | CD11b_T26 | TTATGCTTATATGTTGTGGAAAATGAAATTCTCCTCA |
| | | AAAGGGAAGGAA |
| 134 | CD11b_T27 | TATGCTTATATGTTGTGGAAAATGAAATTCTCCTCAA |
| | | AAGGGAAGGAAA |
| 135 | CD11b_T28 | TGCTTATATGTTGTGGAAAATGAAATTCTCCTCAAAA |
| | | GGGAAGGAAATA |
| 136 | CD11b_T29 | TGGAAAATGAAATTCTCCTCAAAAGGGAAGGAAATA |
| | | CTTGAGAGCTGCA |
| 137 | CD11b_T30 | TACTTGAGAGCTGCATAGGAAGGAAATTATCTAATT |
| | | AAGAATGTATAGA |
| 138 | CD11b_gRNA1 | GGTTGTGCAAAAACAGGTCGTGG |
| 139 | CD11b_gRNA2 | GGGAGGCTGGAATTCAGAGCAGG |
| 140 | CD11b_gRNA3 | GGAGTCAGCAAACAGTGGCCTGG |
| 141 | CD11b_gRNA4 | GAGTCAGCAAACAGTGGCCTGGG |
| 142 | CD11b_gRNA5 | GACCTAAGAACCACCGTCGTGGG |
| 143 | CD11b_gRNA6 | GCAAATCATCGTTGTGACACCGG |
| 144 | CD11b_gRNA7 | GAGACAAATAGAATTCTTTGGGG |
| 145 | CD11b_gRNA8 | GCCCCACGACGGTGGTTCTTAGG |
| 146 | CD11b_gRNA9 | GAAATACTTGAGAGCTGCATAGG |
| 147 | CD11b_gRNA10 | GGTCAGGAGTCAGCAAACAGTGG |
| 149 | S100A9_T01 | TTTCCCCGTTGTATTGGTTGAAATAAGTTTCACTAAT |
| | | TGGTAACCTCCA |
| 150 | S100A9_T02 | TATTGGTTGAAATAAGTTTCACTAATTGGTAACCTCC |
| | | AGAGGGAAGGGA |
| 151 | S100A9_T03 | TTTCACTAATTGGTAACCTCCAGAGGGAAGGGAAGG |
| | | GAGGGCAGGGGAA |
| 152 | S100A9_T04 | TGGAACTGGCCTCTAAGTCAGATCTGAATTTGCATGC |
| | | CCTCAATAGTCA |
| 153 | S100A9_T05 | TCTAAGTCAGATCTGAATTTGCATGCCCTCAATAGTC |
| | | AAGCTGTGAAAA |
| 154 | S100A9_T06 | TGCATGCCCTCAATAGTCAAGCTGTGAAAACTAATG |
| | | ACCCTCTCTAGGA |
| 155 | S100A9_T07 | TGAAAACTAATGACCCTCTCTAGGACTGGTTTCAAGT |
| | | CTTCCTCCAGGA |
| 156 | S100A9_T08 | TCTTCCTCCAGGAAGATACCATTCCTAGCTGTTAAAG |
| | | TTGTTATAAGGA |
| 157 | S100A9_T09 | TCCTCCAGGAAGATACCATTCCTAGCTGTTAAAGTTG |
| | | TTATAAGGACCA |
| 158 | S100A9_T10 | TTCCTAGCTGTTAAAGTTGTTATAAGGACCAAATGAG |
| | | GTGACATTTCCA |
| 159 | S100A9_T11 | TTAAAGTTGTTATAAGGACCAAATGAGGTGACATTT |
| | | CCAGGCTTACTCA |
| 160 | S100A9_T12 | TGACCAGGGCAAGACCCTGGAACTCAGCTTCCTCTTC |
| | | TATAAATAGAGA |
| 161 | S100A9_T13 | TTCCTCTTCTATAAATAGAGAATCAGCACCCAAGTCA |
| | | CAGGGTCATGGA |
| 162 | S100A9_T14 | TCTTCTATAAATAGAGAATCAGCACCCAAGTCACAG |
| | | GGTCATGGAGGGA |
| 163 | S100A9_T15 | TCTATAAATAGAGAATCAGCACCCAAGTCACAGGGT |
| | | CATGGAGGGAATA |
| 164 | S100A9_T16 | TATAAATAGAGAATCAGCACCCAAGTCACAGGGTCA |
| | | TGGAGGGAATAAA |
| 165 | S100A9_T17 | TGGAGAGCGTTTGGTATGTGCTCAGTGTCTGCTCCAT |
| | | TGTGCGCACTCA |
| 166 | S100A9_T18 | TGGTATGTGCTCAGTGTCTGCTCCATTGTGCGCACTC |
| | | AGCCTATGGTCA |
| 167 | S100A9_T19 | TTGTGCGCACTCAGCCTATGGTCATTTTTAATTTTTA |
| | | AATCCAGCCCCA |
| 168 | S100A9_T20 | TTCCCTTGTACATTTGCCAGCTGGTCATTTACTGTGCT |
| | | CCCAGTCCCCA |
| 169 | S100A9_T21 | TTTTGTTTTCTTTTCAAATTTGGGGAAAGTCGGGAAA |
| | | CAGAGGCCTGCA |
| 170 | S100A9_T22 | TTTCTTTTCAAATTTGGGGAAAGTCGGGAAACAGAG |
| | | GCCTGCATTAAGA |
| 171 | S100A9_T23 | TTCTTTTCAAATTTGGGGAAAGTCGGGAAACAGAGG |
| | | CCTGCATTAAGAA |
| 172 | S100A9_T24 | TTGGGGAAAGTCGGGAAACAGAGGCCTGCATTAAGA |
| | | AGGGTGGAACACA |
| 173 | S100A9_T25 | TAGGTCCCCAGCCCTCCCAGTGCCCCTCCCTCCGCCT |
| | | TGGTAAGGTGGA |
| 174 | S100A9_T26 | TTCAGAGTTAGGGGCCCTGACAGCTCTCCATAGGTG |
| | | GAGGCCTCAGGCA |
| 175 | S100A9_T27 | TTAGGGGCCCTGACAGCTCTCCATAGGTGGAGGCCT |
| | | CAGGCAGGCAGGA |
| 176 | S100A9_T28 | TCCATAGGTGGAGGCCTCAGGCAGGCAGGATGCTGG |
| | | GTGGGGTAGGCAA |
| 177 | S100A9_T29 | TAGGTGGAGGCCTCAGGCAGGCAGGATGCTGGGTGG |
| | | GGTAGGCAAGAAA |
| 178 | S100A9_T30 | TGGGTGGGGTAGGCAAGAAAGGGCCCAGCAGAGAG |
| | | GCCGCATGGCAAAA |
| 179 | S100A9_gRNA1 | GCACAGGAGAGTGCTCGCATTGG |
| 180 | S100A9_gRNA2 | GGTACCCCACAGGTTCTGGGAGG |
| 181 | S100A9_gRNA3 | GGAGCCAGACATCCTGGGGTAGG |
| 182 | S100A9_gRNA4 | GGAGAGTGCTCGCATTGGCTGGG |
| 183 | S100A9_gRNA5 | GGAAGCAGAGCCTCATGGATGGG |
| 184 | S100A9_gRNA6 | GGCTTACTCATGCCATGACCAGG |
| 185 | S100A9_gRNA7 | GGGAAACACCTAGAAAAACTAGG |
| 186 | S100A9_gRNA8 | GTGGGGGGTGAAGCGGGCATAGG |
| 187 | S100A9_gRNA9 | GGGGGGTGAAGCGGGCATAGGGG |
| 188 | S100A9_gRNA10 | GAGGGCTGGGGACCTACCCCAGG |

### Exogenous Sequence

The sequence specific endonuclease used according to the invention, which specifically cleaves a sequence within the locus, is used to induce the integration of an exogenous sequence at the locus. “Exogenous sequence” refers to any nucleotide or nucleic acid sequence that was not initially present at the selected locus. In some embodiments, the exogenous sequence preferably comprises a sequence that codes for a therapeutic polypeptide as described herein, e.g., for treating a disease or condition. An endogenous sequence that is genetically modified by the insertion of a polynucleotide according to the method of the present invention, in order to express the polypeptide encoded thereby is broadly referred to as an exogenous coding sequence. In some embodiments, the targeted gene insertion comprises an exogenous sequence encoding a therapeutic polypeptide as described herein.

In some embodiments, the sequence specific endonuclease has cleavage activity for at least 5 hours until the DNA template comprising the exogenous sequence is introduced into the cell. In some embodiments, the sequence-specific endonuclease has cleavage activity for at least about 6, 7, 8, 9, 10, 11, 12, 13, 14, 15, 16, 17, 18, 19, or 20 hours, until the DNA template comprising the exogenous sequence is introduced into the cell. In some embodiments, the sequence-specific endonuclease has cleavage activity preferably for at least 18 hours, more preferably at least 20 hours until the DNA template comprising the exogenous sequence is introduced into the cell.

In some embodiments, the DNA template comprising the exogenous sequence is introduced into the cell between about 10 and about 30 hours after transfection of nucleic acid encoding the sequence-specific endonuclease. In some embodiments, the DNA template comprising the exogenous sequence is introduced into the cell between about 15 and about 25 hours after transfection of nucleic acid encoding the sequence-specific endonuclease. In some embodiments, the DNA template comprising the exogenous sequence is introduced into the cell between about 15 and about 20 hours after transfection of nucleic acid encoding the sequence-specific endonuclease. In some embodiments, the DNA template is introduced about 10, 11, 12, 13, 14, 15, 16, 17, 18, 19, 20, 21, 22, 23, 24, 25, 26, 27, 28, 29, or 30 hours after transfection of nucleic acid encoding the sequence-specific endonuclease.

In some embodiments, the DNA template comprising the exogenous sequence is introduced into the cell between about 5 and about 25 hours after transfection of the sequence-specific endonuclease polypeptide. In some embodiments, the DNA template comprising the exogenous sequence is introduced into the cell between about 10 and about 20 hours after transfection of the sequence-specific endonuclease polypeptide. In some embodiments, the DNA template comprising the exogenous sequence is introduced into the cell between about 10 and about 15 hours after transfection of the sequence-specific endonuclease polypeptide. In some embodiments, the DNA template is introduced about 5, 6, 7, 8, 9, 10, 11, 12, 13, 14, 15, 16, 17, 18, 19, 20, 21, 22, 23, 24, or 25 hours after transfection of the sequence-specific endonuclease polypeptide.

In some embodiments, the DNA template comprising the exogenous sequence is double stranded (dsDNA). In some embodiments, the dsDNA is a PCR product. In some embodiments, the dsDNA has a length of more than 2 kb, preferably more than 2.5 kb, more preferably more than 3 kb, even more preferably between 2 and 10 kb.

In some embodiments, the DNA template is a single stranded polynucleotide. In some embodiments, the DNA template is a short single-stranded oligodeoxynucleotide (ssODN). In some embodiments, the ssODN has homology arms comprised between 50 and 200 bp, preferably between 80 and 150 bp, more preferably between 90 and 120 bp.

The sequence specific endonuclease (e.g., CRISPR/Cas, ZFNS or TALENs) creates a double-stranded break at the locus (e.g., cellular chromatin). The DNA template that comprises the exogenous sequence, e.g., a transgene encoding a therapeutic protein and having homology to the nucleotide sequence flanking the region of the break, is introduced into the cell. The presence of the double-stranded break has been shown to facilitate integration of the DNA template sequence. The DNA template sequence may be physically integrated or, alternatively, the DNA template is used as a template for repair of the break via homologous recombination, resulting in the introduction of all or part of the nucleotide sequence as in the DNA template into the cellular chromatin. Thus, a sequence in cellular chromatin at a genomic locus can be altered and, in certain embodiments, can be modified to comprise a sequence present in the DNA template.

In some embodiments, the exogenous sequence, e.g., comprising a transgene, is not identical over its entire length to sequences within the locus. The DNA template can contain a non-homologous sequence flanked by two regions of homology to allow for efficient HDR at the location of interest. Alternatively, the DNA template may have no regions of homology to the targeted location in the DNA and may be integrated by NHEJ-dependent end joining following cleavage at the target site. The DNA template can contain several, discontinuous regions of homology to cellular chromatin. For example, for targeted insertion of sequences not normally present in a locus, said sequences can be present in a DNA template molecule and flanked by regions of homology to sequence in the locus.

In some embodiments, the exogenous nucleotide sequence can contain sequences that are homologous, but not identical, to genomic sequences in the locus of interest, thereby stimulating homologous recombination to insert a non-identical sequence in the locus of interest. In some embodiments, portions of the DNA template that are homologous to sequences in the locus of interest exhibit between about 70 to 99% (or any integer therebetween) sequence identity to the genomic sequence that is replaced. In other embodiments, the homology between the DNA template and genomic sequence is higher than 99%, for example if only 1 nucleotide differs as between donor and genomic sequences of over 100 contiguous base pairs. A non-homologous portion of the DNA template contains sequences not present in the locus of interest, such that new sequence, viz., sequence encoding the transgene, are introduced into the locus of interest. In some embodiments, the non-homologous sequence is generally flanked by sequences of 50-1,000 base pairs (or any integral value therebetween) or any number of base pairs greater than 1,000, that are homologous or identical to sequences in the locus of interest. In some embodiments, the DNA template is non-homologous to the first sequence, and is inserted into the genome by non-homologous recombination mechanisms.

In some embodiments, the exogenous sequence encodes a polypeptide selected from a Chimeric Antigen Receptor (CAR), a recombinant TCR, dnTGFβRII, sgp130, mutated IL6Ra (mutIL6Ra), HLA-G, IL-2, IL-12, IL-15, IL-18, FOXP3 inhibitor, a secreted inhibitor of Tumor Associated Macrophages (TAM), such as a CCR2/CCL2 neutralization agent, immunogenic peptide(s) or a secreted antibody, such as an anti-IDO1, anti-IL10, anti-PD1, anti-PDL1, anti-IL6, anti-GM-CSF or anti-PGE2 antibody.

In some embodiments, the exogenous sequence comprises a sequence for correcting a mutated endogenous gene present at the locus.

In some embodiments, the exogenous sequence is inserted into an endogenous sequence encoding one or more of the following genes: IL7R, CD45, IL2RG, JAK3, RAG1, RAG2, ARTEMIS, ADA, TRAC, CCR5, RFX5, RFXAP, RFXANK(B), CIITA, ZAP-70, CRAC, ORAI1, STIM1. POLA1, MAP3K14, GATA2, MCM4, IRF8, RTEL1, FCGR3A, Ncr1, TAP1, TAP2, RFX5, RFXAP, RFXANK(B), CIITA, ZAP-70, CRAC, ORAI1 and STIM1 (preferably in NK cells).

In some embodiments, the cell is a hematopoietic stem cell (HSC) or a HSC derived lineage cell. In some embodiments, the exogenous sequence is inserted at a locus expressed in HSC derived lineage cells (e.g., microglial cells), such as CCR5, TMEM119, CD11B, β2m, CX3CR1 or S100A9.

In some embodiments, the cells are immune cells and are modified with an exogenous sequence that reduce the risk of causing cytokine release syndrome (CRS) during the course of a cell therapy treatment by expressing or over expressing soluble polypeptides that interfere with pro-inflammatory cytokine pathways, such as those involving interleukins IL6 and IL18. The soluble polypeptides are preferably not antibodies, to avoid immune rejection, but human polypeptides, such as soluble GP130, IL18-BP and soluble IL6Ra.

The present invention is also drawn to methods for producing therapeutic immune cells expressing transgenes, such as chimeric antigen receptors (CAR), which may not require viral vectors. Replacing viral vectors as per the transformation methods according to the present invention, by linear double or single stranded nucleic acids, is highly beneficial from both the cost and safety perspectives. Manufacturing viral vectors is laborious and expansive, especially in GMP grade, whereas the use of such vectors requires confined spaces. Furthermore, viral integration generally occurs randomly, which may have adverse consequences on the genome and create malignant cells.

The chimeric antigen receptors (CAR) or transgenic TCRs that can be integrated at specific loci into the cell's genome as per the present invention can be any of those reported in the art so far, especially those having shown efficiency against various malignancies as reviewed for instance by Steven Van Schandevyl & Tessa Kerre [Chimeric antigen receptor T-cell therapy: design improvements and therapeutic strategies in cancer treatment, Acta Clinica Belgica, 2020, 75:1, 26-32,].

Preferred CAR structures are those combining an extracellular binding domain against a component present on the target cell, for example an antibody-based specificity for a desired antigen (e.g., tumor antigen) with a T cell receptor-activating intracellular domain to generate a chimeric protein that exhibits a specific anti-target cellular immune activity. Generally, CAR consists of an extracellular single chain antibody (scFv), comprising the light (VL) and the heavy (VH) variable fragment of a target antigen specific monoclonal antibody joined by a flexible linker, fused to the intracellular signalling domain of the T cell antigen receptor complex zeta chain and have the ability, when expressed in immune effector cells, to redirect antigen recognition based on the monoclonal antibody's specificity. CAR can be single-chain or multi-chain as described in WO2014039523.

More preferred CARS according to the present invention are those described in the examples, which more preferably comprise an extracellular binding domain directed against one antigen selected from CD19, CD22, CD33, 5T4, ROR1, CD38, CD52, CD123, CS1, BCMA, Flt3, CD70, EGFRvIII, WT1, HSP-70 and CCL1. Such CARS have preferably one structure involving a signal transduction domain comprising a fragment of 4-1BB (GenBank: AAA53133) or CD28 (NP_006130.1), such as described for instance in WO2016120216.

In some embodiments, the exogenous sequence, preferably encoding a chimeric antigen receptor (CAR), is integrated at the TCR locus or at selected gene loci that are upregulated upon immune cells activation. In some embodiments, the exogenous sequence(s) encoding the CAR and the endogenous gene coding sequence(s) may be co-transcribed, for instance by being separated by cis-regulatory elements (e.g. 2A cis-acting hydrolase elements) or by an internal ribosome entry site (IRES), which are also introduced. For instance, in some embodiments, the exogenous sequences encoding a CAR can be placed under transcriptional control of the promoter of endogenous genes that are activated by the tumor microenvironment, such as HIF1a, transcription factor hypoxia-inducible factor, or the aryl hydrocarbon receptor (AhR), which are gene sensors respectively induced by hypoxia and xenobiotics in the close environment of tumors.

In some embodiments, the exogenous sequence encodes an NK inhibitor, preferably comprising a sequence encoding a non-polymorphic class I molecule or viral evasin such as UL18 [Uniprot #F5HFB4] and UL16 [also called ULBP1—Uniprot #Q9BZM6], fragments or fusions thereof.

In some embodiment, the exogenous sequence encodes a polypeptide displaying at least 80% amino acid sequence identity with HLA-G or HLA-E or a functional variant thereof.

These exogenous sequences can be introduced into the genome by deleting or modifying the endogenous coding sequence(s) present at said locus (knock-out by knock-in), so that a gene inactivation can be combined with transgenesis.

In some embodiments, the exogenous sequence as described herein comprises a transgene encoding a therapeutic protein of a disease associated gene. In some embodiments, the disease or condition to be treated and transgene are shown below in Table 4.

TABLE 4
Diseases and transgenes for their treatment.

| | | Nucleotide | Amino acid |
|---|---|---|---|
| Disease | Transgene | sequence | sequence |
| Mucopoly- | IDUA | SEQ ID NO: 1 | SEQ ID NO: 2 |
| saccharidosis | | | |
| Type I (Scheie, | | | |
| Hurler-Scheie or | | | |
| Hurler syndrome) | | | |
| Mucopoly- | IDS | SEQ ID NO: 3 | SEQ ID NO: 4 |
| saccharidosis | | | |
| Type II (Hunter | | | |
| syndrome) | | | |
| Mucopoly- | ARSB | SEQ ID NO: 5 | SEQ ID NO: 6 |
| saccharidosis | | | |
| Type VI (Maroteaux- | | | |
| Lamy syndrome) | | | |
| Mucopoly- | GUSB | SEQ ID NO: 7 | SEQ ID NO: 8 |
| saccharidosis | | | |
| Type VII (Sly disease) | | | |
| X-linked | ABCD1 | SEQ ID NO: 9 | SEQ ID NO: 10 |
| Adrenoleukodystrophy | | | |
| Globoid Cell | GALC | SEQ ID NO: 11 | SEQ ID NO: 12 |
| Leukodystrophy | | | |
| (Krabbe disease) | | | |
| Metachromatic | ARSA | SEQ ID NO: 13 | SEQ ID NO: 14 |
| Leukodystrophy | | | |
| Metachromatic | PSAP | SEQ ID NO: 15 | SEQ ID NO: 16 |
| Leukodystrophy | | | |
| Gaucher disease | GBA | SEQ ID NO: 17 | SEQ ID NO: 18 |
| Fucosidosis | FUCA1 | SEQ ID NO: 19 | SEQ ID NO: 20 |
| Alpha-mannosidosis | MAN2B1 | SEQ ID NO: 21 | SEQ ID NO: 22 |
| Aspartylgluco- | AGA | SEQ ID NO: 23 | SEQ ID NO: 24 |
| saminuria | | | |
| Farber's disease | ASAH1 | SEQ ID NO: 25 | SEQ ID NO: 26 |
| Tay-Sachs disease | HEXA | SEQ ID NO: 27 | SEQ ID NO: 28 |
| Pompe disease | GAA | SEQ ID NO: 29 | SEQ ID NO: 30 |
| Niemann Pick disease | SMPD1 | SEQ ID NO: 31 | SEQ ID NO: 32 |
| Wolman disease | LIPA | SEQ ID NO: 33 | SEQ ID NO: 34 |
| CDKL5-deficiency | CDKL5 | SEQ ID NO: 35 | SEQ ID NO: 36 |
| related diseases | | | |
| (e.g., Early infantile | | | |
| epileptic | | | |
| encephalopathy (EIEE) | | | |
| disease, Atypical Rett | | | |
| syndrome, CDKL5- | | | |
| related epileptic | | | |
| encephalopathy | | | |
| disease, or West | | | |
| syndrome disease) | | | |
| Sickle Cell Anemia | HBB | SEQ ID NO: 206 | SEQ ID NO: 207 |
| (SCA) | | | |
| X-linked hyper- | CD40L | SEQ ID NO: 208 | SEQ ID NO: 209 |
| immunoglobulin M | | | |
| syndrome | | | |
| Severe obesity | ADCY3 | SEQ ID NO: 210 | SEQ ID NO: 211 |
| | BDNF | SEQ ID NO: 212 | SEQ ID NO: 213 |
| | KSR2 | SEQ ID NO: 214 | SEQ ID NO: 215 |
| | LEP | SEQ ID NO: 216 | SEQ ID NO: 217 |

In some embodiments, the exogenous sequence comprises a sequence encoding or correcting:
HBB for treating Sickle Cell Anemia (SCA);
CD40L for treating X-linked hyper-immunoglobulin M syndrome;
IDUA for treating Mucopolysaccharidosis Type I (Scheie, Hurler-Scheie or Hurler syndrome),
IDS for treating Mucopolysaccharidosis Type II (Hunter),
ARSB for treating Mucopolysaccharidosis Type VI (Maroteaux-Lamy),
GUSB for treating Mucopolysaccharidosis Type VII (Sly),
ABCD1 for treating X-linked Adrenoleukodystrophy,
GALC for treating Globoid Cell Leukodystrophy (Krabbe),
ARSA for treating Metachromatic Leukodystrophy,
GBA for treating Gaucher Disease,
FUCA1 for treating Fucosidosis,
MAN2B1 for treating Alpha-mannosidosis,
AGA for treating Aspartylglucosaminuria,
ASAH1 for treating Farber Disease,
HEXA for treating Tay-Sachs Disease,
GAA for treating Pompe Disease,
SMPD1 for treating Niemann Pick Disease,
DMD for treating Duchenne muscular dystrophy
LIPA for treating Wolman Syndrome,
CDKL5 for treating CDKL5-deficiency related disease, or
ADCY3, BDNF, KSR2, LEP for treating severe obesity.

In some embodiments, the DNA template comprises a coding sequence of a transgene as described herein. In some embodiments, the DNA template comprises a coding region of a gene selected from the group consisting of IDUA, IDS, ARSB, GUSB, ABCD1, GALC, ARSA, PSAP, GBA, FUCA1, MAN2B1, AGA, ASAH1, HEXA, GAA, SMPD1, LIPA, CDKL5, HBB, CD40L, ADCY3, BDNF, KSR2, and LEP.

In some embodiments, the DNA template comprises a nucleotide sequence selected from the group consisting of SEQ ID NOS:1, 3, 5, 7, 9, 11, 13, 15, 17, 19, 21, 23, 25, 27, 29, 31, 33, 35, 206, 208, 210, 212, 214, 216 and variants thereof as described herein.

In some embodiments, the DNA template encodes a therapeutic protein comprising an amino acid sequence selected from any one of SEQ ID NOS:2, 4, 6, 8, 10, 12, 14, 16, 18, 20, 22, 24, 26, 28, 30, 32, 34, 36, 207, 209, 211, 213, 215, 217 and variants thereof as described herein.

In some embodiments, the nucleotide sequence of IDUA comprises SEQ ID NO:1 and the amino acid sequence comprises SEQ ID NO:2.

In some embodiments, the nucleotide sequence of IDS comprises SEQ ID NO:3 and the amino acid sequence comprises SEQ ID NO:4.

In some embodiments, the nucleotide sequence of ARSB comprises SEQ ID NO:5 and the amino acid sequence comprises SEQ ID NO:6.

In some embodiments, the nucleotide sequence of GUSB comprises SEQ ID NO:7 and the amino acid sequence comprises SEQ ID NO:8.

In some embodiments, the nucleotide sequence of ABCD1 comprises SEQ ID NO:9 and the amino acid sequence comprises SEQ ID NO:10.

In some embodiments, the nucleotide sequence of GALC comprises SEQ ID NO:11 and the amino acid sequence comprises SEQ ID NO:12.

In some embodiments, the nucleotide sequence of ARSA comprises SEQ ID NO:13 and the amino acid sequence comprises SEQ ID NO:14.

In some embodiments, the nucleotide sequence of PSAP comprises SEQ ID NO:15 and the amino acid sequence comprises SEQ ID NO:16.

In some embodiments, the nucleotide sequence of GBA comprises SEQ ID NO:17 and the amino acid sequence comprises SEQ ID NO:18.

In some embodiments, the nucleotide sequence of FUCA1 comprises SEQ ID NO:19 and the amino acid sequence comprises SEQ ID NO:20.

In some embodiments, the nucleotide sequence of MAN2B1 comprises SEQ ID NO:21 and the amino acid sequence comprises SEQ ID NO:22.

In some embodiments, the nucleotide sequence of AGA comprises SEQ ID NO:23 and the amino acid sequence comprises SEQ ID NO:24.

In some embodiments, the nucleotide sequence of ASAH1 comprises SEQ ID NO:25 and the amino acid sequence comprises SEQ ID NO:26.

In some embodiments, the nucleotide sequence of HEXA comprises SEQ ID NO:27 and the amino acid sequence comprises SEQ ID NO:28.

In some embodiments, the nucleotide sequence of GAA comprises SEQ ID NO:29 and the amino acid sequence comprises SEQ ID NO:30.

In some embodiments, the nucleotide sequence of SMPD1 comprises SEQ ID NO:31 and the amino acid sequence comprises SEQ ID NO:32.

In some embodiments, the nucleotide sequence of LIPA comprises SEQ ID NO:33 and the amino acid sequence comprises SEQ ID NO:34.

In some embodiments, the nucleotide sequence of CDKL5 comprises SEQ ID NO:35 and the amino acid sequence comprises SEQ ID NO:36.

In some embodiments, the nucleotide sequence of HBB comprises SEQ ID NO:206 and the amino acid sequence comprises SEQ ID NO:207.

In some embodiments, the nucleotide sequence of CD40L comprises SEQ ID NO:208 and the amino acid sequence comprises SEQ ID NO:209.

In some embodiments, the nucleotide sequence of ADCY3 comprises SEQ ID NO:210 and the amino acid sequence comprises SEQ ID NO:211.

In some embodiments, the nucleotide sequence of BDNF comprises SEQ ID NO:212 and the amino acid sequence comprises SEQ ID NO:213.

In some embodiments, the nucleotide sequence of KSR2 comprises SEQ ID NO:214 and the amino acid sequence comprises SEQ ID NO:215.

In some embodiments, the nucleotide sequence of LEP comprises SEQ ID NO:216 and the amino acid sequence comprises SEQ ID NO:217.

In some embodiments, the exogenous sequence comprises one or more copies of a nucleotide sequence selected from any one of SEQ ID NOS:1, 3, 5, 7, 9, 11, 13, 15, 17, 19, 21, 23, 25, 27, 29, 31, 33, 35, 206, 208, 210, 212, 214 and 216.

In some embodiments, the exogenous sequence comprises one or more copies of a nucleotide sequence encoding an amino acid sequence selected from any one of SEQ ID NOS:2, 4, 6, 8, 10, 12, 14, 16, 18, 20, 22, 24, 26, 28, 30, 32, 34, 36, 207, 209, 211, 213, 215, and 217.

In some embodiments, the exogenous sequence comprises a nucleotide sequence encoding a therapeutic protein that is a variant of any one of SEQ ID NOS:2, 4, 6, 8, 10, 12, 14, 16, 18, 20, 22, 24, 26, 28, 30, 32, 34, 36, 207, 209, 211, 213, 215 and 217.

A particular nucleotide sequence encoding a therapeutic protein may be identical over its entire length to the coding sequence in SEQ ID NOS:1, 3, 5, 7, 9, 11, 13, 15, 17, 19, 21, 23, 25, 27, 29, 31, 33, 35, 206, 208, 210, 212, 214 and 216. Alternatively, a particular nucleotide sequence encoding a therapeutic protein may be an alternate form of SEQ ID NOS: 1, 3, 5, 7, 9, 11, 13, 15, 17, 19, 21, 23, 25, 27, 29, 31, 33, 35, 206, 208, 210, 212, 214 and 216, due to degeneracy in the genetic code or variation in codon usage encoding the polypeptides of SEQ ID NOS:2, 4, 6, 8, 10, 12, 14, 16, 18, 20, 22, 24, 26, 28, 30, 32, 34, 36, 207, 209, 211, 213, 215 and 217. In some embodiments, the exogenous sequence comprises a nucleotide sequence that is highly identical, at least 90% identical, with a nucleotide sequence encoding a therapeutic protein or at least 90% identical with the encoding nucleotide sequence set forth in SEQ ID NOS: 1, 3, 5, 7, 9, 11, 13, 15, 17, 19, 21, 23, 25, 27, 29, 31, 33, 35, 206, 208, 210, 212, 214 or 216. In some embodiments, the exogenous sequence comprises a nucleotide sequence that is at least 90%, 91%, 92%, 93%, 94%, 95%, 96%, 97%, 98%, or 99% identical to the nucleotide sequence set forth in SEQ ID NOS: 1, 3, 5, 7, 9, 11, 13, 15, 17, 19, 21, 23, 25, 27, 29, 31, 33, 35, 206, 208, 210, 212, 214 or 216.

“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

When an exogenous sequence comprising a polynucleotide encoding the therapeutic proteins of the invention is used for the recombinant production of a therapeutic protein, the polynucleotide may include the coding sequence for the full-length polypeptide or a fragment thereof, by itself; the coding sequence for the full-length polypeptide or fragment in reading frame with other coding sequences, such as those encoding a leader or secretory sequence, a pre-, or pro or prepro-protein sequence, or other fusion peptide portions. The polynucleotide may also contain non-coding 5′ and 3′ sequences, such as transcribed, non-translated sequences, splicing and polyadenylation signals, ribosome binding sites and sequences that stabilize mRNA.

In some embodiments the therapeutic protein can further comprises secretory signal peptides allowing its secretion by the gene edited cells of the present invention. Some Examples of such signal peptides are listed in Table 5 below:

TABLE 5
Examples of useful signal peptides

| SEQ ID | | |
|---|---|---|
| NO: # | Origin of the peptide | Polypeptide sequence |
| 37 | Human albumin peptide | MKWVTFISLLFLFSSAYS |
| 38 | Human chymotrypsinogen | MAFLWLLSCWALLGTTFG |
| | peptide | |
| 39 | Human interleukin-2 peptide | MQLLSCIALILALV |
| 40 | Human trypsinogen-2 peptide | MNLLLILTFVAAAVA |
| 41 | Human BM40 peptide | MRAWIFFLLCLAGRALA |
| 42 | Secrecon | MWWRLWWLLLLLLLLWPMVWA |
| 43 | Mouse IgKVIII | METDTLLLWVLLLWVPGSTG |
| 44 | Human IgKVIII | MDMRVPAQLLGLLLLWLRGARC |
| 45 | CD33 | MPLLLLLPLLWAGALA |
| 46 | tPA | MDAMKRGLCCVLLLCGAVFVSPS |
| 47 | Consensus | MLLLLLLLLLLALALA |
| 48 | Native | MLLLLLLLGLRLQLSLG |

In some embodiments the therapeutic protein can further comprise peptide allowing cell uptake, such as cell penetrating peptides (CPP) and Apolipoproteins. Examples of cell penetrating peptides and Apolipoproteins are listed in Table 6 below.

TABLE 6
Examples of useful CPP and Apolipoproteins

| SEQ ID | Origin of the | |
|---|---|---|
| NO: # | polypeptide | Polypeptide sequence |
| 49 | Penetratin | RQIKIWFQNRRMKWKK |
| 50 | TAT | YGRKKRRQRRR |
| 51 | SynB1 | RGGRLSYSRRRFSTSTGR |
| 52 | SynB3 | RRLSYSRRRF |
| 53 | PTD-4 | PIRRRKKLRRLK |
| 54 | PTD-5 | RRQRRTSKLMKR |
| 55 | FHV Coat | RRRRNRTRRNRRRVR |
| 56 | BMV Gag | KMTRAQRRAAARRNRWTAR |
| 57 | HTLV-II | TRRQRTRRARRNR |
| | Rex | |
| 58 | D-Tat | GRKKRRQRRRPPQ |
| 59 | R9-Tat | GRRRRRRRRRPPQ |
| 60 | Transportan | GWTLNSAGYLLGKINLKALAALAKKIL |
| 61 | MAP | KLALKLALKLALALKLA |
| 62 | SBP | MGLGLHLLVLAAALQGAWSQPKKKRKV |
| 63 | FBP | GALFLGWLGAAGSTMGAWSQPKKKRKV |
| 64 | MPG ac | GALFLGFLGAAGSTMGAWSQPKKKRKV |
| 65 | MPG(ΔNLS) | GALFLGFLGAAGSTMGAWSQPKSKRKV |
| 66 | Pep-1 | KETWWETWWTEWSQPKKKRKV |
| 67 | Pep-2 | KETWFETWFTEWSQPKKKRKV |
| 68 | ApoE p1 | LRKLRKRLLLRKLRKRLL |
| 69 | ApoE p2 | LRKLRKRLLRDADDLLRKLRKRLLRDADDL |
| 70 | ApoE p3 | LRVRLASHLRKLRKRLL |
| 71 | ApoE p4 | TEELRVRLASHLRKLRKRLL |
| 72 | ApoE p5 | LRVRLASHLRKLRKRLLLRVRLASHLRKLRKRLL |
| 73 | ApoE p6 | TEELRVRLASHLRKLRKRLLTEELRVRLASHLRKLRKRLL |
| 74 | Myc Peptide | EQKLISEEDL |
| 75 | ApoB | SSVIDALQYKLEGTTRLTRKRGLKLATALSLSNKFVEGS |
| | Peptide | |

In some embodiments, the exogenous sequence comprises a polynucleotide having a nucleotide sequence at least 90% identical, and more preferably at least 91%, 92%, 93%, 94%, 95%, 96%, 97%, 98% or 99% identical to a nucleotide sequence encoding a therapeutic protein having the amino acid sequence in SEQ ID NOS:2, 4, 6, 8, 10, 12, 14, 16, 18, 20, 22, 24, 26, 28, 30, 32, 34, 36, 207, 209, 211, 213, 215 or 217.

Conventional means utilizing known computer programs such as the BestFit program (Wisconsin Sequence Analysis Package, Version 10 for Unix, Genetics Computer Group. University Research Park, 575 Science Drive, Madison, Wis. 53711) may be utilized to determine if a particular nucleic acid molecule is at least 90%, 91%, 92%, 93%, 94%, 95%, 96%, 97%, 98% or 99% identical to any one of the nucleotide sequences shown in SEQ ID NOS:1, 3, 5, 7, 9, 11, 13, 15, 17, 19, 21, 23, 25, 27, 29, 31, 33, 35, 206, 208, 210, 212, 214 or 216.

In some embodiments, the exogenous sequence comprises a polynucleotide encoding a therapeutic protein that has an amino acid sequence of the therapeutic protein of SEQ ID NOS:2, 4, 6, 8, 10, 12, 14, 16, 18, 20, 22, 24, 26, 28, 30, 32, 34, 36, 207, 209, 211, 213, 215, or 217, in which several, 1, 1-2, 1-3, 1-5, 5-10, or 10-20 amino acid residues are substituted, deleted or added, in any combination.

In some embodiments, the exogenous sequence comprises a polynucleotide that is at least 90%, 91%, 92%, 93%, 94%, 95%, 96%, 97%, 98% or 99% identical over their entire length to a polynucleotide encoding a therapeutic protein having the amino acid sequence set out in SEQ ID NOS:2, 4, 6, 8, 10, 12, 14, 16, 18, 20, 22, 24, 26, 28, 30, 32, 34, 36, 207, 209, 211, 213, 215 or 217.

In some embodiments, the therapeutic protein expressed by the exogenous sequence is identical to a wild-type amino acid sequence of the protein, e.g., any of SEQ ID NOS:2, 4, 6, 8, 10, 12, 14, 16, 18, 20, 22, 24, 26, 28, 30, 32, 34, 36, 207, 209, 211, 213, 215 or 217.

In some embodiments, the therapeutic protein expressed by the exogenous sequence is a functional fragment or variant of any of SEQ ID NOS:2, 4, 6, 8, 10, 12, 14, 16, 18, 20, 22, 24, 26, 28, 30, 32, 34, 36, 207, 209, 211, 213, 215 or 217.

In some embodiments, the therapeutic protein comprises the polypeptide of SEQ ID NOS:2, 4, 6, 8, 10, 12, 14, 16, 18, 20, 22, 24, 26, 28, 30, 32, 34, 36, 207, 209, 211, 213, 215 or 217, as well as polypeptides and fragments which have activity and comprise at least 90% identity to the polypeptide of SEQ ID NOS:2, 4, 6, 8, 10, 12, 14, 16, 18, 20, 22, 24, 26, 28, 30, 32, 34, 36, 207, 209, 211, 213, 215 or 217, or the relevant portion and more preferably at least 96%, 97% or 98% identity to the polypeptide of SEQ ID NOS:2, 4, 6, 8, 10, 12, 14, 16, 18, 20, 22, 24, 26, 28, 30, 32, 34, 36, 207, 209, 211, 213, 215 or 217, and still more preferably at least 91%, 92%, 93%, 94%, 95%, 96%, 97%, 98%, 99%, or 100% identity to the polypeptide of SEQ ID NOS:2, 4, 6, 8, 10, 12, 14, 16, 18, 20, 22, 24, 26, 28, 30, 32, 34, 36, 207, 209, 211, 213, 215 or 217.

The therapeutic protein may be a part of a larger protein such as a fusion protein. It is often advantageous to include additional amino acid sequence which contains secretory or leader sequences, pro-sequences, or other sequences which may aid in stability.

In some embodiments, the exogenous sequence encodes a biologically active fragment of any of SEQ ID NOS:2, 4, 6, 8, 10, 12, 14, 16, 18, 20, 22, 24, 26, 28, 30, 32, 34, 36, 207, 209, 211, 213, 215 or 217. A fragment is a polypeptide having an amino acid sequence that entirely is the same as part but not all of the amino acid sequence of one of the aforementioned therapeutic protein. As with the full-length therapeutic proteins, fragments may be “free-standing,” or comprised within a larger polypeptide of which they form a part or region, most preferably as a single continuous region. In some embodiments, a fragment can constitute from about 10 contiguous amino acids identified in SEQ ID NOS:2, 4, 6, 8, 10, 12, 14, 16, 18, 20, 22, 24, 26, 28, 30, 32, 34, 36, 207, 209, 211, 213, 215 or 217.

In some embodiments, fragments include, for example, truncation polypeptides having the amino acid sequence of the therapeutic protein, except for deletion of a continuous series of residues that includes the amino terminus, or a continuous series of residues that includes the carboxyl terminus or deletion of two continuous series of residues, one including the amino terminus and one including the carboxyl terminus. Also preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Functional fragments are those that mediate protein activity of the wild type protein, including those with a similar activity or an improved activity.

In some embodiments, the fragments can lack from 1-20 amino acids (i.e., 1, 2, 3, 4, 5, 6, 7, 8, 9, 10, 11, 12, 13, 14, 15, 16, 17, 18, 19, or 20 amino acids) of the N-terminus and/or C-terminus of any of SEQ ID NOS:2, 4, 6, 8, 10, 12, 14, 16, 18, 20, 22, 24, 26, 28, 30, 32, 34, 36, 207, 209, 211, 213, 215, 217, 219, or 221.

In some embodiments, the exogenous sequence encodes a polypeptide having an amino acid sequence at least 90% identical to that of SEQ ID NOS:2, 4, 6, 8, 10, 12, 14, 16, 18, 20, 22, 24, 26, 28, 30, 32, 34, 36, 207, 209, 211, 213, 215 or 217, or functional fragments thereof with at least 90% identity to the corresponding fragment of SEQ ID NOS:2, 4, 6, 8, 10, 12, 14, 16, 18, 20, 22, 24, 26, 28, 30, 32, 34, 36, 207, 209, 211, 213, 215 or 217, all of which retain the biological activity of the therapeutic protein. Included in this group are variants of the defined sequence and fragment. In some embodiments, variants are those that vary from the reference sequence by conservative amino acid substitutions, i.e. those that substitute a residue with another of like characteristics. Typical substitutions are among Ala, Val, Leu and Ile; among Ser and Thr; among the acidic residues Asp and Glu; among Asn and Gln; and among the basic residues Lys and Arg, or aromatic residues Phe and Tyr. In some embodiments, the exogenous sequence encodes a polypeptide variants in which 1-20 amino acids are substituted, deleted, or added in any combination.

In some embodiments, the exogenous sequence is inserted at the genomic locus in a cell by homologous recombination, NHEJ, HDR, MMEJ or HMEJ.

In some embodiments, the exogenous sequence is inserted at said locus by homologous recombination.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology and generally uses a “donor” molecule (also referred as “polynucleotide template”) to be integrated into the endogenous locus (“target” sequence) by homologous recombination or NHEJ repair. This leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

### Cells

In some embodiments, the invention provides genetically modified cells obtainable according to any one of the embodiments of the methods described herein.

In some embodiments, the cell is a mammalian cell, preferably a primate cell, more preferably a human cell. In some embodiments, the cell is a primary cell. In some embodiments, the cell is an immune cell, preferably a T-cell or a NK cell. In some embodiments, the cell is a primary T-cell, more preferably a primary T-cell from a patient, such as a tumor infiltrating lymphocyte (TIL), or a primary T-cell from a donor.

By “immune cell” is meant a cell of hematopoietic origin functionally involved in the initiation and/or execution of innate and/or adaptive immune response, such as typically CD3 or CD4 positive cells. The immune cell according to the present invention can be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and from tumors, such as tumor infiltrating lymphocytes. In some embodiments, said immune cell can be derived from a healthy donor, from a patient diagnosed with cancer or from a patient diagnosed with an infection. In another embodiment, said cell is part of a mixed population of immune cells which present different phenotypic characteristics, such as comprising CD4, CD8 and CD56 positive cells.

By “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRCS cells; IMR90 cells; Jurkat cells; HepG2 cells; HeLa cells; HT-1080 cells; HCT-116 cells; Hu-h7 cells; Huvec cells; Molt 4 cells. Primary cells are generally used in cell therapy as they are deemed more functional and less tumorigenic.

In general, primary immune cells are provided from donors or patients through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28(3):145-284).

The primary immune cells according to the present invention can also be differentiated from stem cells, such as cord blood stem cells, progenitor cells, bone marrow stem cells, hematopoietic stem cells (HSC) and induced pluripotent stem cells (iPS).

In some embodiments, the cell is a hematopoietic stem cell. As used herein, the term “hematopoietic stem cells” (or “HSC”) refer to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSC are CD34−. In addition, HSC also refer to long term repopulating HSC (LT-HSC) and short term repopulating HSC (ST-HSC). LT-HSC and ST-HSC are differentiated, based on functional potential and on cell surface marker expression. For example, in some embodiments, human HSC are a CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSC are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSC are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSC are less quiescent (i.e., more active) and more proliferative than LT-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSC can be used in any of the methods described herein. In some embodiments, ST-HSC are useful because they are highly proliferative and thus, can more quickly give rise to differentiated progeny.

In some embodiments, the hematopoietic stem cells for use in genetic modification herein are isolated from bone marrow. In some embodiments, HSC can be taken from the pelvis, at the iliac crest, using a needle or syringe.

In some embodiments, the hematopoietic stem cells can be derived from human cord blood or mobilized peripheral blood. Hematopoietic stem cells obtained from human peripheral blood may be mobilized by one of a variety of strategies. Exemplary agents that can be used to induce mobilization of hematopoietic stem cells from the bone marrow into peripheral blood include chemokine (C-X-C motif) receptor 4 (CXCR4) antagonists, such as AMD3100 (also known as Plerixafor and MOZOBIL (Genzyme, Boston, Mass.)) and granulocyte colony-stimulating factor (GCSF), the combination of which has been shown to rapidly mobilize CD34+ cells in clinical experiments. Additionally, chemokine (C-X-C motif) ligand 2 (CXCL2, also referred to as GROβ) represents another agent capable of inducing hematopoietic stem cell mobilization to from bone marrow to peripheral blood. Agents capable of inducing mobilization of hematopoietic stem cells for use with the compositions and methods of the invention may be used in combination with one another. For instance, CXCR4 antagonists (e.g., AMD3100), CXCL2, and/or GCSF may be administered to a subject sequentially or simultaneously in a single mixture in order to induce mobilization of hematopoietic stem cells from bone marrow into peripheral blood. The use of these agents as inducers of hematopoietic stem cell mobilization is described, e.g., in Pelus, Current Opinion in Hematology 15:285 (2008), the disclosure of which is incorporated herein by reference.

In some embodiments, HSC are harvested from the circulating peripheral blood, while the blood donor is injected with an agent that mobilizes the HSC from the bone marrow. In some embodiments, the agent that mobilizes the HSC from the bone marrow to the peripheral blood is a cytokine, such as granulocyte-colony stimulating factor (G-CSF). In some embodiments, populations of HSC isolated from the peripheral blood are enriched in CD34+ cells, and comprise at least 50%, at least 70%, or at least 90% of CD34+ cells.

In some embodiments, for mobilized peripheral blood (MPB) leukapheresis, CD34+ cells can generally be processed and enriched using immunomagnetic beads such as CliniMACS, Purified CD34+ cells can be seeded on culture bags at 1×10⁶ cells/ml in serum-free medium in the presence of cells culture grade Stem Cell Factor (SCF), preferably 300 ng/ml (Amgen Inc., Thousand Oaks, Calif., USA), preferably with FMS-like tyrosine kinase 3 ligand (FLT3L) 300 ng/ml, and Thrombopoietin (TPO), preferably around 100 ng/ml and further interleukine IL-3, preferably more than 60 ng/ml (all from Cell Genix Technologies) during between preferably 12 and 24 hours before being transferred to an electroporation buffer comprising the sequence specific reagent (e.g., mRNA). Upon electroporation, the cells are transferred back to the culture medium prior to being resuspended in saline and transferred in a syringe for infusion.

Methods for enriching or depleting specific cell populations in a mixture of cells are well known in the art. For example, cell populations can be enriched or depleted by density separation, rosetting tetrameric antibody complex mediated enrichment/depletion, magnetic activated cell sorting (MACS), multi-parameter fluorescence based molecular phenotypes such as fluorescence-activated cell sorting (FACS), or any combination thereof. Collectively, these methods of enriching or depleting cell populations may be referred to generally herein as “sorting” the cell populations or contacting the cells “under conditions” to form or produce an enriched (+) or depleted (−) cell population.

Upon collection of the mobilized cells, the withdrawn hematopoietic stem cells can be genetically modified as described herein and then infused into a patient in need thereof, which may be the donor or another subject, such as a subject that is at least partially HLA-matched to the donor, for the treatment of disease as described herein.

In some embodiments, these cells form a population of cells, which can originate from a single donor or patient. These populations of cells can be expanded under closed culture recipients to comply with highest manufacturing practices requirements and can be frozen prior to infusion into a patient, thereby providing “off the shelf” or “ready to use” therapeutic compositions.

In some embodiments, the HSC are CD34+. In some embodiments, the HSC can further be described as CD133+, CD90+, CD38−, CD45RA-, Lin-, or any combination thereof.

In some embodiments, the cells are induced pluripotent stem cells (iPS). In some embodiments, the HSC capable of differentiating into cells such as microglial cells are derived from pluripotent stem cells, such as induced pluripotent stem cells (iPS). See, e.g., Abud et al., Neuron 94, 278-293 (2017). In some embodiments, the iPS cells are genetically modified as described herein and then differentiated into HSC cells. In some embodiments, the iPS cells are differentiated into HSC and then the HSC are genetically modified as described herein. In further embodiments, cells can be genetically modified as described herein before being reprogrammed into iPS cells and HSCs as described for instance in Int. Appl. No. PCT/EP2018/083180. In some embodiments, the hematopoietic stem cells can be isolated from the patient to be treated or isolated from a compatible donor.

In some embodiments, hematopoietic stem cells are obtained from induced pluripotent stem (iPS) cells derived from cells of the patient to be treated or from a compatible donor.

In some embodiments, the HSC can be expanded ex vivo prior to genetic modification and/or infusion of these cells into the patient. See, e.g., U.S. Pat. Nos. 9,580,426; 9,956,249; 9,527,828; 9,428,748; 9,394,520; 9,328,085; 9,226,942; 9,115,341; 8,927,281.

In some embodiments, the cells are isolated from a donor that is an HLA matched sibling donor, an HLA matched unrelated donor, a partially matched unrelated donor, a haploidentical related donor, autologous donor, an HLA unmatched donor, a pool of donors or any combination thereof. In some embodiments, the population of therapeutic cells is allogeneic. In some embodiments, the population of therapeutic cells is autologous. In some embodiments, the population of therapeutic cells is haploidentical.

As used herein, a “donor” is a human or animal from which one or more cells are isolated prior to the modification of the cells or progeny thereof, and administration into a recipient. The one or more cells may be, e.g., a population of hematopoietic stem cells to be modified, expanded, enriched, or maintained according to the methods of the invention prior to administration of the cells or the progeny thereof into a recipient.

As used herein, a “recipient” is a patient that receives a transplant, such as a transplant containing a population of modified hematopoietic stem cells or a population of differentiated cells. The transplanted cells administered to a recipient may be, e.g., autologous, syngeneic, or allogeneic cells.

“Expansion” in the context of cells refers to increase in the number of a characteristic cell type, or cell types, from an initial cell population of cells, which may or may not be identical. The initial cells used for expansion may not be the same as the cells generated from expansion.

“Cell population” refers to eukaryotic mammalian, preferably human, cells isolated from biological sources, for example, blood product or tissues and derived from more than one cell.

“Enriched” when used in the context of cell population refers to a cell population selected based on the presence of one or more markers, for example, CD34+.

The term “CD34+ cells” refers to cells that express at their surface CD34 marker. CD34+ cells can be detected and counted using for example flow cytometry and fluorescently labeled anti-CD34 antibodies.

“Enriched in CD34+ cells” means that a cell population has been selected based on the presence of CD34 marker. Accordingly, the percentage of CD34+ cells in the cell population after selection method is higher than the percentage of CD34+ cells in the initial cell population before selecting step based on CD34 markers. For example, CD34+ cells may represent at least 50%, 60%, 70%, 80% or at least 90% of the cells in a cell population enriched in CD34+ cells.

### Therapeutic Methods

In another embodiment, the invention provides a method of treating a disease or condition in a subject comprising administering to the subject an effective amount of a pharmaceutical composition comprising cells modified according to the methods herein.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The term “subject” or “patient” as used herein includes all members of the animal kingdom including non-human primates and humans.

As used herein, the terms “administering,” or “providing” refer to the placement of a compound, cell, population of cells, or composition as disclosed herein into a subject or to a cell by a method or route which results in at least partial delivery of the agent at a desired site. Pharmaceutical compositions comprising the compounds or cells disclosed herein can be administered or provided by any appropriate route which results in an effective treatment in the subject or effect on the cells.

An “effective amount” or “therapeutically effective amount” refers to that amount of a composition described herein which, when administered to a subject (e.g., human), is sufficient to aid in treating a disease. The amount of a composition that constitutes a “therapeutically effective amount” will vary depending on the cell preparations, the condition and its severity, the manner of administration, and the age of the subject to be treated, but can be determined routinely by one of ordinary skill in the art having regard to his own knowledge and to this disclosure. When referring to an individual active ingredient or composition, administered alone, a therapeutically effective dose refers to that ingredient or composition alone. When referring to a combination, a therapeutically effective dose refers to combined amounts of the active ingredients, compositions or both that result in the therapeutic effect, whether administered serially, concurrently or simultaneously.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention aims to produce genetically engineered therapeutic cells, especially cells from hematopoietic lineages, stem cells or differentiated cells, such as T-cells, for treating disease by gene repair, cross-correction and/or expression of therapeutic molecules. The therapeutic effects are generally obtained by the targeted insertion, as per the methods described herein of exogenous DNA templates, leading to the expression by the cells of supplementary or corrected alleles.

In some embodiments, the present invention is useful for treating diseases characterized by systemic red blood cells dysfunction, such as those due to mutations into HBB, in particular sickle cell anemia and beta-thalassemia.

In some embodiments, the present invention is useful for treating auto-immune diseases characterized by systemic T-cells dysfunction, such as those due to mutations into STAT3.

In some embodiments, transgenes are integrated into immune cells to restore their functionalities or redirect their immune properties against pathological cells. Engineered T-cells or NK cells according to the present invention can express CARs or recombinant TCRs to target various types of cancer, especially malignancies conditions expressing markers such as CD19, in particular Acute Lymphoblastic Leukemia and non-hodgkin lymphoma, CD22, in particular Acute Lymphoblastic Leukemia, CD123 and CD33, in particular in Acute Myeloid Lymphoma, CS1 or BCMA, in particular in Multiple myeloma, mesothelin and ROR1, in carcinomas such as breast tumors, CD70 in gliomas, 5T4 in ovarian cancer and also CD7 in leukemias. The present invention can also be used to produce engineered tumor infiltrating lymphocytes (TILs), which are also active against tumors in order to improve their potencies.

In some embodiments, the patient has a monogenic disease or condition. In some embodiments, the patient has a deficiency in the expression of an endogenous gene homologous to the transgene. In some embodiments, the patient has a lysosomal storage disease. In some embodiments, disease or condition is selected from Mucopolysaccharidosis Type I (Scheie, Hurler-Scheie or Hurler syndrome), Mucopolysaccharidosis Type II (Hunter syndrome), Mucopolysaccharidosis Type VI (Maroteaux-Lamy syndrome), Mucopolysaccharidosis Type VII (Sly disease), X-linked Adrenoleukodystrophy, Globoid Cell Leukodystrophy (Krabbe disease), Metachromatic Leukodystrophy, Gaucher disease, Fucosidosis, Alpha-mannosidosis, Aspartylglucosaminuria, Farber's disease, Tay-Sachs disease, Pompe disease, Niemann Pick disease and Wolman disease. In some embodiments, the patient has a Central Nervous System (CNS) disease. In some embodiments the CNS disease is selected from Alzheimer disease, Parkinson disease, Huntington's disease, multiple sclerosis disease. In some embodiments, the patient has a CDKL5-deficiency related disease. In some embodiments the CDKL5-deficiency disease is selected from Early infantile epileptic encephalopathy (EIEE), Atypical Rett syndrome, CDKL5-related epileptic encephalopathy, and West syndrome.

### CDKL5-Deficiency Related Diseases:

Early Infantile Epileptic Encephalopathy (EIEE) Disease

Early Infantile Epileptic Encephalopathy (EIEE) is a neurological disorder characterized by seizures. The disorder affects newborns, usually within the first three months of life (most often within the first 10 days) in the form of epileptic seizures. Infants have primarily tonic seizures (which cause stiffening of muscles of the body, generally those in the back, legs, and arms), but may also experience partial seizures, and rarely, myoclonic seizures (which cause jerks or twitches of the upper body, arms, or legs). Episodes may occur more than a hundred times per day. Most infants with the disorder show underdevelopment of part or all of the cerebral hemispheres or structural anomalies. Some cases are caused by metabolic disorders or by mutations in several different genes. The cause for many cases can't be determined. There are several types of early infantile epileptic encephalopathy. The EEGs reveal a characteristic pattern of high voltage spike wave discharge followed by little activity. This pattern is known as “burst suppression.” The seizures associated with this disease are difficult to treat and the syndrome is severely progressive. Some children with this condition go on to develop other epileptic disorders such as West syndrome and Lennox-Gestaut syndrome.

EIEE may be the result of different etiologies. Many cases have been associated with structural brain abnormalities. Some cases are due to metabolic disorders (cytochrome C oxidase deficiency, carnitine palmitoyl transferase II deficiency) or brain malformations (such as porencephaly, or hemimegalencephaly) that may or not be genetic in origin. Genetic variants of EIEE have been associated with mutations in certain genes such as ARX (Xp22.13), CDKL5 (Xp22), SL25A22 (11p15.5) and STXBP1 (9q34.1), among others. The genetic abnormalities are thought to lead to EIEE as they are related to neuronal dysfunction or brain dysgenesis.

Atypical Rett Syndrome

Atypical Rett syndrome is a neurodevelopmental disorder that is diagnosed when a child has some of the symptoms of Rett syndrome but does not meet all the diagnostic criteria. Like the classic form of Rett syndrome, atypical Rett syndrome mostly affects girls. Children with atypical Rett syndrome can have symptoms that are either milder or more severe than those seen in Rett syndrome. Several subtypes of atypical Rett syndrome have been defined. The early-onset seizure type is characterized by seizures in the first months of life with later development of Rett features (including developmental problems, loss of language skills, and repeated hand wringing or hand washing movements). It is frequently caused by mutations in the X-linked CDKL5 gene (Xp22).

CDKL5-Related Epileptic Encephalopathy Disease

CDKL5-related epileptic encephalopathy is characterized by a 3-stage evolution consisting of early epilepsy (stage 1), then infantile spasms (stage 2) and, finally, multifocal and refractory myoclonic epilepsy (stage 3). See, e.g., Bahi-Buisson et al. Epilepsia. 49:1027-1037 (2008). Genetic abnormalities of cyclin-dependent kinase-like 5 (CDKL5) cause an early-onset epileptic encephalopathy.

West Syndrome Disease

West syndrome is a type of epilepsy characterized by spasms, abnormal brain wave patterns called hypsarrhythmia and sometimes intellectual disability. The spasms that occur may range from violent jackknife or “salaam” movements where the whole body bends in half, or they may be no more than a mild twitching of the shoulder or eye changes. These spasms usually begin in the early months after birth and can sometimes be helped with medication. There are many different causes of West syndrome and if a specific cause can be identified, a diagnosis of symptomatic West syndrome can be made. If a cause cannot be determined, a diagnosis of cryptogenic West syndrome is made. A specific cause for West syndrome can be identified in approximately 70-75% of those affected. X-linked West syndrome (X-linked infantile spasm syndrome or ISSX) can be caused by a mutation in the CDKL5 gene or the ARX gene on the X chromosome.

Mucopolysaccharidoses

Mucopolysaccharidoses (MPSs) are degenerative genetic diseases linked to an enzymatic defect. In particular, MPSs are caused by the deficiency or the inactivity of lysosomal enzymes which catalyze the gradual metabolism of complex sugar molecules called glycosaminoglycans (GAGs). These enzymatic deficiencies cause an accumulation of GAGS in the cells, the tissues and, in particular, the cell lysosomes of affected subjects, leading to permanent and progressive cell damage which affects the appearance, the physical capacities, the organ function and, in most cases, the mental development of affected subjects.

Eleven distinct enzymatic defects have been identified, corresponding to seven distinct clinical categories of MPS. Each MPS is characterized by a deficiency or inactivity of one or more enzymes which degrade mucopolysaccharides, namely heparan sulfate, dermatan sulfate, chondroitin sulfate and keratan sulfate.

MPS I is divided into three subtypes based on severity of symptoms. All three types result from an absence of, or insufficient levels of, the enzyme alpha-L-iduronidase (IDUA). Children born to an MPS I parent carry the defective gene.

MPS I H (also called Hurler syndrome or alpha-L-iduronidase deficiency), is the most severe of the MPS I subtypes. Developmental delay is evident by the end of the first year, and patients usually stop developing between ages 2 and 4. This is followed by progressive mental decline and loss of physical skills. Language may be limited due to hearing loss and an enlarged tongue. In time, the clear layers of the cornea become clouded and retinas may begin to degenerate. Carpal tunnel syndrome (or similar compression of nerves elsewhere in the body) and restricted joint movement are common. Affected children may be quite large at birth and appear normal but may have inguinal (in the groin) or umbilical (where the umbilical cord passes through the abdomen) hernias. Growth in height may be faster than normal but begins to slow before the end of the first year and often ends around age 3. Many children develop a short body trunk and a maximum stature of less than 4 feet. Distinct facial features (including flat face, depressed nasal bridge, and bulging forehead) become more evident in the second year. By age 2, the ribs have widened and are oar-shaped. The liver, spleen, and heart are often enlarged. Children may experience noisy breathing and recurring upper respiratory tract and ear infections. Feeding may be difficult for some children, and many experience periodic bowel problems. Children with Hurler syndrome often die before age 10 from obstructive airway disease, respiratory infections, and cardiac complications.

MPS I S, Scheie syndrome, is the mildest form of MPS 1. Symptoms generally begin to appear after age 5, with diagnosis most commonly made after age 10. Children with Scheie syndrome have normal intelligence or may have mild learning disabilities; some may have psychiatric problems. Glaucoma, retinal degeneration, and clouded corneas may significantly impair vision. Other problems include carpal tunnel syndrome or other nerve compression, stiff joints, claw hands and deformed feet, a short neck, and aortic valve disease. Some affected individuals also have obstructive airway disease and sleep apnea. Persons with Scheie syndrome can live into adulthood.

MPS I H-S, Hurler-Scheie syndrome, is less severe than Hurler syndrome alone. Symptoms generally begin between ages 3 and 8. Children may have moderate intellectual disability and learning difficulties. Skeletal and systemic irregularities include short stature, marked smallness in the jaws, progressive joint stiffness, compressed spinal cord, clouded corneas, hearing loss, heart disease, coarse facial features, and umbilical hernia. Respiratory problems, sleep apnea, and heart disease may develop in adolescence. Some persons with MPS I H-S need continuous positive airway pressure during sleep to ease breathing. Life expectancy is generally into the late teens or early twenties.

MPS II, also known as Hunter syndrome, is caused by lack of the enzyme iduronate sulfatase. Hunter syndrome has two clinical subtypes and (since it shows X-linked recessive inheritance) is the only one of the mucopolysaccharidoses in which the mother alone can pass the defective gene to a son. The incidence of Hunter syndrome is estimated to be 1 in 100,000 to 150,000 male births.

Mutations in the IDS gene cause MPS II. The IDS gene provides instructions for producing the I2S enzyme, which is involved in the breakdown of large sugar molecules called glycosaminoglycans (GAGs). Specifically, 12S removes a chemical group known as a sulfate from a molecule called sulfated alpha-L-iduronic acid, which is present in two GAGS called heparan sulfate and dermatan sulfate. I2S is located in lysosomes, compartments within cells that digest and recycle different types of molecules.

Mucopolysaccharidosis type VI (MPS VI) or Maroteaux-Lamy disease is a lysosomal storage disease, of the mucopolysaccharidosis group, characterized by severe somatic involvement and an absence of psycho-intellectual regression. The prevalence of this rare mucopolysaccharidosis is between 1/250,000 and 1/600,000 births. In the severe forms, the first clinical manifestations occur between 6 and 24 months and are gradually accentuated: facial dysmorphia (macroglossia, mouth constantly half open, thick features), joint limitations, very severe dysostosis multiplex (platyspondyly, kyphosis, scoliosis, pectus carinatum, genu valgum, long bone deformation), small size (less than 1.10 m), hepatomegaly, heart valve damage, cardiomyopathy, deafness, corneal opacities. Intellectual development is usually normal or virtually normal, but the auditory and ophthalmological damage can cause learning difficulties. The symptoms and the severity of the disease vary considerably from one patient to the other and intermediate forms, or even very moderate forms also exist (spondyloepiphyseal-metaphyseal dysplasia associated with cardiovascular involvement). Like the other mucopolysaccharidoses, Maroteaux-Lamy disease is linked to the defect of an enzyme of mucopolysaccharide metabolism, in the case in point N-acetylgalactosamine-4-sulfatase (also called arylsulfatase B)(ARSB). This enzyme metabolizes the sulfate group of dermatan sulfate (Neufeld et al.: “The mucopolysaccharidoses” The Metabolic Basis of Inherited Diseases, eds. Scriver et al, New York, McGraw-Hill, 1989, p. 1565-1587). This enzymatic defect blocks the gradual degradation of dermatan sulfate, thereby leading to an accumulation of dermatan sulfate in the lysosomes of the storage tissues.

Mucopolysaccharidosis type VII (MPS VII) or Sly disease is a very rare lysosomal storage disease of the mucopolysaccharidosis group. The symptomology is extremely heterogeneous: antenatal forms (nonimmune fetoplacental anasarca), severe neonatal forms (with dysmorphia, hernias, hepatosplenomegaly, club feet, dysostosis, significant hypotonia and neurological problems evolving to retarded growth and a profound intellectual deficiency in the event of survival) and very moderate forms discovered at adolescence or even at adult age (thoracic kyphosis). The disease is due to a defect in beta-D-glucuronidase (GUSB) responsible for accumulation, in the lysosomes, of various glycosaminoglycans: dermatan sulfate, heparan sulfate and chondroitin sulfate. There is at the current time no effective treatment for this disease.

X-Linked Adrenoleukodystrophy

Adrenoleukodystrophy (ALD) is an X-linked disease affecting 1/20,000 males either as cerebral ALD in childhood or as adrenomyleneuropathy (AMN) in adults. Childhood ALD is the more severe form, with onset of neurological symptoms between 5-12 years of age. Central nervous system demyelination progresses rapidly and death occurs within a few years. AMN is a milder form of the disease with onset at 15-30 years of age and a more progressive course. Adrenal insufficiency (Addison's disease) may remain the only clinical manifestation of ALD. The principal biochemical abnormality of ALD is the accumulation of very long chain fatty acids (VLCFA) because of impaired β-oxidation in peroxisomes.

More than 650 mutations in the ABCD1 gene have been found to cause X-linked adrenoleukodystrophy. This condition is characterized by varying degrees of cognitive and movement problems as well as hormone imbalances. The mutations that cause X-linked adrenoleukodystrophy prevent the production of any ALDP in about 75 percent of people with this disorder. Other people with X-linked adrenoleukodystrophy can produce ALDP, but the protein is not able to perform its normal function. With little or no functional ALDP, VLCFAs are not broken down, and they build up in the body. The accumulation of these fats may be toxic to the adrenal glands (small glands on top of each kidney) and to the fatty layer of insulation (myelin) that surrounds many nerves in the body. Research suggests that the accumulation of VLCFAs triggers an inflammatory response in the brain, which could lead to the breakdown of myelin. The destruction of these tissues leads to the signs and symptoms of X-linked adrenoleukodystrophy.

Globoid Cell Leukodystrophy

Infantile globoid cell leucodystrophy (GLD, galactosylceramide lipidosis or Krabbe's disease) is a rare, autosomal recessive hereditary degenerative disorder in the central and peripheral nervous systems. The incidence in the US is estimated to 1:100.000. It is characterized by the presence of globoid cells (cells with multiple nuclei), degeneration of the protective myelin layer of the nerves and loss of cells in the brain. GLD causes severe mental reduction and motoric delay. It is caused by a deficiency in galactocerebroside-β-galactosidase (GALC), which is an essential enzyme in the metabolism of myelin. The disease often affects infants prior to the age of 6 months, but it can also appear during youth or in adults. The symptoms include irritability, fever without any known cause, stiffness in the limbs (hypertony), seizures, problems associated with food intake, vomiting and delayed development of mental and motoric capabilities. Additional symptoms include muscular weakness, spasticity, deafness and blindness.

The galactosylceramidase gene (GALC) is about 60 kb in length and consists of 17 exons. Numerous mutations and polymorphisms have been identified in the murine and human GALC gene, causing GLD with different degrees of severity.

Metachromatic Leukodystrophy

Metachromatic leukodystrophy is an inherited disorder characterized by the accumulation of fats called sulfatides in cells. This accumulation especially affects cells in the nervous system that produce myelin, the substance that insulates and protects nerves. Nerve cells covered by myelin make up a tissue called white matter. Sulfatide accumulation in myelin-producing cells causes progressive destruction of white matter (leukodystrophy) throughout the nervous system, including in the brain and spinal cord (the central nervous system) and the nerves connecting the brain and spinal cord to muscles and sensory cells that detect sensations such as touch, pain, heat, and sound (the peripheral nervous system).

In people with metachromatic leukodystrophy, white matter damage causes progressive deterioration of intellectual functions and motor skills, such as the ability to walk. Affected individuals also develop loss of sensation in the extremities (peripheral neuropathy), incontinence, seizures, paralysis, an inability to speak, blindness, and hearing loss. Eventually they lose awareness of their surroundings and become unresponsive. While neurological problems are the primary feature of metachromatic leukodystrophy, effects of sulfatide accumulation on other organs and tissues have been reported, most often involving the gallbladder.

The most common form of metachromatic leukodystrophy, affecting about 50 to 60 percent of all individuals with this disorder, is called the late infantile form. This form of the disorder usually appears in the second year of life. Affected children lose any speech they have developed, become weak, and develop problems with walking (gait disturbance). As the disorder worsens, muscle tone generally first decreases, and then increases to the point of rigidity. Individuals with the late infantile form of metachromatic leukodystrophy typically do not survive past childhood.

In 20 to 30 percent of individuals with metachromatic leukodystrophy, onset occurs between the age of 4 and adolescence. In this juvenile form, the first signs of the disorder may be behavioral problems and increasing difficulty with schoolwork. Progression of the disorder is slower than in the late infantile form, and affected individuals may survive for about 20 years after diagnosis.

Most individuals with metachromatic leukodystrophy have mutations in the ARSA gene, which provides instructions for making the enzyme arylsulfatase A. This enzyme is located in cellular structures called lysosomes, which are the cell's recycling centers. Within lysosomes, arylsulfatase A helps break down sulfatides. A few individuals with metachromatic leukodystrophy have mutations in the PSAP gene. This gene provides instructions for making a protein that is broken up (cleaved) into smaller proteins that assist enzymes in breaking down various fats. One of these smaller proteins is called saposin B; this protein works with arylsulfatase A to break down sulfatides.

Mutations in the ARSA or PSAP genes result in a decreased ability to break down sulfatides, resulting in the accumulation of these substances in cells. Excess sulfatides are toxic to the nervous system. The accumulation gradually destroys myelin-producing cells, leading to the impairment of nervous system function that occurs in metachromatic leukodystrophy.

In some cases, individuals with very low arylsulfatase A activity show no symptoms of metachromatic leukodystrophy. This condition is called pseudoarylsulfatase deficiency.

The adult form of metachromatic leukodystrophy affects approximately 15 to 20 percent of individuals with the disorder. In this form, the first symptoms appear during the teenage years or later. Often behavioral problems such as alcoholism, drug abuse, or difficulties at school or work are the first symptoms to appear. The affected individual may experience psychiatric symptoms such as delusions or hallucinations. People with the adult form of metachromatic leukodystrophy may survive for 20 to 30 years after diagnosis. During this time there may be some periods of relative stability and other periods of more rapid decline.

Metachromatic leukodystrophy gets its name from the way cells with an accumulation of sulfatides appear when viewed under a microscope. The sulfatides form granules that are described as metachromatic, which means they pick up color differently than surrounding cellular material when stained for examination.

Gaucher Disease

Gaucher disease is an inherited disorder that affects many of the body's organs and tissues. The signs and symptoms of this condition vary widely among affected individuals. Researchers have described several types of Gaucher disease based on their characteristic features.

Type 1 Gaucher disease is the most common form of this condition. Type 1 is also called non-neuronopathic Gaucher disease because the brain and spinal cord (the central nervous system) are usually not affected. The features of this condition range from mild to severe and may appear anytime from childhood to adulthood. Major signs and symptoms include enlargement of the liver and spleen (hepatosplenomegaly), a low number of red blood cells (anemia), easy bruising caused by a decrease in blood platelets (thrombocytopenia), lung disease, and bone abnormalities such as bone pain, fractures, and arthritis.

Types 2 and 3 Gaucher disease are known as neuronopathic forms of the disorder because they are characterized by problems that affect the central nervous system. In addition to the signs and symptoms described above, these conditions can cause abnormal eye movements, seizures, and brain damage. Type 2 Gaucher disease usually causes life-threatening medical problems beginning in infancy. Type 3 Gaucher disease also affects the nervous system, but it tends to worsen more slowly than type 2.

The most severe type of Gaucher disease is called the perinatal lethal form. This condition causes severe or life-threatening complications starting before birth or in infancy. Features of the perinatal lethal form can include extensive swelling caused by fluid accumulation before birth (hydrops fetalis); dry, scaly skin (ichthyosis) or other skin abnormalities; hepatosplenomegaly; distinctive facial features; and serious neurological problems. As its name indicates, most infants with the perinatal lethal form of Gaucher disease survive for only a few days after birth.

Another form of Gaucher disease is known as the cardiovascular type because it primarily affects the heart, causing the heart valves to harden (calcify). People with the cardiovascular form of Gaucher disease may also have eye abnormalities, bone disease, and mild enlargement of the spleen (splenomegaly).

Mutations in the GBA gene cause Gaucher disease. The GBA gene provides instructions for making an enzyme called beta-glucocerebrosidase. This enzyme breaks down a fatty substance called glucocerebroside into a sugar (glucose) and a simpler fat molecule (ceramide). Mutations in the GBA gene greatly reduce or eliminate the activity of beta-glucocerebrosidase. Without enough of this enzyme, glucocerebroside and related substances can build up to toxic levels within cells. Tissues and organs are damaged by the abnormal accumulation and storage of these substances, causing the characteristic features of Gaucher disease.

Fucosidosis

Fucosidosis is a condition that affects many areas of the body, especially the brain. Affected individuals have intellectual disability that worsens with age, and many develop dementia later in life. People with this condition often have delayed development of motor skills such as walking; the skills they do acquire deteriorate over time. Additional signs and symptoms of fucosidosis include impaired growth; abnormal bone development (dysostosis multiplex); seizures; abnormal muscle stiffness (spasticity); clusters of enlarged blood vessels forming small, dark red spots on the skin (angiokeratomas); distinctive facial features that are often described as “coarse”; recurrent respiratory infections; and abnormally large abdominal organs (visceromegaly).

In severe cases, symptoms typically appear in infancy, and affected individuals usually live into late childhood. In milder cases, symptoms begin at age 1 or 2, and affected individuals tend to survive into mid-adulthood.

In the past, researchers described two types of this condition based on symptoms and age of onset, but current opinion is that the two types are actually a single disorder with signs and symptoms that range in severity.

Mutations in the FUCA1 gene cause fucosidosis. The FUCA1 gene provides instructions for making an enzyme called alpha-L-fucosidase. This enzyme plays a role in the breakdown of complexes of sugar molecules (oligosaccharides) attached to certain proteins (glycoproteins) and fats (glycolipids). Alpha-L-fucosidase is responsible for cutting (cleaving) off a sugar molecule called fucose toward the end of the breakdown process.

FUCA1 gene mutations severely reduce or eliminate the activity of the alpha-L-fucosidase enzyme. A lack of enzyme activity results in an incomplete breakdown of glycolipids and glycoproteins. These partially broken down compounds gradually accumulate within various cells and tissues throughout the body and cause cells to malfunction. Brain cells are particularly sensitive to the buildup of glycolipids and glycoproteins, which can result in cell death. Loss of brain cells is thought to cause the neurological symptoms of fucosidosis. Accumulation of glycolipids and glycoproteins also occurs in other organs such as the liver, spleen, skin, heart, pancreas, and kidneys, contributing to the additional symptoms of fucosidosis.

Alpha-Mannosidosis

Alpha-mannosidosis is an autosomal, recessively inherited lysosomal storage disorder that has been clinically well characterized (M. A. Chester et al., 1982, in Genetic Errors of Glycoprotein Metabolism pp 90-119, Springer Verlag, Berlin). Glycoproteins are normally degraded stepwise in the lysosome and one of the steps, namely the cleavage of .alpha.-linked mannose residues from the non-reducing end during the ordered degradation of N-linked glycoproteins is catalysed by the enzyme lysosomal α-mannosidase (EC 3.2.1.24). However, in alpha-mannosidosis, a deficiency of the enzyme α-mannosidase results in the accumulation of mannose rich oligosaccharides. As a result, the lysosomes increase in size and swell, which impairs cell functions.

The symptoms of α-mannosidosis include psychomotor retardation, ataxia, impaired hearing, vacuolized lymphocytes in the peripheral blood and skeletal changes.

Mutations in the MAN2B1 gene cause alpha-mannosidosis. This gene provides instructions for making the enzyme alpha-mannosidase. This enzyme works in the lysosomes, which are compartments that digest and recycle materials in the cell. Within lysosomes, the enzyme helps break down complexes of sugar molecules (oligosaccharides) attached to certain proteins (glycoproteins). In particular, alpha-mannosidase helps break down oligosaccharides containing a sugar molecule called mannose.

Mutations in the MAN2B1 gene interfere with the ability of the alpha-mannosidase enzyme to perform its role in breaking down mannose-containing oligosaccharides. These oligosaccharides accumulate in the lysosomes and cause cells to malfunction and eventually die. Tissues and organs are damaged by the abnormal accumulation of oligosaccharides and the resulting cell death, leading to the characteristic features of alpha-mannosidosis.

Aspartylglucosaminuria

Aspartylglucosaminuria is a condition that causes a progressive decline in mental functioning. Infants with aspartylglucosaminuria appear healthy at birth, and development is typically normal throughout early childhood. The first sign of this condition, evident around the age of 2 or 3, is usually delayed speech. Mild intellectual disability then becomes apparent, and learning occurs at a slowed pace. Intellectual disability progressively worsens in adolescence. Most people with this disorder lose much of the speech they have learned, and affected adults usually have only a few words in their vocabulary. Adults with aspartylglucosaminuria may develop seizures or problems with movement.

People with this condition may also have bones that become progressively weak and prone to fracture (osteoporosis), an unusually large range of joint movement (hypermobility), and loose skin. Affected individuals tend to have a characteristic facial appearance that includes widely spaced eyes (ocular hypertelorism), small ears, and full lips. The nose is short and broad and the face is usually square-shaped. Children with this condition may be tall for their age, but lack of a growth spurt in puberty typically causes adults to be short. Affected children also tend to have frequent upper respiratory infections. Individuals with aspartylglucosaminuria usually survive into mid-adulthood.

Mutations in the AGA gene cause aspartylglucosaminuria. The AGA gene provides instructions for producing an enzyme called aspartylglucosaminidase. This enzyme is active in lysosomes, which are structures inside cells that act as recycling centers. Within lysosomes, the enzyme helps break down complexes of sugar molecules (oligosaccharides) attached to certain proteins (glycoproteins).

AGA gene mutations result in the absence or shortage of the aspartylglucosaminidase enzyme in lysosomes, preventing the normal breakdown of glycoproteins. As a result, glycoproteins can build up within the lysosomes. Excess glycoproteins disrupt the normal functions of the cell and can result in destruction of the cell. A buildup of glycoproteins seems to particularly affect nerve cells in the brain; loss of these cells causes many of the signs and symptoms of aspartylglucosaminuria.

Farber's Disease

Farber's disease is an inherited condition involving the breakdown and use of fats in the body (lipid metabolism). People with this condition have an abnormal accumulation of lipids (fat) throughout the cells and tissues of the body, particularly around the joints. Farber's disease is characterized by three classic symptoms: a hoarse voice or weak cry, small lumps of fat under the skin and in other tissues (lipogranulomas), and swollen and painful joints. Other symptoms may include difficulty breathing, an enlarged liver and spleen (hepatosplenomegaly), and developmental delay. Researchers have described seven types of Farber's disease based on their characteristic features. This condition is caused by mutations in the ASAH1 gene and is inherited in an autosomal recessive manner.

Tay-Sachs Disease

Tay-Sachs disease is a rare inherited disorder that progressively destroys nerve cells (neurons) in the brain and spinal cord.

The most common form of Tay-Sachs disease becomes apparent in infancy. Infants with this disorder typically appear normal until the age of 3 to 6 months, when their development slows and muscles used for movement weaken. Affected infants lose motor skills such as turning over, sitting, and crawling. They also develop an exaggerated startle reaction to loud noises. As the disease progresses, children with Tay-Sachs disease experience seizures, vision and hearing loss, intellectual disability, and paralysis. An eye abnormality called a cherry-red spot, which can be identified with an eye examination, is characteristic of this disorder. Children with this severe infantile form of Tay-Sachs disease usually live only into early childhood.

Other forms of Tay-Sachs disease are very rare. Signs and symptoms can appear in childhood, adolescence, or adulthood and are usually milder than those seen with the infantile form. Characteristic features include muscle weakness, loss of muscle coordination (ataxia) and other problems with movement, speech problems, and mental illness. These signs and symptoms vary widely among people with late-onset forms of Tay-Sachs disease.

Mutations in the HEXA gene cause Tay-Sachs disease. The HEXA gene provides instructions for making part of an enzyme called beta-hexosaminidase A, which plays a critical role in the brain and spinal cord. This enzyme is located in lysosomes, which are structures in cells that break down toxic substances and act as recycling centers. Within lysosomes, beta-hexosaminidase A helps break down a fatty substance called GM2 ganglioside.

Mutations in the HEXA gene disrupt the activity of beta-hexosaminidase A, which prevents the enzyme from breaking down GM2 ganglioside. As a result, this substance accumulates to toxic levels, particularly in neurons in the brain and spinal cord. Progressive damage caused by the buildup of GM2 ganglioside leads to the destruction of these neurons, which causes the signs and symptoms of Tay-Sachs disease.

Because Tay-Sachs disease impairs the function of a lysosomal enzyme and involves the buildup of GM2 ganglioside, this condition is sometimes referred to as a lysosomal storage disorder or a GM2-gangliosidosis.

Pompe Disease

Pompe disease (also known as glycogen storage disease type II; acid alpha-glucosidase deficiency; acid maltase deficiency; GAA deficiency; GSD II; glycogenosis type II; glycogenosis, generalized, cardiac form; cardiomegalia glycogenica diffusa; acid maltase deficiency; AMD; or alpha-1,4-glucosidase deficiency) is an autosomal recessive metabolic genetic disorder characterized by mutations in the gene for the lysomsomal enzyme acid alpha-glucosidase (GAA) (also known as acid maltase). Mutations in the GAA gene eliminate or reduce the ability of the GAA enzyme to hydrolyze the a-1,4 and a-1,6 linkages in glycogen, maltose and isomaltose. As a result, glycogen accumulates in the lysosomes and cytoplasm of cells throughout the body leading to cell and tissue destruction. Tissues that are particularly affected include skeletal muscle and cardiac muscle. The accumulated glycogen causes progressive muscle weakness leading to cardiomegaly, ambulatory difficulties and respiratory insufficiency.

Three forms of Pompe disease have been identified, including the classic infantile-onset disease, non-classic infantile-onset disease and late onset disease. The classic infantile-onset form is characterized by muscle weakness, poor muscle tone, hepatomegaly and cardiac defects. The incidence of the disease is approximately 1 in 140,000 individuals. Patients with this form of the disease often die of heart failure in the first year of life. The non-classic infantile-onset form of the disease is characterized by delayed motor skills, progressive muscle weakness and in some instances cardiomegaly. Patients with this form of the disease often live only into early childhood due to respiratory failure. The late-onset form of the disease may present in late childhood, adolescence or adulthood and is characterized by progressive muscle weakness of the legs and trunk.

Niemann Pick Disease

Niemann-Pick disease is a condition that affects many body systems. It has a wide range of symptoms that vary in severity. Niemann-Pick disease is divided into four main types: type A, type B, type C1, and type C2. These types are classified on the basis of genetic cause and the signs and symptoms of the condition.

Infants with Niemann-Pick disease type A usually develop an enlarged liver and spleen (hepatosplenomegaly) by age 3 months and fail to gain weight and grow at the expected rate (failure to thrive). The affected children develop normally until around age 1 year when they experience a progressive loss of mental abilities and movement (psychomotor regression). Children with Niemann-Pick disease type A also develop widespread lung damage (interstitial lung disease) that can cause recurrent lung infections and eventually lead to respiratory failure. All affected children have an eye abnormality called a cherry-red spot, which can be identified with an eye examination. Children with Niemann-Pick disease type A generally do not survive past early childhood.

Niemann-Pick disease type B usually presents in mid-childhood. The signs and symptoms of this type are similar to type A, but not as severe. People with Niemann-Pick disease type B often have hepatosplenomegaly, recurrent lung infections, and a low number of platelets in the blood (thrombocytopenia). They also have short stature and slowed mineralization of bone (delayed bone age). About one-third of affected individuals have the cherry-red spot eye abnormality or neurological impairment. People with Niemann-Pick disease type B usually survive into adulthood.

Niemann-Pick disease types A and B is caused by mutations in the SMPD1 gene. This gene provides instructions for producing an enzyme called acid sphingomyelinase. This enzyme is found in lysosomes, which are compartments within cells that break down and recycle different types of molecules. Acid sphingomyelinase is responsible for the conversion of a fat (lipid) called sphingomyelin into another type of lipid called ceramide. Mutations in SMPD1 lead to a shortage of acid sphingomyelinase, which results in reduced break down of sphingomyelin, causing this fat to accumulate in cells. This fat buildup causes cells to malfunction and eventually die. Over time, cell loss impairs function of tissues and organs including the brain, lungs, spleen, and liver in people with Niemann-Pick disease types A and B.

Wolman Disease

Lysosomal acid lipase deficiency is an inherited condition characterized by problems with the breakdown and use of fats and cholesterol in the body (lipid metabolism). In affected individuals, harmful amounts of fats (lipids) accumulate in cells and tissues throughout the body, which typically causes liver disease. There are two forms of the condition. The most severe and rarest form begins in infancy. The less severe form can begin from childhood to late adulthood.

In the severe, early-onset form of lysosomal acid lipase deficiency, lipids accumulate throughout the body, particularly in the liver, within the first weeks of life. This accumulation of lipids leads to several health problems, including an enlarged liver and spleen (hepatosplenomegaly), poor weight gain, a yellow tint to the skin and the whites of the eyes (jaundice), vomiting, diarrhea, fatty stool (steatorrhea), and poor absorption of nutrients from food (malabsorption). In addition, affected infants often have calcium deposits in small hormone-producing glands on top of each kidney (adrenal glands), low amounts of iron in the blood (anemia), and developmental delay. Scar tissue quickly builds up in the liver, leading to liver disease (cirrhosis). Infants with this form of lysosomal acid lipase deficiency develop multi-organ failure and severe malnutrition and generally do not survive past 1 year.

In the later-onset form of lysosomal acid lipase deficiency, signs and symptoms vary and usually begin in mid-childhood, although they can appear anytime up to late adulthood. Nearly all affected individuals develop an enlarged liver (hepatomegaly); an enlarged spleen (splenomegaly) may also occur. About two-thirds of individuals have liver fibrosis, eventually leading to cirrhosis. Approximately one-third of individuals with the later-onset form have malabsorption, diarrhea, vomiting, and steatorrhea. Individuals with this form of lysosomal acid lipase deficiency may have increased liver enzymes and high cholesterol levels, which can be detected with blood tests.

Some people with this later-onset form of lysosomal acid lipase deficiency develop an accumulation of fatty deposits on the artery walls (atherosclerosis). Although these deposits are common in the general population, they usually begin at an earlier age in people with lysosomal acid lipase deficiency. The deposits narrow the arteries, increasing the chance of heart attack or stroke. The expected lifespan of individuals with later-onset lysosomal acid lipase deficiency depends on the severity of the associated health problems.

The two forms of lysosomal acid lipase deficiency were once thought to be separate disorders. The early-onset form was known as Wolman disease, and the later-onset form was known as cholesteryl ester storage disease. Although these two disorders have the same genetic cause and are now considered to be forms of a single condition, these names are still sometimes used to distinguish between the forms of lysosomal acid lipase deficiency.

Mutations in the LIPA gene cause lysosomal acid lipase deficiency. The LIPA gene provides instructions for producing an enzyme called lysosomal acid lipase. This enzyme is found in cell compartments called lysosomes, which digest and recycle materials the cell no longer needs. The lysosomal acid lipase enzyme breaks down lipids such as cholesteryl esters and triglycerides. The lipids produced through these processes, cholesterol and fatty acids, are used by the body or transported to the liver for removal.

Mutations in the LIPA gene lead to a shortage (deficiency) of functional lysosomal acid lipase. The severity of the condition depends on how much working enzyme is available. Individuals with the early-onset form of lysosomal acid lipase deficiency have no normal enzyme activity. Those with the later-onset form are thought to have some enzyme activity remaining, and the amount generally determines the severity of signs and symptoms.

Decreased lysosomal acid lipase activity results in the accumulation of cholesteryl esters, triglycerides, and other lipids within lysosomes, causing fat buildup in multiple tissues. The body's inability to produce cholesterol from the breakdown of these lipids leads to an increase in alternative methods of cholesterol production and higher-than-normal levels of cholesterol in the blood. The excess lipids are transported to the liver for removal. Because many of them are not broken down properly, they cannot be removed from the body; instead they accumulate in the liver, resulting in liver disease. The progressive accumulation of lipids in tissues results in organ dysfunction and the signs and symptoms of lysosomal acid lipase deficiency.

Sickle Cell Disease

Sickle cell disease is a group of disorders that affects hemoglobin, the molecule in red blood cells that delivers oxygen to cells throughout the body. People with this disorder have atypical hemoglobin molecules called hemoglobin S, which can distort red blood cells into a sickle, or crescent, shape.

The signs and symptoms of sickle cell disease are caused by the sickling of red blood cells. When red blood cells sickle, they break down prematurely, which can lead to anemia. Mutations in the HBB gene cause sickle cell disease.

X-Linked Hyper-Immunoglobulin M Syndrome

X-linked hyper IgM syndrome is a condition that affects the immune system and occurs almost exclusively in males. People with this disorder have abnormal levels of proteins called antibodies or immunoglobulins. Antibodies help protect the body against infection by attaching to specific foreign particles and germs, marking them for destruction. There are several classes of antibodies, and each one has a different function in the immune system. Although the name of this condition implies that affected individuals always have high levels of immunoglobulin M (IgM), some people have normal levels of this antibody. People with X-linked hyper IgM syndrome have low levels of three other classes of antibodies: immunoglobulin G (IgG), immunoglobulin A (IgA), and immunoglobulin E (IgE). The lack of certain antibody classes makes it difficult for people with this disorder to fight off infections. Mutations in the CD40LG gene cause X-linked hyper IgM syndrome.

Duchenne Muscular Dystrophy

Muscular dystrophies are a group of genetic conditions characterized by progressive muscle weakness and wasting (atrophy). The Duchenne and Becker types of muscular dystrophy are two related conditions that primarily affect skeletal muscles, which are used for movement, and heart (cardiac) muscle. These forms of muscular dystrophy occur almost exclusively in males.

Duchenne and Becker muscular dystrophies have similar signs and symptoms and are caused by different mutations in the same gene. Mutations in the DMD gene cause the Duchenne and Becker forms of muscular dystrophy.

Severe Obesity

Obesity is very common, and accompanied by high rates of serious, life-threatening, complications such as type 2 diabetes, cardiovascular disease and cancer. Severe obesity is frequently defined with the broader meaning of having a BMI of greater than 35 kg/m². Genes that have been implicated in obesity include ADCY3, BDNF, KSR2 and LEP.

The methods can be part of an autologous or part of an allogenic treatment. By autologous, it is meant that cells used for treating patients are originating from said patient. By allogeneic is meant that the cells or population of cells used for treating patients are not originating from said patient but from a donor.

In some embodiments, the cells are administrated to patients undergoing an immunosuppressive treatment. In one embodiment, the administered cells have been made resistant to at least one immunosuppressive agent. In some embodiments, the immunosuppressive treatment helps the selection and expansion of the modified cells within the patient.

The administration of the cells may be carried out in any convenient manner, including by aerosol inhalation, injection, ingestion, transfusion, implantation or transplantation. The compositions described herein may be administered to a patient, e.g., subcutaneously, intradermally, intratumorally, intranodally, intramedullary, intramuscularly, by intravenous or intralymphatic injection, or intraperitoneally. In one embodiment, the cell compositions are administered by intravenous injection, where there are capable of migrating to the desired location such as the bone marrow.

While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, the administration of the cells or population of cells comprises administration of about 10⁴-10⁹ cells per kg body weight. In some embodiments, about 10⁵ to 10⁶ cells/kg body weight are administered. All integer values of cell numbers within those ranges are contemplated.

The cells can be administrated in one or more doses. In another embodiment, am effective amount of cells are administrated as a single dose. In another embodiment, an effective amount of cells are administrated as more than one dose over a period of time. Timing of administration is within the judgment of managing physician and depends on the clinical condition of the patient.

In some embodiments, administering genetically modified HSC cells can include treating the patient with a myeloablative and/or immune suppressive regimen to deplete host bone marrow stem cells and prevent rejection. In some embodiments, the patient is administered chemotherapy and/or radiation therapy. In some embodiments, the patient is administered a reduced dose chemotherapy regimen. In some embodiments, reduced dose chemotherapy regimen with busulfan at 25% of standard dose can be sufficient to achieve significant engraftment of modified cells while reducing conditioning-related toxicity (Aiuti A. et al. (2013), Science 23; 341 (6148)). A stronger chemotherapy regimen can be based on administration of both busulfan and fludarabine as depleting agents for endogenous HSC. In some embodiments, the dose of busulfan and fludarabine are approximately 50% and 30% of the ones employed in standard allogeneic transplantation. In another embodiment, the cells are administered following B-cell ablative therapy such as agents that react with CD20, e.g., Rituxan. In some embodiments, the patient is administered chemotherapy agents such as fludarabine, external-beam radiation therapy (XRT), cyclophosphamide, or antibodies such as OKT3 or CAMPATH.

In certain embodiments, the genetically modified cells are administered to the subject as combination therapy comprising immunosuppressive agents. Exemplary immunosuppressive agents include sirolimus, tacrolimus, cyclosporine, mycophenolate, anti-thymocyte globulin, corticosteroids, calcineurin inhibitor, anti-metabolite, such as methotrexate, post-transplant cyclophosphamide or any combination thereof. In some embodiments, the subject is pretreated with only sirolimus or tacrolimus as prophylaxis against GVHD. In some embodiments, the cells are administered to the subject before an immunosuppressive agent. In some embodiments, the cells are administered to the subject after an immunosuppressive agent. In some embodiments, the cells are administered to the subject concurrently with an immunosuppressive agent. In some embodiments, the cells are administered to the subject without an immunosuppressive agent. In some embodiments, the patient receiving genetically modified cells receives immunosuppressive agent for less than 6 months, 5 months, 4 months, 3 months, 2 months, 1 month, 3 weeks, 2 weeks, or 1 week.

### Delivery Methods

The sequence-specific endonucleases, nucleic acids encoding these nucleases, and DNA template comprising the exogenous sequence and compositions comprising the proteins and/or polynucleotides described herein for modifying the cells may be delivered in vivo or ex vivo by any suitable means.

In some embodiments, the methods comprise at least two transfection steps, wherein a first transfection step introduces the sequence-specific endonuclease into the cell, as a polypeptide or polynucleotide, and a second transfection step introduces the DNA template comprising said exogenous sequence to be inserted. In some embodiments, the first transfection step is by electroporation or nanoparticle transformation. In some embodiments, the second transfection step is by electroporation, nanoparticle or viral transformation. In preferred embodiments, the methods of the invention do not comprise a step involving a viral vector.

In some instances, integration defective or non-integrative viral vectors, which do not integrate into the genome on their own, may be used as DNA templates to perform the present invention. In such cases, the viral sequences are not regarded as constituting “viral vectors” because their expression, if any, do not participate to the exogenous gene targeted integration.

In some embodiments, polypeptides may be synthesized in situ in the cell as a result of the introduction of nucleic acids encoding the polypeptides into the cell. In some embodiments, the polypeptides can be produced outside the cell and then introduced into the cell. Methods for introducing a polynucleotide construct into cells are known in the art and include, as non-limiting examples, stable transformation methods wherein the polynucleotide construct is integrated into the genome of the cell, transient transfection methods wherein the polynucleotide construct is not integrated into the genome of the cell and virus mediated methods. In some embodiments, the polynucleotides may be introduced into a cell by recombinant viral vectors (e.g. retroviruses, adenoviruses), liposomes and the like. In one embodiment, the transient transformation methods include, for example microinjection, electroporation or particle bombardment. The polynucleotides can be included in vectors, more particularly plasmids or virus, in view of being expressed in cells.

In some embodiments, the cells are transiently transfected with a nucleic acid encoding a sequence specific endonuclease reagent. In some embodiments, about 80% of the endonuclease reagent is degraded by 30 hours, preferably by 24, more preferably by 20 hours after transfection.

In some embodiments, a sequence specific endonuclease encoded by mRNA can be synthetized with a cap to enhance its stability according to techniques well known in the art, as described, for instance, by Kore A. L., et al. (Locked nucleic acid (LNA)-modified dinucleotide mRNA cap analogue: synthesis, enzymatic incorporation, and utilization (2009) J Am Chem Soc. 131 (18):6364-5).

In some embodiments, sequence specific endonucleases as described herein may also be delivered using vectors containing sequences encoding one or more of the CRISPR/Cas system(s), zinc finger or TALEN protein(s). Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties.

Conventional viral and non-viral based gene transfer methods can be used to introduce nucleic acids encoding sequence specific endonucleases and DNA templates comprising exogenous sequences in cells (e.g., mammalian cells) and target tissues. In particular, nanoparticles and ribonucleoprotein complexes (RNP) can be used to introduce the sequence specific nuclease reagents into the cells as described for instance by Vakulskas, C. A., et al. [A high-fidelity Cas9 mutant delivered as a ribonucleoprotein complex enables efficient gene editing in human hematopoietic stem and progenitor cells (2018) Nat Med 24, 1216-1224.]

Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds.) (1995); and Yu et al., Gene Therapy 1:13-26 (1994).

In some embodiments, methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

In some embodiments, electroporation steps can be used to transfect cells. In some embodiments, these steps are typically performed in closed chambers comprising parallel plate electrodes producing a pulse electric field between said parallel plate electrodes greater than 100 volts/cm and less than 5,000 volts/cm, substantially uniform throughout the treatment volume such as described in WO 2004/083379, which is incorporated by reference, especially from page 23, line 25 to page 29, line 11. One such electroporation chamber preferably has a geometric factor (cm⁻¹) defined by the quotient of the electrode gap squared (cm²) divided by the chamber volume (cm³), wherein the geometric factor is less than or equal to 0.1 cm⁻¹, wherein the suspension of the cells and the sequence specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

In some embodiments, different exogenous sequences or multiple copies of the exogeneous sequence can be included in one DNA template. In some embodiments, the DNA template can comprise a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82: 1013-1025 (2001); Donnelly et al., J. of Gen. Virology 78: 13-21 (1997); Doronina et al., Mol. And. Cell. Biology 28(13): 4227-4239 (2008); Atkins et al., RNA 13: 803-810 (2007)).

By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

In one embodiment, a polynucleotide encoding a sequence specific endonuclease according to the present invention can be mRNA which is introduced directly into the cells, for example by electroporation. In some embodiments, the cells can be electroporated using cytoPulse technology which allows, by the use of pulsed electric fields, to transiently permeabilize living cells for delivery of material into the cells. The technology, based on the use of PulseAgile (BTX Havard Apparatus, 84 October Hill Road, Holliston, Mass. 01746, USA) electroporation waveforms grants the precise control of pulse duration, intensity as well as the interval between pulses (see U.S. Pat. No. 6,010,613 and published International Application WO 2004/083379). All these parameters can be modified in order to reach the best conditions for high transfection efficiency with minimal mortality. The first high electric field pulses allow pore formation, while subsequent lower electric field pulses allow moving the polynucleotide into the cell.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam and Lipofectin). Cationic and neutral lipids that are suitable for efficient receptor-recognition lipofection of polynucleotides include those of Felgner, WO 91/17424, WO 91/16024.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In some embodiments, the DNA template and/or sequence specific endonuclease is encoded by a viral vector. In some embodiments, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including by non-limiting example, AAV1, AAV3, AAV4, AAV5, AAV6, AAV8, AAV 8.2, AAV9, and AAV rh10 and pseudotyped AAV such as AAV2/8, AAV2/5 and AAV2/6 can also be used in accordance with the present invention.

In some embodiments, the cells are administered an effective amount of one or more caspase inhibitors in combination with an AAV vector.

The sequence specific endonuclease and DNA template constructs can be delivered using the same or different systems. For example, the DNA template polynucleotide can be provided as a PCR product, while the sequence specific endonuclease can be delivered as a mRNA composition.

In some embodiments, one or more reagents can be delivered to cells using nanoparticles. In some embodiments, nanoparticles are coated with ligands, such as antibodies, having a specific affinity towards cell surface proteins, such as CD105 (Uniprot #P17813). In some embodiments, the nanoparticles are biodegradable polymeric nanoparticles in which the sequence specific endonuclease under polynucleotide form are complexed with a polymer of polybeta amino ester and coated with polyglutamic acid (PGA).

### Compositions

The invention is also drawn to a composition comprising an effective amount of genetically modified cells prepared by the methods as described herein. In some embodiments, the invention provides a pharmaceutical composition comprising an effective amount of genetically modified cells as described herein.

In some embodiments, the composition can be used as a medicament. In some embodiments, the composition can be used for treating a disease as described herein. In some embodiments, the composition can be useful for treating cancer in a subject in need thereof.

In some embodiments, the composition comprises a population of cells, wherein at least 40% of the cells in the population have been modified according to any one the methods described herein. In some embodiments, at least 50%, 60%, 70%, 80%, 85%, 90%, 91%, 92%, 93%, 94%, 95%, 96%, 97%, 98%, 99% of the cells in the population have been modified according to any one the methods described herein. In some embodiments, the composition comprises a pure population of cells wherein 100% of the cells have been genetically modified as described herein.

The genetically modified cells can be administered either alone, or as a pharmaceutical composition in combination with diluents and/or with other components. In some embodiments, pharmaceutical compositions can comprise genetically modified cells (such as immune cells, HSC, or iPS cells) as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g. aluminum hydroxide); and preservatives. In some embodiments, compositions are formulated for intravenous administration.

In some embodiments, the genetically modified cells as described herein can be cryopreserved. In some embodiments, the cells can be cryopreserved after their isolation from subjects and prior to any genetic modification. In some embodiments, the genetically modified cells are cryopreserved after genetic modification and prior to infusion in subjects. In some embodiments, the genetically modified cells are cryopreserved after they have been expanded ex vivo.

In one embodiment, the invention provides a cryopreserved pharmaceutical composition comprising: (a) a viable composition of genetically modified cells as described herein (b) an amount of cryopreservative sufficient for the cryopreservation of the cells; and (c) a pharmaceutically acceptable carrier.

As used herein, “cryopreservation” refers to the preservation of cells by cooling to low sub-zero temperatures, such as (typically) 77 K or −196° C. (the boiling point of liquid nitrogen). Cryopreservation also refers to storing the cells at a temperature between 0°-10° C. in the absence of any cryopreservative agents. At these low temperatures, any biological activity, including the biochemical reactions that would lead to cell death, is effectively stopped. Cryoprotective agents are often used at sub-zero temperatures to preserve the cells from damage due to freezing at low temperatures or warming to room temperature.

In some embodiments, the injurious effects associated with freezing can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO), glycerol, polyvinylpyrrolidine, polyethylene glycol, albumin, dextran, sucrose, ethylene glycol, i-erythritol, D-Sorbitol, D-mannitol, D-sorbitol, i-inositol, D-lactose, choline chloride, amino acids, methanol, acetamide, glycerol monoacetate, and inorganic salts. In a preferred embodiment, DMSO is used, a liquid which is nontoxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After the addition of DMSO, cells should be kept at 0-4° C. until freezing, since DMSO concentrations of about 1% are toxic at temperatures above 4° C.

Different cryoprotective agents (Rapatz, G., et al., 1968, Cryobiology 5(1):18-25) and different cell types have different optimal cooling rates (see e.g., Rowe, A. W. and Rinfret, A. P., 1962, Blood 20:636; Rowe, A. W., 1966, Cryobiology 3(1):12-18; Lewis, J. P., et al., 1967, Transfusion 7(1):17-32; and Mazur, P., 1970, Science 168:939-949 for effects of cooling velocity on survival of marrow-stem cells and on their transplantation potential). The heat of fusion phase where water turns to ice should be minimal. The cooling procedure can be carried out by use of, e.g., a programmable freezing device or a methanol bath procedure.

After thorough freezing, cells can be rapidly transferred to a long-term cryogenic storage vessel. In one embodiment, the expanded HSC or IPs cells can be cryogenically stored in liquid nitrogen (−196° C.) or its vapor (−165° C.). Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum

In a particular embodiment, the cryopreservation procedure described in Current Protocols in Stem Cell Biology, 2007, (Mick Bhatia, et. al., ed., John Wiley and Sons, Inc.) is used and is hereby incorporated by reference. Mainly when the cells (such as HSC) on a 10-cm tissue culture plate have reached approximately 50% confluency, the media within the plate is aspirated and the cells are rinsed with phosphate buffered saline. The adherent cells are then detached by 3 ml of 0.025% trypsin/0.04% EDTA treatment. The trypsin/EDTA is neutralized by 7 ml of media and the detached cells are collected by centrifugation at 200×g for 2 min. The supernatant is aspirated off and the pellet of cells is resuspended in 1.5 ml of media. An aliquot of 1 ml of 100% DMSO is added to the suspension of cells and gently mixed. Then 1 ml aliquots of this suspension of HSC in DMSO are dispensed into CRYULES in preparation for cryopreservation. The sterilized storage CRYULES preferably have their caps threaded inside, allowing easy handling without contamination. Suitable racking systems are commercially available and can be used for cataloguing, storage, and retrieval of individual specimens.

Considerations and procedures for the manipulation, cryopreservation, and long-term storage of cells, particularly from bone marrow or peripheral blood can be found, for example, in the following references, incorporated by reference herein: Gorin, N. C., 1986, Clinics In Haematology 15(1):19-48; Bone-Marrow Conservation, Culture and Transplantation, Proceedings of a Panel, Moscow, Jul. 22-26, 1968, International Atomic Energy Agency, Vienna, pp. 107-186.

Other methods of cryopreservation of viable cells, or modifications thereof, are available and envisioned for use (e.g., cold metal-minor techniques; Livesey, S. A. and Linner, J. G., 1987, Nature 327:255; Linner, J. G., et al., 1986, J. Histochem. Cytochem. 34(9):1123-1135; U.S. Pat. Nos. 4,199,022, 3,753,357, and 4,559,298 and all of these are incorporated hereby reference in their entirety.

In some embodiments, the frozen cells are thawed quickly (e.g., in a water bath maintained at 37°-41° C.) and chilled on ice immediately upon thawing. In particular, the cryogenic vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In one embodiment, the thawing procedure after cryopreservation is described in Current Protocols in Stem Cell Biology 2007 (Mick Bhatia, et al., ed., John Wiley and Sons, Inc.) and is hereby incorporated by reference. Immediately after removing the cryogenic vial from the cryo-freezer, the vial is rolled between the hands for 10 to 30 sec until the outside of the vial is frost free. The vial is then held upright in a 37° C. water-bath until the contents are visibly thawed. The vial is immersed in 95% ethanol or sprayed with 70% ethanol to kill microorganisms from the water-bath and air dry in a sterile hood. The contents of the vial are then transferred to a 10-cm sterile culture containing 9 ml of media using sterile techniques. The cells can then be cultured and further expanded in an incubator at 37° C. with 5% humidified CO₂.

It may be desirable to treat the cells in order to prevent cellular clumping upon thawing. To prevent clumping, various procedures can be used, including but not limited to, the addition before and/or after freezing of DNase (Spitzer, G., et al., 1980, Cancer 45:3075-3085), low molecular weight dextran and citrate, hydroxyethyl starch (Stiff, P. J., et al., 1983, Cryobiology 20:17-24).

The cryoprotective agent, if toxic in humans, should be removed prior to therapeutic use of the thawed cells. In an embodiment employing DMSO as the cryopreservative, it is preferable to omit this step in order to avoid cell loss, since DMSO has no serious toxicity. However, where removal of the cryoprotective agent is desired, the removal is preferably accomplished upon thawing.

One way in which to remove the cryoprotective agent is by dilution to an insignificant concentration. This can be accomplished by addition of medium, followed by, if necessary, one or more cycles of centrifugation to pellet the cells, removal of the supernatant, and resuspension of the cells. For example, the intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, R. J. 1977, Biochemical Methods in Cell Culture and Virology, Dowden, Hutchinson & Ross, Stroudsburg, Pa., pp. 18-19; 1964, Methods in Medical Research, Eisen, H. N., et al., eds., Vol. 10, Year Book Medical Publishers, Inc., Chicago, pp. 39-47) can be done to confirm cell survival.

In one embodiment, thawed cells are tested by standard assays of viability (e.g., trypan blue exclusion) and of microbial sterility as described herein, and tested to confirm and/or determine their identity relative to the recipient.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### EXAMPLES

### Example 1: Materials and Methods

The Sequences used in the following examples are recapitulated in Table 7.

### Cells

Cryopreserved human PBMCs were used in accordance with Cellectis IRB/IEC-approved protocols. PBMCs were cultured in X-vivo-15 media (Lonza Group), containing IL-2 (Miltenyi Biotech,), and human serum AB (Seralab). Human T activator CD3/CD28 dynabeads (Thermo Fisher Scientific) were used, according to the provider's protocol, to activate T-cells for 3 days. Human hemopoietic stem cells (HSC) were purchased from New York Blood Center and cultured in HSC expansion media (StemSpan SFEM II and StemSpan CD34+ expansion supplement, StemCell Technologies). The HSCs were passaged at 3.36E5 cell/ml every 3rd day.

### TALE-Nucleases and CRISPR

TALEN designate heterodimeric TALE-nucleases as described by Voytas et al. in WO2011072246 using Fok-1 as a nuclease domain produced by Cellectis (8, rue de la Croix Jarry, 75013 Paris, France). TRAC and B2M TALEN mRNAs were produced according to previously described protocol (Poirot et al. 2015). The target sequence for TRAC and B2M TALEN TTCCTCCTACTCACCATcagcctcctggttatGGTACAGGTAAGAGCAA (SEQ ID NO. 218), and TCCGTGGCCTTAGCTGTgctcgcgctactcTCTCTTTCTGGCCTGGA (SEQ ID NO. 219) respectively, where two 17-bp recognition sites (upper case letters) are separated by a 15-bp spacer.

HBB TALEN mRNA were produced using in vitro transcription using NEB HiScribe ARCA (NEB) kit according to manufacturer protocol. The HBB TALEN target sequence is TTGCTTACATTTGCTTCTgacacaactgtgttcACTAGCAACCTCAAACA (SEQ ID NO. 239), with upper cases indicating the TALEN binding sequences and the lower case representing the spacer sequence.

The mRNA encoding CAS9 protein (SEQ ID NO: 246) were produced using mMACHINE T7 Transcription Kit kit (Invitrogen AM1344). sgRNA (SEQ ID NO: 246) targeting the first exon of TCR-alpha constant region (TRAC) was synthesized by IDT.

### Production of Double Strand DNA Repair Template

Plasmid containing CAR matrix with homology arms (SEQ ID NO. 220 to the target site was used as PCR template. Phosphorothioate modified primers were used to amplify the target region (SEQ ID NO. 221 and SEQ ID NO. 222). PCR reaction was performed using PrimeSTAR Max Premix (TaKaRa) system according to manufacturer's protocol. The PCR product were then purified with AMpure beads (Beckman Coulter) and eluted into ddH₂O.

ssODN

ssODN used in this study was custom synthesized by Integrate DNA Technology. The ssODN used to introduce 20 bp insertion at TRAC locus contains 20 bp random sequence in the center, flanking by 75 bp homology arm to TRAC TALEN target site (SEQ ID NO. 240). Two ssODNs (SEQ ID NO. 237 and 238) were used to introduce point mutation into EBB locus. The ssODNs had phosphothioate modifications at their extremities.

### Targeted Integration of CAR Construct or 20 bp Insertion in Primary T-Cells

Activated T-cells were split into fresh complete media and cultured in fresh media for 6 to 24 hrs. T-cells were transfected according to the following procedure. For TALEN mRNA transfection, the cells were first de-beaded by magnetic separation (EasySep), washed twice in Cytoporation buffer T (BTX Harvard Apparatus), and 5 million cells were then resuspended in Cytoporation buffer T. This cellular suspension was mixed with mRNA encoding TRAC TALEN at 1 μg mRNA per TALEN arm per million cells. Transfection was performed using Pulse Agile technology by applying two 0.1 mS pulses at 3,000 V/cm followed by four 0.2 mS pulses at 325 V/cm in 0.4 cm gap cuvettes (BTX Harvard Apparatus). The electroporated cells were then immediately transferred to a 12-well plate containing 2 mL of prewarmed X-vivo-15 serum-free media and incubated at 37° C. for 15 min. The cells were then incubated at 30° C. for various length of time before the second transfection with dsDNA or ssODN repair temple. For dsDNA transfection for target integration, the TALEN mRNA transfected cells were harvested, washed once with warm PBS. Five million cells were then pelleted and resuspended in 100 μl Lonza Human T cell buffer (Lonza, VPA-1002, 82 μl Human T cell buffer+18 μl Supplement). 2 μg dsDNA repair template was mixed to the cells and electroporation was performed using Lonza Nucleofector II. After electroporation, 500 μl warm growth media was added to the cuvette to dilute the electroporation buffer, the mixture was then carefully transferred to 2 ml pre-warmed growth media in 12-well plate. 5 unit/ml benzonase was supplemented to the cell culture to remove extracellular DNA.

For CRISPR-Cas9 transfection, the cells were washed twice in Cytoporation buffer T (BTX Harvard Apparatus), and 5 million cells were then resuspended in Cytoporation buffer T. This cellular suspension was mixed with 10 μg mRNA encoding Cas9 and 10 μg sgRNA targeting to TRAC locus (per million cells). Transfection was performed using Pulse Agile technology by applying two 0.1 mS pulses at 3,000 V/cm followed by four 0.2 mS pulses at 325 V/cm in 0.4 cm gap cuvettes (BTX Harvard Apparatus). The electroporated cells were then transferred to a 12-well plate containing 2 mL of prewarmed X-vivo-15 serum-free media and incubated at 37° C. for 15 min. The cells were then incubated at 37° C. for various length of time before the second transfection with dsDNA encoding the CD22CAR (SEQ ID NO. 220). After incubation, the CRISPR-Cas9 transfected cells were harvested, washed once with warm PBS. Five million cells were then pelleted and resuspended in 100 μl Lonza Human T cell buffer (Lonza, VPA-1002, 820 Human T cell buffer+18 μSupplement). 2 μg dsDNA repair template was mixed to the cells and electroporation was performed using Lonza Nucleofector II. After electroporation, 500 μl warm growth media was added to the cuvette to dilute the electroporation buffer, the mixture was then carefully transferred to 2 ml pre-warmed growth media in 12-well plate. 5 unit/ml Bezonase was supplemented to the cell culture to remove extracellular DNA.

For ssODN transfection allowing 20 bp insertion, the TALEN mRNA transfected cells were harvested, washed once with warm PBS. One million cells were then pelleted and resuspended in 20 μI Lonza P3 buffer (Lonza, V4SP-3096). 200nmol ssODN was then mixed to the cells for electroporation on Lonza 4D. After electroporation, 80 μl warm growth media was added to the cuvette to dilute the electroporation buffer, the mixture was then carefully transferred to 0.5 ml pre-wared growth media in 48-well plate.

ssODN Transfection to HSCs

CD34⁺ HSC were expanded for 5 days HSC expansion media before electroporation. 1E6 HSCs were harvested at 300 g for 10 min and washed one time in PBS. The cells were resuspended in BTXpress high performance buffer (Harvard Apparatus).

For co-transfection, 1E6 HSC were electroporated with 10 μg/arm of TALEN with ssODN1 or 2 (1000pmol) using BTX Pulse Agile (Harvard Apparatus). HSC expansion media was added to electroporated HSC and the cells were seeded in 24-well plate and incubated at 30° C. for 20 hrs. The cells were supplemented with additional HSC media and transferred to 37° C. For the 20 hr delay transfection, 10 μg/arm of TALEN, was first electroporated into 1E6 HSC, the cells were incubated at 30° C. for 20 hrs. Then a second electroporation with 1000pmol of either ssODN1 or ssODN2 was performed. The cells subject to the second transfection were then resuspended in HSC media and let recover at 37° C. overnight before supplemented with additional HSC media.

The electroporation were performed on BTX Pulse Agile (BTX Harvard Apparatus) with two pulses at 1000V and four pulses at 130V.

gDNA Extraction and qPCR

Cells were harvested and washed once with PBS. The cell pellets were then subject to gDNA extraction using Mag-Bind Blood & Tissue DNA HDQ kits (Omega Bio-Tek). For DSB detection, qPCR primers were designed to amplify the genomic sequence containing TALEN target sites, or away from the TALEN target sites as control using primers (SEQ ID NO. 223 to SEQ ID NO. 230). To determine exogenous dsDNA half-life, the qPCR primers (SEQ ID NO. 231 and SEQ ID NO. 232) were designed to specifically amplify the CAR sequence, which was the insertion template. The qPCR reaction was setup with PowerUp SYBR Green Master Mix (Thermo Fisher, A25742) analyzed on Bio-Rad CFX

### Western Blot and Flow Cytometry

To detect the expression of TALEN in western blot, an anti-RVD antibody and an anti-Rabbit secondary antibody (Cell Signally Technology) were used as described in Menger et al. [TALEN-Mediated Inactivation of PD-1 in Tumor-Reactive Lymphocytes Promotes Intratumoral T-cell Persistence and Rejection of Established Tumors Cancer. Res. (2016) 76(8):2087-2093]. The ECL (Thermo Scientific) signal was detected on Li—COR.

To detect CD22CAR expression on the surface of the edited T cells, a CD22Fc recombinant protein and an anti-Fcγ secondary antibody conjugated with PE fluorophore was used to stain the T cells. The cells were then analyzed on MacsQuant (Miltenyi Biotech) to detect PE positive cells.

### Deep-Sequencing Indel Analysis

PCR amplifications spanning TRAC or B2M targets were performed from gDNA harvested at the indicated time points post-transfection using primers (SEQ ID NO. 233 to SEQ ID NO. 236). Purified PCR products were sequenced using the Illumina method (Miseq 2×250 nano V2). At least 150,000 sequences were obtained per PCR product for Illumina, and sequences were analyzed for the presence of site-specific mutations.

### Example 2. Understanding TALEN-Induced Double Strand Break Kinetics

TALEN are TALE-nucleases designed by Cellectis (8, rue de la Croix Jarry, 75013 PARIS) using Fok1 nuclease catalytic domains. It is a widely used engineered nuclease format for precise and specific genome editing in many fields, in particular to genetically engineer “off-the-shelf” CAR-T cells. However, little is known about the kinetics of the TALE-nuclease induced double-strand break (DSB) generation and the DSB repair process. Here, we measured the kinetics of DSB generation and repair for single loci in human T cells and observed the maximum abundance of un-joined DSB at 20 hrs after TALEN mRNA was transfected to the cells. With the understanding of TALEN-induced DSB kinetics, we designed a two-step transfection procedure that greatly improved targeted integration rate using dsDNA or ssODN as repair DNA template.

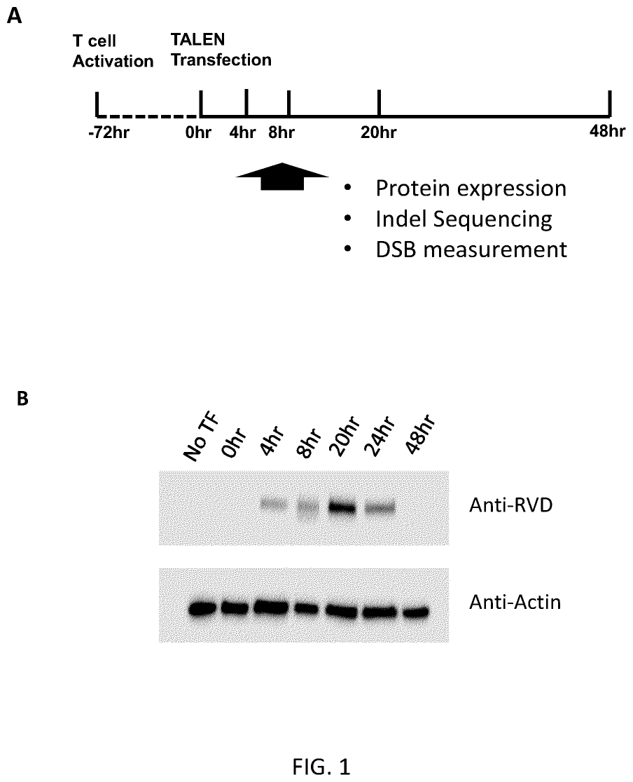

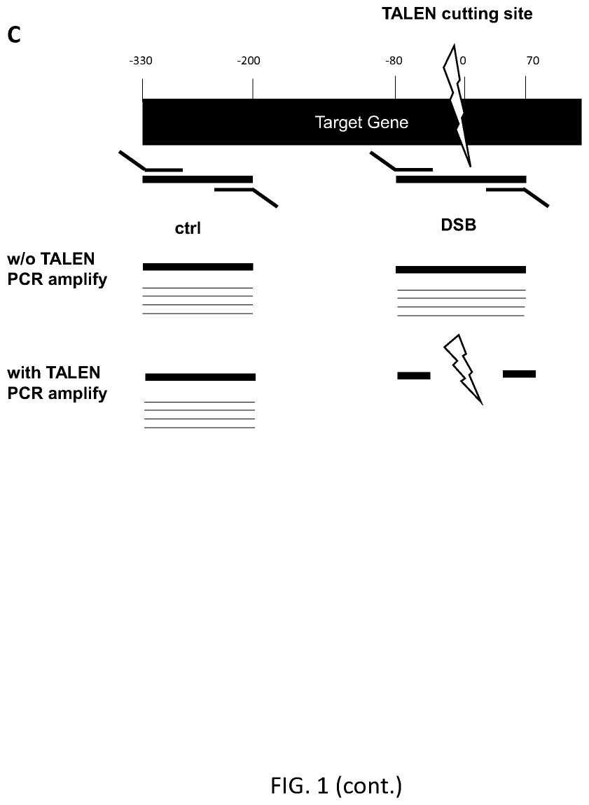

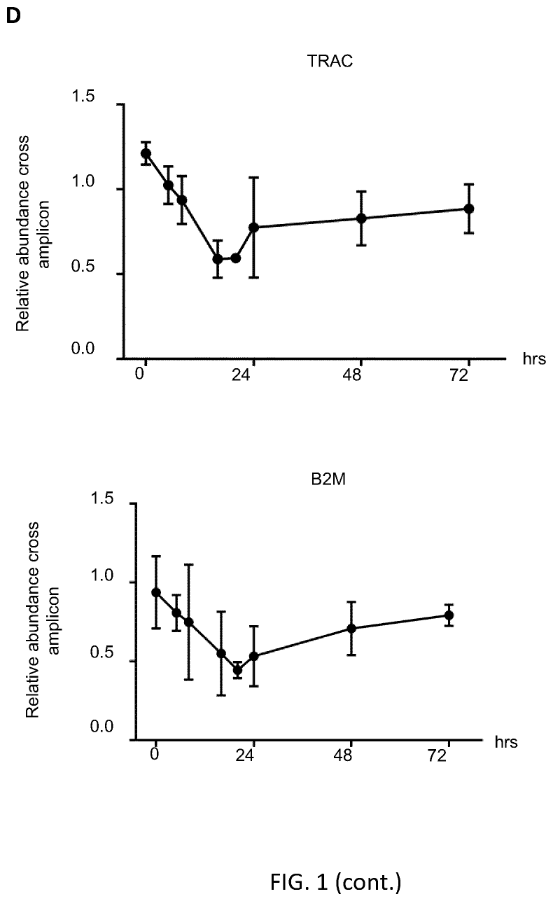

TALEN mRNA from example 1 were transfected into activated human T cells to perform gene editing to understand the timing of the events that happen after TALEN mRNA transfection. The TALEN transfected cells were collected at different time points indicated in FIG. 1A for different analysis: TALEN protein expression, cleavage of genomic DNA and the repair of TALEN induced double-strand break (DSB).

### TALEN Protein Expression

In order to understand and characterize the different steps of the gene editing process TALEN protein expression was first measured following mRNA transfection. The cells were harvested at different time points after TRAC TALEN mRNA transfection for total cell lysate extraction. The lysates were then resolved by SDS-PAGE and an anti-RVD antibody, which recognize specifically the DNA binding domain of TALEN, was used to detect the specific expression of the TALEN protein by western blotting (FIG. 1B). The result showed that the TALEN protein was detectable by immunoblotting at 4 hrs after TALEN mRNA transfection. The amount of TALEN protein continued to accumulate until 20 hrs post-transfection. At 24 hrs post-transfection, the TALEN protein quantity reduced and the protein level fell below detectable level at 48 hrs, possibly due to the combination degradation of the mRNA template and TALEN protein itself.

### Cleavage Kinetics

In such TALEN mediated T-cell editing system where the TALEN enter the cells as mRNA, the TALEN protein took time to accumulate to the maximum amount and started to disappear only after 20 hrs. As the creation of double-strand break (DSB) is related to the nuclease activity, it was thus hypothesized that the accumulation and degradation of TALEN protein will affect the kinetic of DSB generation. Therefore, the kinetics of un joined DSB creation by TALEN were investigated. A pair of primers was designed that would amplify +/−100 bp across the TALEN cutting site and another pair of primers that amplified around −300 to −200 bp upstream of the TALEN cutting site (FIG. 1C). With this design, it was hypothesized that as the TALEN generates DSB at its target site, the abundance of intact DNA across the TALEN cutting site would decrease, whereas, the DNA stretch upstream of the cutting site would remain largely unchanged. By comparing the relative abundance of “cross” amplicon vs the “upstream” amplicon, the change in intact “cross” amplicon, and inversely the increase of un-joined end of TALEN-cut DNA could be determined.

The genomic DNA (gDNA) from cells treated with a TALEN targeting either the TRAC or the B2M loci was extracted at various timepoints after transfection and subject qPCR analysis. Our data shows that transfection with either of the two TALEN, led to a decrease in the abundance of “cross” amplicon to a lowest at around 20 hrs, indicating that at this timepoint the largest portion of cells would have an un-joined DSB DNA ends at their TALEN target site and ready for the repair (FIG. 1D).

### DSB Repair Kinetics

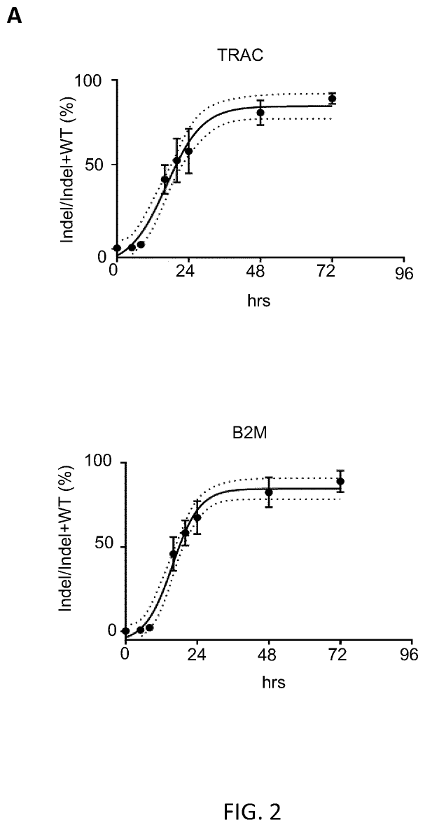

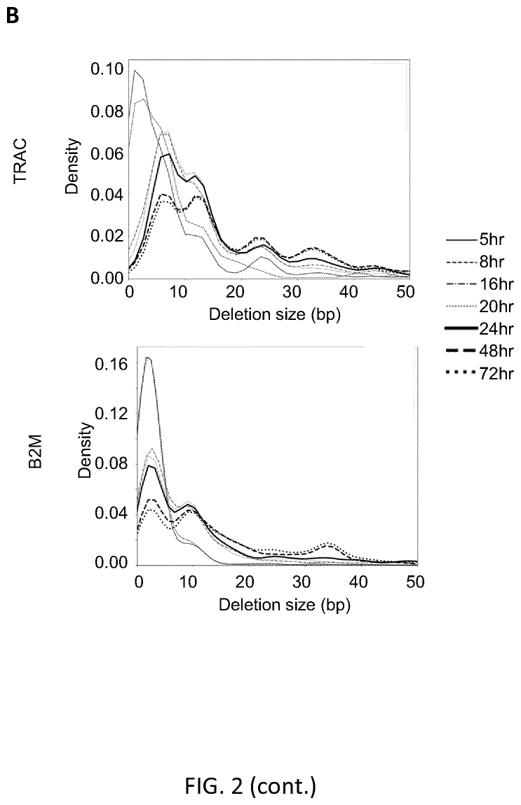

To further characterize the NHEJ repair kinetics to the TALEN induced DSB, targeted deep-sequencing method was used to determine the rate of indel accumulation. The cells transfected with either the TRAC or B2M TALEN were harvested at various time points, up to 72 hrs after TALEN mRNA electroporation. After gDNA extraction, a region of ˜300-bp around the TALEN target sites was amplified by PCR and the resulting products were subjected to high-throughput sequencing to determine the intact and Indel fractions. The results show (FIG. 2A), a gradual accumulation of indels over time, indicating that DSBs were introduced and eventually repaired with mutations. Toward the end of the time course, the indel frequency reached a plateau of around 90% for both TALEN.

The sigmoid appearance of the measured indel time curves suggested a delayed onset of indel accumulation, which is related to the timing of TALEN protein expression being low at the early time points. These curves also suggest that the indel accumulation rate was the highest from 10 hr to 20 hr post TALEN mRNA transfection. This phenomenon might be related to higher amount of TALEN protein being present in the cells, which translates into higher nuclease activity.

### DSB Signature Over Time

Further, the change in deletion sizes over time within the Indel pattern was examined. Our sequencing data depicted that at earlier time points (<8 hrs) the majority species were small deletions (<5 bp), whereas at later time points, the abundance of small deletion decreased and the larger deletion started to appear (FIG. 2B). This shift towards the bigger deletion is because TALEN can tolerate and “re-cut” the smaller (<5 bp) deletion at the spacer region. Thus, the small deletions created at early time points were likely re-cut afterwards, which produced larger deletions. After the deletion size became large enough to strongly affect the TALEN activity or even disrupt TALEN binding sequence the re-cutting was be fully prevented. Indeed, the accumulation of larger deletions at later time points was observed. In addition, the quantity of TALEN protein was not detectable after 48 hrs of transfection, as previously shown in FIG. 1B, suggesting the minimal TALEN nuclease activity after 48 hrs. As expected, the data, showed no significant difference of deletion size observed between 48 hr and 72 hr.

Taken together the data suggest that the DSB was generated, repaired by the NHEJ pathway leading to small deletion event that can still be cut by the TALEN protein still present in the cell. Once the DSB is repaired by NHEJ, leading to a large deletion event, the repaired sequence could no longer be recut by TALEN.

### Example 3: Optimizing Targeted Integration in T Cells

ssODN Mediated Gene Insertion

Integration of exogenous DNA molecule within the cellular genome requires the cells to use the less efficient HDR pathway for DSB repair instead of NHEJ. With the aim to perform gene insertion, TALEN were used a to create a DSB at desired locus and to precisely integrate the gene of interest at this locus. The knowledge on the TALEN behavior and DSB repair kinetics was used to improve target gene insertion.

Short single strand DNA (ssODN) were first used as repair DNA donor template to direct target insertion. The introduction of short single-stranded oligodeoxynucleotide (ssODN) HDR templates does not cause significant T cell toxicity.

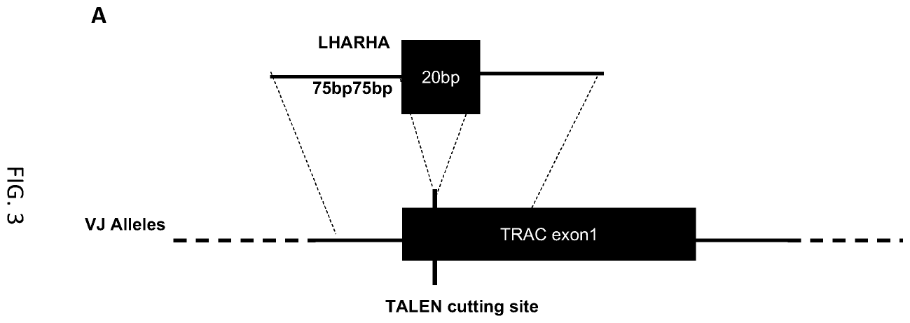

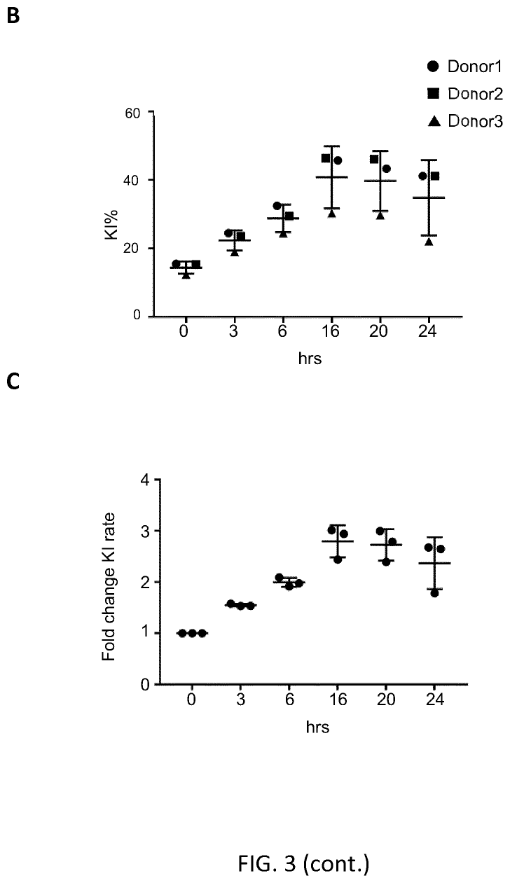

In order to insert, in edited cells, a specific 20 bp sequence at the TRAC locus (FIG. 3A), 170 bp ssODN was designed containing 70 bp homology arms to TRAC locus on each of 5- and 3-prime ends. At the center of the ssODN, in the spacer sequence between the two half TALEN binding sequences, was inserted a 20 bp scramble sequence.

ssODN has been shown to have a half-life of 1.5 hrs after electroporation to the cells. The rapid degradation of ssODN in-cell would mean that the time window for its effective direction of homologous recombination, is relatively narrow. Our hypothesis is that the best timing to deliver the DNA repair/donor template would be around the time when most of the cells have a TALEN target site cut “open”. To test this hypothesis, ssODN were electroporated into to the cells at different time points (0, 3, 6, 16, 20, or 24 hrs) after TRAC TALEN mRNA transfection.

The cells were harvested five days after transfection for genomic DNA extraction. The TRAC locus sequence was amplified and subjected to deep-sequencing analysis to detect the 20 bp insertion efficiency. The result showed that the 20 bp exogenous sequence knock-in rate increased as the ssODN transfection timing was delayed. Maximum integration of the 20 bp exogenous sequence was observed at TRAC TALEN edited locus (FIG. 3 B and C) when transfected the mRNA with a 16-20 hr delay, with KI rates varying from 30% to 46% at 16 hrs time point (FIG. 3B). Whereas the when ssODN template was transfected immediately after TALEN transfection, the insertion rate was only around 10-15%. The fold increase of KI rate was significantly higher at 16 hrs, around 3-fold of that at 0 hr (FIG. 3C). This observation confirmed the hypothesis that delivering ssODN template at 16 hrs, when most of the cells have un-joined TALEN cut site, resulted in the highest target insertion rate. This result also suggested that the timing of repair template delivery will be important to achieve high frequencies of KI in TALEN mediated gene insertion experiments.

### Large Knock-In Optimization

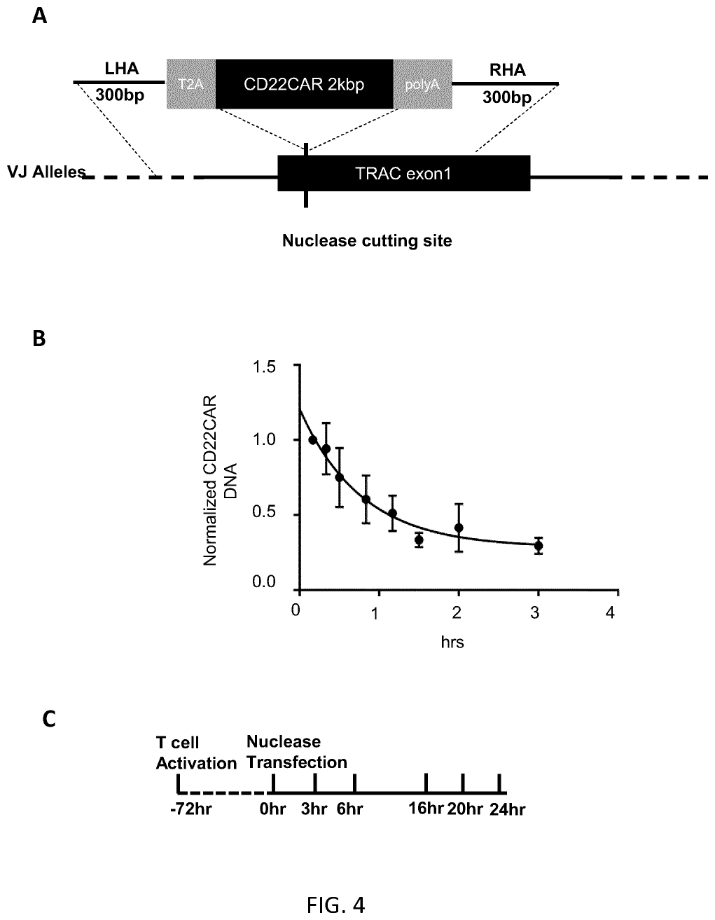

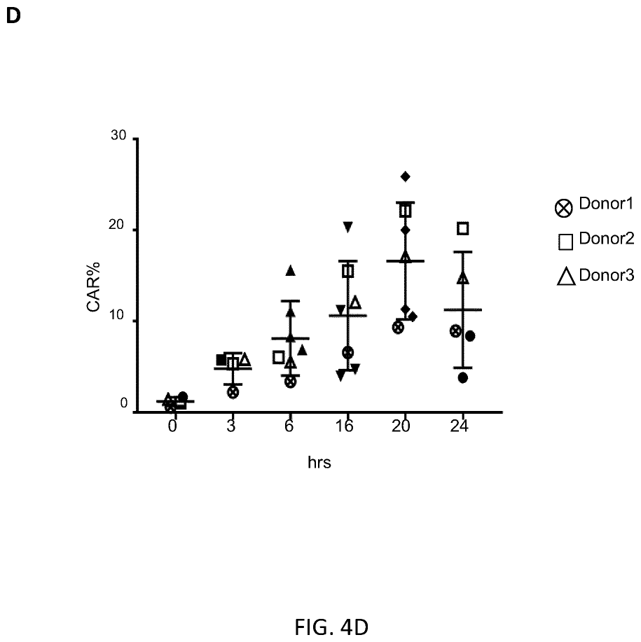

Inserting large DNA template to introduce a functional gene to a specific genomic locus was investigated. Compared to the random gene insertion (via retrovirus), targeted gene insertion can avoid clonal expansion, oncogenic transformation, variegated transgene expression and transcriptional silencing. Unlike short single-stranded oligodeoxynucleotide (ssODN), large linear double stranded (dsDNA) HDR templates has been toxic to primary cells at high concentrations. Balancing the toxicity caused by high concentration of dsDNA and the insertion efficiency is one of the major challenges to gene insertion. A DNA repair template was designed to integrate an anti-CD22 CAR expression cassette at the TRAC locus, using an T2A self-cleaving element and keeping the open reading frame of the TCRalpha gene, in order to place CAR expression under TCRalpha regulation. (FIG. 4A). The dsDNA repair template, obtained by PCR in example 1, has a total size of 2.5 kb.

Half-life of dsDNA template in transfected T cells was first evaluated. PCR product was delivered into TRAC TALEN treated T-cells by electroporation. The amount of PCR product in the cells at various time points after electroporation was determined by qPCR. Results shows in FIG. 4B that the half-life the linear dsDNA (PCR product) has a short half-life of less than an hour (T½=54 mins).

As demonstrated previously, at 20 hr after TALEN transfection, highest portion of the cells have an un-joined DSB at the TALEN cutting site. It was therefore hypothesized that transfecting dsDNA at 20 hrs after TALEN mRNA transfection would produce the highest ratio of target integration.

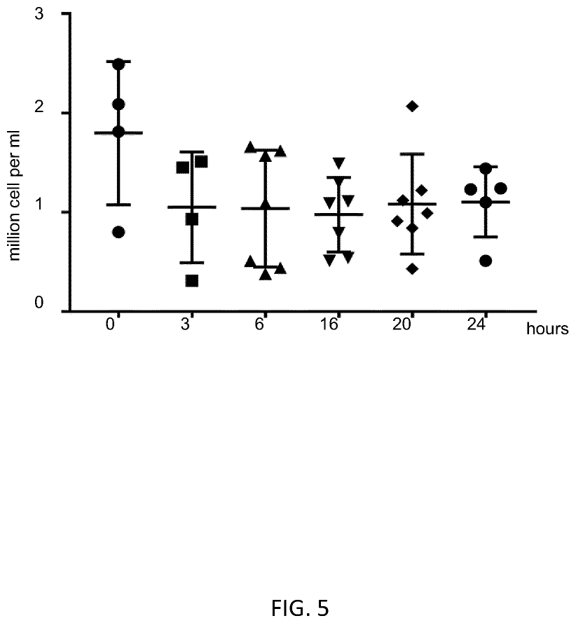

dsDNA template encoding CD22CAR was transfected either together with TRAC TALEN mRNA or at different time points after TALEN mRNA transfection (FIG. 4C). The cells were then cultured for five days before flow cytometry analysis for the CD22CAR expression at cell surface. The result demonstrated that dsDNA transfection carried out at 20 hrs after TALEN mRNA transfection produced highest CD22CAR integration rate (FIG. 4D). Importantly, this transfection procedure did not cause increased toxicity to the edited T cells (FIG. 5).

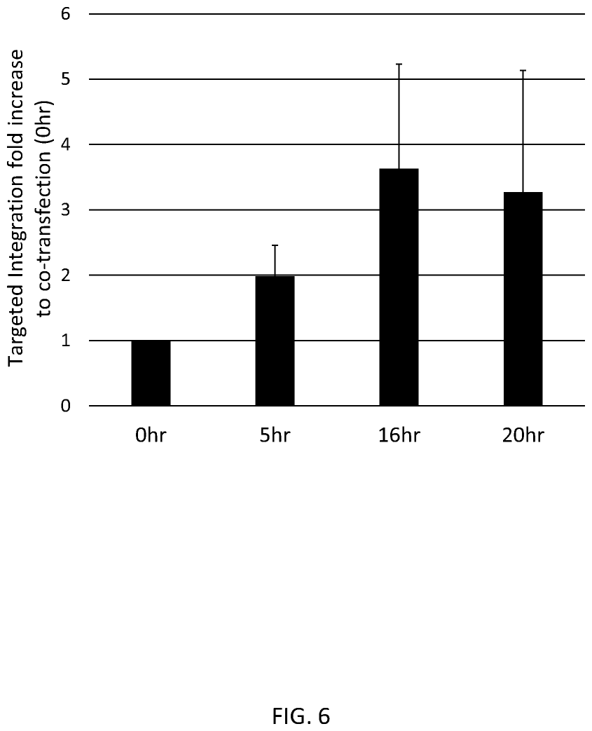

CRISPR-Cas9 mediated targeted integration was also evaluated. dsDNA template encoding CD22CAR was transfected either at 0 hr (cells seeded after CRISPR-Cas9 electroporated were immediately harvested and transfected a second time with dsDNA or at the different indicated time points after Cas9 mRNA and sgRNA transfection. The cells were then cultured for five days before flow cytometry analysis for the CD22CAR expression at cell surface. Our result demonstrated that dsDNA transfection carried out at 16 hrs after Cas9 mRNA and gRNA transfection produced highest CD22CAR integration rate (FIG. 6).

### Example 4: Optimizing Targeted Integration in HSCs

ssODN mediated Knock-In is particularly attractive because it can be used to introduce single base-pair substitution into the genome. Since point mutations are the largest class of known pathogenic genetic variants, a major application ssODN mediated single base-pair mutation is the study or treatment of disease-associated point mutations.

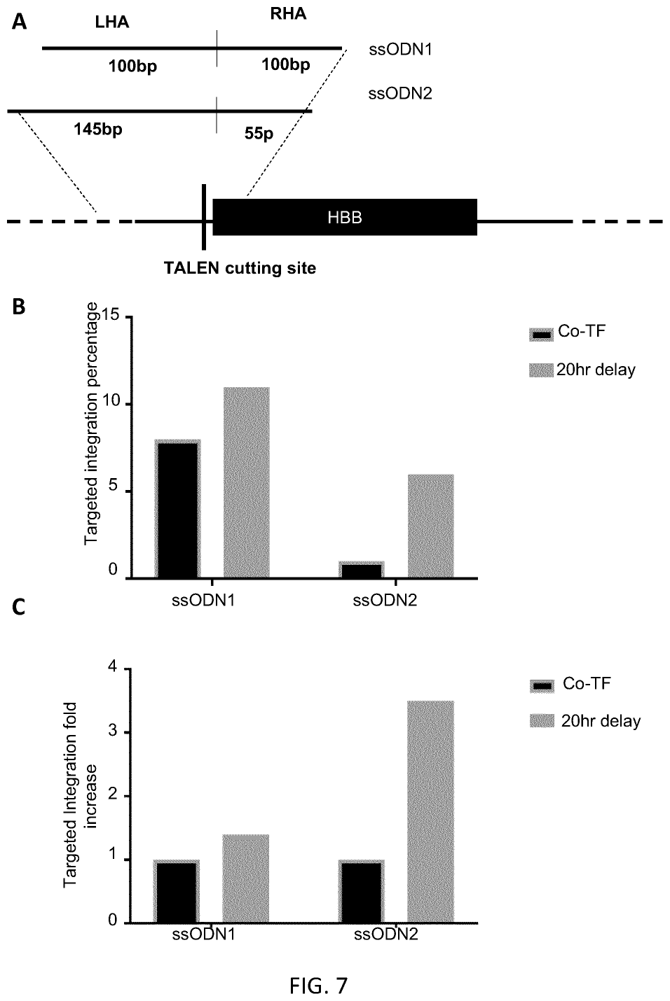

ssODN were used to introduce a point mutation using TALEN in HSC. The mutation is designed to introduce the sickling mutation at the Hemoglobin subunit beta (HBB) gene. In the experiment, was compared the mutation induction efficiency using two different ssODN ssODN1 and ssODN2 (SEQ ID NO. 237 and SEQ ID NO. 238) respectively, see FIG. 7A). In addition, was compared the mutation induction efficiency when the ssODN were transfected to the HSCs at different time points. With the co-transfection (co-TF) condition, the ssODN was mixed with HBB TALEN mRNA and electroporated to the HSCs at the same time. Whereas in the 20 hr delay condition, the TALEN mRNA was first electroporated into the HSCs, followed by a second transfection that delivered the ssODN 20 hrs after the mRNA transfection. The cells were then harvested to assess the percentages of genome presenting the desired point mutation. The result showed that with the 20 hr delay of ssODN1 transfection, it was possible to introduce point mutation up to 10% of the alleles (FIG. 7B). Importantly, a delayed delivery of the ssODN1 or ssODN2 increased 30% or more than 3 fold of point mutation rates respectively (FIG. 7C).

This result confirms our previous observations that a delayed delivery of the DNA repair template improved its targeted integration rate. Introducing point mutation using this method holds great potential in correcting many diseases that are caused by point mutations.

TABLE 7
polynucleotide and polypeptide sequences used in the examples.

| SEQ ID | Sequence | |
|---|---|---|
| NO. # | designation | Nucleic or amino acid sequences |
| #218 | TRAC TALEN target | TTCCTCCTACTCACCATcagcctcctggttatGGTACAGGTAAGA |
| | | GCAA |
| #219 | B2M TALEN target | TCCGTGGCCTTAGCTGTgctcgcgctactcTCTCTTTCTGGCCTG |
| | | GA |
| #220 | CD22CAR | GGTTTTCCCAGTCACGACGTTGTAAAACGACGGCCAGTG |
| | repair template | AATTCGAGCTCGGTACCTCGCGAATGCATCTAGATGCGG |
| | | CCGCAAGTAGCCCTGCATTTCAGGTTTCCTTGAGTGGCA |
| | | GGCCAGGCCTGGCCGTGAACGTTCACTGAAATCATGGCC |
| | | TCTTGGCCAAGATTGATAGCTTGTGCCTGTCCCTGAGTCC |
| | | CAGTCCATCACGAGCAGCTGGTTTCTAAGATGCTATTTC |
| | | CCGTATAAAGCATGAGACCGTGACTTGCCAGCCCCACAG |
| | | AGCCCCGCCCTTGTCCATCACTGGCATCTGGACTCCAGC |
| | | CTGGGTTGGGGCAAAGAGGGAAATGAGATCATGTCCTA |
| | | ACCCTGATCCTCTTGTCCCACAGATATCCAGTCCGGTGA |
| | | GGGCAGAGGAAGTCTTCTAACATGCGGTGACGTGGAGG |
| | | AGAATCCGGGCCCCGGATCCGCTCTGCCCGTCACCGCTC |
| | | TGCTGCTGCCACTGGCACTGCTGCTGCACGCTGCTAGGC |
| | | CCCAGGTGCAGCTGCAGCAGAGCGGCCCTGGCCTGGTGA |
| | | AGCCAAGCCAGACACTGTCCCTGACCTGCGCCATCAGCG |
| | | GCGATTCCGTGAGCTCCAACTCCGCCGCCTGGAATTGGA |
| | | TCAGGCAGTCCCCTTCTCGGGGCCTGGAGTGGCTGGGAA |
| | | GGACATACTATCGGTCTAAGTGGTACAACGATTATGCCG |
| | | TGTCTGTGAAGAGCAGAATCACAATCAACCCTGACACCT |
| | | CCAAGAATCAGTTCTCTCTGCAGCTGAATAGCGTGACAC |
| | | CAGAGGACACCGCCGTGTACTATTGCGCCAGGGAGGTG |
| | | ACCGGCGACCTGGAGGATGCCTTTGACATCTGGGGCCAG |
| | | GGCACAATGGTGACCGTGTCTAGCGGAGGCGGAGGCTC |
| | | CGGAGGCGGAGGATCTGGCGGAGGCGGAAGCGATATCC |
| | | AGATGACACAGTCCCCATCCTCTCTGAGCGCCTCCGTGG |
| | | GCGACAGAGTGACAATCACCTGTAGGGCCTCCCAGACCA |
| | | TCTGGTCTTACCTGAACTGGTATCAGCAGAGGCCCGGCA |
| | | AGGCCCCTAATCTGCTGATCTACGCAGCAAGCTCCCTGC |
| | | AGAGCGGAGTGCCATCCAGATTCTCTGGCAGGGGCTCCG |
| | | GCACAGACTTCACCCTGACCATCTCTAGCCTCCAGGCCG |
| | | AGGACTTCGCCACCTACTATTGCCAGCAGTCTTATAGCA |
| | | TCCCCCAGACATTTGGCCAGGGCACCAAGCTGGAGATCA |
| | | AGGCTCCCACCACAACCCCCGCTCCAAGGCCCCCTACCC |
| | | CCGCACCAACTATTGCCTCCCAGCCACTCTCACTGCGGC |
| | | CTGAGGCCTGTCGGCCCGCTGCTGGAGGCGCAGTGCATA |
| | | CAAGGGGCCTCGATTTCGCCTGCGATATTTACATCTGGG |
| | | CACCCCTCGCCGGCACCTGCGGGGTGCTTCTCCTCTCCCT |
| | | GGTGATTACCCTGTATTGCAGACGGGGCCGGAAGAAGCT |
| | | CCTCTACATTTTTAAGCAGCCTTTCATGCGGCCAGTGCAG |
| | | ACAACCCAAGAGGAGGATGGGTGTTCCTGCAGATTCCCT |
| | | GAGGAAGAGGAAGGCGGGTGCGAGCTGAGAGTGAAGTT |
| | | CTCCAGGAGCGCAGATGCCCCCGCCTATCAACAGGGCCA |
| | | GAACCAGCTCTACAACGAGCTTAACCTCGGGAGGCGCG |
| | | AAGAATACGACGTGTTGGATAAGAGAAGGGGGCGGGAC |
| | | CCCGAGATGGGAGGAAAGCCCCGGAGGAAGAACCCTCA |
| | | GGAGGGCCTGTACAACGAGCTGCAGAAGGATAAGATGG |
| | | CCGAGGCCTACTCAGAGATCGGGATGAAGGGGGAGCGG |
| | | CGCCGCGGGAAGGGGCACGATGGGCTCTACCAGGGGCT |
| | | GAGCACAGCCACAAAGGACACATACGACGCCTTGCACA |
| | | TGCAGGCCCTTCCACCCCGGTGAAGATACATTGATGAGT |
| | | TTGGACAAACCACAACTAGAATGCAGTGAAAAAAATGC |
| | | TTTATTTGTGAAATTTGTGATGCTATTGCTTTATTTGTAA |
| | | CCATTATAAGCTGCAATAAACAAGTTAACAACAACAATT |
| | | GCATTCATTTTATGTTTCAGGTTCAGGGGGAGGTGTGGG |
| | | AGGTTTTTTAAAGCAAGTAAAACCTCTACAAATGTGGTA |
| | | CGGAATTCAGTCAATATGTTCACCGTGTACCAGCTGAGA |
| | | GACTCTAAATCCAGTGACAAGTCTGTCTGCCTATTCACC |
| | | GATTTTGATTCTCAAACAAATGTGTCACAAAGTAAGGAT |
| | | TCTGATGTGTATATCACAGACAAAACTGTGCTAGACATG |
| | | AGGTCTATGGACTTCAAGAGCAACAGTGCTGTGGCCTGG |
| | | AGCAACAAATCTGACTTTGCATGTGCAAACGCCTTCAAC |
| | | AACAGCATTATTCCAGAAGACACCTTCTTCCCCAGCCCA |
| | | GGTAAGGGCAGCTTTGGTGCCTTCGCAGGCTGTTTCCTT |
| | | GCTTCAGGAACTCGAGTATCGGATCCCGGGCCCGTCGAC |
| | | TGCAGAGGCCTGCATGCAAGCTTGGCGTAATCATGGTCA |
| | | TAGCTGTTTCCTGTGTGAAATTGTT |
| #221 | M13F | CCCAGTCACGACGTTGTAAAACG |
| #222 | M13R | CCTGTGTGAAATTGTTATCCGCT |
| #223 | B2M cross_L | CATTCCTGAAGCTGACAGCATTCGGG |
| #224 | B2M cross_R | GGGTAGGAGAGACTCACGCTGGATAG |
| #225 | B2M up_L | CGTGACTTCCCTTCTCCAAGTTCTCC |
| #226 | B2M up_R | ACGCTTATCGACGCCCTAAACTTTGT |
| #227 | TRAC cross_L | GCATTTCAGGTTTCCTTGAGTGGCAG |
| #228 | TRAC cross_R | TGGCAAGTCACGGTCTCATGCTTTAT |
| #229 | TRAC up_L | CTTGTCCATCACTGGCATCTGGACTC |
| #230 | TRAC up_R | ATCGGTGAATAGGCAGACAGACTTGT |
| #231 | CD22CAR_F | AAGATGTACAGTTTGCTTTGCTGGGC |
| #232 | CD22CAR_R | ACGTCACCGCATGTTAGAAGACTTCC |
| #233 | B2M_NGS_L | CTACACGACGCTCTTCCGATCTGTCCCTCTCTCTAACCTG |
| | | GC |
| #234 | B2M_NGS_R | GTGACTGGAGTTCAGACGTGTGCTCTTCCGATCTGAAGG |
| | | GAAGTCACGGAGCGA |
| #235 | TRAC_NGS_L | GTCGACTAGGGATAACAGGGTAATTATCCAGAACCCTGA |
| | | CCCTGCCGTGTACCA |
| #236 | TRAC_NGS_R | AAACTGTATTATAAGTAAATGCATTGGATTTAGAGTCTC |
| | | TCAGCTGGTACACGG |
| #237 | HSC ssODNl | A*C*TTCATCCACGTTCACCTTGCCCCACAGGGCAGTAACGGCAGACT |
| | | TCTCCTCcctAGGAGTCAGATGCACCATGGTGTCGGCTTGAGGTTGAC |
| | | AGTGAACACAGTTGTGTCAGAAGCAAATGTAAGCAATAGATGGCTCT |
| | | GCCCTGACTTTTATGCCCAGCCCTGGCTCCTGCCCTCCCTGCTCCTGGG |
| | | AGTAGATTGGC*C*A |
| #238 | HSC ssODN2 | G*A*TACCAACCTGCCCAGGGCCTCACCACCAACTTCATCCACGTTCAC |
| | | CTTGCCCCACAGGGCAGTAACGGCAGACTTCTCCTCcctAGGAGTCAG |
| | | ATGCACCATGGTGTCGGCTTGAGGTTGACAGTGAACACAGTTGTGTC |
| | | AGAAGCAAATGTAAGCAATAGATGGCTCTGCCCTGACTTTTATGCCCA |
| | | GCCCTGGCTCC*T*G |
| #239 | HBB Target | TTGCTTACATTTGCTTCTgacacaactgtgttcACTAGCAACCTCAAACA |
| #240 | TRAC ssODN | CCTGGGTTGGGGCAAAGAGGGAAATGAGATCATGTCCTAACCCTGAT |
| | | CCTCTTGTCCCACAGATATCCAGAACCCTAGGTGAAAGCTTAGACTAG |
| | | TGACCCTGCCGTGTACCAGCTGAGAGACTCTAAATCCAGTGACAAGTC |
| | | TGTCTGCCTATTCACCGATTTTGATTC |
| #241 | HBB TALEN LEFT | ATGGGCGATCCTAAAAAGAAACGTAAGGTCATCGATATCGCCGATCT |
| | | ACGCACGCTCGGCTACAGCCAGCAGCAACAGGAGAAGATCAAACCGA |
| | | AGGTTCGTTCGACAGTGGCGCAGCACCACGAGGCACTGGTCGGCCAC |
| | | GGGTTTACACACGCGCACATCGTTGCGTTAAGCCAACACCCGGCAGC |
| | | GTTAGGGACCGTCGCTGTCAAGTATCAGGACATGATCGCAGCGTTGC |
| | | CAGAGGCGACACACGAAGCGATCGTTGGCGTCGGCAAACAGTGGTCC |
| | | GGCGCACGCGCTCTGGAGGCCTTGCTCACGGTGGCGGGAGAGTTGA |
| | | GAGGTCCACCGTTACAGTTGGACACAGGCCAACTTCTCAAGATTGCAA |
| | | AACGTGGCGGCGTGACCGCAGTGGAGGCAGTGCATGCATGGCGCAA |
| | | TGCACTGACGGGTGCCCCGCTCAACTTGACCCCCCAGCAGGTGGTGG |
| | | CCATCGCCAGCAATAATGGTGGCAAGCAGGCGCTGGAGACGGTCCAG |
| | | CGGCTGTTGCCGGTGCTGTGCCAGGCCCACGGCTTGACCCCGGAGCA |
| | | GGTGGTGGCCATCGCCAGCCACGATGGCGGCAAGCAGGCGCTGGAG |
| | | ACGGTCCAGCGGCTGTTGCCGGTGCTGTGCCAGGCCCACGGCTTGAC |
| | | CCCCCAGCAGGTGGTGGCCATCGCCAGCAATGGCGGTGGCAAGCAG |
| | | GCGCTGGAGACGGTCCAGCGGCTGTTGCCGGTGCTGTGCCAGGCCCA |
| | | CGGCTTGACCCCCCAGCAGGTGGTGGCCATCGCCAGCAATGGCGGTG |
| | | GCAAGCAGGCGCTGGAGACGGTCCAGCGGCTGTTGCCGGTGCTGTGC |
| | | CAGGCCCACGGCTTGACCCCGGAGCAGGTGGTGGCCATCGCCAGCAA |
| | | TATTGGTGGCAAGCAGGCGCTGGAGACGGTGCAGGCGCTGTTGCCG |
| | | GTGCTGTGCCAGGCCCACGGCTTGACCCCGGAGCAGGTGGTGGCCAT |
| | | CGCCAGCCACGATGGCGGCAAGCAGGCGCTGGAGACGGTCCAGCGG |
| | | CTGTTGCCGGTGCTGTGCCAGGCCCACGGCTTGACCCCGGAGCAGGT |
| | | GGTGGCCATCGCCAGCAATATTGGTGGCAAGCAGGCGCTGGAGACG |
| | | GTGCAGGCGCTGTTGCCGGTGCTGTGCCAGGCCCACGGCTTGACCCC |
| | | CCAGCAGGTGGTGGCCATCGCCAGCAATGGCGGTGGCAAGCAGGCG |
| | | CTGGAGACGGTCCAGCGGCTGTTGCCGGTGCTGTGCCAGGCCCACGG |
| | | CTTGACCCCCCAGCAGGTGGTGGCCATCGCCAGCAATGGCGGTGGCA |
| | | AGCAGGCGCTGGAGACGGTCCAGCGGCTGTTGCCGGTGCTGTGCCAG |
| | | GCCCACGGCTTGACCCCCCAGCAGGTGGTGGCCATCGCCAGCAATGG |
| | | CGGTGGCAAGCAGGCGCTGGAGACGGTCCAGCGGCTGTTGCCGGTG |
| | | CTGTGCCAGGCCCACGGCTTGACCCCCCAGCAGGTGGTGGCCATCGC |
| | | CAGCAATAATGGTGGCAAGCAGGCGCTGGAGACGGTCCAGCGGCTG |
| | | TTGCCGGTGCTGTGCCAGGCCCACGGCTTGACCCCGGAGCAGGTGGT |
| | | GGCCATCGCCAGCCACGATGGCGGCAAGCAGGCGCTGGAGACGGTC |
| | | CAGCGGCTGTTGCCGGTGCTGTGCCAGGCCCACGGCTTGACCCCCCA |
| | | GCAGGTGGTGGCCATCGCCAGCAATGGCGGTGGCAAGCAGGCGCTG |
| | | GAGACGGTCCAGCGGCTGTTGCCGGTGCTGTGCCAGGCCCACGGCTT |
| | | GACCCCCCAGCAGGTGGTGGCCATCGCCAGCAATGGCGGTGGCAAGC |
| | | AGGCGCTGGAGACGGTCCAGCGGCTGTTGCCGGTGCTGTGCCAGGCC |
| | | CACGGCTTGACCCCGGAGCAGGTGGTGGCCATCGCCAGCCACGATGG |
| | | CGGCAAGCAGGCGCTGGAGACGGTCCAGCGGCTGTTGCCGGTGCTGT |
| | | GCCAGGCCCACGGCTTGACCCCTCAGCAGGTGGTGGCCATCGCCAGC |
| | | AATGGCGGCGGCAGGCCGGCGCTGGAGAGCATTGTTGCCCAGTTATC |
| | | TCGCCCTGATCCGGCGTTGGCCGCGTTGACCAACGACCACCTCGTCGC |
| | | CTTGGCCTGCCTCGGCGGGCGTCCTGCGCTGGATGCAGTGAAAAAGG |
| | | GATTGGGGGATCCTATCAGCCGTTCCCAGCTGGTGAAGTCCGAGCTG |
| | | GAGGAGAAGAAATCCGAGTTGAGGCACAAGCTGAAGTACGTGCCCC |
| | | ACGAGTACATCGAGCTGATCGAGATCGCCCGGAACAGCACCCAGGAC |
| | | CGTATCCTGGAGATGAAGGTGATGGAGTTCTTCATGAAGGTGTACGG |
| | | CTACAGGGGCAAGCACCTGGGCGGCTCCAGGAAGCCCGACGGCGCC |
| | | ATCTACACCGTGGGCTCCCCCATCGACTACGGCGTGATCGTGGACACC |
| | | AAGGCCTACTCCGGCGGCTACAACCTGCCCATCGGCCAGGCCGACGA |
| | | AATGCAGAGGTACGTGGAGGAGAACCAGACCAGGAACAAGCACATC |
| | | AACCCCAACGAGTGGTGGAAGGTGTACCCCTCCAGCGTGACCGAGTT |
| | | CAAGTTCCTGTTCGTGTCCGGCCACTTCAAGGGCAACTACAAGGCCCA |
| | | GCTGACCAGGCTGAACCACATCACCAACTGCAACGGCGCCGTGCTGT |
| | | CCGTGGAGGAGCTCCTGATCGGCGGCGAGATGATCAAGGCCGGCAC |
| | | CCTGACCCTGGAGGAGGTGAGGAGGAAGTTCAACAACGGCGAGATC |
| | | AACTTCGCGGCCGACTGATAA |
| #242 | HBB TALEN LEFT | MGDPKKKRKVIDIADLRTLGYSQQQQEKIKPKVRSTVAQHHEALVGHGF |
| | PRT | THAHIVALSQHPAALGTVAVKYQDMIAALPEATHEAIVGVGKQWSGAR |
| | | ALEALLTVAGELRGPPLQLDTGQLLKIAKRGGVTAVEAVHAWRNALTGA |
| | | PLNLTPQQVVAIASNNGGKQALETVQRLLPVLCQAHGLTPEQVVAIASH |
| | | DGGKQALETVQRLLPVLCQAHGLTPQQVVAIASNGGGKQALETVQRLLP |
| | | VLCQAHGLTPQQVVAIASNGGGKQALETVQRLLPVLCQAHGLTPEQVV |
| | | AIASNIGGKQALETVQALLPVLCQAHGLTPEQVVAIASHDGGKQALETVQ |
| | | RLLPVLCQAHGLTPEQVVAIASNIGGKQALETVQALLPVLCQAHGLTPQQ |
| | | WAIASNGGGKQALETVQRLLPVLCQAHGLTPQQVVAIASNGGGKQALE |
| | | TVQRLLPVLCQAHGLTPQQVVAIASNGGGKQALETVQRLLPVLCQAHGL |
| | | TPQQWAIASNNGGKQALETVQRLLPVLCQAHGLTPEQVVAIASHDGGK |
| | | QALETVQRLLPVLCQAHGLTPQQVVAIASNGGGKQALETVQRLLPVLCQ |
| | | AHGLTPQQVVAIASNGGGKQALETVQRLLPVLCQAHGLTPEQVVAIASH |
| | | DGGKQALETVQRLLPVLCQAHGLTPQQVVAIASNGGGRPALESIVAQLS |
| | | RPDPALAALTNDHLVALACLGGRPALDAVKKGLGDPISRSQLVKSELEEKK |
| | | SELRHKLKYVPHEYIELIEIARNSTQDRILEMKVMEFFMKVYGYRGKHLGG |
| | | SRKPDGAIYTVGSPIDYGVIVDTKAYSGGYNLPIGQADEMQRYVEENQTR |
| | | NKHINPNEWWKVYPSSVTEFKFLFVSGHFKGNYKAQLTRLNHITNCNGA |
| | | VLSVEELLIGGEMIKAGTLTLEEVRRKFNNGEINFAAD |
| #243 | HBB TALEN RIGHT | ATGGGCGATCCTAAAAAGAAACGTAAGGTCATCGATATCGCCGATCT |
| | | ACGCACGCTCGGCTACAGCCAGCAGCAACAGGAGAAGATCAAACCGA |
| | | AGGTTCGTTCGACAGTGGCGCAGCACCACGAGGCACTGGTCGGCCAC |
| | | GGGTTTACACACGCGCACATCGTTGCGTTAAGCCAACACCCGGCAGC |
| | | GTTAGGGACCGTCGCTGTCAAGTATCAGGACATGATCGCAGCGTTGC |
| | | CAGAGGCGACACACGAAGCGATCGTTGGCGTCGGCAAACAGTGGTCC |
| | | GGCGCACGCGCTCTGGAGGCCTTGCTCACGGTGGCGGGAGAGTTGA |
| | | GAGGTCCACCGTTACAGTTGGACACAGGCCAACTTCTCAAGATTGCAA |
| | | AACGTGGCGGCGTGACCGCAGTGGAGGCAGTGCATGCATGGCGCAA |
| | | TGCACTGACGGGTGCCCCGCTCAACTTGACCCCCCAGCAGGTGGTGG |
| | | CCATCGCCAGCAATAATGGTGGCAAGCAGGCGCTGGAGACGGTCCAG |
| | | CGGCTGTTGCCGGTGCTGTGCCAGGCCCACGGCTTGACCCCCCAGCA |
| | | GGTGGTGGCCATCGCCAGCAATGGCGGTGGCAAGCAGGCGCTGGAG |
| | | ACGGTCCAGCGGCTGTTGCCGGTGCTGTGCCAGGCCCACGGCTTGAC |
| | | CCCCCAGCAGGTGGTGGCCATCGCCAGCAATGGCGGTGGCAAGCAG |
| | | GCGCTGGAGACGGTCCAGCGGCTGTTGCCGGTGCTGTGCCAGGCCCA |
| | | CGGCTTGACCCCCCAGCAGGTGGTGGCCATCGCCAGCAATGGCGGTG |
| | | GCAAGCAGGCGCTGGAGACGGTCCAGCGGCTGTTGCCGGTGCTGTGC |
| | | CAGGCCCACGGCTTGACCCCCCAGCAGGTGGTGGCCATCGCCAGCAA |
| | | TAATGGTGGCAAGCAGGCGCTGGAGACGGTCCAGCGGCTGTTGCCG |
| | | GTGCTGTGCCAGGCCCACGGCTTGACCCCGGAGCAGGTGGTGGCCAT |
| | | CGCCAGCAATATTGGTGGCAAGCAGGCGCTGGAGACGGTGCAGGCG |
| | | CTGTTGCCGGTGCTGTGCCAGGCCCACGGCTTGACCCCCCAGCAGGT |
| | | GGTGGCCATCGCCAGCAATAATGGTGGCAAGCAGGCGCTGGAGACG |
| | | GTCCAGCGGCTGTTGCCGGTGCTGTGCCAGGCCCACGGCTTGACCCCC |
| | | CAGCAGGTGGTGGCCATCGCCAGCAATAATGGTGGCAAGCAGGCGCT |
| | | GGAGACGGTCCAGCGGCTGTTGCCGGTGCTGTGCCAGGCCCACGGCT |
| | | TGACCCCCCAGCAGGTGGTGGCCATCGCCAGCAATGGCGGTGGCAAG |
| | | CAGGCGCTGGAGACGGTCCAGCGGCTGTTGCCGGTGCTGTGCCAGGC |
| | | CCACGGCTTGACCCCCCAGCAGGTGGTGGCCATCGCCAGCAATGGCG |
| | | GTGGCAAGCAGGCGCTGGAGACGGTCCAGCGGCTGTTGCCGGTGCT |
| | | GTGCCAGGCCCACGGCTTGACCCCCCAGCAGGTGGTGGCCATCGCCA |
| | | GCAATAATGGTGGCAAGCAGGCGCTGGAGACGGTCCAGCGGCTGTT |
| | | GCCGGTGCTGTGCCAGGCCCACGGCTTGACCCCGGAGCAGGTGGTGG |
| | | CCATCGCCAGCCACGATGGCGGCAAGCAGGCGCTGGAGACGGTCCA |
| | | GCGGCTGTTGCCGGTGCTGTGCCAGGCCCACGGCTTGACCCCCCAGC |
| | | AGGTGGTGGCCATCGCCAGCAATGGCGGTGGCAAGCAGGCGCTGGA |
| | | GACGGTCCAGCGGCTGTTGCCGGTGCTGTGCCAGGCCCACGGCTTGA |
| | | CCCCGGAGCAGGTGGTGGCCATCGCCAGCAATATTGGTGGCAAGCAG |
| | | GCGCTGGAGACGGTGCAGGCGCTGTTGCCGGTGCTGTGCCAGGCCCA |
| | | CGGCTTGACCCCCCAGCAGGTGGTGGCCATCGCCAGCAATAATGGTG |
| | | GCAAGCAGGCGCTGGAGACGGTCCAGCGGCTGTTGCCGGTGCTGTGC |
| | | CAGGCCCACGGCTTGACCCCTCAGCAGGTGGTGGCCATCGCCAGCAA |
| | | TGGCGGCGGCAGGCCGGCGCTGGAGAGCATTGTTGCCCAGTTATCTC |
| | | GCCCTGATCCGGCGTTGGCCGCGTTGACCAACGACCACCTCGTCGCCT |
| | | TGGCCTGCCTCGGCGGGCGTCCTGCGCTGGATGCAGTGAAAAAGGGA |
| | | TTGGGGGATCCTATCAGCCGTTCCCAGCTGGTGAAGTCCGAGCTGGA |
| | | GGAGAAGAAATCCGAGTTGAGGCACAAGCTGAAGTACGTGCCCCAC |
| | | GAGTACATCGAGCTGATCGAGATCGCCCGGAACAGCACCCAGGACCG |
| | | TATCCTGGAGATGAAGGTGATGGAGTTCTTCATGAAGGTGTACGGCT |
| | | ACAGGGGCAAGCACCTGGGCGGCTCCAGGAAGCCCGACGGCGCCAT |
| | | CTACACCGTGGGCTCCCCCATCGACTACGGCGTGATCGTGGACACCAA |
| | | GGCCTACTCCGGCGGCTACAACCTGCCCATCGGCCAGGCCGACGAAA |
| | | TGCAGAGGTACGTGGAGGAGAACCAGACCAGGAACAAGCACATCAA |
| | | CCCCAACGAGTGGTGGAAGGTGTACCCCTCCAGCGTGACCGAGTTCA |
| | | AGTTCCTGTTCGTGTCCGGCCACTTCAAGGGCAACTACAAGGCCCAGC |
| | | TGACCAGGCTGAACCACATCACCAACTGCAACGGCGCCGTGCTGTCC |
| | | GTGGAGGAGCTCCTGATCGGCGGCGAGATGATCAAGGCCGGCACCCT |
| | | GACCCTGGAGGAGGTGAGGAGGAAGTTCAACAACGGCGAGATCAAC |
| | | TTCGCGGCCGACTGATAA |
| #244 | HBB TALEN RIGHT | MGDPKKKRKVIDIADLRTLGYSQQQQEKIKPKVRSTVAQHHEALVGHGF |
| | PRT | THAHIVALSQHPAALGTVAVKYQDMIAALPEATHEAIVGVGKQWSGAR |
| | | ALEALLTVAGELRGPPLQLDTGQLLKIAKRGGVTAVEAVHAWRNALTGA |
| | | PLNLTPQQVVAIASNNGGKQALETVQRLLPVLCQAHGLTPQQVVAIASN |
| | | GGGKQALETVQRLLPVLCQAHGLTPQQVVAIASNGGGKQALETVQRLLP |
| | | VLCQAHGLTPQQVVAIASNGGGKQALETVQRLLPVLCQAHGLTPQQVV |
| | | AIASNNGGKQALETVQRLLPVLCQAHGLTPEQVVAIASNIGGKQALETVQ |
| | | ALLPVLCQAHGLTPQQVVAIASNNGGKQALETVQRLLPVLCQAHGLTPQ |
| | | QWAIASNNGGKQALETVQRLLPVLCQAHGLTPQQVVAIASNGGGKQA |
| | | LETVQRLLPVLCQAHGLTPQQVVAIASNGGGKQALETVQRLLPVLCQAH |
| | | GLTPQQVVAIASNNGGKQALETVQRLLPVLCQAHGLTPEQVVAIASHDG |
| | | GKQALETVQRLLPVLCQAHGLTPQQVVAIASNGGGKQALETVQRLLPVL |
| | | COAHGLTPEQVVAIASNIGGKQALETVQALLPVLCQAHGLTPQQVVAIA |
| | | SNNGGKQALETVQRLLPVLCQAHGLTPQQVVAIASNGGGRPALESIVAQ |
| | | LSRPDPALAALTNDHLVALACLGGRPALDAVKKGLGDPISRSQLVKSELEE |
| | | KKSELRHKLKYVPHEYIELIEIARNSTQDRILEMKVMEFFMKVYGYRGKHL |
| | | GGSRKPDGAIYTVGSPIDYGVIVDTKAYSGGYNLPIGQADEMQRYVEEN |
| | | QTRNKHINPNEWWKVYPSSVTEFKFLFVSGHFKGNYKAQLTRLNHITNC |
| | | NGAVLSVEELLIGGEMIKAGTLTLEEVRRKFNNGEINFAAD |
| #245 | CAS9 | MDYKDHDGDYKDHDIDYKDDDDKMAPKKKRKVGIHGVPAADKKYSIGL |
| | | DIGTNSVGWAVITDEYKVPSKKFKVLGNTDRHSIKKNLIGALLFDSGETAE |
| | | ATRLKRTARRRYTRRKNRICYLQEIFSNEMAKVDDSFFHRLEESFLVEEDK |
| | | KHERHPIFGNIVDEVAYHEKYPTIYHLRKKLVDSTDKADLRLIYLALAHMIK |
| | | FRGHFLIEGDLNPDNSDVDKLFIQLVQTYNQLFEENPINASGVDAKAILSA |
| | | RLSKSRRLENLIAQLPGEKKNGLFGNLIALSLGLTPNFKSNFDLAEDAKLQL |
| | | SKDTYDDDLDNLLAQIGDQYADLFLAAKNLSDAILLSDILRVNTEITKAPLS |
| | | ASMIKRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHLGELH |
| | | AILRRQEDFYPFLKDNREKIEKILTFRIPYYVGPLARGNSRFAWMTRKSEET |
| | | ITPWNFEEWDKGASAQSFIERMTNFDKNLPNEKVLPKHSLLYEYFTVYN |
| | | ELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFE |
| | | DREMIEERLKTYAHLFDDKVMKQLKRRRYTGWGRLSRKLINGIRDKQSG |
| | | KTILDFLKSDGFANRNFMQLIHDDSLTFKEDIQKAQVSGQGDSLHEHIAN |
| | | LAGSPAIKKGILQTVKVVDELVKVMGRHKPENIVIEMARENQTTQKGQK |
| | | NSRERMKRIEEGIKELGSQILKEHPVENTQLQNEKLYLYYLQNGRDMYVD |
| | | QELDINRLSDYDVDHIVPQSFLADDSIDNKVLTRSDKNRGKSDNVPSEEV |
| | | VKKMKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYKVR |
| | | EINNYHHAHDAYLNAVVGTALIKKYPALESEFVYGDYKVYDVRKMIAKSE |
| | | QEIGKATAKYFFYSNIMNFFKTEITLANGEIRKAPLIETNGETGEIVWDKGR |
| | | DFATVRKVLSMPQVNIVKKTEVQTGGFSKESILPKRNSDKLIARKKDWDP |
| | | KKYGGFDSPTVAYSVLWAKVEKGKSKKLKSVKELLGITIMERSSFEKNPID |
| | | FLEAKGYKEVKKDLIIKLPKYSLFELENGRKRMLASAGELQKGNELALPSKY |
| | | VNFLYLASHYEKLKGSPEDNEQKQLFVEQHKHYLDEIIEQISEFSKRVILAD |
| | | ANLDKVLSAYNKHRDKPIREQAENIIHLFTLTNLGAPAAFKYFDTTIDRKRY |
| | | TSTKEVLDATLIHQSITGLYETRIDLSQLGGDKRPAATKKAGQAKKKK |
| #246 | TRAC gRNA | C*A*G*GGUUCUGGAUAUCUGUGUUUUAGAGC |
| | | UAGAAAUAGCAAGUUAAAAUAAGGCUAGUCCGUUAUCAACUUGA |
| | | AAA AGUGGCACCGAGUCGGUGCU*U*U*U |
| | | *phosphorothioate bond |

### REFERENCES

Aird, E. J., Lovendahl, K. N., St. Martin, A., Harris, R. S., & Gordon, W. R. (2018). Increasing Cas9-mediated homology-directed repair efficiency through covalent tethering of DNA repair template. Communications Biology, 1(1), 54. https://doi.org/10.1038/s42003-018-0054-2
Brinkman, E. K., Chen, T., Haas, M. de, Holland, H. A., Akhtar, W., & Steensel, B. van. (2018). Kinetics and Fidelity of the Repair of Cas9-Induced Double-Strand DNA Breaks. Molecular Cell, 70(5), 801. https://doi.org/10.1016/J.MOLCEL.2018.04.016
Eyquem, J., Mansilla-Soto, J., Giavridis, T., van der Stegen, S. J. C., Hamieh, M., Cunanan, K. M., . . . Sadelain, M. (2017). Targeting a CAR to the TRAC locus with CRISPR/Cas9 enhances tumour rejection. Nature, 543(7643), 113-117. https://doi.org/10.1038/nature21405
Gaj, T., Gersbach, C. A., Barbas, C. F., & III. (2013). ZFN, TALEN, and CRISPR/Cas-based methods for genome engineering. Trends in Biotechnology, 31(7), 397-405. https://doi.org/10.1016/j.tibtech.2013. 04.004
Hornung, V., & Latz, E. (2010). Intracellular DNA recognition. Nature Reviews Immunology, 10(2), 123-130. https://doi.org/10.1038/nri2690
Hsu, P. D., Lander, E. S., & Zhang, F. (2014). Development and applications of CRISPR-Cas9 for genome engineering. Cell, 157(6), 1262-1278. https://doi.org/10.1016/j.cell.2014.05.010
Juillerat, A., Dubois, G., Valton, J., Thomas, S., Stella, S., Maréchal, A., . . . Duchateau, P. (2014). Comprehensive analysis of the specificity of transcription activator-like effector nucleases. Nucleic Acids Research, 42(8), 5390-5402. https://doi.org/10.1093/nar/gku155
Lechardeur, D., Sohn, K.-J., Haardt, M., Joshi, P. B., Monck, M., Graham, R. W., . . . Lukacs, G. L. (1999). Metabolic instability of plasmid DNA in the cytosol: a potential barrier to gene transfer. Gene Therapy, 6(4), 482-497. https://doi.org/10.1038/sj.gt.3300867
Liang, F., Han, M., Romanienko, P. J., & Jasin, M. (1998). Homology-directed repair is a major double-strand break repair pathway in mammalian cells. PNAS, 95(9), 5172-5177. https://doi.org/10.1073/pnas.95.9.5172
Pâques, F., & Haber, J. E. (1999). Multiple Pathways of Recombination Induced by Double-Strand Breaks in Saccharomyces cerevisiae. Microbiology and Molecular Biology Reviews, 63(2), 349-404. Retrieved from https://mmbr.asm.org/content/63/2/349.abstract?ijkey=aa6b58852a13cd4beb7bde4426a8be9994 8cdf38&keytype2=tf_ipsecsha
Poirot, L., Philip, B., Schiffer-Mannioui, C., Le Clerre, D., Chion-Sotinel, I., Derniame, S., . . . Smith, J. (2015). Multiplex Genome-Edited T-cell Manufacturing Platform for “Off-the-Shelf” Adoptive T-cell Immunotherapies. Cancer Research, 75(18), 3853-3864. https://doi.org/10.1158/0008-5472.CAN-14-3321
Richardson, C., Jasin, M., Dujon, B., & Nicolas, J.-F. (2000). Coupled homologous and nonhomologous repair of a double-strand break preserves genomic integrity in mammalian cells. Molecular and Cellular Biology, 20(23), 9068-9075. https://doi.org/10.1128/mcb.20.23.9068-9075.2000
Roth, T. L., Puig-Saus, C., Yu, R., Shifrut, E., Carnevale, J., Li, P. J., . . . Marson, A. (2018). Reprogramming human T cell function and specificity with non-viral genome targeting. Nature, 559(7714), 405-409. https://doi.org/10.1038/s41586-018-0326-5
Schumann, K., Lin, S., Boyer, E., Simeonov, D. R., Subramaniam, M., Gate, R. E., . . . Marson, A. (2015). Generation of knock-in primary human T cells using Cas9 ribonucleoproteins. PNAS, 112(33), 10437-10442. https://doi.org/10.1073/pnas.1512503112
Valton, J., Guyot, V., Marechal, A., Filhol, J.-M., Juillerat, A., Duclert, A., . . . Poirot, L. (2015). A Multidrug-resistant Engineered CAR T Cell for Allogeneic Combination Immunotherapy. Molecular Therapy, 23(9), 1507-1518. https://doi.org/10.1038/mt.2015.104

1. A method for targeted insertion of an exogenous sequence at a genomic locus in a immune cell, wherein said insertion is induced by a sequence-specific endonuclease that has cleavage activity at said locus, the DNA template comprising the exogenous sequence being introduced into the cell between at least 5 and 20 hours after transfection of the sequence-specific endonuclease and wherein the sequence-specific endonuclease has cleavage activity at said locus for at least 5 hours before the introduction into said cell of a DNA template comprising said exogenous sequence.

2. (canceled)

3. (canceled)

4. The method of claim 1, wherein said sequence-specific endonuclease has cleavage activity for at least 15 hours, preferably for at least 18 hours, more preferably at least 20 hours before said DNA template is introduced into said cell.

5. A method of claim 1, wherein said method comprises at least the steps of:
a) transfecting said cell with a sequence-specific endonuclease polypeptide having cleavage activity at the genomic locus;
b) introducing into said cell, between 5 and 20 hours after said transfecting step of a), a DNA template comprising the exogenous sequence to be inserted at said locus by homologous recombination, NHEJ, HDR, MMEJ or HMEJ; and
c) culturing and selecting the cells, in which said exogenous sequence has been inserted at said locus.

6. A method of claim 1, where in said method comprises at least the steps of:
a) transfecting said cell with a sequence-specific endonuclease polynucleotide having cleavage activity at the genomic locus;
b) introducing into said cell, between 10 and 20 hours after said transfecting step of a), a DNA template comprising the exogenous sequence to be inserted at said locus by homologous recombination, NHEJ, HDR, MMEJ or HMEJ, and
c) culturing and selecting the cells, in which said exogenous sequence has been inserted at said locus.

7. The method according to claim 6, wherein said sequence-specific endonuclease polynucleotide is transfected as a mRNA.

8. (canceled)

9. The method of claim 1, wherein said endonuclease is a TALE-nuclease.

10. The method of claim 1, wherein said endonuclease is a RNA-guided endonuclease, such as Cas9 or Cpf1.

11. (canceled)

12. The method of claim 1, wherein said exogenous sequence is inserted at said locus by homologous recombination.

13. (canceled)

14. (canceled)

15. (canceled)

16. The method of claim 1, wherein said DNA template is a single stranded polynucleotide.

17. The method of claim 1, wherein said DNA template is a short single-stranded oligodeoxynucleotide (ssODN).

18. The method according to claim 17, wherein said ssODN has homology arms comprised between 50 and 200 bp, preferably between 80 and 150 bp, more preferably between 90 and 120 bp.

19. The method of claim 1, wherein said method comprises at least two transfection steps, wherein a first transfection step introduces said sequence-specific endonuclease into said cell, and a second transfection step introduces said DNA template comprising said exogenous sequence to be inserted, said second transfection step being by electroporation.

20. (canceled)

21. (canceled)

22. (canceled)

23. The method of claim 1, wherein said cell is a primary cell.

24. The method of claim 1, wherein said immune cell is a HSC or a progeny thereof, such as a T-cell or a NK cell.

25. The method of claim 1, wherein said cell is a primary T-cell, more preferably a primary T-cell from a patient, such as a tumor infiltrating lymphocyte (TIL), or a primary T-cell from a donor.

26-34. (canceled)

35. A method for producing therapeutic cells, comprising the steps of:
providing primary immune cells from a donor or a patient or derived from human iPS or hES cells;
performing a targeted insertion according to the method of claim 1;
purifying and freezing the cells for subsequent use as a therapeutic composition.

36. The method of claim 1, wherein said method does not comprise a step involving a viral vector.
